(19) United States
(12) Patent Application Publication
(10) Pub. No.: US 2021/0388443 A1
(43) Pub. Date: Dec. 16, 2021

(54) SEPSIS BIOMARKER PANELS AND METHODS OF USE

(71) Applicants: Institute for Systems Biology, Seattle, WA (US); The Secretary of State for Defence, Salisbury (GB)

(72) Inventors: Kai Wang, Seattle, WA (US); Taek-Kyun Kim, Seattle, WA (US); Minyoung Lee, Seattle, WA (US); Kathie Walters, Seattle, WA (US); Roman Anton Lukaszewski, Salisbury (GB)

(73) Assignees: Institute for Systems Biology, Seattle, WA (US); The Secretary of State for Defence, Salisbury (GB)

(21) Appl. No.: 17/290,931

(22) PCT Filed: Nov. 4, 2019
(86) PCT No.: PCT/US2019/059707

§ 371 Date: May 3, 2021

Related U.S. Application Data
(60) Provisional application No. 62/911,603, filed on Oct. 7, 2019.
(60) Provisional application No. 62/755,834, filed on Nov. 5, 2018.

(51) Int. Cl. C12Q 1/6883

(52) U.S. Cl. CPC C12Q 1/6883; C12Q 2600/158

(57) ABSTRACT

Methods for diagnosing and/or predicting presence of sepsis in a subject using a gene signature of three or more genes are provided. Also provided are sets containing specific binding molecules for each of the three or more genes, and kits containing such sets.

### CROSS REFERENCE TO RELATED APPLICATIONS

This application claims the benefit of U.S. Provisional Application No. 62/755,834, filed Nov. 5, 2018, and U.S. Provisional Application No. 62/911,603, filed Oct. 7, 2019, both of which are incorporated by reference herein in their entirety.

### ACKNOWLEDGMENT OF GOVERNMENT SUPPORT

This invention was made with government support under Contract number HDTRA1-13-C-0055 awarded by Defense Threat Reduction Agency. The government has certain rights in the invention.

### FIELD

This disclosure relates to biomarkers for prediction and/or diagnosis of sepsis and methods of their use, including methods for treating sepsis in a subject.

### BACKGROUND

Sepsis occurs when the body's response to infection damages its own tissues and organs, leading to shock, multi-organ failure, and potentially death. In the United States, the incidence of severe sepsis is about 300 cases per 100,000 population with a mortality rate estimated between 28 and 50 percent (Ullah et al., Pakistan J. Med. Sci. 32:688-693, 2016). Diagnosis of sepsis is difficult during early stages (e.g., pre-symptomatic phase), which makes it challenging to intervene therapeutically until after the onset of symptoms. Since progression of the condition, once it takes hold, is rapid and often aggressive, effective and early intervention is critical to control the development of sepsis. Among clinical parameters, C-reactive protein (CRP) and procalcitonin (PCT) are established markers to reflect infection or inflammation (Faix, Crit. Rev. Clin. Lab. Sci. 50:23-36, 2013). However, due to the lack of specificity, the use of these markers for sepsis diagnosis is limited. For example, both CRP and PCT levels are elevated in sepsis patients as well as in stress, severe trauma, and surgery patients (Matson et al., Anaesth. Intensive Care 19:182-186, 1991; Yentis et al., Intensive Care Med. 21:602-605, 1995; Aabenhus and Jensen, Prim. Care Respir. J. 20:360-367, 2011; Schuetz et al., BMC Med. 9:107, 2011).

The current gold standard for sepsis diagnosis is positive identification of pathogenic bacteria through culture. Unfortunately, such culture-based methods typically take at least 2-5 days to complete, depending on the causative pathogen and often provide negative or inconclusive results (Kim and Weinstein, Clin. Microbial. Infect. 19:513-520, 2013; Ruiz-Giardin et al., Int. J Infect. Dis. 41:6-10, 2015). Patients are often treated with antibiotics prior to definitive identification of the infective agent, which can result in inappropriate or inadequate treatment. The delay of proper intervention likely contributes to the high sepsis mortality rate.

### SUMMARY

There is a need to accurately identify patients who have sepsis, are at risk of developing sepsis, or who are in the early or pre-symptomatic phase of sepsis, so that appropriate treatment can be initiated as early as possible.

In some embodiments, disclosed herein are methods of detecting a plurality of markers, such as detecting or measuring expression of a set of at least six genes in a sample from a subject having sepsis, suspected to have sepsis, or at risk of sepsis. The methods include detecting or measuring expression of a set of at least six genes, wherein the set includes CCR1, HLA-DPB1, BATF, C3AR1, ARHGEFI8, and C9orf95; CCR1, HLA-DPB1, BATF, C3AR1, MTCH1, and C9orf103; CCR1, HLA-DPB1, BATF, C3AR1, MTCH1, and C9orf95; CCR1, HLA-DPB1, BATF, GNA15, MTCH1, and C9orf95; CD177, HLA-DPB1, BATF, C3AR1, MTCH1, and C9orf95; CD63, HLA-DPB1, PLAC8, FCER1G, MTCH1, and C9orf95; CD63, HLA-DPB1, PLAC8, GNA15, ARHGEFI8, and C9orf95; CD63, HLA-DPB1, PLAC8, GNA15, MTCH1, and C9orf95; CD63, HLA-DPB1, BATF, C3AR1, MTCH1, and C9orf95; CD63, HLA-DPB1, BATF, GNA15, MTCH1, and C9orf95; EMR1, HLA-DPB1, BATF, C3AR1, MTCH1, and C9orf95; EMR1, HLA-DPB1, BATF, GNA15, MTCH1, and C9orf95; FCER1G, HLA-DPB1, PLAC8, C3AR1, MTCH1, and C9orf95; FCER1G, HLA-DPB1, PLAC8, GNA15, ARHGEF18, and C9orf95; FCER1G, HLA-DPB1, PLAC8, GNA15, MTCH1, and C9orf95; FCER1G, HLA-DPB1, BATF, C3AR1, MTCH1, and SEPHS2; FCER1G, HLA-DPB1, BAIT, C3AR1, MTCH1, and C9orf95; FES, HLA-DPB1, PLAC8, FCER1G, MTCH1, and C9orf95; FES, HLA-DPB1, PLAC8, GNA15, ARHGEF18, and C9orf103; FES, HLA-DPB1, PLAC8, GNA15, MTCH1, and C9orf95; FES, HLA-DPB1, BATF, C3AR1, MTCH1, and C9orf95; or C3AR1, HLA-DPB1, BATF, GNA15, MTCH1, and C9orf95. In some examples, the expression of CCR1, C3AR1, C9orf95, BATF, C9orf103, GNA15, CD177, CD63, EMR1, FCER1G, FES, PLAC8, and/or SEPHS2 is increased compared to a control, and/or expression of HLA-DPB1, ARHGEF18, and/or MTCH1 is decreased compared to a control.

Also provided are methods of diagnosing sepsis in a subject including detecting or measuring expression of the set of at least six genes in a sample from a subject, wherein the subject is diagnosed with sepsis when expression of CCR1, C3AR1, C9orf95, BATF, C9orf103, GNA15, CD177, CD63, EMR1, FCER1G, FES, PLAC8, and/or SEPHS2 is increased compared to a control, and/or expression of HLA-DPB1, ARHGEF18, and/or MTCH1 is decreased compared to a control. Further provided are methods of treating a subject with sepsis including detecting or measuring expression of the set of at least six genes in a sample from a subject, wherein expression of CCR1, C3AR1, C9orf95, BATF, C9orf103, GNA15, CD177, CD63, EMR1, FCER1G, FES, PLAC8, and/or SEPHS2 is increased compared to a control, and/or expression of HLA-DPB1, ARHGEF18, and/or MTCH1 is decreased compared to a control and administering one or more antibiotics to the subject.

In other embodiments, disclosed herein are methods of detecting a plurality of markers, such as detecting or measuring expression of a set of at least three genes in a sample from a subject having sepsis or suspected to have sepsis. In some examples, the methods include detecting or measuring expression of a set of at least three (such as at least 3, 4, 5, or more) genes, wherein the set includes a set of 3, 4, or 5 genes provided in Table 16, for example, LCN2, SLC2A3, and BMX; LCN2, SLC2A3, and GRB10; LCN2, PFKFB3, and GRB10; LCN2, PFKFB3, and BMX; IL1R2, HK3, and BMX; LCN2, HK3, and BMX; LCN2, HK3, and GRB10; GZMA, HK3, and BMX; FCAR, PFKFB2, and BMX; LCN2, PFKFB3, and IL18R1; LCN2, PFKFB3, GRB10, and ST6GALNAC3; LCN2, SLC2A3, BMX, and LGALS2; IL1R2, SLC2A3, BMX, and TCN1; LCN2, SLC2A3, GRB10, and ST6GALNAC3; FCAR, PFKFB2, BMX, and CEACAM1; IL1R2, HK3, BMX, and CD24; IL1R2, PFKFB3, BMX, and CD24; BMX, SLC2A3, GRB10, and CD24; IL1R2, HK3, BMX, and CEACAM1; GZMA, SLC2A3, BMX, and CD24; LCN2, PFKFB3, GRB10, ST6GALNAC3, and RNASE3; LCN2, PFKFB3, GRB10, RNASE2, and ST6GALNAC3; IL1R2, PFKFB3, GRB10, CD24, and ST6GALNAC3; GZMA, PFKFB3, GRB10, ST6GALNAC3, and CD24; HK3, PFKFB3, GRB10, ST6GALNAC3, and CD24; IL1R2, PFKFB3, GRB10, ST6GALNAC3, and TCN1; SLC2A3, PFKFB3, GRB10, ST6GALNAC3, and TCN1, LCN2, PFKFB3, GRB10, ST6GALNAC3, and DACH1; SLC2A3, PFKFB3, GRB10, ST6GALNAC3, and CD24; or SLC2A3, HK3, BMX, SPOCD1, and LGALS2, wherein expression of the three or more genes is altered compared to a control.

In other examples, the methods include detecting or measuring expression of a set of at least six genes (such as 6, 7, 8, or more), wherein the set includes a set of 6, 7, or 8 genes provided in Table 17, for example, RPS6KA3, BCL6, TPM3, GNG10, STOM, and MPO; RPS6KA3, MAVS, TPM3, BCL6, STOM, and MPO; RPS6KA3, BCL6, TPM3, BMX, STOM, and MPO; RPS6KA3, LTF, TPM3, BCL6, STOM, and PDGFC; RPS6KA3, BCL6, TPM3, CD55, STOM, and MPO; RPS6KA3, BCL6, TPM3, GNG10, STOM, and PDGFC; RPS6KA3, LTF, TPM3, BCL6, STOM, and CYP1B1; LTF, BCL6, TPM3, CD55, STOM, and PDGFC; RPS6KA3, BCL6, TPM3, TLR8, STOM, and MPO; RPS6KA3, BCL6, TPM3, HLA-DRA, GNG10, and MPO; RPS6KA3, MLLT1, TPM3, BCL6, GNG10, CYP27A1, and YOD1; RPS6KA3, MLLT1, TPM3, BCL6, GNG10, PDGFC, and YOD1; RPS6KA3, GAS7, TPM3, BCL6, RRBP1, MPO, and YOD1; RPS6KA3, MLLT1, TPM3, BCL6, GNG10, MPO, and LGALS2; RPS6KA3, GAS7, TPM3, BCL6, GNG10, MPO, and YOD1; LTF, MLLT1, TPM3, BCL6, GNG10, PDGFC, and YOD1; RPS6KA3, BCL6, TPM3, GNG10, LILRA2, CYP27A1, and YOD1; RPS6KA3, BCL6, TPM3, GNG10, STOM, MPO, and LGALS2; RPS6KA3, MLLT1, TPM3, BCL6, GNG10, MPO, and YOD1; RPS6KA3, BCL6, TPM3, CD55, STOM, MPO, and IL17RA; RPS6KA3, MLLT1, TPM3, BCL6, GNG10, PDGFC, YOD1, and LGALS2; RPS6KA3, MLLT1, TPM3, BCL6, GNG10, PDGFC, YOD1, and LILRA4; RPS6KA3, MLLT1, TPM3, BCL6, GNG10, PDGFC, YOD1, and IL17RA; RPS6KA3, MLLT1, TPM3, BCL6, GNG10, PDGFC, YOD1, and TCN1; RPS6KA3, MLLT1, TPM3, BCL6, GNG10, PDGFC, YOD1, and RNASE3; RPS6KA3, MLLT1, TPM3, BCL6, GNG10, PDGFC, YOD1, and NOV; RPS6KA3, MLLT1, TPM3, BCL6, GNG10, PDGFC, YOD1, and RNASE2; LTF, MLLT1, TPM3, BCL6, GNG10, PDGFC, YOD1, and FAM105A; RPS6KA3, BCL6, TPM3, HLA-DRA, RRBP1, MPO, ERO1L, and YOD1; or LTF, MLLT1, TPM3, BCL6, GNG10, PDGFC, YOD1, and C14orf101, wherein expression of the six or more genes is altered compared to a control. An additional set of six or more genes that can be used in the disclosed methods includes LCN2, BMX, MPO, RPS6KA3, BCL6, and MAPK14, wherein expression of the six or more genes is altered compared to a control.

Also provided are methods of diagnosing sepsis in a subject, including detecting or measuring expression of the set of at least three or at least six genes (such as the sets of genes in Tables 15 and 16) or the set of at least six genes including LCN2, BMX, MPO, RPS6KA3, BCL6, and MAPK14 in a sample from a subject, wherein the subject is diagnosed with sepsis when expression of the set of at least three genes or at least six genes is altered compared to a control. Further provided are methods of treating a subject with sepsis including detecting or measuring expression of the set of at least three genes or at least six genes (such as the sets of genes in Tables 15 and 16) or the set of at least six genes including LCN2, BMX, MPO, RPS6KA3, BCL6, and MAPK14 in a sample from a subject, wherein expression is altered compared to a control, and administering one or more antibiotics to the subject. In some examples, the subject does not exhibit symptoms of sepsis (for example, is pre-symptomatic or pre-clinical for sepsis). Thus, in some examples, the methods including predicting risk of developing sepsis in a subject.

Also disclosed herein are solid supports including a solid support (such as an array or bead) including binding agents specific for a set of markers disclosed herein. In some embodiments, the solid support includes at least one probe, primer, and/or antibody specific for a set including each of CCR1, HLA-DPB1, BATF, C3AR1, ARHGEF18, and C9orf95; CCR1, HLA-DPB1, BATF, C3AR1, MTCH1, and C9orf103; CCR1, HLA-DPB1, BATF, C3AR1, MTCH1, and C9orf95; CCR1, HLA-DPB1, BATF, GNA15, MTCH1, and C9orf95; CD177, HLA-DPB1, BATF, C3AR1, MTCH1, and C9orf95; CD63, HLA-DPB1, PLAC8, FCER1G, MTCH1, and C9orf95; CD63, HLA-DPB1, PLAC8, GNA15, ARHGEF18, and C9orf95; CD63, HLA-DPB1, PLAC8, GNA15, MTCH1, and C9orf95; CD63, HLA-DPB1, BATF, C3AR1, MTCH1, and C9orf95; CD63, HLA-DPB1, BATF, GNA15, MTCH1, and C9orf95; EMR1, HLA-DPB1, BATF, C3AR1, MTCH1, and C9orf95; EMR1, HLA-DPB1, BATF, GNA15, MTCH1, and C9orf95; FCER1G, HLA-DPB1, PLAC8, C3AR1, MTCH1, and C9orf95; FCER1G, HLA-DPB1, PLAC8, GNA15, ARHGEF18, and C9orf95; FCER1G, HLA-DPB1, PLAC8, GNA15, MTCH1, and C9orf95; FCER1G, HLA-DPB1, BATF, C3AR1, MTCH1, and SEPHS2; FCER1G, HLA-DPB1, BATF, C3AR1, MTCH1, and C9orf95; FES, HLA-DPB1, PLAC8, FCER1G, MTCH1, and C9orf95; FES, HLA-DPB1, PLAC8, GNA15, ARHGEF18, and C9orf103; FES, HLA-DPB1, PLAC8, GNA15, MTCH1, and C9orf95; FES, HLA-DPB1, BATF, C3AR1, MTCH1, and C9orf95; or C3AR1, HLA-DPB1, BATF, GNA15, MTCH1, and C9orf95. In other embodiments, the solid support includes at least one probe, primer, and/or antibody specific for each gene in a set including 3, 4, or 5 genes provided in Table 16, for example, LCN2, SLC2A3, and BMX; LCN2, SLC2A3, and GRB10; LCN2, PFKFB3, and GRB10; LCN2, PFKFB3, and BMX; IL1R2, HK3, and BMX; LCN2, HK3, and BMX; LCN2, HK3, and GRB10; GZMA, HK3, and BMX; FCAR, PFKFB2, and BMX; LCN2, PFKFB3, and IL18R1; LCN2, PFKFB3, GRB10, and ST6GALNAC3; LCN2, SLC2A3, BMX, and LGALS2; IL1R2, SLC2A3, BMX, and TCN1; LCN2, SLC2A3, GRB10, and ST6GALNAC3; FCAR, PFKFB2, BMX, and CEACAM1; IL1R2, HK3, BMX, and CD24; IL1R2, PFKFB3, BMX, and CD24; BMX, SLC2A3, GRB10, and CD24; IL1R2, HK3, BMX, and CEACAM1; GZMA, SLC2A3, BMX, and CD24; LCN2, PFKFB3, GRB10, ST6GALNAC3, and RNASE3; LCN2, PFKFB3, GRB10, RNASE2, and ST6GALNAC3; IL1R2, PFKFB3, GRB10, CD24, and ST6GALNAC3; GZMA, PFKFB3, GRB10, ST6GALNAC3, and CD24; HK3, PFKFB3, GRB10, ST6GALNAC3, and CD24; IL1R2, PFKFB3, GRB10, ST6GALNAC3, and TCN1; SLC2A3, PFKFB3, GRB10, ST6GALNAC3, and TCN1; LCN2, PFKFB3, GRB10, ST6GALNAC3, and DACH1; SLC2A3, PFKFB3, GRB10, ST6GALNAC3, and CD24; or SLC2A3, HK3, BMX, SPOCD1, and LGALS2. In other embodiments, the solid support includes at least one probe, primer, and/or antibody specific for a set including each gene in a set including 6, 7, or 8 genes provided in Table 17, for example, RPS6KA3, BCL6, TPM3, GNG10, STOM, and MPO; RPS6KA3, MAVS, TPM3, BCL6, STOM, and MPO; RPS6KA3, BCL6, TPM3, BMX, STOM, and MPO; RPS6KA3, LIT, TPM3, BCL6, STOM, and PDGFC; RPS6KA3, BCL6, TPM3, CD55, STOM, and MPO; RPS6KA3, BCL6, TPM3, GNG10, STOM, and PDGFC; RPS6KA3, LTF, TPM3, BCL6, STOM, and CYP1B1; LTF, BCL6, TPM3, CD55, STOM, and PDGFC; RPS6KA3, BCL6, TPM3, TLR8, STOM, and MPO; RPS6KA3, BCL6, TPM3, HLA-DRA, GNG10, and MPO; RPS6KA3, MLLT1, TPM3, BCL6, GNG10, CYP27A1, and YOD1; RPS6KA3, MLLT1, TPM3, BCL6, GNG10, PDGFC, and YOD1; RPS6KA3, GAS7, TPM3, BCL6, RRBP1, MPO, and YOD1; RPS6KA3, MLLT1, TPM3, BCL6, GNG10, MPO, and LGALS2; RPS6KA3, GAS7, TPM3, BCL6, GNG10, MPO, and YOD1; LTF, MLLT1, TPM3, BCL6, GNG10, PDGFC, and YOD1; RPS6KA3, BCL6, TPM3, GNG10, LILRA2, CYP27A1, and YOD1; RPS6KA3, BCL6, TPM3, GNG10, STOM, MPO, and LGALS2; RPS6KA3, MLLT1, TPM3, BCL6, GNG10, MPO, and YOD1; RPS6KA3, BCL6, TPM3, CD55, STOM, MPO, and IL17RA; RPS6KA3, MLLT1, TPM3, BCL6, GNG10, PDGFC, YOD1, and LGALS2; RPS6KA3, MLLT1, TPM3, BCL6, GNG10, PDGFC, YOD1, and LILRA4; RPS6KA3, MLLT1, TPM3, BCL6, GNG10, PDGFC, YOD1, and IL17RA; RPS6KA3, MLLT1, TPM3, BCL6, GNG10, PDGFC, YOD1, and TCN1; RPS6KA3, MLLT1, TPM3, BCL6, GNG10, PDGFC, YOD1, and RNASE3; RPS6KA3, MLLT1, TPM3, BCL6, GNG10, PDGFC, YOD1, and NOV; RPS6KA3, MLLT1, TPM3, BCL6, GNG10, PDGFC, YOD1, and RNASE2; LTF, MLLT1, TPM3, BCL6, GNG10, PDGFC, YOD1, and FAM105A; RPS6KA3, BCL6, TPM3, HLA-DRA, RRBP1, MPO, ERO1L, and YOD1; or LTF, MLLT1, TPM3, BCL6, GNG10, PDGFC, YOD1, and C14orf101. In other embodiments, the solid support includes at least one probe, primer, and/or antibody specific for a set of genes including each LCN2, BMX, MPO, RPS6KA3, BCL6, and MAPK14

Kits that include a disclosed set of specific binding agents (such as a set of primers or probes) are also provided. Such a kit can further include other reagents, such as a buffer, such as a hybridization buffer. Such sets of specific binding agents and kits can be used to perform steps of the disclosed methods.

The foregoing and other features of the disclosure will become more apparent from the following detailed description, which proceeds with reference to the accompanying figures.

### BRIEF DESCRIPTION OF THE DRAWINGS

FIGS. 1A and 1B are schematic diagrams showing identification of substitutes for the Stanford11 panel. FIG. 1A is a schematic of the overall procedure for identification of substitutions for the Stanford11 panel. FIG. 1B is a schematic diagram showing the six key biological processes represented by the Stanford11 panel.

FIGS. 2A-2C are diagrams showing the role of biological processes in classification performance. FIG. 2A is a plot showing the distribution of classification performance of 100,000 random gene sets sorted based on performance. FIG. 2B is a plot showing the number of genes of the top and bottom 250 gene sets in 97 gene ontology biological processes (GOBPs) represented by the Stanford11 panel. FIG. 2C provides clusters of GOBPs in the top 250 and bottom 250 gene sets. Count and percent indicate the average number and percentage of genes in each GOBP cluster.

FIGS. 3A-3F are a series of plots showing the performance of panels with one gene substitution. The plots show the distribution of area under the curve (AUC) in the 12 microarray datasets when BATF (FIG. 3A), C3AR1 (FIG. 3B), C9orf95 (FIG. 3C), CEACAM1 (FIG. 3D), GNA15 (FIG. 3E), or MTCH1 (FIG. 3F) was replaced with a substitute gene. *indicates P-value less than 0.05 from DeLong test comparing a substituted panel (box plots) with the median AUC and the original Stanford11 (dots).

FIG. 4 is a plot showing the AUCs in the 12 microarray datasets when genes representing all five functional categories were replaced with substitute genes. * and ** indicate P-value less than 0.05 and 0.01, respectively, from DeLong test comparing a substituted panel with the median AUC and the original Stanford11 (filled dots).

FIGS. 5A-5C are a series of plots showing AUCs in the 12 microarray datasets when only one gene in PLC, phosphorylation, platelet activation function (FIG. 5A), chemotaxis, angiogenesis, adhesion, migration function (FIG. 5B), or in both processes (FIG. 5C) was retained. * and ** indicate P-value less than 0.05 and 0.01, respectively, from DeLong test comparing a substituted panel with the median AUC and the original Stanford11 (filled dots).

FIG. 6 is a plot showing performance of 1,482 six-gene combinations. * and ** indicate P-value less than 0.05 and 0.01, respectively, from DeLong test comparing a substituted panel with the median AUC and the original Stanford11 (filled dots).

FIGS. 7A-7F are a series of panels showing the impact of biological function information. Biological function information was evaluated by three different approaches. FIG. 7A shows the 11 highest correlated genes with Stanford11 and the Pearson's correlation coefficient (panel-HC). 0 in Stanford82 column indicates the gene of Stanford82. FIG. 7B is a plot showing the AUCs of panel-HC (front bars). Back bars show the AUCs of Stanford11. FIG. 7C shows the 14 genes involved in adhesion/migration processes (panel-AM). FIG. 7D is a plot showing the AUCs of panel-AM (front bars). Back bars show the AUCs of Stanford11. FIG. 7E shows the top-scoring pairs identified by k-TSP algorithm (panel-kTSP). FIG. 7F is a plot showing the AUCs of panel-kTSP. * and ** indicate P-value less than 0.05 and 0.01, respectively, from DeLong test.

FIG. 8 is a heat map showing overall expression profile of genes in ISB58 panel. Each column represents samples from patients and each row represent different gene in the set. The sepsis (left) and control (right) patients are indicated on top with the time prior to sepsis diagnosis (Day-3 to Day-1). The red color represents higher expression level than mean while the green represents lower expression level.

FIG. 9 is a heat map showing overall expression profile of genes in ISB355 panel. Each column represents samples from patients and each row represent different gene in the set. The sepsis (left) and control (right) patients are indicated on top with the time prior to sepsis diagnosis (Day-3 to Day-1). The red color represents higher expression level than mean while the green represents lower expression level.

FIG. 10 is a Venn diagram showing overlap of genes between ISB58 and ISB355.

FIGS. 11A and 11B are heat maps showing overall expression profile of genes in predictive panels ISB19 (FIG. 11A) (derived from ISB58 gene set) and ISB63 (FIG. 11B) (derived from ISB355 gene set).

FIG. 12 is a graph summarizing classification performance (Area Under the Curve—AUC) for ISB19 and ISB63 panels. The left bar in each bar represents the performance for the ISB19 gene panel and the right bar in each pair are the results of ISB63. The dataset ID and pathology group are indicated below the X-axis.

FIG. 13 is a graph showing performance comparison between ISB panels with known sepsis diagnosis panels—Stanford11 and Septicyte4. The Y-axis represents the AUC and the X-axis represents the date in relation to sepsis diagnosis (Day 0).

FIG. 14 is a graph showing average AUCs of ISB19 and ISB19-derived 3-, 4-, and 5-gene panels. The Y axis represents the average performance (AUC) of top 3-gene, 4-gene and 5-gene panels, and the X axis indicates the time before sepsis was diagnosed.

FIG. 15 is a graph showing average AUCs of ISB63 and ISB63-derived 6-, 7-, and 8-gene panels. The Y axis represents the average performance (AUC) of top 6-gene, 7-gene and 8-gene panels, and the X axis indicates the time before sepsis was diagnosed.

FIG. 16 is a graph showing average diagnostic performance of sepsis predictive panels in 19 public domain datasets. The AUC is indicated on the bars and the biomarker panels are listed on the X-axis.

FIG. 17 is a graph showing averaged diagnostic performance of 30 smaller panels (top performing 3 genes, 4 genes and 5 genes) derived from ISB19 and (top performing 6 genes, 7 genes and 8 genes) ISB63 (dark color); and with integrated clinical information (bright color) between mild and severe sepsis patients.

FIG. 18 is a summary of analysis approaches, DEGs identified, and performance of the classification panels.

FIGS. 19A-19I show selection of core biological processes that have higher discriminatory power. The procedure for selecting core biological processes is shown in FIG. 19A. Distribution of classification performances of 100,000 random 19 (FIG. 19B) or 63 gene sets (FIG. 19C). Selection of core biological functions associated with ISB19 (FIG. 19D) or ISB63 panels (FIG. 19E). Color scale indicates the number of genes involved in each GOBP among the top 500 and bottom 500 panels (FIGS. 19F-19G). Three and six representative functional terms determined by EnrichmentMap (FIGS. 19H-19I).

FIGS. 20A-20G show study design and patient information. Number of sepsis patients and matched controls (Y-axis) from each recruitment site (X-axis; Heartlands: Heartlands Hospital, LTHT: The Leeds Teaching Hospitals Trust, NBNT: North Bristol NHS Trust, Frankfurt: University Hospital Frankfurt, RLU: The Royal Liverpool University Hospital, UCH: University College Hospital, QEH: Queen Elizabeth Hospital, GST: Guys' and St Thomas' Hospital) (FIG. 20A). Number of patients diagnosed with sepsis each day after surgery (FIG. 20B). Organization of samples based on the date sepsis was diagnosed (FIG. 20C). The timeline based on surgery date is indicated on top blue line and the number indicates day after surgery. The sepsis patients and matched controls are indicated in yellow and gray, respectively. The date sepsis was diagnosed is indicated in red as Day0. The distribution of SOFA score (FIG. 20D), CRP concentration (FIG. 20E), and lactate level (FIG. 20F) (Y-axis) across different time points among different sample groups (top) before sepsis was diagnosed (X-axis). The yellow lines represent the mean values for sepsis patients and the black lines represent the mean values for matched controls. The gray areas are the standard distribution. The yellow (Sepsis) and black (Control) dots are levels for individual samples. Overview of the study design to discover, optimize, and validate pre-symptomatic biomarker for sepsis (FIG. 20G).

FIGS. 21A-21C show differentially expressed genes and performance of biomarker panels. Number of DEGs identified in Approach 2 (yellow) and Approach 6 (blue) (FIG. 21A). Number of genes in the biomarker panels (FIG. 21B). The overall expression level changes of genes included in the classification panels: ISB19 and ISB63 (FIG. 21C).

FIGS. 22A-22C show performance of optimized top performing 3-gene and 6-gene panels. Performance comparison of the optimized panels with the original panels; ISB19 and ISB63, and published panels; SMS, SeptiCyte Lab at different time points (indicated on the bottom of the panel) based on Validation sample set (FIG. 22A), or with integration of SOFA or CRP (FIG. 22B), or in patients who developed different severity of sepsis (FIG. 22C). The Y-axis is the AUC and different biomarker panel is indicated on the X-axis.

FIGS. 23A-23E show assessing the performance of 3- and 6-gene panels in diverse immune-related public datasets. Performance of the panels at Day 0 (FIG. 23A). List of public microarray data from sepsis related studies (adult sepsis, pediatric sepsis and neonatal sepsis (FIG. 23B). Classification performances of 3- and 6-gene panels in individual datasets in FIG. 23B (FIG. 23C). List of public microarray data from bacterial/viral infection and auto immune disease studies (FIG. 23D). Classification performances of 3- and 6-gene panels in individual datasets in FIG. 23D (FIG. 23E).

FIGS. 24A-241 show qPCR validation. List of 3-gene and 6-gene panels representing each biological function (FIG. 24A). Correlation between microarray and qPCR for 3-gene panel (FIG. 24B). Log 2 fold change in sepsis when compared to control samples for 3-gene panel in three time points, Day-3, Day-2 and Day-1 (FIG. 24C). Correlation between microarray and qPCR for 6-gene panel and MAPK14 which is a replaceable gene with TPM3. Red arrows indicate the replaceable genes with the original genes in ISB6 (FIG. 24D). Log 2 fold change in sepsis when compared to control samples for 6-gene panel in three time points, Day-3, Day-2 and Day-1 (FIG. 24E). AUC of 3-gene and 6-gene panels in the Validation set using qPCR of all sepsis patients (FIGS. 24F-24G). AUC of 3-gene and 6-gene panels in Septic shock and Sepsis patients (FIGS. 24H-241).

### DETAILED DESCRIPTION

It is a challenge to develop large multi-feature diagnostic panels for clinical use. Most of the clinical gene panels in current use are relatively small. For example, recently developed influenza and tuberculosis panels each utilize six analytes. Developing large multi-feature panels is expensive and time consuming, including the need to optimize measurements for individual features. In addition, some of the features may show different results due to measurement platform differences. The inventors therefore in some embodiments have reduced the number of features in sepsis diagnosis and/or prognosis panels without sacrificing performance in order to further develop panels for clinical use.

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank Accession numbers (for the sequence present on Nov. 5, 2018). In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Altered expression: A difference, such as an increase or decrease, in the conversion of the information encoded in a gene (for example, one or more of those in Tables 5, 6, 11, 15, and/or 16) into messenger RNA, the conversion of mRNA to a protein, or both. In some examples, the difference is relative to a control or reference value (or range of values), such as the average expression value of a group of healthy subjects, such as subjects who do not have sepsis. Detecting altered expression can include measuring a change in gene or protein expression, such as an increase of at least 20%, at least 50%, at least 75%, at least 90%, at least 100%, at least 200%, at least 300% at least 400%, or at least 500%, or a decrease of at least 20%, at least 50%, at least 75%, at least 90%, or at least 95%.

Array: An arrangement of nucleic acids (such as DNA or RNA) or proteins (such as antibodies) in assigned locations on a matrix or substrate. In some examples, the nucleic acid molecules or proteins are attached covalently to the matrix or substrate.

Control: A sample or standard used for comparison with an experimental sample, such as a sample from a healthy subject, for example, a subject who does not have sepsis. In some embodiments, the control is a historical control or standard reference value or range of values (e.g., a previously tested control sample, such as a group of healthy subjects, or group of samples that represent baseline or normal values, such as the level of expression of one or more genes listed in Tables 5, 6, 11, 15, and/or 16). Laboratory standards and values can be set based on a known or determined population value and can be supplied in the format of a graph or table that permits comparison of measured, experimentally determined values.

Detecting or measuring expression: Determining the level expression in either a qualitative, semi-quantitative, or quantitative manner by detection of nucleic acid molecules (e.g., DNA, RNA, and/or mRNA) or proteins. Exemplary methods include microarray analysis, PCR (such as RT-PCR, real-time PCR, or qRT-PCR), Northern blot, Western blot, ELISA, and mass spectrometry.

Sample (or biological sample): A biological specimen containing nucleic acids (for example, DNA, RNA, and/or mRNA), proteins, or combinations thereof, obtained from a subject. Examples include, but are not limited to, peripheral blood, serum, plasma, urine, saliva, tissue biopsy, fine needle aspirate, surgical specimen, and autopsy material. In some examples, a sample includes blood, serum, or plasma.

Sepsis: A condition where a subject has an infection, and the subject's immune response to the infection damages the subject's own tissue(s). As used herein, “sepsis” can refer to sepsis, severe sepsis, or septic shock. Sepsis is typically diagnosed by presence of infection in combination with one or more of altered body temperature (e.g., above 101° F. or below 98.6° F.), increased respiratory rate (e.g., >20 breaths/minute), and increased heart rate (e.g., >90 beats per minute). Severe sepsis includes sepsis and one or more of altered mental state, low blood pressure (e.g., <100 mm Hg systolic pressure), decreased platelet count, respiratory distress, abnormal heart function, and abdominal pain. Septic shock is sepsis or severe sepsis with low blood pressure due to sepsis that does not improve after treatment (such as fluid support). Sepsis, severe sepsis, and septic shock are life-threatening conditions that are usually treated with antibiotics, intravenous fluids, and other supportive measures, such as oxygen, mechanical ventilation and/or dialysis.

Subject: A living multi-cellular vertebrate organism, a category that includes human and non-human mammals. In one example, a subject has or is suspected to have sepsis or is at risk for sepsis. In other example, a subject does not exhibit symptoms of sepsis.

### II. Sepsis Biomarkers and Methods

Provided herein are biomarker panels and methods of their use. In some embodiments, the biomarker panels are used in methods of predicting development of sepsis in a subject and/or diagnosing sepsis in a subject. In embodiments, the methods include measuring the expression of three or more (such as 3, 4, 5, 6, 7, 8, 9, 10, or more) genes in a sample from a subject. The methods also include determining whether expression of the three or more genes is altered (such as increased or decreased) compared to a control. In some examples, altered expression of the three or more genes indicates that the subject is predicted to develop or has developed sepsis.

In some embodiments, the methods disclosed herein can predict that a subject will develop sepsis, for example, can diagnose a subject with sepsis when they are pre-symptomatic or pre-clinical (e.g., do not exhibit symptoms of sepsis). In some examples, the methods can diagnose a subject as having sepsis when they do not exhibit symptoms of sepsis, such as at least 1 day (for example, at least 1, 2, 3, 4, 5, or more days) prior to diagnosis of sepsis using current clinical criteria. Thus, in some embodiments, the methods utilize a sample that is from a subject who does not exhibit symptoms of sepsis. Subjects and samples are discussed in greater detail below.

A. ISB6 Panels

Disclosed herein are methods that include measuring expression of six or more (for example, 6, 7, 8, 9, 10, or more) genes in a sample from a subject having or suspected to have sepsis and determining whether expression of the six or more genes is altered (for example, increased or decreased) compared to a control. In some embodiments, the set of six or more genes includes or consists of a set of genes provided in Table 5. In other embodiments, the set of six or more genes includes or consists of a set of genes provided in Table 6, for example, CCR1, HLA-DPB1, BATF, C3AR1, ARHGEF18, and C9orf95; CCR1, HLA-DPB1, BATF, C3AR1, MTCH1, and C9orf103; CCR1, HLA-DPB1, BATF, C3AR1, MTCH1, and C9orf95; CCR1, HLA-DPB1, BATF, GNA15, MTCH1, and C9orf95; CD177, HILA-DPB1, BATF, C3AR1, MTCH1, and C9orf95; CD63, HLA-DPB1, PLAC8, FCER1G, MTCH1, and C9orf95; CD63, HLA-DPB1, PLAC8, GNA15, ARHGEF18, and C9orf95; CD63, HLA-DPB1, PLAC8, GNA15, MTCH1, and C9orf95; CD63, HLA-DPB1, BATF, C3AR1, MTCH1, and C9orf95; CD63, HLA-DPB1, BATF, GNA15, MTCH1, and C9orf95; EMR1, HLA-DPB1, BAIT, C3AR1, MTCH1, and C9orf95; EMR1, HLA-DPB1, BATF, GNA15, MTCH1, and C9orf95; FCER1G, HLA-DPB1, PLAC8, C3AR1, MTCH1, and C9orf95; FCER1G, HLA-DPB1, PLAC8, GNA15, ARHGEF18, and C9orf95; FCER1G, HLA-DPB1, PLAC8, GNA15, MTCH1, and C9orf95; FCER1G, HLA-DPB1, BATF, C3AR1, MTCH1, and SEPHS2; FCER1G, HLA-DPB1, BATF, C3AR1, MTCH1, and C9orf95; FES, HLA-DPB1, PLAC8, FCER1G, MTCH1, and C9orf95; FES, HLA-DPB1, PLAC8, GNA15, ARHGEF18, and C9orf103; FES, HLA-DPB1, PLAC8, GNA15, MTCH1, and C9orf95; FES, HLA-DPB1, BATF, C3AR1, MTCH1, and C9orf95; or C3AR1, HLA-DPB1, BATF, GNA15, MTCH1, and C9orf95.

In some examples, expression of one or more of the genes in the set is increased compared to a control and/or expression of one or more of the genes in the set is decreased compared to a control. In particular examples, expression of CCR1, C3AR1, C9orf95, BATF, C9orf103, GNA15, CD177, CD63, EMR1, FCER1G, FES, PLAC8, and/or SPEHS2 is increased compared to the control. In other particular examples, expression of HLA-DPB1, ARHGEF18, and/or MTCH1 is decreased compared to the control.

Also disclosed herein are methods of diagnosing sepsis in a subject that include measuring expression of six or more (for example, 6, 7, 8, 9, 10, or more) genes in a sample from a subject and diagnosing the subject with sepsis when the expression of the six or more genes is altered (for example, increased or decreased) compared to a control. In some embodiments, the set of six or more genes includes or consists of a set of genes provided in Table 5. In other embodiments, the set of six or more genes includes or consists of a set of genes provided in Table 6, for example, CCR1, HLA-DPB1, BATF, C3AR1, ARHGEF18, and C9orf95; CCR1, HLA-DPB1, BATF, C3AR1, MTCH1, and C9orf103; CCR1, HLA-DPB1, BATF, C3AR1, MTCH1, and C9or195; CCR1, HLA-DPB1, BATF, GNA15, MTCH1, and C9orf95; CD177, HLA-DPB1, BATF, C3AR1, MTCH1, and C9orf95; CD63, HLA-DPB1, PLAC8, FCER1G, MTCH1, and C9orf95; CD63, HLA-DPB1, PLAC8, GNA15, ARHGEF18, and C9orf95; CD63, HLA-DPB1, PLAC8, GNA15, MTCH1, and C9orf95; CD63, HLA-DPB1, BATF, C3AR1, MTCH1, and C9orf95; CD63, HLA-DPB1, BATF, GNA15, MTCH1, and C9orf95; EMR1, HLA-DPB1, BATF, C3AR1, MTCH1, and C9orf95; EMR1, HLA-DPB1, BATF, GNA15, MTCH1, and C9orf95; FCER1G, HLA-DPB1, PLAC8, C3AR1, MTCH1, and C9orf95; FCER1G, HLA-DPB1, PLAC8, GNA15, ARHGEF18, and C9orf95; FCER1G, HLA-DPB1, PLAC8, GNA15, MTCH1, and C9orf95; FCER1G, HLA-DPB1, BATF, C3AR1, MTCH1, and SEPHS2; FCER1G, HLA-DPB1, BATF, C3AR1, MTCH1, and C9orf95; FES, HLA-DPB1, PLAC8, FCER1G, MTCH1, and C9orf95; FES, HLA-DPB1, PLAC8, GNA15, ARHGEF18, and C9orf103; FES, HLA-DPB1, PLAC8, GNA15, MTCH1, and C9orf95; FES, HLA-DPB1, BATF, C3AR1, MTCH1, and C9orf95; or C3AR1, HLA-DPB1, BATF, GNA15, MTCH1, and C9orf95.

In some examples, the subject is diagnosed with sepsis when expression of one or more of the genes in the set is increased compared to a control and/or expression of one or more of the genes in the set is decreased compared to a control. In particular examples, expression of CCR1, C3AR1, C9orf95, BATF, C9orf103, GNA15, CD177, CD63, EMR1, FCER1G, FES, PLAC8, and/or SPEHS2 is increased compared to the control. In other particular examples, expression of HLA-DPB1, ARHGEF18, and/or MTCH1 is decreased compared to the control.

Also disclosed herein are methods of treating sepsis in a subject that include measuring expression of six or more (for example, 6, 7, 8, 9, 10, or more) genes in a sample from a subject and administering one or more treatments for sepsis (such as administering one or more antibiotics) when the expression of the six or more genes is altered (for example, increased or decreased) compared to a control. In some embodiments, the set of six or more genes includes or consists of a set of genes provided in Table 5. In other embodiments, the set of six or more genes includes or consists of a set of genes provided in Table 6, for example, CCR1, HLA-DPB1, BATF, C3AR1, ARHGEF18, and C9orf95; CCR1, HLA-DPB1, BATF, C3AR1, MTCH1, and C9orf103; CCR1, HLA-DPB1, BATF, C3AR1, MTCH1, and C9orf95; CCR1, HLA-DPB1, BATF, GNA15, MTCH1, and C9orf95; CD177, HLA-DPB1, BATF, C3AR1, MTCH1, and C9orf95; CD63, HLA-DPB1, PLAC8, FCER1G, MTCH1, and C9orf95; CD63, HLA-DPB1, PLAC8, GNA15, ARHGEF18, and C9orf95; CD63, HLA-DPB1, PLAC8, GNA15, MTCH1, and C9orf95; CD63, HLA-DPB1, BATF, C3AR1, MTCH1, and C9orf95; CD63, HLA-DPB1, BATF, GNA15, MTCH1, and C9orf95; EMR1, HLA-DPB1, BAIT, C3AR1, MTCH1, and C9orf95; EMR1, HLA-DPB1, BATF, GNA15, MTCH1, and C9orf95; FCER1G, HLA-DPB1, PLAC8, C3AR1, MTCH1, and C9orf95; FCER1G, HLA-DPB1, PLAC8, GNA15, ARHGEF18, and C9orf95; FCER1G, HLA-DPB1, PLAC8, GNA15, MTCH1, and C9orf95; FCER1G, HLA-DPB1, BATF, C3AR1, MTCH1, and SEPHS2; FCER1G, HLA-DPB1, BATF, C3AR1, MTCH1, and C9orf95; FES, HLA-DPB1, PLAC8, FCER1G, MTCH1, and C9orf95; FES, HLA-DPB1, PLAC8, GNA15, ARHGEF18, and C9orf103; FES, HLA-DPB1, PLAC8, GNA15, MTCH1, and C9orf95; FES, HLA-DPB1, BATF, C3AR1, MTCH1, and C9orf95; or C3AR1, HLA-DPB1, BATF, GNA15, MTCH1, and C9orf95. In particular examples, expression of CCR1, C3AR1, C9orf95, BATF, C9orf103, GNA15, CD177, CD63, EMR1, FCER1G, FES, PLAC8, and/or SPEHS2 is increased compared to the control. In other particular examples, expression of HLA-DPB1, ARHGEF18, and/or MTCH1 is decreased compared to the control.

In some embodiments of the methods (such as methods of measuring expression of the six or more genes, methods of diagnosing a subject with sepsis, and/or methods of treating sepsis) further include measuring expression of RPGRIP1 in the sample and determining whether expression of RPGRIP1 is altered compared to a control. In some examples, expression of RPGRIP1 is decreased compared to a control. In other examples, the subject is diagnosed with sepsis when expression of RPGRIP1 is decreased compared to a control.

In other embodiments, the methods further include measuring expression of one or more control genes (such as 1, 2, 3, 4, 5, 6, 7, 8, 9, or 10 control genes), such as one or more housekeeping or internal control genes. In some examples, a control gene is expressed at a constant level among different tissues and/or is unaffected by sepsis or an experimental treatment and can be used to normalize patterns of gene expression. Exemplary control or housekeeping genes include GAPDH, β-action, 18S ribosomal RNA, tubulin, BRK1, RNF181, GPR155, SUPT4H1, and FAM74A4.

B. ISB 19-Derived Panels

Disclosed herein are methods that include measuring expression of three or more (for example, 3, 4, 5, 6, or more) genes in a sample from a subject having or suspected to have sepsis or at risk of sepsis and determining whether expression of the three or more genes is altered (for example, increased or decreased) compared to a control. In some embodiments, the set of three or more genes includes or consists of a three or more genes selected from the set provided in Table 12 as the “ISB 19” panel. In other embodiments, the set of three or more genes includes or consists of a set of 3, 4, or 5 genes provided in Table 16, for example, LCN2, SLC2A3, and BMX; LCN2, SLC2A3, and GRB10; LCN2, PFKFB3, and GRB10; LCN2, PFKFB3, and BMX; IL1R2, HK3, and BMX; LCN2, HK3, and BMX; LCN2, HK3, and GRB10; GZMA, HK3, and BMX; FCAR, PFKFB2, and BMX; LCN2, PFKFB3, and IL18R1; LCN2, PFKFB3, GRB10, and ST6GALNAC3; LCN2, SLC2A3, BMX, and LGALS2; IL1R2, SLC2A3, BMX, and TCN1; LCN2, SLC2A3, GRB10, and ST6GALNAC3; FCAR, PFKFB2, BMX, and CEACAM1; IL1R2, HK3, BMX, and CD24; IL1R2, PFKFB3, BMX, and CD24; BMX, SLC2A3, GRB10, and CD24; IL1R2, HK3, BMX, and CEACAM1; GZMA, SLC2A3, BMX, and CD24; LCN2, PFKFB3, GRB10, ST6GALNAC3, and RNASE3; LCN2, PFKFB3, GRB10, RNASE2, and ST6GALNAC3; IL1R2, PFKFB3, GRB10, CD24, and ST6GALNAC3; GZMA, PFKFB3, GRB10, ST6GALNAC3, and CD24; HK3, PFKFB3, GRB10, ST6GALNAC3, and CD24; IL1R2, PFKFB3, GRB10, ST6GALNAC3, and TCN1; SLC2A3, PFKFB3, GRB10, ST6GALNAC3, and TCN1; LCN2, PFKFB3, GRB10, ST6GALNAC3, and DACH1; SLC2A3, PFKFB3, GRB10, ST6GALNAC3, and CD24; or SLC2A3, HK3, BMX, SPOCD1, and LGALS2.

In some examples, expression of one or more of the genes in the set is increased compared to a control and/or expression of one or more of the genes in the set is decreased compared to a control. In particular examples, expression of one or more of LCN2, SLC2A3, BMX, GRB10, PFKFB3, IL1R2, HK3, FCAR, PFKFB2, IL18R1, ST6GALNAC3, TCN1, CEACAM1, CD24, RNASE3, RNASE2, DACH1 and/or SPOCD1 is increased compared to the control. In other particular examples, expression of GZMA and/or LGALS2 is decreased compared to the control.

Also disclosed herein are methods of diagnosing or predicting sepsis in a subject that include measuring expression of three or more (for example, 3, 4, 5, 6, or more) genes in a sample from a subject and diagnosing the subject with sepsis or predicting risk of sepsis when the expression of the three or more genes is altered (for example, increased or decreased) compared to a control. In some embodiments, the three or more genes includes or consists of three or more genes selected from the set provided in Table 12 as the “ISB 19” panel. In other embodiments, the three or more genes includes or consists of a set of 3, 4, or 5 genes provided in Table 16, for example, LCN2, SLC2A3, and BMX; LCN2, SLC2A3, and GRB10; LCN2, PFKFB3, and GRB10; LCN2, PFKFB3, and BMX; IL1R2, HK3, and BMX; LCN2, HK3, and BMX; LCN2, HK3, and GRB10; GZMA, HK3, and BMX; FCAR, PFKFB2, and BMX; LCN2, PFKFB3, and IL18R1; LCN2, PFKFB3, GRB10, and ST6GALNAC3; LCN2, SLC2A3, BMX, and LGALS2; IL1R2, SLC2A3, BMX, and TCN1; LCN2, SLC2A3, GRB10, and ST6GALNAC3; FCAR, PFKFB2, BMX, and CEACAM1; IL1R2, HK3, BMX, and CD24; IL1R2, PFKFB3, BMX, and CD24; BMX, SLC2A3, GRB10, and CD24; IL1R2, HK3, BMX, and CEACAM1; GZMA, SLC2A3, BMX, and CD24; LCN2, PFKFB3, GRB10, ST6GALNAC3, and RNASE3; LCN2, PFKFB3, GRB10, RNASE2, and ST6GALNAC3; IL1R2, PFKFB3, GRB10, CD24, and ST6GALNAC3; GZMA, PFKFB3, GRB10, ST6GALNAC3, and CD24; HK3, PFKFB3, GRB10, ST6GALNAC3, and CD24; IL1R2, PFKFB3, GRB10, ST6GALNAC3, and TCN1; SLC2A3, PFKFB3, GRB10, ST6GALNAC3, and TCN1; LCN2, PFKFB3, GRB10, ST6GALNAC3, and DACH1; SLC2A3, PFKFB3, GRB10, ST6GALNAC3, and CD24; or SLC2A3, HK3, BMX, SPOCD1, and LGALS2.

In some examples, the subject is diagnosed with sepsis or predicted to develop sepsis when expression of one or more of the genes in the set is increased compared to a control and/or expression of one or more of the genes in the set is decreased compared to a control. In particular examples, expression of one or more of LCN2, SLC2A3, BMX, GRB10, PFKFB3, IL1R2, HK3, FCAR, PFKFB2, IL18R1, ST6GALNAC3, TCN1, CEACAM1, CD24, RNASE3, RNASE2, DACH1 and/or SPOCD1 is increased compared to the control. In other particular examples, expression of GZMA and/or LGALS2 is decreased compared to the control.

Also disclosed herein are methods of treating sepsis in a subject that include measuring expression of three or more (for example, 3, 4, 5, 6, or more) genes in a sample from a subject and administering one or more treatments for sepsis (such as administering one or more antibiotics) when the expression of the three or more genes is altered (for example, increased or decreased) compared to a control. In some embodiments, the three or more genes includes or consists of three or more genes selected from the set provided in Table 12 as the “ISB 19” panel. In other embodiments, the three or more genes includes or consists of a set of 3, 4, or 5 genes provided in Table 16, for example, LCN2, SLC2A3, and BMX; LCN2, SLC2A3, and GRB10; LCN2, PFKFB3, and GRB10; LCN2, PFKFB3, and BMX; IL1R2, HK3, and BMX; LCN2, HK3, and BMX; LCN2, HK3, and GRB10; GZMA, HK3, and BMX; FCAR, PFKFB2, and BMX; LCN2, PFKFB3, and IL18R1; LCN2, PFKFB3, GRB10, and ST6GALNAC3; LCN2, SLC2A3, BMX, and LGALS2; IL1R2, SLC2A3, BMX, and TCN1; LCN2, SLC2A3, GRB10, and ST6GALNAC3; FCAR, PFKFB2, BMX, and CEACAM1; IL1R2, HK3, BMX, and CD24; IL1R2, PFKFB3, BMX, and CD24; BMX, SLC2A3, GRB10, and CD24; IL1R2, HK3, BMX, and CEACAM1; GZMA, SLC2A3, BMX, and CD24; LCN2, PFKFB3, GRB10, ST6GALNAC3, and RNASE3; LCN2, PFKFB3, GRB10, RNASE2, and ST6GALNAC3; IL1R2, PFKFB3, GRB10, CD24, and ST6GALNAC3; GZMA, PFKFB3, GRB10, ST6GALNAC3, and CD24; HK3, PFKFB3, GRB10, ST6GALNAC3, and CD24; IL1R2, PFKFB3, GRE 10, ST6GALNAC3, and TCN1; SLC2A3, PFKFB3, GRB10, ST6GALNAC3, and TCN1; LCN2, PFKFB3, GRB10, ST6GALNAC3, and DACH1; SLC2A3, PFKFB3, GRB10, ST6GALNAC3, and CD24; or SLC2A3, HK3, BMX, SPOCD1, and LGALS2. In particular examples, expression of one or more of LCN2, SLC2A3, BMX, GRB10, PFKFB3, IL1R2, HK3, FCAR, PFKFB2, IL18R1, ST6GALNAC3, TCN1, CEACAM1, CD24, RNASE3, RNASE2, DACH1 and/or SPOCD1 is increased compared to the control. In other particular examples, expression of GZMA and/or LGALS2 is decreased compared to the control. In some embodiments, the subject does not exhibit symptoms of sepsis.

In other embodiments, the methods further include measuring expression of one or more control genes (such as 1, 2, 3, 4, 5, 6, 7, 8, 9, or 10 control genes), such as one or more housekeeping or internal control genes. In some examples, a control gene is expressed at a constant level among different tissues and/or is unaffected by sepsis or an experimental treatment and can be used to normalize patterns of gene expression. Exemplary control or housekeeping genes include GAPDH, β-action, 18S ribosomal RNA, tubulin, BRK1, RNF181, GPR155, SUPT4H1, and FAM74A4.

C. ISB63-Derived Panels

Disclosed herein are methods that include measuring expression of six or more (for example, 6, 7, 8, 9, or more) genes in a sample from a subject having or suspected to have sepsis and determining whether expression of the six or more genes is altered (for example, increased or decreased) compared to a control. In some embodiments, the six or more genes includes or consists of six or more genes selected from the set provided in Table 12 as the “ISB 63” panel. In other embodiments, the six or more genes includes or consists of a set of 6, 7, or 8 genes provided in Table 17, for example, RPS6KA3, BCL6, TPM3, GNG10, STOM, and MPO; RPS6KA3, MAVS, TPM3, BCL6, STOM, and MPO; RPS6KA3, BCL6, TPM3, BMX, STOM, and MPO; RPS6KA3, LTF, TPM3, BCL6, STOM, and PDGFC; RPS6KA3, BCL6, TPM3, CD55, STOM, and MPO; RPS6KA3, BCL6, TPM3, GNG10, STOM, and PDGFC; RPS6KA3, LTF, TPM3, BCL6, STOM, and CYP1B1; LTF, BCL6, TPM3, CD55, STOM, and PDGFC; RPS6KA3, BCL6, TPM3, TLR8, STOM, and MPO; RPS6KA3, BCL6, TPM3, HLA-DRA, GNG10, and MPO; RPS6KA3, MLLT1, TPM3, BCL6, GNG10, CYP27A1, and YOD1; RPS6KA3, MLLT1, TPM3, BCL6, GNG10, PDGFC, and YOD1; RPS6KA3, GAS7, TPM3, BCL6, RRBP1, MPO, and YOD1; RPS6KA3, MLLT1, TPM3, BCL6, GNG10, MPO, and LGALS2; RPS6KA3, GAS7, TPM3, BCL6, GNG10, MPO, and YOD1; LTF, MLLT1, TPM3, BCL6, GNG10, PDGFC, and YOD1; RPS6KA3, BCL6, TPM3, GNG10, LILRA2, CYP27A1, and YOD1; RPS6KA3, BCL6, TPM3, GNG10, STOM, MPO, and LGALS2; RPS6KA3, MLLT1, TPM3, BCL6, GNG10, MPO, and YOD1; RPS6KA3, BCL6, TPM3, CD55, STOM, MPO, and IL17RA; RPS6KA3, MLLT1, TPM3, BCL6, GNG10, PDGFC, YOD1, and LGALS2; RPS6KA3, MLLT1, TPM3, BCL6, GNG10, PDGFC, YOD1, and LILRA4; RPS6KA3, MLLT1, TPM3, BCL6, GNG10, PDGFC, YOD1, and IL17RA; RPS6KA3, MLLT1, TPM3, BCL6, GNG10, PDGFC, YOD1, and TCN1; RPS6KA3, MLLT1, TPM3, BCL6, GNG10, PDGFC, YOD1, and RNASE3; RPS6KA3, MLLT1, TPM3, BCL6, GNG10, PDGFC, YOD1, and NOV; RPS6KA3, MLLT1, TPM3, BCL6, GNG10, PDGFC, YOD1, and RNASE2; LTF, MLLT1, TPM3, BCL6, GNG10, PDGFC, YOD1, and FAM105A; RPS6KA3, BCL6, TPM3, HLA-DRA, RRBP1, MPO, ERO1L, and YOD1; or LTF, MLLT1, TPM3, BCL6, GNG10, PDGFC, YOD1, and C14orf101.

In some examples, expression of one or more of the genes in the set is increased compared to a control and/or expression of one or more of the genes in the set is decreased compared to a control. In particular examples, expression of one or more of RPS6KA3, BCL6, TPM3, GNG10, STOM, MPO, BMX, LTF, PDGFC, CD55, CYP1B1, TLR8, MLLT1, YOD1, GAS7, RRBP1, LILRA2, IL17RA, LILRA4, TCN1, RNASE3, RNASE2, FAM105A, ERO1L, and/or C14orf101 is increased compared to the control. In other particular examples, expression of one or more of MAVS, HLA-DRA, CYP27A1, LGALS2, and/or NOV is decreased compared to the control.

Also disclosed herein are methods of measuring expression of a modified set of six or more ISB63 genes comprising LCN2, BMX, MPO, RPS6KA3, BCL6, and MAPK14 in a sample from a subject having or suspected to have sepsis and determining whether expression of the six or more genes is altered (for example, increased or decreased) compared to a control. In particular examples, expression of one or more of LCN2, BMX, MPO, RPS6KA3, BCL6, and MAPK14 is increased compared to the control.

Also disclosed herein are methods of diagnosing or predicting sepsis in a subject that include measuring expression of six or more (for example, 6, 7, 8, 9, or more) genes in a sample from a subject and diagnosing the subject with sepsis or predicting risk of sepsis when the expression of the six or more genes is altered (for example, increased or decreased) compared to a control. In some embodiments, the six or more genes includes or consists of six or more genes selected from the set provided in Table 12 as the “ISB 63” panel. In other embodiments, the six or more genes includes or consists of a set of 6, 7, or 8 genes provided in Table 17, for example, RPS6KA3, BCL6, TPM3, GNG10, STOM, and MPO; RPS6KA3, MAVS, TPM3, BCL6, STOM, and MPO; RPS6KA3, BCL6, TPM3, BMX, STOM, and MPO; RPS6KA3, LTF, TPM3, BCL6, STOM, and PDGFC; RPS6KA3, BCL6, TPM3, CD55, STOM, and MPO; RPS6KA3, BCL6, TPM3, GNG10, STOM, and PDGFC; RPS6KA3, LTF, TPM3, BCL6, STOM, and CYP1B1; LTF, BCL6, TPM3, CD55, STOM, and PDGFC; RPS6KA3, BCL6, TPM3, TLR8, STOM, and MPO; RPS6KA3, BCL6, TPM3, HLA-DRA, GNG10, and MPO; RPS6KA3, MLLT1, TPM3, BCL6, GNG10, CYP27A1, and YOD1; RPS6KA3, MLLT1, TPM3, BCL6, GNG10, PDGFC, and YOD1; RPS6KA3, GAS7, TPM3, BCL6, RRBP1, MPO, and YOD1; RPS6KA3, MLLT1, TPM3, BCL6, GNG10, MPO, and LGALS2; RPS6KA3, GAS7, TPM3, BCL6, GNG10, MPO, and YOD1; LIT, MLLT1, TPM3, BCL6, GNG10, PDGFC, and YOD1; RPS6KA3, BCL6, TPM3, GNG10, LILRA2, CYP27A1, and YOD1; RPS6KA3, BCL6, TPM3, GNG10, STOM, MPO, and LGALS2; RPS6KA3, MLLT1, TPM3, BCL6, GNG10, MPO, and YOD1; RPS6KA3, BCL6, TPM3, CD55, STOM, MPO, and IL17RA; RPS6KA3, MLLT1, TPM3, BCL6, GNG10, PDGFC, YOD1, and LGALS2; RPS6KA3, MLLT1, TPM3, BCL6, GNG10, PDGFC, YOD1, and LILRA4; RPS6KA3, MLLT1, TPM3, BCL6, GNG10, PDGFC, YOD1, and IL17RA; RPS6KA3, MLLT1, TPM3, BCL6, GNG10, PDGFC, YOD1, and TCN1; RPS6KA3, MLLT1, TPM3, BCL6, GNG10, PDGFC, YOD1, and RNASE3; RPS6KA3, MLLT1, TPM3, BCL6, GNG10, PDGFC, YOD1, and NOV; RPS6KA3, MLLT1, TPM3, BCL6, GNG10, PDGFC, YOD1, and RNASE2; LTF, MLLT1, TPM3, BCL6, GNG10, PDGFC, YOD1, and FAM105A; RPS6KA3, BCL6, TPM3, HLA-DRA, RRBP1, MPO, ERO1L, and YOD1; or LTF, MLLT1, TPM3, BCL6, GNG10, PDGFC, YOD1, and C14orf101.

In some examples, the subject is diagnosed with sepsis or predicted to develop sepsis when expression of one or more of the genes in the set is increased compared to a control and/or expression of one or more of the genes in the set is decreased compared to a control. In particular examples, the subject is diagnosed with sepsis when expression of one or more of RPS6KA3, BCL6, TPM3, GNG10, STOM, MPO, BMX, LTF, PDGFC, CD55, CYP1B1, TLR8, MLLT1, YOD1, GAS7, RRBP1, LILRA2, IL17RA, LILRA4, TCN1, RNASE3, RNASE2, FAM105A, ERO1L, and/or C14orf101 is increased compared to the control. In other particular examples, expression of one or more of MAVS, HLA-DRA, CYP27A1, LGALS2, and/or NOV is decreased compared to the control.

Also disclosed herein are methods of diagnosing or predicting sepsis in a subject that include measuring expression of a modified set of six or more ISB63 genes comprising LCN2, BMX, MPO, RPS6KA3, BCL6, and MAPK14 in a sample from a subject and diagnosing the subject with sepsis or predicting risk of sepsis when the expression of the six or more genes is altered (for example, increased or decreased) compared to a control. In particular examples, expression of one or more of LCN2, BMX, MPO, RPS6KA3, BCL6, and MAPK14 is increased compared to the control.

Also disclosed herein are methods of treating sepsis in a subject that include measuring expression of six or more (for example, 6, 7, 8, 9, or more) genes in a sample from a subject and administering one or more treatments for sepsis (such as administering one or more antibiotics) when the expression of the six or more genes is altered (for example, increased or decreased) compared to a control. In some embodiments, the six or more genes includes or consists of six or more genes selected from the set provided in Table 12 as the “ISB 63” panel. In other embodiments, the six or more genes includes or consists of a set of 6, 7, or 8 genes provided in Table 17, for example, RPS6KA3, BCL6, TPM3, GNG10, STOM, and MPO; RPS6KA3, MAVS, TPM3, BCL6, STOM, and MPO; RPS6KA3, BCL6, TPM3, BMX, STOM, and MPO; RPS6KA3, LTF, TPM3, BCL6, STOM, and PDGFC; RPS6KA3, BCL6, TPM3, CD55, STOM, and MPO; RPS6KA3, BCL6, TPM3, GNG10, STOM, and PDGFC; RPS6KA3, LTF, TPM3, BCL6, STOM, and CYP1B1; LTF, BCL6, TPM3, CD55, STOM, and PDGFC; RPS6KA3, BCL6, TPM3, TLR8, STOM, and MPO; RPS6KA3, BCL6, TPM3, HLA-DRA, GNG10, and MPO; RPS6KA3, MLLT1, TPM3, BCL6, GNG10, CYP27A1, and YOD1; RPS6KA3, MLLT1, TPM3, BCL6, GNG10, PDGFC, and YOD1; RPS6KA3, GAS7, TPM3, BCL6, RRBP1, MPO, and YOD1; RPS6KA3, MLLT1, TPM3, BCL6, GNG10, MPO, and LGALS2; RPS6KA3, GAS7, TPM3, BCL6, GNG10, MPO, and YOD1; LTF, MLLT1, TPM3, BCL6, GNG1C, PDGFC, and YOD1; RPS6KA3, BCL6, TPM3, GNG10, LILRA2, CYP27A1, and YOD1; RPS6KA3, BCL6, TPM3, GNG10, STOM, MPO, and LGALS2; RPS6KA3, MLLT1, TPM3, BCL6, GNG10, MPO, and YOD1; RPS6KA3, BCL6, TPM3, CD55, STOM, MPO, and IL17RA; RPS6KA3, MLLT1, TPM3, BCL6, GNG10, PDGFC, YOD1, and LGALS2; RPS6KA3, MLLT1, TPM3, BCL6, GNG10, PDGFC, YOD1, and LILRA4; RPS6KA3, MLLT1, TPM3, BCL6, GNG10, PDGFC, YOD1, and IL17RA; RPS6KA3, MLLT1, TPM3, BCL6, GNG10, PDGFC, YOD1, and TCN1; RPS6KA3, MLLT1, TPM3, BCL6, GNG10, PDGFC, YOD1, and RNASE3; RPS6KA3, MLLT1, TPM3, BCL6, GNG10, PDGFC, YOD1, and NOV; RPS6KA3, MLLT1, TPM3, BCL6, GNG10, PDGFC, YOD1, and RNASE2; LTF, MLLT1, TPM3, BCL6, GNG10, PDGFC, YOD1, and FAM105A; RPS6KA3, BCL6, TPM3, HLA-DRA, RRBP1, MPO, ERO1L, and YOD1; or LTF, MLLT1, TPM3, BCL6, GNG10, PDGFC, YOD1, and C14orf101. In particular examples, expression of one or more of RPS6KA3, BCL6, TPM3, GNG10, STOM, MPO, BMX, LTF, PDGFC, CD55, CYP1B1, TLR8, MLLT1, YOD1, GAS7, RRBP1, LILRA2, IL17RA, LILRA4, TCN1, RNASE3, RNASE2, FAM105A, ERO1L, and/or C14orf101 is increased compared to the control. In other particular examples, expression of one or more of MAVS, HLA-DRA, CYP27A1, LGALS2, and/or NOV is decreased compared to the control. In some embodiments, the subject does not exhibit symptoms of sepsis.

Also disclosed herein are methods of treating sepsis in a subject that include measuring expression of a modified set of six or more ISB63 genes comprising LCN2, BMX, MPO, RPS6KA3, BCL6, and MAPK14 in a sample from a subject and administering one or more treatments for sepsis (such as administering one or more antibiotics) when the expression of the six or more genes is altered (for example, increased or decreased) compared to a control. In particular examples, expression of one or more of LCN2, BMX, MPO, RPS6KA3, BCL6, and MAPK14 is increased compared to the control.

In other embodiments, the methods further include measuring expression of one or more control genes (such as 1, 2, 3, 4, 5, 6, 7, 8, 9, or 10 control genes), such as one or more housekeeping or internal control genes. In some examples, a control gene is expressed at a constant level among different tissues and/or is unaffected by sepsis or an experimental treatment and can be used to normalize patterns of gene expression. Exemplary control or housekeeping genes include GAPDH, β-action, 18S ribosomal RNA, tubulin, BRK1, RNF181, GPR155, SUPT4H1, and FAM74A4.

D. Subjects and Samples

The methods disclosed herein utilize a sample from a subject. In some embodiments, a subject has or is suspected to have sepsis. In other embodiments, a subject does not exhibit symptoms of sepsis (e.g., does not have sepsis and/or has sepsis, but is pre-symptomatic). In some examples, the subject may be at risk for development of sepsis, for example, a subject with an infection (e.g., a bacterial, viral, fungal, or parasitic infection), a subject who has experienced trauma (e.g., wound, injury, or burn), a subject who has undergone a surgical procedure, a subject who has received an organ transplant, or a subject with an invasive device (e.g., a catheter or breathing tube). In some examples, the subject has an infection with Enterococcus, Pseudomonas aeruginosa, Staphylococcus aureus, or Enterobacteriaceae. In additional examples, a subject at risk for sepsis may be younger than 1 year or older than 65, have diabetes, and/or have a compromised immune system (e.g., subjects with AIDS (or HIV positive), subjects with severe combined immunodeficiency, subjects who have had transplants and who are taking immunosuppressants, subjects who have cancer or who are receiving chemotherapy for cancer, subjects who do not have a spleen, subjects with end stage kidney disease, and subjects who have been taking corticosteroids or other immune suppressing therapy). In particular examples, a subject at risk for sepsis includes a subject with pneumonia, an abdominal infection (e.g., peritonitis), a kidney infection or urinary tract infection, a subject who has undergone surgery, and/or a subject who has experienced trauma.

In some examples, the subject has experienced a risk factor for development of sepsis (including but not limited to infection, surgery, or trauma) within 1-14 days of collecting a sample from the subject. For example, one or more samples is collected from a subject 1, 2, 3, 4, 5, 6, 7, 8, 9, 10, 11, 12, 13, or 14 days after undergoing surgery or experiencing a trauma. In other examples, one or more samples are collected from a subject prior to a risk factor for developing sepsis (including but not limited to surgery, placement of an invasive device, or immunosuppressant or chemotherapy), such as 1-14 days prior to the risk factor. In some examples, one or more samples are collected from the subject 1, 2, 3, 4, 5, 6, 7, 8, 9, 10, 11, 12, 13, or 14 days before the risk factor. Samples may be collected from a subject both before (such as 1-14 days before) and after (such as 1-14 days after) the risk factor, in some examples.

Samples that can be used in the disclosed methods include biological specimens containing nucleic acids (for example, DNA, RNA, and/or mRNA), proteins, or combinations thereof, obtained from a subject. Examples include, but are not limited to, peripheral blood, peripheral blood mononuclear cells (PBMCs), serum, plasma, urine, saliva, sputum, wound secretions, pus, tissue biopsy, fine needle aspirate, surgical specimen, and autopsy material. In particular examples, the sample is peripheral blood from a subject. In some examples, samples are used directly in the methods provided herein. In other examples, samples are manipulated prior to analysis using the disclosed methods, such as through concentrating, filtering, centrifuging, diluting, desalting, denaturing, reducing, alkylating, proteolyzing, or combinations thereof. In some examples, components of the samples (for example, nucleic acids and/or proteins) are isolated or purified from the sample prior to analysis using the disclosed methods, such as isolating cells, proteins, and/or nucleic acid molecules from the samples.

In some embodiments, one or more samples (such as 1, 2, 3, 4, 5, 6, 7, 8, 9, 10, or more samples) are collected from a subject. In some examples, multiple samples are collected and tested, for example, to detect changes in gene expression prior to development of symptoms of sepsis. The samples may be collected about every 2 hours, about every 4 hours, about every 8 hours, about every 12 hours, about every 18 hours, about once per day, about every other day, or combinations thereof. The samples may be collected prior to diagnosis with sepsis, after diagnosis with sepsis, or a combination thereof. In some examples, samples are collected from a subject who does not exhibit symptoms of sepsis, such as at least 1 day (for example, at least 1, 2, 3, 4, 5, or more days) prior to diagnosis of sepsis using current clinical criteria. In some examples, one or more samples are collected from a subject after an event that increases their risk for developing sepsis, including but not limited to surgery, trauma or other wound, burn(s), infection (such as pneumonia, meningitis, urinary tract infection, peritonitis, or skin infection), or presence of an invasive device (such as a catheter or ventilator).

E. Detecting Gene Expression

As described herein, expression of the sepsis biomarkers disclosed herein (e.g., one or more panels provided herein, including in any one of Tables 5, 6, 12, 16, and 17) can be measured or detected using any one of a number of methods. Detecting or measuring expression of nucleic acid molecules (e.g., mRNA, cDNA) or protein is contemplated herein. The detection methods can be qualitative, semi-quantitative, or quantitative.

1. Methods for Detecting mRNA

Gene expression can be evaluated by detecting RNA (e.g., mRNA or cDNA) encoding the genes of interest. Thus, the disclosed methods can include evaluating mRNA(s) encoding the genes or sets of genes provided herein (including in any one of Tables 5, 6, 12, 16, and 17). In other examples, mRNA encoding the gene(s) of interest is reverse transcribed to cDNA and the cDNA is measured or detected. In some examples, the mRNA (or cDNA) is quantified. The amount of the mRNA (or cDNA) can be assessed in a sample from a subject and optionally in a control sample (such as a sample from a healthy subject). The amounts of mRNA (or cDNA) can be compared to levels of the mRNA (or cDNA) found in sample(s) from healthy subjects or other controls (such as a standard value or reference value). A significant increase or decrease in the amount can be evaluated using statistical methods. An alteration in the amount of the mRNA or cDNA in a sample from the subject relative to a control, such as an increase or decrease in expression, indicates whether the subject has sepsis or is likely to develop sepsis, as described herein.

RNA can be isolated from a sample from a subject, for example using commercially available kits, such as those from QIAGEN®. General methods for mRNA extraction are disclosed in, for example, including Ausubel et al., Current Protocols of Molecular Biology, John Wiley and Sons (1997). In some examples, RNA can also be extracted from paraffin embedded tissues (e.g., see Rupp and Locker, Lab Invest. 56:A67, 1987 and De Andres et al., BioTechniques 18:42044, 1995). Total RNA from cells (such as those obtained from a subject) can be isolated using QIAGEN® RNeasy mini-columns. Other commercially available RNA isolation kits include MASTERPURE® Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from biological samples can also be isolated, for example, by cesium chloride density gradient centrifugation.

Methods of measuring or detecting gene expression include methods based on hybridization analysis of polynucleotides, methods based on sequencing of polynucleotides, and proteomics-based methods. In some examples, mRNA (or cDNA) expression in a sample is quantified using northern blotting or in situ hybridization (Parker & Barnes, Methods in Molecular Biology 106:247-283, 1999); RNAse protection assays (Hod, Biotechniques 13:852-4, 1992); and/or PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

In one example, RT-PCR can be used. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif, USA), or other commercially available kits, following the manufacturer's instructions. The derived cDNA can then be used as a template in the subsequent PCR reaction.

Although the PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is not extendable by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments dissociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

To minimize errors and the effect of sample-to-sample variation, RT-PCR can be performed using an internal standard. The ideal internal standard is expressed at a constant level among different tissues, and/or is unaffected by the experimental treatment. RNAs commonly used to normalize patterns of gene expression are mRNAs for the housekeeping genes glyceraldehyde-3-phosphate-dehydrogenase (GAPDH), beta-actin, tubulin, and 18S ribosomal RNA. Additional internal control genes include BRK1, RNF181, GPR155, SUPT4H1, and FAM74A4

A variation of RT-PCR is real time quantitative RT-PCR (qRT-PCR), which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986 994, 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are available from PE Applied Biosystems, 850 Lincoln Centre Drive, Foster City, Calif. 94404 under the trademark ABI PRISM® 7700.

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some embodiments of this method, the expression of a housekeeping gene or internal control can also be evaluated. These terms include any constitutively or globally expressed gene whose presence enables an assessment of a sepsis-associated mRNA levels. Such an assessment includes a determination of the overall constitutive level of gene transcription and a control for variations in RNA recovery. Internal control (or internal reference) also refers to genes that show little or minimal change between different conditions (such as presence or absence of sepsis). In one example, an internal reference gene is one that shows <1.1-fold change and p-value >0.05 between sepsis and control (no sepsis) samples. Exemplary housekeeping genes include but are not limited to GAPDH, 18S ribosomal RNA, β-actin and tubulin. Exemplary internal control genes include but are not limited to BRK1, RNF181, GPR155, SUPT4H1, and FAM74A4. In particular examples, BRK1 and RNF181 are used as internal controls.

In other examples, gene expression is identified or confirmed using a microarray technique. Thus, the expression profile can be measured in a sample from a subject using microarray technology. In this method, nucleic acid sequences from one or more of the sepsis panels disclosed herein, including but not limited to those included in any one of Tables 5, 6, 12, 16, and 17 (including cDNAs and/or oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from a sample from a subject, and optionally from corresponding non-sepsis (e.g., healthy) samples.

In a specific embodiment of the microarray technique, PCR amplified inserts of cDNA clones are applied to a substrate in a dense array. At least probes specific for one or more panels disclosed herein, such as those in any one of Tables 5, 6, 12, 16, and 17 (and in some examples one or more housekeeping and/or internal control genes) are applied to the substrate, and the array can include, consist essentially of, or consist of these nucleic acids. The microarrayed nucleic acids are suitable for hybridization under stringent conditions. Fluorescently labeled cDNA probes may be generated through incorporation of fluorescent nucleotides by reverse transcription of RNA extracted from a sample of interest (such as a sample from a subject with sepsis, suspected to have sepsis, or at risk of sepsis). Labeled cDNA probes applied to the chip hybridize with specificity to each spot of DNA on the array. After stringent washing to remove non-specifically bound probes, the chip is scanned by confocal laser microscopy or by another detection method. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for the panels disclosed herein. Microarray analysis can be performed by commercially available equipment, following manufacturer's protocols.

Serial analysis of gene expression (SAGE) allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-7, 1995; and Velculescu et al., Cell 88:243-51, 1997, herein incorporated by reference).

In situ hybridization (ISH) is another method for detecting and comparing expression of the panels described herein, including but not limited to those disclosed in any one of Tables 5, 6, 12, 16, and 17. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH).

Sample cells or tissues can be treated to increase their permeability to allow one or more gene-specific probes to enter the cells. The one or more probes are added to the treated cells, allowed to hybridize at pertinent temperature, and excess probe is washed away. The probe can be labeled, for example with a radioactive, fluorescent or antigenic tag, so that the probe's location and in some examples quantity, in the tissue can be determined, for example using autoradiography, fluorescence microscopy or immunoassay. Probes can be designed based on the known sequences of the genes (such as the GenBank accession numbers provided herein) such that the probes specifically bind the gene of interest.

In situ PCR is the PCR based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples can be fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyders. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products can be achieved by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

Gene expression can also be detected and quantitated using the nCounter® technology developed by NanoString (Seattle, Wash.; see, for example, U.S. Pat. Nos. 7,473,767; 7,919,237; and 9,371,563, which are herein incorporated by reference in their entirety). The nCounter® analysis system utilizes a digital color-coded barcode technology that is based on direct multiplexed measurement of gene expression. The technology uses molecular “barcodes” and single molecule imaging to detect and count hundreds of unique transcripts in a single reaction. Each color-coded barcode is attached to a single target-specific probe corresponding to a gene of interest. Mixed together with controls, they form a multiplexed CodeSet.

Each color-coded barcode represents a single target molecule. Barcodes hybridize directly to target molecules and can be individually counted without the need for amplification. The method includes three steps: (1) hybridization; (2) purification and immobilization; and (3) counting. The technology employs two approximately 50 base probes per mRNA that hybridize in solution. The reporter probe carries the signal; the capture probe allows the complex to be immobilized for data collection. After hybridization, the excess probes are removed and the probe/target complexes are aligned and immobilized in the nCounter® cartridge. Sample cartridges are placed in the digital analyzer for data collection. Color codes on the surface of the cartridge are counted and tabulated for each target molecule. This method is described in, for example, U.S. Pat. No. 7,919,237; and U.S. Patent Application Publication Nos. 20100015607; 20100112710; 20130017971, which are herein incorporated by reference in their entirety.

2. Arrays for Profiling Gene Expression

In particular embodiments, arrays (such as a solid support, for example a multi-well plate, a membrane, a bead, or flow cell) are provided that can be used to evaluate gene expression, for example to determine if a subject has or is likely to develop symptoms of sepsis. Such arrays can include a set of specific binding agents (such as nucleic acid probes and/or primers) specific for genes of one or more panels described herein, including but not limited to those in any one of Tables 5, 6, 12, 16, and 17. When describing an array that consists essentially of probes or primers specific for a panel provided herein, including those in any one of Tables 5, 6, 12, 16, and 17, such an array includes probes or primers specific for the genes of the panel, and can further include control probes or primers, such as 1-10 control probes or primers (for example to confirm the incubation conditions are sufficient). In some examples, the array may further comprise additional, such as 1, 2, 3, 4 or 5 additional probes for other genes. In some examples, the array includes 1-10 housekeeping- and/or internal control-specific probes or primers. In one example, an array is a multi-well plate (e.g., 98 or 364 well plate).

In one example, the array includes, consists essentially of, or consists of probes or primers (such as an oligonucleotide or antibody) that can recognize the genes in the panels listed in any one of Tables 5, 6, 12, 16, and 17 (and in some examples also 1-10 housekeeping or control genes). In another example, the array includes, consists essentially of, or consists of probes or primers (such as an oligonucleotide or antibody) that can recognize each of LCN2, BMX, MPO, RPS6KA3, BCL6, and MAPK14 (and in some examples also 1-10 housekeeping or control genes). The oligonucleotide probes or primers can further include one or more detectable labels, to permit detection of hybridization signals between the probe and its target sequence.

3. Methods for Detecting Protein Expression

In some examples, expression of proteins of the panels provided herein, including but not limited to those listed in any one of Tables 5, 6, 12, 16, and 17 is analyzed. Suitable biological samples include samples containing protein obtained from a subject with sepsis, a subject suspected to have sepsis, or a subject at risk for sepsis. An alteration in the amount of the proteins in a sample from the subject relative to a control, such as an increase or decrease in protein expression, indicates whether the subject has sepsis or is likely to develop sepsis, as described herein.

Antibodies specific for the panels of proteins provided herein, including but not limited to those listed in any one of Tables 5, 6, 12, 16, and 17 can be used for protein detection and quantification, for example using an immunoassay method, such as those presented in Harlow and Lane (Antibodies, A Laboratory Manual, CSHL, New York, 1988). Exemplary immunoassay formats include ELISA, Western blot, and RIA assays. Thus, protein levels in a sample can be evaluated using these methods. Immunohistochemical techniques can also be utilized protein detection and quantification. General guidance regarding such techniques can be found in Bancroft and Stevens (Theory and Practice of Histological Techniques, Churchill Livingstone, 1982) and Ausubel et al, (Current Protocols in Molecular Biology, John Wiley & Sons, New York, 1998).

To quantify proteins, a biological sample of the subject that includes cellular proteins can be used. Quantification of the proteins of the panels provided herein, including but not limited to those listed in any one of Tables 5, 6, 12, 16, and 17 can be achieved by immunoassay methods. The amount of the proteins can be assessed in a sample from a subject and optionally in a control sample (such as a sample from a healthy subject). The amounts of protein can be compared to levels of the protein found in sample(s) from healthy subjects or other controls (such as a standard value or reference value). A significant increase or decrease in the amount can be evaluated using statistical methods.

Quantitative spectroscopic approaches, such as SELDI, can be used to analyze gene expression in a sample (such as a sample from a subject with sepsis or suspected to have or develop sepsis). In one example, surface-enhanced laser desorption-ionization time-of-flight (SELDI-TOF) mass spectrometry is used to detect protein expression, for example by using the ProteinChip™ (Ciphergen Biosystems, Palo Alto, Calif.). Such methods are well known in the art (for example see U.S. Pat. Nos. 5,719,060; 6,897,072; and 6,881,586). SELDI is a solid phase method for desorption in which the analyte is presented to the energy stream on a surface that enhances analyte capture or desorption.

The surface chemistry allows the bound analytes to be retained and unbound materials to be washed away. Subsequently, analytes bound to the surface (such as tumor-associated proteins) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in a particular example, the chromatographic surface includes antibodies that specifically bind to proteins of the panels listed in any one of Tables 5, 6, 11, 15, or 16. In other examples, the chromatographic surface consists essentially of, or consists of, antibodies that specifically bind to proteins of the panels listed in any one of Tables 5, 6, 11, 15, or 16. In further examples, the chromatographic surface includes antibodies that specifically bind to proteins LCN2, BMX, MPO, RPS6KA3, BCL6, and MAPK14. In some examples, the chromatographic surface includes antibodies that bind other molecules, such as housekeeping proteins (e.g., tubulin, β-actin, GAPDH, or 18S ribosomal RNA) or internal control proteins (e.g., BRK1, RNF181, GPR155, SUPT4H1, or FAM74A4). In another example, antibodies are immobilized onto the surface using a bacterial Fc binding support.

The chromatographic surface is incubated with a sample. The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

F. Controls

The methods disclosed herein include determining expression of one or more genes (such as a panel of genes provided herein, including but not limited to those listed in any one of Tables 5, 6, 11, 15, or 16) is altered compared to a control. In some examples the expression is compared to a control, such as compared to expression in a sample from a subject known not to have sepsis, for example, a healthy subject (or compared to a reference value or range of values representing such).

The control can be any suitable control against which to compare expression of any one of the panels of genes disclosed herein (including in any one of Tables 5, 6, 11, 15, or 16) in a sample from a subject. In some embodiments, the control sample is a sample, or plurality of samples, from a subject(s) known not to have sepsis (e.g., one or more “healthy” subjects). In other examples, the control sample is a sample, or plurality of samples, from a subject(s) known to have sepsis (in which case the increase or decrease in expression correlation to sepsis is reversed). In some embodiments, the control is a reference value. For example, the reference value can be derived from the average expression values obtained from a group of subjects known not to have (or known to have) sepsis.

An increase in expression includes any detectable increase in the production of a gene product, for example, compared to a control. In certain examples, production of a gene product increases by at least 20%, at least 50%, at least 75%, at least 90%, at least 2-fold, at least 3-fold or at least 4-fold, as compared to a control (such as a sample from a subject known not to have sepsis or compared to a reference value or range of values representing such). In one example, a control is a relative amount of gene expression in a biological sample, such as a sample from a subject known not to have sepsis. In some examples, the control is a reference value (or range of values) for an expected amount of expression of each of the panel of genes from a subject known not to have sepsis. In some examples, the control is a sample from a subject known not to have sepsis (which can be analyzed in parallel with a test sample).

A decrease in expression includes any detectable decrease in the production of a gene product, for example, compared to a control. In certain examples, production of a gene product decreases by at least 20%, at least 50%, at least 75%, at least 90%, at least 2-fold, at least 3-fold or at least 4-fold, as compared to a control (such as a sample from a subject known not to have sepsis or compared to a reference value or range of values representing such). In one example, a control is a relative amount of gene expression in a biological sample, such as a sample from a subject known not to have sepsis. In some examples, the control is a reference value (or range of values) for an expected amount of expression of each of the panel of genes from a subject known not to have sepsis. In some examples, the control is a sample from a subject known not to have sepsis (which can be analyzed in parallel with a test sample).

G. Additional Features

In some embodiments, the methods provided herein further include analyzing one or more additional clinical parameters and integrating the information with the panels disclosed herein (such as one or more panels provided herein, including those in any one of Tables 5, 6, 11, 15, or 16). In some examples, the additional clinical parameters alone do not provide good performance for diagnosing or predicting sepsis, however, they can provide improved performance in combination with the panels disclosed herein.

In one example, the methods include determining a Sequential Organ Failure Assessment (SOFA) score for the subject. The SOFA score is a mortality prediction score measurement used to determine the extent of organ failure in a subject based on degree of function/dysfunction of six organ systems. It includes PaO₂, FiO₂, platelet count, Glasgow coma scale, bilirubin level, mean arterial pressure and administration of vasoactive agents, creatinine level, and urine output to generate a score (Vincent et al., Crit. Care Med. 26, 1793-1800, 1998; Ferreira et al., JAMA 286:1754-1758, 2001). Thus, in some examples, one or more SOFA scores are obtained for a subject having sepsis, suspected to have sepsis, or at risk for sepsis (for example, one or more times per day). The SOFA score is then integrated with the gene panel to determine whether the subject has or is likely to develop sepsis.

In another example, the level of C-reactive protein (CRP) is determined in a sample from the subject. CRP level is commonly used as a non-specific indicator of infection in a subject. Thus, in some examples, a level of CRP is measured in a sample from a subject having sepsis, suspected to have sepsis, or at risk for sepsis (for example, one or more times per day). The CRP level is then integrated with the gene panel to determine whether the subject has or is likely to develop sepsis.

In some examples, both a SOFA score and level of CRP are measured or determined for a subject having sepsis, suspected to have sepsis, or at risk for sepsis (for example, one or more times per day). The SOFA score and CRP level are then integrated with the gene panel to determine whether the subject has or is likely to develop sepsis.

### III. Kits

Also provided are kits including sets of specific binding agents, such as sets of nucleic acid probes, nucleic acid primers, and/or antibodies (or antibody fragments) specific for each of the genes in one or more panels provided herein (including those listed in any one of Tables 5, 6, 11, 15, or 16). In some examples, a kit includes a nucleic acid probe specific for each of the genes in one or more panels listed in any one of Tables 5, 6, 11, 15, or 16, one or more nucleic acid primers (e.g., 1, 2, 3, 4, or more primers) specific for each of the genes in one or more panels listed in any one of Tables 5, 6, 11, 15, or 16, an antibody specific for proteins encoded by each of the genes in one or more panels listed in any one of Tables 5, 6, 11, 15, or 16, or combinations thereof. In other examples, a kit includes a nucleic acid probe specific for each of the LCN2, BMX, MPO, RPS6KA3, BCL6, and MAPK14; one or more nucleic acid primers (e.g., 1, 2, 3, 4, or more primers) specific for each of LCN2, BMX, MPO, RPS6KA3, BCL6, and MAPK14; an antibody specific for proteins encoded by each of LCN2, BMX, MPO, RPS6KA3, BCL6, and MAPK14; or combinations thereof. Such probes, primers, and/or antibodies can be in vials, such as a glass or plastic container, or attached or conjugated to an array (e.g., a solid substrate). In some examples, the probes, primers, and/or antibodies are in a carrier, such as a buffer (e.g., saline). In some examples, such sets further include a nucleic acid probe, one or more nucleic acid primers, or an antibody, specific for at least one housekeeping and/or internal control molecule, such as 1, 2, 3, 4, 5, 6, 7, 8, 9 or 10, or about 1 to about 3, or about 1 to about 5, housekeeping and/or internal control molecules (e.g., β-actin, GAPDH, tubulin, BRK1, RNF181, GPR155, SUPT4H1, or FAM74A4). Such kits can include other components, such as a buffer (e.g., hybridization buffer), enzyme(s), and/or one or more detection reagents.

In some embodiments, the kit includes at least one surface with at least one nucleic acid probe, nucleic acid primer, and/or antibody immobilized on the surface (for example on an array, bead, or flow cell). The disclosed kits can include at least one surface with at least one nucleic acid probe, nucleic acid primer, and/or antibody immobilized on the surface in an addressable manner. Some of the surfaces (or substrates) which can be used in the disclosed kits (or methods) are readily available from commercial suppliers. In some embodiments, the surface is a 96-, 384-, or 1536-well microliter plate, such as modified plates sold by Corning Costar or BD Biosciences (for example, gamma-irradiated plates). In other embodiments, a substrate includes one or more beads (such as a population of beads that can be differentiated by size or color, for example by flow cytometry). In some embodiments, a substrate includes a flow cell (such as a flow cell or a microfluidic ship with a plurality of channels). Alternatively, a surface comprising wells which, in turn, comprise indentations or “dimples” can be formed by micromachining a substance such as aluminum or steel to prepare a mold, then microinjecting plastic or a similar material into the mold to form a structure. Alternatively, a structure comprised of glass, plastic, ceramic, or the like, can be assembled. In some examples, the base is a flat piece of material (for example glass or plastic), in, for example, the shape of the lower portion of a typical microplate used for a biochemical assay. The top surface of the base can be either flat or formed with indentations that will align with a subdivider to provide full subdivisions, or wells, within each sample well. The pieces can be joined by standard procedures, for example the procedures used in the assembly of silicon wafers.

A wide variety of array formats for arrangement of the nucleic acid probes, nucleic acid primers, and/or antibodies can be employed. One suitable format includes a two-dimensional pattern of discrete cells (such as 4096 squares in a 64 by 64 array). Other suitable array formats include but are not limited to slot (rectangular) and circular arrays (see U.S. Pat. No. 5,981,185). In some examples, the array is a multi-well plate. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit low background fluorescence. In another example, a surface activated organic polymer is used as the solid support surface. One example of a surface activated organic polymer is a polypropylene material aminated via radio frequency plasma discharge. Other reactive groups can also be used, such as carboxylated, hydroxylated, thiolated, or active ester groups.

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluoride, polyfluoroethylene-propylene, polyethylenevinyl alcohol, polymethylpentene, polycholorotrifluoroethylene, polysulfones, hydroxylated biaxially oriented polypropylene, aminated biaxially oriented polypropylene, thiolated biaxially oriented polypropylene, ethyleneacrylic acid, ethylene methacrylic acid, and blends of copolymers thereof (see U.S. Pat. No. 5,985,567).

In general, suitable characteristics of the material that can be used to form the solid support surface include: being amenable to surface activation such that upon activation, the surface of the support is capable of covalently attaching a biomolecule such as an oligonucleotide or antibody thereto; amenability to in situ synthesis of biomolecules; being chemically inert such that at the areas on the support not occupied by the oligonucleotides or proteins (such as antibodies) are not amenable to non-specific binding, or when non-specific binding occurs, such materials can be readily removed from the surface without removing the oligonucleotides or proteins (such as antibodies).

The array formats of the present disclosure can be included in a variety of different types of formats. A “format” includes any format to which the solid support can be affixed, such as microtiter plates (e.g., multi-well plates), test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and reaction solutions).

In one embodiment, preformed nucleic acid probes, nucleic acid primers, and/or antibodies can be situated on or within the surface of a test region by any of a variety of conventional techniques, including photolithographic or silkscreen chemical attachment, disposition by ink jet technology, capillary, screen or fluid channel chip, electrochemical patterning using electrode arrays, contacting with a pin or quill, or denaturation followed by baking or UV-irradiating onto filters (see, e.g., Rava et al. (1996). U.S. Pat. No. 5,545,531; Fodor et al. (1996). U.S. Pat. No. 5,510,270; Zanzucchi et al. (1997). U.S. Pat. No. 5,643,738; Brennan (1995). U.S. Pat. No. 5,474,796; PCT WO 92/10092; PCT WO 90/15070).

In one embodiment, preformed nucleic acid probes or nucleic acid primers are derivatized at the 5′ end with a free amino group; dissolved at a concentration routinely determined empirically (e.g., about 1 μM) in a buffer such as 50 mM phosphate buffer, pH 8.5 and 1 mM EDTA; and distributed with a Pixus nanojet dispenser (Cartesian Technologies) in droplets of about 10.4 nanoliters onto specific locations within a test well whose upper surface is that of a fresh, dry DNA Bind plate (Corning Costar). In another embodiment, preformed nucleic acid probes or nucleic acid primers are derivatized at the 3′ end with a free amino group and optionally include a carbon spacer. Oligonucleotides are dissolved at 20 μM in 0.5 M Phosphate buffer at pH 8.5 and are contact printed on Falcon 1172 plates, gamma irradiated (BD Biosciences) using capillary pins in a humidified chamber. Depending on the relative rate of attachment and evaporation, it may be required to control the humidity in the wells during preparation.

In another embodiment, nucleic acid probes or nucleic acid primers can be synthesized directly on the surface of a test region, using methods such as, for example, light-activated deprotection of growing oligonucleotide chains (for example, in conjunction with the use of a site directing “mask”) or by patterned dispensing of nanoliter droplets of deactivating compound using a nanojet dispenser. Deprotection of all growing oligonucleotides that are to receive a single nucleotide can be done, for example, and the nucleotide then added across the surface. In another embodiment, oligonucleotide anchors are attached to the surface via the 3′ ends of the oligonucleotides, using conventional methodology.

In particular examples, the nucleic acid probe(s), nucleic acid primer(s), and/or antibodies are immobilized in an array (such as a microarray) and a label is detected using a microarray imager. Microarray imagers are commercially available and include OMIX, OMIX HD, CAPELLA, or SUPERCAPELLA imagers (HTG Molecular Diagnostics, Tucson, Ariz.), TYPHOON imager (GE Life Sciences, Piscataway, N.J.), GENEPIX microarray scanner (Molecular Devices, Sunnyvale, Calif.), or GENECHIP scanner (Affymetrix, Santa Clara, Calif.). In other examples, the nucleic acid probes, nucleic acid primers, and/or antibodies can be immobilized on a bead and the label is detected by flow cytometry or related methods (such as utilizing a LUMINEX 200 or FLEXMAP 3D (Luminex Corporation, Austin, Tex.), or other suitable instrument).

### IV. Methods of Treatment

Subjects with sepsis (whether symptomatic or pre-symptomatic) identified using the disclosed methods can be treated for sepsis. Thus, in some embodiments, the disclosed methods include administering one or more treatments for sepsis. The methods disclosed herein can be used to treat a subject who does not exhibit symptoms of sepsis, thereby decreasing the severity and/or duration of sepsis in the subject, or inhibiting development of sepsis. In other embodiments, the methods disclosed herein can be used to treat a subject who exhibits symptoms of sepsis and/or is diagnosed with sepsis by one or more other criteria, such as Sepsis-2 or Sepsis-3 criteria (e.g., Levy et al., Crit. Care Med. 31:1250-1256, 2003; Singer et al., JAMA 315:801-810, 2016).

In some examples, one or more therapies for sepsis, such as antibiotic therapy (for example, oral, intravenous, and/or intramuscular antibiotics), intravenous fluids, vasopressors (e.g., epinephrine, norepinephrine, and/or vasopressin), or other supportive therapies, can be administered to the subject. In additional examples, treatment can include surgery, for example to treat or remove infected tissue (including amputation of one or more affected extremities). Additional treatments, including corticosteroids, insulin, blood transfusion, dialysis, and/or mechanical ventilation may be administered in some cases. In other examples, the subject does not exhibit symptoms of sepsis, but is predicted to develop sepsis by the methods disclosed herein, and the subject is monitored for development of symptoms of sepsis. The subject may also be retested one or more times using the methods disclosed herein to diagnose sepsis at a later time point, and may be placed on a treatment protocol if sepsis is diagnosed.

Exemplary antibiotics that can be administered include ceftriaxone, azithromycin, ciprofloxacin, vancomycin, aztreonam, moxifloxacin, nafcillin, daptomycin, meropenem, ceftazidime, cefotaxime, cefepime, piperacillin-tazobactam, ampicillin-sulbactam, imipenem/cilastatin, levofloxacin), clindamycin, and combinations of two or more thereof. Specific antibiotics can be selected if the organism(s) causing the infection are identified. In some examples, the subject is treated with one or more broad-spectrum antibiotics immediately upon diagnosis, for example, prior to identifying a causative agent. The subject can then be administered one or more additional or different antibiotics when a specific causative agent is identified.

In other examples, the subject can be administered antiviral therapy, such as acyclovir, pocapavir or ganciclovir, if the underlying infection is known or suspected to be viral (such as herpes virus or enterovirus).

### EXAMPLES

The following examples are provided to illustrate certain particular features and/or embodiments. These examples should not be construed to limit the disclosure to the particular features or embodiments described.

### Example 1

### Development of a Biological Function-Based Sepsis Panel

### Materials and Methods

A previous 11 gene panel for sepsis prediction (CEACAM1, ZDHHC19, C9orf95, GNA15, BATF, C3AR1, KIAA1370, TGFB1, MTCH1, RPGRIP1, and HLA-DPB1; referred to as the “Stanford11” panel) was previously developed from a panel of 82 differentially expressed genes using the greedy forward search method (Sweeney et al., Sci. Transl. Med. 7:287ra71, 2015). This set of 82 genes (Stanford82; Table 1) was used as the basis for developing new biological-function based sepsis panels. Exemplary, non-limiting GenBank Accession numbers are provided for each gene in Table 1. Additional sequences for each gene can be identified using publicly available databases.

TABLE 1
The list of 82 differentially expressed genes (Stanford82)

| Gene | GenBank Acc. | Summary | Gene | GenBank Acc. | Summary |
|---|---|---|---|---|---|
| symbol | No. | Effect | symbol | No. | Effect* |
| ADAMTS3 | NM_014243 | 1 | PLB1 | NM_153021 | 1 |
| ANKRD22 | NM_114590 | 1 | PNPLA1 | NM_173676 | 1 |
| ANXA3 | NM_005139 | 1 | PPM1M | NM_144641 | 1 |
| AP3B2 | NM_004644 | 1 | PSTPIP2 | NM_024430 | 1 |
| ARL8A | NM_138795 | 1 | RETN | NM_020415 | 1 |
| B3GNT8 | NM_198540 | 1 | RGL4 | NM_153615 | 1 |
| BATF | NM_006399 | 1 | S100A12 | NM_005621 | 1 |
| BPI | NM_001725 | 1 | SEPHS2 | NM_012248 | 1 |
| BST1 | NM_004334 | 1 | SETD8 | NM_020382 | 1 |
| C1orf162 | NM_174896 | 1 | SGSH | NM_000199 | 1 |
| C3AR1 | NM_004054 | 1 | SIGLEC9 | NM_014441 | 1 |
| C9orf103 | NM_001256915 | 1 | SLC26A8 | NM_052961 | 1 |
| C9orf95 | NM_017881 | 1 | SPPL2A | NM_032802 | 1 |
| CCR1 | NM_001295 | 1 | SQRDL | NM_021199 | 1 |
| CD177 | NM_020406 | 1 | TCN1 | NM_001062 | 1 |
| CD63 | NM_001780 | 1 | ZDHHC19 | NM_001039617 | 1 |
| CD82 | NM_002231 | 1 | ZDHHC3 | NM_016598 | 1 |
| CEACAM1 | NM_001712 | 1 | ARHGEF18 | NM_015318 | −1 |
| CLEC5A | NM_013252 | 1 | CACNA2D3 | NM_018398 | −1 |
| DHRS9 | NM_199204 | 1 | CNNM3 | NM_017623 | −1 |
| EMR1 | NM_001974 | 1 | GLO1 | NM_006708 | −1 |
| FAM89A | NM_198552 | 1 | GRAMD1C | NM_017577 | −1 |
| FCER1G | NM_004106 | 1 | HACL1 | NM_012260 | −1 |
| FCGR1B | NM_001017986 | 1 | HLA-DPB1 | NM_002121 | −1 |
| FES | NM_002005 | 1 | KIAA1370 | NM_019600 | −1 |
| FFAR3 | NM_005304 | 1 | KLHDC2 | NM_014315 | −1 |
| FIG4 | NM_014845 | 1 | METAP1 | NM_015143 | −1 |
| GNA15 | NM_002068 | 1 | MRPS35 | NM_021821 | −1 |
| GPR84 | NM_020370 | 1 | MTCH1 | NM_014341 | −1 |
| HK3 | NM_002115 | 1 | NOC3L | NM_022451 | −1 |
| HP | NM_005143 | 1 | ODC1 | NM_002539 | −1 |
| IL10 | NM_000572 | 1 | PRKRIR | NM_004705 | −1 |
| IL18R1 | NM_003855 | 1 | RPGRIP1 | NM_020366 | −1 |
| KCNE1 | NM_000219 | 1 | RPUSD4 | NM_032795 | −1 |
| LCN2 | NM_005564 | 1 | SETD1B | NM_001353345 | −1 |
| LIN7A | NM_004664 | 1 | TBC1D4 | NM_014832 | −1 |
| OSCAR | NM_130771 | 1 | TGFBI | NM_000358 | −1 |
| OSTalpha | NM_152672 | 1 | TOMM20 | NM_014765 | −1 |
| P2RX1 | NM_002558 | 1 | UBE2Q2 | NM_173469 | −1 |
| PADI2 | NM_007365 | 1 | WDR75 | NM_032468 | −1 |
| PECR | NM_018441 | 1 | PLB1 | NM_153021 | 1 |
| PLAC8 | NM_016619 | 1 | PNPLA1 | NM_173676 | 1 |

*Summary Effect indicates the direction of fold changes. ‘1’ and ‘−1’ mean up- and down-regulation in sepsis compared to SIRS/trauma, respectively.

A list of substitutable genes in Stanford11 was generated based on gene ontology biological processes (GOBPs). The biological functions associated with the Stanford82 genes was first analyzed through functional enrichment analyses of GOBPs using DAVID (Database for Annotation Visualization and Integrated Discovery) (Huang et al., Nature Protocols 4:44-57, 2009). GO Direct category, which provides GO mappings directly annotated by the source database, was used. The terms with >1 genes were selected as “Stanford82-associated GOBPs.” The GOBPs that were represented by at least two genes, at least one of which derived from the Stanford11 genes were selected and visualized as a network using Enrichment Map v2.1.0, a Cytoscape plugin (Merico et al., PLoS One 5:e13984, 2010). The connected GOBP terms were merged and defined as a single functional category. Genes involved in the merged GOBPs and having the same directional changes were defined as substitutable candidates in the same GOBP.

Twelve microarray datasets that were used in two studies by Sweeney et al. (Table 2; Sweeney et al., Critical Care Medicine 45:1-10, 2017) were utilized. The first nine datasets are the discovery set that was used to identify the Stanford11 panel and the last three datasets are independent validation sets. The microarray datasets were normalized by the same methods used by Sweeney et al. Briefly, Affymetrix datasets were normalized using RMA or gcRMA (R package affy) (Gautier et al., Bioinformatics 20:307-315, 2004). Agilent and Illumina datasets were background corrected based on normal-exponential convolution model and then between-arrays quantile normalized using R package limma (Ritchie et al., Nucleic Acids Research 43:e47, 2015). The mean of multiple probes for common genes was used as the gene expression level after normalization. In the case of GSE74224, there was no probe for KIAA1370 of Stanford11; therefore, 10 genes were used to compute the performance of Stanford11 for this data.

TABLE 2
The 9 discovery and 3 independent microarray datasets and the performance of Stanford11.

| | | | | | | | | Lower | Upper |
|---|---|---|---|---|---|---|---|---|---|
| | | | | | | | | bound | bound |
| | | | | | # | # | | of 95% | of 95% |
| | Dataset | Platform | Control | Case | Control | Case | AUC | CI | CI |
| Discovery | GSE28750 | GPL570 | 24 hours | Community- | 11 | 10 | 0.96 | 0.89 | 1 |
| set | | | post-major | acquired | | | | | |
| | | | surgery | sepsis | | | | | |
| | GSE32707 | GPL10558 | Medical | Sepsis, | 55 | 48 | 0.8 | 0.71 | 0.88 |
| | | | ICU± | sepsis + | | | | | |
| | | | SIRS, | ARDS | | | | | |
| | | | nonseptic | | | | | | |
| | GSE40012 | GPL6947 | ICU SIRS | Sepsis from | 24 | 52 | 0.71 | 0.59 | 0.83 |
| | | | (66% | CAP | | | | | |
| | | | trauma) | | | | | | |
| | GSE66099 | GPL570 | Pediatric | Sepsis and | 30 | 199 | 0.79 | 0.73 | 0.86 |
| | | | ICU SIRS | septic shock | | | | | |
| | Glue Grant | GPL570 | Trauma | Trauma | 65 | 9 | 0.91 | 0.83 | 1 |
| | Buffy coat, | | patients | patients ± | | | | | |
| | day [1-3) | | without | 24 hours | | | | | |
| | | | infection | from | | | | | |
| | | | | diagnosis of | | | | | |
| | | | | infection | | | | | |
| | Glue Grant | GPL570 | Trauma | Trauma | 63 | 17 | 0.89 | 0.8 | 0.98 |
| | Buffy coat, | | patients | patients ± | | | | | |
| | day [3-6) | | without | 24 hours | | | | | |
| | | | infection | from | | | | | |
| | | | | diagnosis of | | | | | |
| | | | | infection | | | | | |
| | Glue Grant | GPL570 | Trauma | Trauma | 50 | 15 | 0.92 | 0.84 | 1 |
| | Buffy coat, | | patients | patients ± | | | | | |
| | Day [6-10) | | without | 24 hours | | | | | |
| | | | infection | from | | | | | |
| | | | | diagnosis of | | | | | |
| | | | | infection | | | | | |
| | Glue Grant | GPL570 | Trauma | Trauma | 22 | 4 | 0.85 | 0.7 | 1 |
| | Buffy coat, | | patients | patients ± | | | | | |
| | day [10-18) | | without | 24 hours | | | | | |
| | | | infection | from | | | | | |
| | | | | diagnosis of | | | | | |
| | | | | infection | | | | | |
| | Glue Grant | GPL570 | Trauma | Trauma | 6 | 4 | 0.96 | 0.84 | 1 |
| | Buffy coat, | | patients | patients ± | | | | | |
| | day [18-24) | | without | 24 hours | | | | | |
| | | | infection | from | | | | | |
| | | | | diagnosis of | | | | | |
| | | | | infection | | | | | |
| Validation | GSE65682 | GPL13667 | ICU | CAP | 33 | 101 | 0.78 | 0.68 | 0.87 |
| set | | | noninfected | | | | | | |
| | GSE74224 | GPL5175 | Postop | Sepsis | 31 | 74 | 0.88 | 0.82 | 0.95 |
| | E-MEXP- | GPL10332 | Hospitalized | Infection | 14 | 14 | 0.74 | 0.55 | 0.93 |
| | 3589 | | COPD | | | | | | |

Dataset and platform are NCBI GEO accession numbers.
CAP is community acquired pneumonia.
COPD is chronic obstructive pulmonary disease.
ARDS is acute respiratory distress syndrome.

In order to systematically evaluate the effect of gene substitution and reduction on classification performance, four different procedures were used as follows: 1) substitute one gene at a time; 2) substitute all possible genes; 3) retain only one gene for a GOBP where more than two genes are involved; 4) reduce panel by selecting one gene in each GOBP.

In order to evaluate the impact of biological function information in classification performance, three different approaches were used as follows: 1) use the 11 highest correlated genes based on expression profile with Stanford11 regardless of their biological functions; 2) use 11 randomly selected genes involved in chemotaxis, adhesion and migration biological function—one of GOBP terms associated with Stanfordl 1; and 3) k-Top Scoring Pairs classifier (kTSP) using switchbox R package to identify a small set of paired genes (Afsari et al., Bioinformatics 31:273-274, 2015). The AUC of kTSP was calculated by defining the number of votes among k pairs as a diagnostic score (Marchionni et al., BMC Genomics 14:336, 2014). The chemotaxis, adhesion and migration GOBP terms were selected as they contain more than 11 genes that can be fully substituted for Stanford11 genes. The classification performance of randomly selected gene sets was also tested by generating 100,000 combinations of 11 genes randomly selected from the Stanford82 genes. Classification performance was analyzed, along with biological processes that the 100,000 gene sets are involved in. The top and bottom 250 gene sets in order of performance were selected. The hierarchical clustering was applied to cluster GOBPs based on the number of genes in each GOBP from the top and bottom 250 gene sets.

### Results

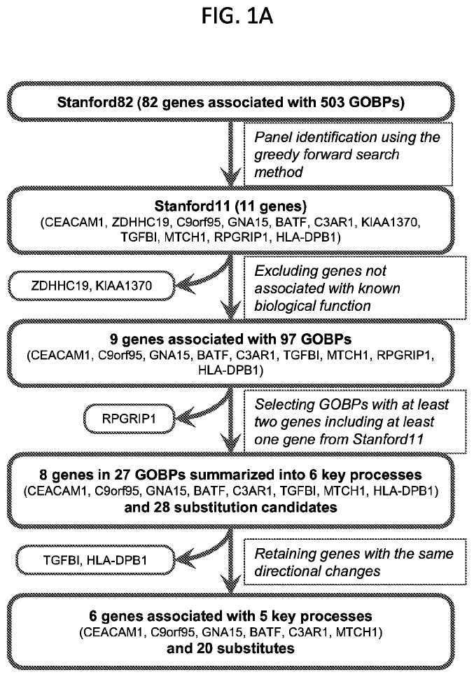

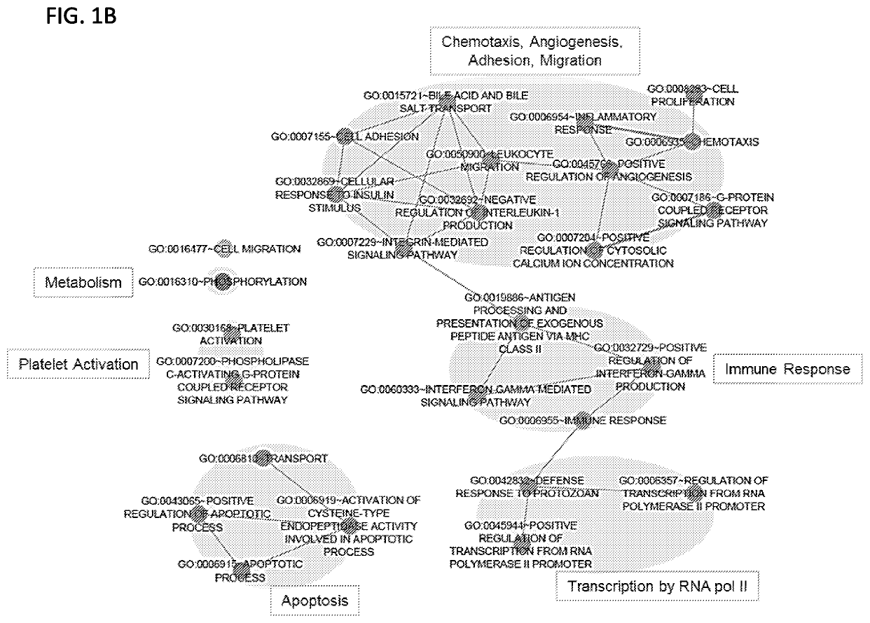

The overall procedure and the results of identifying substitutable genes for Stanford11 are summarized in FIG. 1A. There were 503 GOBPs represented by at least one of the Stanford82 genes. The genes in the Stanford11 panel were involved in 97 different GOBPs, with the exception of ZDHHC19 and KIAA1370 which are not associated with known biological function. Among the 97 GOBPs associated with Stanford11, 27 of them had at least one additional gene from the Stanford82 gene pool that is not in the Stanford11 panel and can be used as potential substitution candidates. These 27 GOBPs are represented by six key biological processes; 1) chemotaxis, angiogenesis, adhesion, migration; 2) immune response; 3) transcription by pol II; 4) platelet activation; 5) apoptosis; and 6) metabolism (FIG. 1B). Substitution candidates that are involved in these six key biological processes associated with the Stanford11 panel are summarized in Table 3. Among the six key biological processes, two processes, adhesion/migration and platelet activation, have more than two genes in the panel and the remaining four processes have just one gene. RPGR1P1, a gene associated with cell development and proliferation, was substitutable only with the genes already included in the Stanford11 panel; therefore, it was excluded for gene substitution. Among the substitutable genes, only genes with the same directional changes as genes in the Stanford11 panel were tested, e.g., increased or decreased expression level in sepsis patients compared to controls. Among the 28 substitution candidates, 20 genes were retained for six genes in the Stanford11 panel (CEACAM1, C3AR1, GNA15, BATF, MTCH1, and C9orf95) representing five biological functions. TGFB1 for the GOBP chemotaxis, angiogenesis, adhesion, migration was removed since no substitutable gene with the same directional change was retained. There was also no substitutable gene for HLA-DPB1 for immune response function; therefore, HLA-DPB1 was retained to keep all six biological processes during substitution and reduction procedures. Most of the six genes that can be substituted in the Stanford11 panel showed increased expression level in sepsis patients except MTCH1, a gene involved in apoptosis (Table 3).

TABLE 3
The substitutable genes of Stanford11. ↑and ↓ indicates up or down regulation in sepsis,
respectively. No substitutable gene was retained for TGFBI and HLA-DPB1 after considering consistency
in directional changes. Finally, 20 genes were retained for replacing six original Stanford11 genes.

| | | Interferon- | | | | |
|---|---|---|---|---|---|---|
| | | gamma, | | | | |
| | Chemotaxis, | antigen | | PLC, | | |
| | angiogenesis, | processing, | Transcription | phosphorylation, | | |
| | adhesion, | immune | by RNA | platelet | | |
| | migration | response | pol II | activation | Apoptosis | Metabolism |
| CEACAM1↑ | ADAMTS3↑, | | | | | |
| | CCR1↑, | | | | | |
| | CD177↑, | | | | | |
| | CD63↑, | | | | | |
| | EMR1↑, | | | | | |
| | FCER1G↑, | | | | | |
| | IL10↑, | | | | | |
| | OSTalpha↑, | | | | | |
| | PSTPIP2↑, | | | | | |
| | SIGLEC9↑, | | | | | |
| | TBC1D4↓, | | | | | |
| C3AR1↑ | ANXA3↑, | | | GPR84↑ | | |
| | CCR1↑, | | | | | |
| | CD177↑, | | | | | |
| | EMR1↑, | | | | | |
| | FCER1G↑, | | | | | |
| | FES↑, | | | | | |
| | FFAR3↑, | | | | | |
| | IL10↑, | | | | | |
| | S100A12↑ | | | | | |
| TGFBI↓ | CCR1↑, | | | | | |
| | EMR1↑, | | | | | |
| | FES↑, | | | | | |
| | SIGLEC9↑ | | | | | |
| GNA15↑ | CCR1↑, | | | FCER1G↑, | | |
| | EMR1↑, | | | P2RX1↑ | | |
| | FFAR3↑ | | | | | |
| HLA-DPB1↓ | | BPI↑, | | | | |
| | | CCR1↑, | | | | |
| | | FCER1G↑, | | | | |
| | | FCGR1B↑, | | | | |
| | | IL10↑, | | | | |
| | | IL18R1↑ | | | | |
| BATF↑ | | | IL10↑, | | | |
| | | | PLAC8↑, | | | |
| | | | GLO1↓, | | | |
| | | | PRKRIR↓, | | | |
| | | | WDR75↓ | | | |
| MTCH1↓ | | | | | LCN2↑, | |
| | | | | | OSTalpha↑, | |
| | | | | | P2RX1↑, | |
| | | | | | ARHGEF18↓ | |
| C9orf95↑ | | | | | | C9orf103↑, |
| | | | | | | SEPHS2↑ |

Since three genes (ZDHHC19, KIAA1370, and RPGRIP1) were excluded during the process of identification of substitutable genes, their contribution to the performance of Stanford11 was assessed. Excluding these genes from Stanford11 did not significantly affect the performance in both discovery and validation sets (Table 4). In the case of excluding all three genes, the performance decreased in one discovery and one validation set (Glue grant day [1-3] from 0.9145 to 0.865 and GSE74224 from 0.8814 to 0.8544), while increasing in one discovery set (GSE40012 from 0.7091 to 0.774). Therefore, these three genes contributed marginally to the diagnostic performance of Stanford11.

TABLE 4
The performances of reduced Stanford11 panels by excluding genes not associated
to known biological processes or with no substitutable genes.

| | | AUC | | | | P-value* | | |
|---|---|---|---|---|---|---|---|---|
| | | | | Exclude both | Exclude all | | Exclude both | Exclude all |
| | | | | KIAA1370 | RPGRIP1, | | KIAA1370 | RPGRIP1, |
| | | | Exclude | and | KIAA1370, | Exclude | and | KIAA1370, |
| | Dataset | Stanford11 | RPGRIP1 | ZDHHC19 | ZDHHC19 | RPGRIP1 | ZDHHC19 | ZDHHC19 |
| Discovery | GSE28750 | 0.9636 | 0.9636 | 0.8273 | 0.7818 | 1 | 0.1 | 0.0672 |
| set | GSE32707 | 0.7962 | 0.7894 | 0.7894 | 0.7678 | 0.4702 | 0.5455 | 0.0715 |
| | GSE40012 | 0.7091 | 0.7147 | 0.7588^{#} | 0.774^{#} | 0.7457 | 0.0174** | 0.0123** |
| | GSE66099 | 0.7948 | 0.8028^{#} | 0.799 | 0.8054 | 0.044** | 0.7218 | 0.3916 |
| | Glue Grant | 0.9145 | 0.9179 | 0.8632^{#} | 0.865^{#} | 0.2997 | 0.044** | 0.0434** |
| | Buffy coat, | | | | | | | |
| | day [1-3) | | | | | | | |
| | Glue Grant | 0.8898 | 0.8693 | 0.8898 | 0.8665 | 0.1139 | 1 | 0.2353 |
| | Buffy coat, | | | | | | | |
| | day [3-6) | | | | | | | |
| | Glue Grant | 0.9213 | 0.9107 | 0.8947 | 0.8613 | 0.502 | 0.2846 | 0.1136 |
| | Buffy coat, | | | | | | | |
| | Day [6-10) | | | | | | | |
| | Glue Grant | 0.8523 | 0.8295 | 0 | 0.7159 | 0.3865 | 0.8076 | 0.0784 |
| | Buffy coat, | | | | | | | |
| | day [10-18) | | | | | | | |
| | Glue Grant | 0.9583 | 0.875 | 0.9583 | 0.875 | 0.398 | 1 | 0.398 |
| | Buffy coat, | | | | | | | |
| | day [18-24) | | | | | | | |
| Validation | GSE65682 | 0.7792 | 0.7843 | 0.7384^{#} | 0.7459 | 0.3798 | 0.0118** | 0.398 |
| set | GSE74224 | 0.8814 | 0.8483^{#} | 0.8932 | 0.8544^{#} | 0.0018** | 0.0838 | 0.0186** |
| | E-MEXP- | 0.7398 | 0.7449 | 0.7296 | 0.7602 | 0.9025 | 0.6772 | 0.6443 |
| | 3589 | | | | | | | |

*The p-values were calculated by DeLong's test.
**indicates p-value less than 0.05.
^{#}indicates significantly higher/lower AUC than Stanford11 (P-value ≤ 0.05).

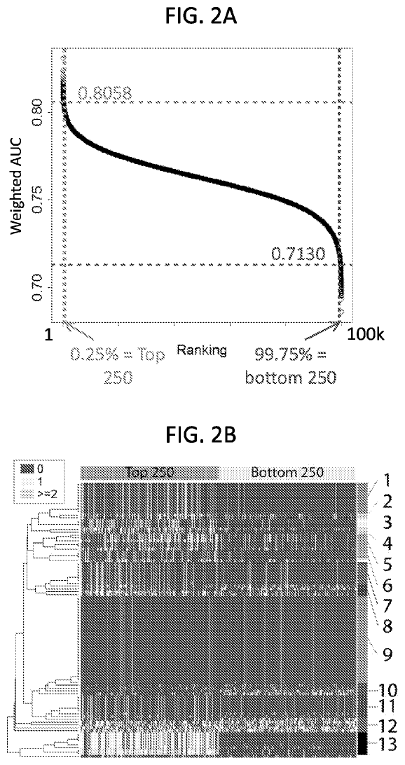

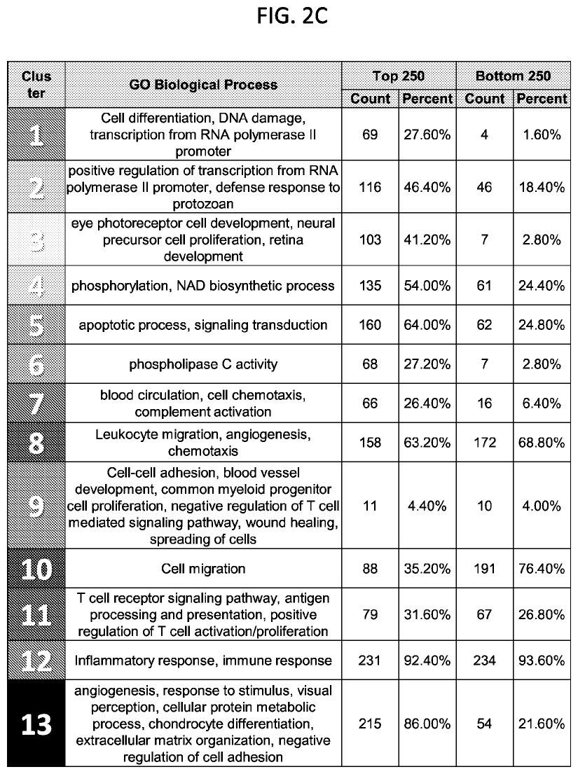

To determine the impact of biological processes in the performance of a diagnostic panel, 100,000 panels consisting of 11 genes randomly selected from the Stanford82 list were generated. The classification performances of the 100,000 random 11 member gene sets in the 9 discovery datasets were computed and sorted based on the performance as measured by AUC (FIG. 2A). The GOBP associated with genes in the top 250 and bottom 250 gene sets are summarized in FIG. 2B. The GOBPs represented by the top 250 gene sets were similar to the Stanford11 six key processes (FIG. 2B), such as transcription by pol II (cluster 1 and 2 in FIGS. 2B and 2C), phosphorylation (cluster 4), apoptosis (cluster 5), PLC (cluster 6), chemotaxis (cluster 7), antigen processing and presentation (cluster 11), and metabolic process (cluster 13). The cell development and proliferation process in cluster 3 was also frequently involved in high performing gene sets. RPGRIP1, which has no substitutable gene, was involved in this biological process. However, removing RPGRIP1 from Stanford11 did not significantly decrease diagnostic performance, as shown in Table 4.

A total of 12 microarray datasets from the public domain were used to evaluate the effect of gene substitution/reduction on the diagnostic performances of Stanford11. Nine of the datasets were used in the original discovery of the Stanford11 gene panel. The other three, GSE65682, GSE74224, and E-MTAB-3589, were not used in the development of Stanford11 panel; therefore, they were used as independent validation data in the study.

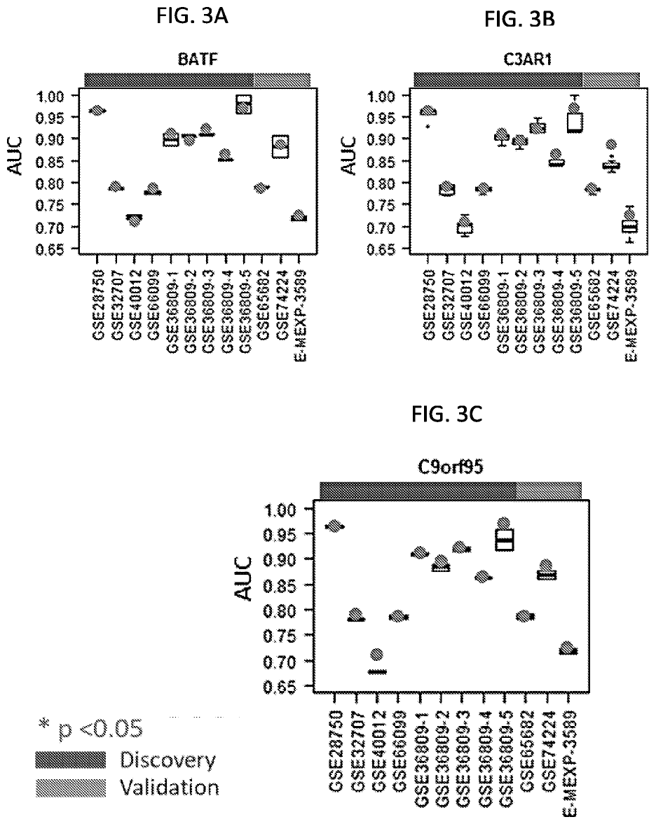

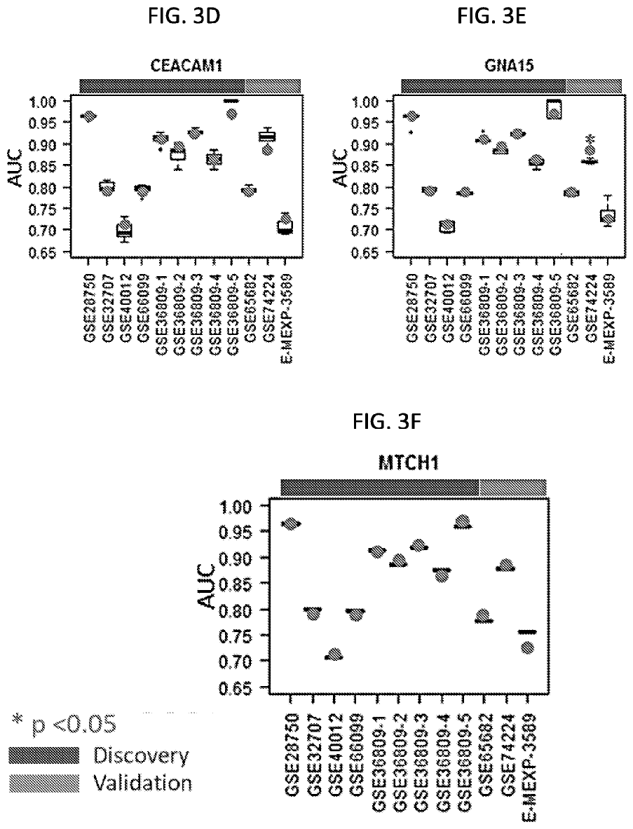

Based on the substitution candidates listed in Table 3, one gene was changed at a time for the six substitutable genes in the Stanford11 panel. As shown in FIGS. 3A-3F, one gene substitution did not affect the overall diagnostic performance in the nine discovery and three validation datasets. In the discovery datasets, the average AUCs of the substitutions were not significantly lower than the original Stanford11 panel, except using the GSE74224 dataset when replacing GNA15 (FIG. 3E).

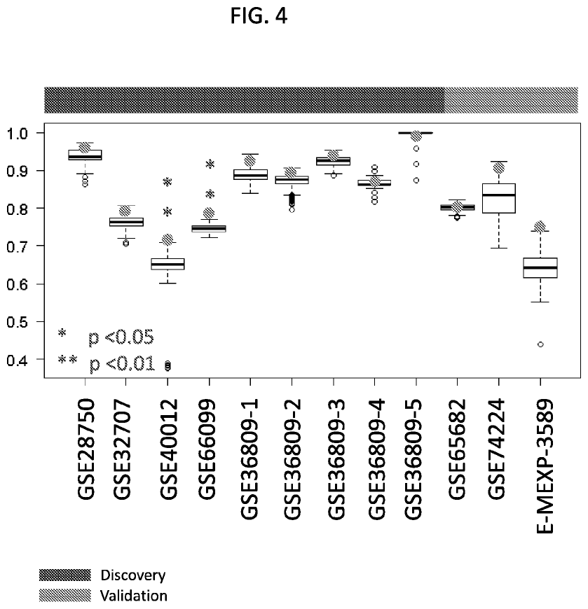

The effect of substituting genes representing all functional categories simultaneously was then tested. If a function had more than one substitutable gene, all combinations of genes were enumerated and their classification performances were tested and are summarized in FIG. 4. Except for the GSE40012 and GSE66099 datasets used in the Stanford11 discovery process, there were multiple five gene substitutions that showed similar performance as the original Stanford11. In summary, gene substitution based on the same biological process and direction of concentration changes can provide alternative panels that have similar diagnostic performance.

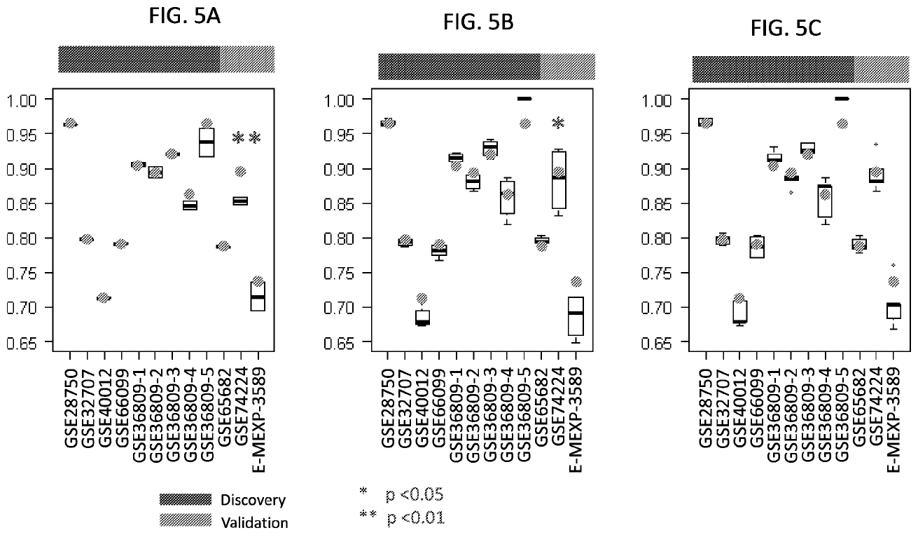

The possibility of using representative genes from each biological process to reduce the number of features in the diagnostic panel was investigated. Among the six representative biological processes of the Stanford11 panel, two biological functions have more than one gene in the panel. The GOBP “PLC, phosphorylation, platelet activation function” has two genes (C3AR1 and GNA15) and “chemotaxis, angiogenesis, adhesion, migration function” has four genes (CEACAM1, C3AR1, TGFB1, and GNA15) in the panel. We calculated AUCs of panels where only one gene was retained. As shown in FIG. 5A, the two panels with only one gene retained from “PLC, phosphorylation, platelet activation function” had similar performance to the Stanford11. The panels with only one gene from the GOBP “chemotaxis, angiogenesis, adhesion, migration function” also have similar performance in the datasets used to identify Stanford11 (FIG. 5B). The impact of retaining only one gene from both GOPBs was tested and the average diagnostic performance was not significantly different from the original Stanford11 panel in all the discovery and validation sets (FIG. 5C). In all cases, there were multiple panels with reduced features that delivered better performances in more than half of the independent validation datasets.

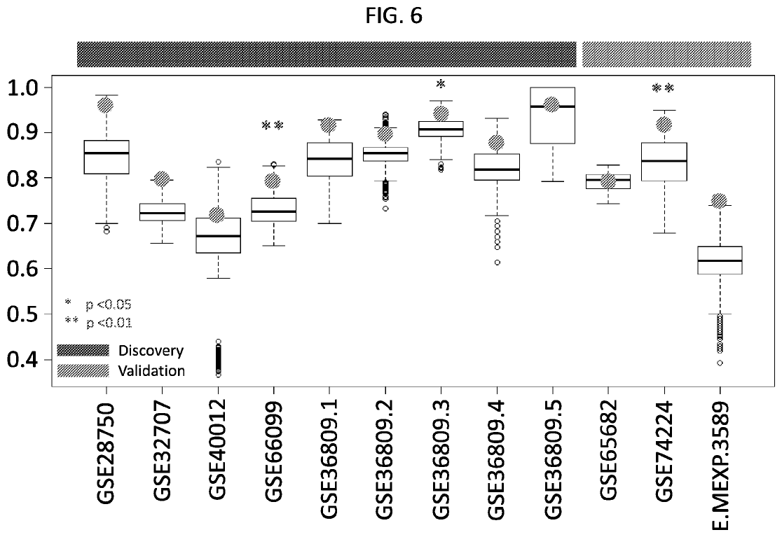

To explore the possibility of just using representative genes from key biological processes associated with Stanford11, all possible combinations of 6 gene panels were generated from genes mapped to those six biological processes. Diagnostic performance for all six-gene combinations was computed (FIG. 6). Among the 1,482 six-gene combinations, 73 new panels that have higher performance than the lower bound of 95% confidence intervals in all discovery datasets than the original panel were selected (Table 5). Among the 73 new panels, 22 panels had higher performance even in the two validation sets (GSE65682 and GSE74224, Table 6).

TABLE 5
73 new six-gene panels including six genes from the six key biological processes. The 73 panels have higher performance
than the lower bound of 95% confidence intervals in all discovery datasets than the original Stanford11.

| | Interferon- | | | | | | | | |
|---|---|---|---|---|---|---|---|---|---|
| | gamma, | | | | | | | | |
| Chemotaxis, | antigen | | PLC, | | | | | | |
| angiogenesis, | processing, | Transcription | phosphorylation, | | | | | | |
| adhesion, | immune | by RNA | platelet | | | Discovery set | | | |
| migration | response | pol II | activation | Apoptosis | Metabolism | GSE | GSE | GSE | GSE |
| Gene1 | Gene2 | Gene3 | Gene4 | Gene5 | Gene6 | 28750 | 32707 | 40012 | 66099 |
| ADAMTS3 | HLA-DPB1 | BATF | FCER1G | ARHGEF18 | C9orf95 | 0.8909 | 0.7648 | 0.6354 | 0.7648 |
| ADAMTS3 | HLA-DPB1 | BATF | FCER1G | MTCH1 | C9orf95 | 0.9182 | 0.7602 | 0.6587 | 0.7665 |
| CCR1 | HLA-DPB1 | BATF | GPR84 | MTCH1 | C9orf103 | 0.8909 | 0.7348* | 0.6338* | 0.7372* |
| CCR1 | HLA-DPB1 | BATF | FCER1G | ARHGEF18 | C9orf103 | 0.9000 | 0.7117* | 0.6042* | 0.7504* |
| CCR1 | HLA-DPB1 | BATF | FCER1G | ARHGEF18 | C9orf95 | 0.9364 | 0.7250* | 0.6795 | 0.7786 |
| CCR1 | HLA-DPB1 | BATF | FCER1G | MTCH1 | C9orf95 | 0.9455 | 0.7269* | 0.7003 | 0.7826 |
| CCR1 | HLA-DPB1 | BATF | C3AR1 | ARHGEF18 | C9orf95 | 0.8909 | 0.7568 | 0.6995 | 0.7896 |
| CCR1 | HLA-DPB1 | BATF | C3AR1 | MTCH1 | C9orf103 | 0.9000 | 0.7227* | 0.6675 | 0.7742 |
| CCR1 | HLA-DPB1 | BATF | C3AR1 | MTCH1 | C9orf95 | 0.9091 | 0.7542 | 0.7204 | 0.7940 |
| CCR1 | HLA-DPB1 | BATF | GNA15 | MTCH1 | C9orf95 | 0.9545 | 0.7352* | 0.7260 | 0.8062 |
| CD177 | HLA-DPB1 | BATF | GPR84 | MTCH1 | C9orf95 | 0.8909 | 0.7470* | 0.6274* | 0.739* |
| CD177 | HLA-DPB1 | BATF | FCER1G | ARHGEF18 | C9orf95 | 0.9182 | 0.7159* | 0.6050* | 0.7653 |
| CD177 | HLA-DPB1 | BATF | FCER1G | MTCH1 | C9orf95 | 0.9273 | 0.7182* | 0.6242* | 0.7635 |
| CD177 | HLA-DPB1 | BATF | C3AR1 | MTCH1 | C9orf95 | 0.8909 | 0.7458* | 0.6442* | 0.7784 |
| CD177 | HLA-DPB1 | BATF | GNA15 | ARHGEF18 | C9orf95 | 0.9273 | 0.7379* | 0.6202* | 0.7822 |
| CD177 | HLA-DPB1 | BATF | GNA15 | MTCH1 | C9orf95 | 0.9455 | 0.7333* | 0.6378* | 0.7874 |
| CD63 | HLA-DPB1 | PLAC8 | FCER1G | MTCH1 | C9orf95 | 0.9000 | 0.7231* | 0.7244 | 0.7387* |
| CD63 | HLA-DPB1 | PLAC8 | GNA15 | ARHGEFL8 | C9orf95 | 0.9273 | 0.7163* | 0.7372 | 0.7611 |
| CD63 | HLA-DPB1 | PLAC8 | GNA15 | MTCH1 | C9orf95 | 0.9636 | 0.7186* | 0.7620 | 0.7670 |
| CD63 | HLA-DPB1 | BATF | GPR84 | ARHGEF18 | C9orf95 | 0.8909 | 0.7496* | 0.6587 | 0.7466* |
| CD63 | HLA-DPB1 | BATF | GPR84 | MTCH1 | C9orf103 | 0.8909 | 0.7223* | 0.6386 | 0.7328* |
| CD63 | HLA-DPB1 | BATF | GPR84 | MTCH1 | C9orf95 | 0.8909 | 0.7481* | 0.6803 | 0.7497* |
| CD63 | HLA-DPB1 | BATF | FCER1G | ARHGEF18 | C9orf103 | 0.8909 | 0.7208* | 0.6090* | 0.7474* |
| CD63 | HLA-DPB1 | BATF | FCER1G | ARHGEF18 | C9orf95 | 0.9000 | 0.7420 | 0.6771 | 0.7784 |
| CD63 | HLA-DPB1 | BATF | FCER1G | MTCH1 | C9orf103 | 0.9091 | 0.7231* | 0.6346* | 0.7422* |
| CD63 | HLA-DPB1 | BATF | FCER1G | MTCH1 | C9orf95 | 0.9273 | 0.7424 | 0.6979 | 0.7789 |
| CD63 | HLA-DPB1 | BATF | C3AR1 | MTCH1 | C9orf95 | 0.8909 | 0.7431 | 0.7147 | 0.7953 |
| CD63 | HLA-DPB1 | BATF | GNA15 | ARHGEF18 | C9orf95 | 0.9182 | 0.7322 | 0.7188 | 0.7963 |
| CD63 | HLA-DPB1 | BATF | GNA15 | MTCH1 | C9orf95 | 0.9455 | 0.7299* | 0.7324 | 0.8005 |
| EMR1 | HLA-DPB1 | BATF | FCER1G | ARHGEF18 | C9orf95 | 0.9182 | 0.7216* | 0.6500 | 0.7658 |
| EMR1 | HLA-DPB1 | BATF | FCER1G | MTCH1 | C9orf103 | 0.8909 | 0.7117* | 0.5962* | 0.7353* |
| EMR1 | HLA-DPB1 | BATF | FCER1G | MTCH1 | C9orf95 | 0.9182 | 0.7159* | 0.6675 | 0.7675 |
| EMR1 | HLA-DPB1 | BATF | C3AR1 | ARHGEF18 | C9orf95 | 0.8909 | 0.7398* | 0.6707 | 0.7791 |
| EMR1 | HLA-DPB1 | BATF | C3AR1 | MTCH1 | C9orf95 | 0.8909 | 0.7341* | 0.6931 | 0.7851 |
| EMR1 | HLA-DPB1 | BATF | GNA15 | MTCH1 | C9orf95 | 0.9273 | 0.7159* | 0.6915 | 0.7844 |
| FCER1G | HLA-DPB1 | PLAC8 | C3AR1 | MTCH1 | C9orf95 | 0.9000 | 0.7273* | 0.7356 | 0.7628 |
| FCER1G | HLA-DPB1 | PLAC8 | GNA15 | ARHGEF18 | C9orf95 | 0.9182 | 0.7330 | 0.7220 | 0.7688 |
| FCER1G | HLA-DPB1 | PLAC8 | GNA15 | MTCH1 | C9orf95 | 0.9545 | 0.7322* | 0.7460 | 0.7719 |
| FCER1G | HLA-DPB1 | BATF | GPR84 | ARHGEF18 | C9orf95 | 0.8909 | 0.7508 | 0.6587 | 0.7521* |
| FCER1G | HLA-DPB1 | BATF | GPR84 | MTCH1 | SEPHS2 | 0.8909 | 0.7337* | 0.6234* | 0.7437* |
| FCER1G | HLA-DPB1 | BATF | GPR84 | MTCH1 | C9orf95 | 0.8909 | 0.7424* | 0.6755 | 0.7514* |
| FCER1G | HLA-DPB1 | BATF | C3AR1 | MTCH1 | SEPHS2 | 0.9000 | 0.7273* | 0.6619 | 0.7995 |
| FCER1G | HLA-DPB1 | BATF | C3AR1 | MTCH1 | C9orf95 | 0.9000 | 0.7413 | 0.7083 | 0.7980 |
| FCER1G | HLA-DPB1 | BATF | GNA15 | ARHGEF18 | C9orf95 | 0.9455 | 0.7413 | 0.6947 | 0.8027 |
| FCER1G | HLA-DPB1 | BATF | GNA15 | MTCH1 | SEPHS2 | 0.9727 | 0.7242* | 0.6747 | 0.7982 |
| FCER1G | HLA-DPB1 | BATF | GNA15 | MTCH1 | C9orf95 | 0.9818 | 0.7352 | 0.7123 | 0.8040 |
| OSTalpha | HLA-DPB1 | BATF | GNA15 | ARHGEF18 | C9orf95 | 0.8909 | 0.7701 | 0.6338* | 0.7496* |
| OSTalpha | HLA-DPB1 | BATF | QNA15 | MTCH1 | SEPHS2 | 0.8909 | 0.7295* | 0.6242* | 0.7367* |
| OSTalpha | HLA-DPB1 | BATF | GNA15 | MTCH1 | C9orf95 | 0.9091 | 0.7640 | 0.6474 | 0.7487* |
| SIGLEC9 | HLA-DPB1 | BATF | GNA15 | MTCH1 | C9orf95 | 0.9455 | 0.7799 | 0.6907 | 0.7506* |
| ANXA3 | HLA-DPB1 | BATF | GNA15 | MTCH1 | C9orf95 | 0.9091 | 0.7152* | 0.6530 | 0.7930 |
| FES | HLA-DPB1 | PLAC8 | GPR84 | MTCH1 | C9orf95 | 0.8909 | 0.7583 | 0.7027 | 0.7320* |
| FES | HLA-DPB1 | PLAC8 | FCER1G | MTCH1 | C9orf95 | 0.9091 | 0.7318* | 0.7204 | 0.7521 |
| FES | HLA-DPB1 | PLAC8 | GNA15 | ARHGEF18 | C9orf103 | 0.8909 | 0.7292* | 0.6643 | 0.7441* |
| FES | HLA-DPB1 | PLAC8 | GNA15 | ARHGEF18 | C9orf95 | 0.9000 | 0.7470 | 0.7364 | 0.7735 |
| FES | HLA-DPB1 | PLAC8 | GNA15 | MTCH1 | C9orf95 | 0.9545 | 0.7428 | 0.7500 | 0.7760 |
| FES | HLA-DPB1 | BATF | GPR84 | MTCH1 | C9orf95 | 0.8909 | 0.7765 | 0.6851 | 0.7553* |
| FES | HLA-DPB1 | BATF | FCER1G | ARHGEF18 | C9orf103 | 0.8909 | 0.7348* | 0.6050* | 0.7513 |
| FES | HLA-DPB1 | BATF | FCER1G | MTCH1 | C9orf103 | 0.9000 | 0.7341* | 0.6258* | 0.7497 |
| FES | HLA-DPB1 | BATF | C3AR1 | MTCH1 | C9orf95 | 0.9000 | 0.7799 | 0.7115 | 0.7970 |
| FES | HLA-DPB1 | BATF | GNA15 | ARHGEF18 | C9orf95 | 0.9273 | 0.7742 | 0.7011 | 0.7968 |
| FES | HLA-DPB1 | BATF | GNA15 | MTCH1 | C9orf95 | 0.9455 | 0.7670 | 0.7260 | 0.8022 |
| S100A12 | HLA-DPB1 | PLAC8 | FCER1G | MTCH1 | C9orf95 | 0.9000 | 0.7174* | 0.6907 | 0.7367* |
| S100A12 | HLA-DPB1 | BATF | GPR84 | ARHGEF18 | C9orf95 | 0.8909 | 0.7367* | 0.6482 | 0.7476* |
| S100A12 | HLA-DPB1 | BATF | QPR84 | MTCH1 | C9orf95 | 0.8909 | 0.7307* | 0.6603 | 0.7504* |
| S100A12 | HLA-DPB1 | BATF | FCER1G | ARHGEF18 | C9orf95 | 0.9000 | 0.7280* | 0.6482* | 0.7782 |
| S100A12 | HLA-DPB1 | BATF | FCER1G | MTCH1 | C9orf95 | 0.9182 | 0.7242* | 0.6611 | 0.7839 |
| S100A12 | HLA-DPB1 | BATF | C3AR1 | MTCH1 | C9orf95 | 0.9000 | 0.7311* | 0.6939 | 0.7998 |
| S100A12 | HLA-DPB1 | BATF | GNA15 | ARHGEF18 | C9orf95 | 0.9182 | 0.7197* | 0.6747 | 0.7963 |
| S100A12 | HLA-DPB1 | BATF | GNA15 | MTCH1 | C9orf103 | 0.9000 | 0.7106* | 0.6202* | 0.7693 |
| S100A12 | HLA-DPB1 | BATF | GNA15 | MTCH1 | C9orf95 | 0.9455 | 0.7201* | 0.6947 | 0.8044 |
| C3AR1 | HLA-DPB1 | BATF | GNA15 | ARHGEF18 | C9orf95 | 0.9182 | 0.7583 | 0.7091 | 0.8092 |
| C3AR1 | HLA-DPB1 | BATF | GNA15 | MTCH1 | C9orf95 | 0.9273 | 0.7542 | 0.7228 | 0.8136 |

| Discovery set | | | | | | | |
|---|---|---|---|---|---|---|---|
| Glue | Glue | Glue | Glue | Glue | | | |
| Grant | Grant | Grant | Grant | Grant | | | |
| Buffy | Buffy | Buffy | Buffy | Buffy | Validation set | | |
| coat, | coat, | coat, | coat, | coat, | | | E- |
| day | day | Day | day | day | GSE | GSE | MEXP- |
| [1-3) | [3-6) | [6-10) | [10-18) | [18-24) | 65682 | 74224 | 3589 |
| 0.8359* | 0.8711 | 0.9400 | 0.7386 | 0.9583 | 0.7789 | 0.7912* | 0.5612 |
| 0.8513* | 0.8711 | 0.9360 | 0.7386 | 0.9583 | 0.8119 | 0.8156* | 0.6378 |
| 0.8462* | 0.8936 | 0.9600 | 0.8182 | 0.9167 | 0.8218 | 0.8553 | 0.6378 |
| 0.8735 | 0.8627 | 0.9507 | 0.8750 | 0.9167 | 0.7951 | 0.8535 | 0.6378 |
| 0.8650* | 0.8655 | 0.9587 | 0.8295 | 0.9167 | 0.7840 | 0.8435 | 0.5969 |
| 0.8667* | 0.8861 | 0.9640 | 0.8523 | 0.9167 | 0.8128 | 0.8588 | 0.6684 |
| 0.8410* | 0.8768 | 0.9387 | 0.8750 | 0.9583 | 0.7807 | 0.8854 | 0.6633 |
| 0.8821 | 0.8814 | 0.9400 | 0.8750 | 0.9167 | 0.8170 | 0.9058 | 0.6990 |
| 0.8410* | 0.8945 | 0.9440 | 0.8409 | 0.9583 | 0.8095 | 0.8967 | 0.6786 |
| 0.8342* | 0.9094 | 0.9627 | 0.8636 | 0.9167 | 0.8080 | 0.8893 | 0.6378 |
| 0.8615 | 0.8534 | 0.9373 | 0.7955 | 1.0000 | 0.7933 | 0.8191* | 0.6327 |
| 0.8906 | 0.8142 | 0.9293 | 0.8182 | 1.0000 | 0.7762 | 0.8178* | 0.6429 |
| 0.8974 | 0.8161 | 0.9307 | 0.8182 | 1.0000 | 0.8032 | 0.8304 | 0.6378 |
| 0.8838 | 0.8366 | 0.9227 | 0.8295 | 1.0000 | 0.8014 | 0.8827 | 0.6684 |
| 0.8718 | 0.8403 | 0.9413 | 0.8409 | 1.0000 | 0.7699 | 0.8413 | 0.6429 |
| 0.8752 | 0.8450 | 0.9507 | 0.8295 | 1.0000 | 0.7972 | 0.8570 | 0.6531 |
| 0.8957 | 0.8973 | 0.9067 | 0.7500 | 0.9167 | 0.8158 | 0.9128 | 0.6122 |
| 0.8427 | 0.9328* | 0.9307 | 0.7955 | 1.0000 | 0.7852 | 0.9220 | 0.5663 |
| 0.8444 | 0.9300 | 0.9427 | 0.7727 | 1.0000 | 0.8107 | 0.9333* | 0.5918 |
| 0.8530 | 0.8758 | 0.9320 | 0.7955 | 1.0000 | 0.7705 | 0.8400 | 0.5969 |
| 0.8769 | 0.8739 | 0.9453 | 0.7841 | 0.8750 | 0.8116 | 0.8505 | 0.6327 |
| 0.8547 | 0.8711 | 0.9427 | 0.7841 | 1.0000 | 0.7993 | 0.8553 | 0.6071 |
| 0.8974 | 0.8478 | 0.9387 | 0.7955 | 0.8750 | 0.7942 | 0.8492 | 0.6224 |
| 0.8957 | 0.8347 | 0.9440 | 0.7273 | 1.0000 | 0.7780 | 0.8566 | 0.6122 |
| 0.9060 | 0.8609 | 0.9387 | 0.7614 | 0.8750 | 0.8179 | 0.8636 | 0.6684 |
| 0.9043 | 0.8478 | 0.9400 | 0.7386 | 0.9583 | 0.8107 | 0.8705 | 0.6276 |
| 0.8838 | 0.8609 | 0.9227 | 0.7614 | 1.0000 | 0.8080 | 0.9067 | 0.6633 |
| 0.8598 | 0.8702 | 0.9520 | 0.7955 | 0.9583 | 0.7726 | 0.8788 | 0.5969 |
| 0.8701 | 0.8805 | 0.9547 | 0.7727 | 1.0000 | 0.8029 | 0.8915 | 0.6378 |
| 0.8667 | 0.8581 | 0.9440 | 0.8636 | 1.0000 | 0.7768 | 0.8483 | 0.6071 |
| 0.8632 | 0.8599 | 0.9453 | 0.8636 | 0.8750 | 0.8161 | 0.8496 | 0.6429 |
| 0.8701 | 0.8646 | 0.9507 | 0.8636 | 1.0000 | 0.8137 | 0.8596 | 0.6071 |
| 0.8462 | 0.8665 | 0.9227 | 0.8750 | 1.0000 | 0.7795 | 0.8867 | 0.6327 |
| 0.8444 | 0.8702 | 0.9333 | 0.8409 | 1.0000 | 0.8149 | 0.8963 | 0.6582 |
| 0.8342 | 0.8833 | 0.9467 | 0.8523 | 1.0000 | 0.8044 | 0.8836 | 0.6071 |
| 0.8615* | 0.8599 | 0.8747 | 0.8068 | 1.0000 | 0.8080 | 0.9241 | 0.6531 |
| 0.8496 | 0.9104 | 0.9173 | 0.8182 | 1.0000 | 0.7861 | 0.9098 | 0.5408 |
| 0.8547 | 0.9188 | 0.9293 | 0.7841 | 1.0000 | 0.8092 | 0.9185 | 0.5816* |
| 0.8598 | 0.8609 | 0.9307 | 0.8182 | 1.0000 | 0.7747 | 0.8147* | 0.5816 |
| 0.8359* | 0.8459 | 0.9360 | 0.8068 | 1.0000 | 0.8038 | 0.8095* | 0.6122 |
| 0.8615 | 0.8665 | 0.9387 | 0.7727 | 0.9583 | 0.8041 | 0.833* | 0.6071 |
| 0.8667 | 0.8030* | 0.9187 | 0.8409 | 1.0000 | 0.8086 | 0.8867 | 0.6582 |
| 0.8889 | 0.8413 | 0.9227 | 0.8068 | 1.0000 | 0.8071 | 0.8963 | 0.6480 |
| 0.8735 | 0.8553 | 0.9520 | 0.7841 | 0.9583 | 0.7699 | 0.8640 | 0.5867 |
| 0.8359* | 0.8329 | 0.9480 | 0.8409 | 1.0000 | 0.8077 | 0.8614 | 0.6224 |
| 0.8872 | 0.8599 | 0.9547 | 0.7841 | 0.9583 | 0.8026 | 0.8749 | 0.6122 |
| 0.8940 | 0.8852 | 0.9280 | 0.8409 | 0.9167 | 0.7690 | 0.8147* | 0.5918 |
| 0.8855 | 0.8693 | 0.9320 | 0.8523 | 0.8750 | 0.8065 | 0.7960* | 0.6276 |
| 0.8991 | 0.8805 | 0.9307 | 0.8295 | 0.8750 | 0.8035 | 0.8326 | 0.6224 |
| 0.8906 | 0.8123* | 0.9320 | 0.8182 | 1.0000 | 0.8125 | 0.8745 | 0.6582 |
| 0.8581 | 0.8702 | 0.9373 | 0.8182 | 1.0000 | 0.7993 | 0.8383 | 0.6531 |
| 0.8427 | 0.8553 | 0.9160 | 0.7841 | 1.0000 | 0.7939 | 0.8758 | 0.6020 |
| 0.8906 | 0.8385 | 0.8920 | 0.7841 | 1.0000 | 0.8089 | 0.8945 | 0.6173 |
| 0.8701 | 0.8749 | 0.9187 | 0.8409 | 0.8750 | 0.7870 | 0.9098 | 0.6122 |
| 0.8427 | 0.8693 | 0.9147 | 0.8182 | 0.9583 | 0.7768 | 0.9102 | 0.5867 |
| 0.8513 | 0.8655 | 0.9120 | 0.8068 | 0.9583 | 0.8005 | 0.9150 | 0.6071 |
| 0.8479 | 0.8487* | 0.9227 | 0.7955 | 1.0000 | 0.7999 | 0.8309* | 0.6327 |
| 0.8855 | 0.8114* | 0.9347 | 0.7841 | 0.8750 | 0.7897 | 0.8069* | 0.6633 |
| 0.8889 | 0.8226* | 0.9333 | 0.8068 | 0.8750 | 0.8158 | 0.8291 | 0.6633 |
| 0.8667 | 0.8161* | 0.9160 | 0.7841 | 1.0000 | 0.8026 | 0.8806 | 0.6786 |
| 0.8564 | 0.8226 | 0.9320 | 0.7955 | 0.9583 | 0.7672 | 0.8470 | 0.6173 |
| 0.8598 | 0.8245 | 0.9427 | 0.7841 | 0.9583 | 0.7990 | 0.8627 | 0.6480 |
| 0.8855 | 0.8926 | 0.9053 | 0.7955 | 0.9583 | 0.8155 | 0.8718 | 0.6122 |
| 0.8479* | 0.8768 | 0.9280 | 0.7955 | 1.0000 | 0.7717 | 0.7951* | 0.6122 |
| 0.8513 | 0.8758 | 0.9320 | 0.7841 | 1.0000 | 0.8002 | 0.8112* | 0.6122 |
| 0.8940 | 0.8599 | 0.9507 | 0.7614 | 1.0000 | 0.7786 | 0.7942* | 0.6020 |
| 0.8957 | 0.8693 | 0.9493 | 0.7500 | 1.0000 | 0.8086 | 0.8134* | 0.6378 |
| 0.8752 | 0.8730 | 0.9093 | 0.7955 | 1.0000 | 0.8074 | 0.8670 | 0.6531 |
| 0.8564 | 0.8945 | 0.9453 | 0.7841 | 1.0000 | 0.7726 | 0.8352 | 0.6071 |
| 0.8855 | 0.9010 | 0.9547 | 0.8523 | 0.8750 | 0.8128 | 0.8405 | 0.6327 |
| 0.8684 | 0.8973 | 0.9560 | 0.8068 | 1.0000 | 0.8035 | 0.8461 | 0.6276 |
| 0.8513 | 0.8693 | 0.9187 | 0.8182 | 1.0000 | 0.7678 | 0.9050 | 0.6276 |
| 0.8479 | 0.8711 | 0.9267 | 0.8182 | 1.0000 | 0.7999 | 0.9133 | 0.6480 |

*indicates p-value from DeLong's test in comparison with the Stanford11 less than 0.05.

TABLE 7
Effect of adding RPGRIP1 to each of the 73 new six-gene panels.

| Chemotax., | | | PLC, | | | | | | |
|---|---|---|---|---|---|---|---|---|---|
| angiogen., | | Transcr. | phosphorylation, | | | | | | |
| adhesion, | Immune | by RNA | platelet | | | | Discovery set | | |
| migration | response | pol II | activation | Apoptosis | Metabolism | | GSE | GSE | GSE |
| Gene1 | Gene2 | Gene3 | Gene4 | Gene5 | Gene6 | Gene7 | 28750 | 32707 | 40012 |
| ADAMTS3 | HLA-DPB1 | BATF | FCER1G | ARHGEF18 | C9orf95 | RPGRIP1 | 0.9182 | 0.7932 | 0.6530 |
| ADAMTS3 | HLA-DPB1 | BATF | FCER1G | MTCH1 | C9orf95 | RPGRIP1 | 0.9273 | 0.7886 | 0.6659 |
| CCR1 | HLA-DPB1 | BATF | GPR84 | MTCH1 | C9orf103 | RPGRIP1 | 0.9182 | 0.7496 | 0.6458 |
| CCR1 | HLA-DPB1 | BATF | FCER1G | ARHGEF18 | C9orf103 | RPGRIP1 | 0.9455 | 0.7261 | 0.6338 |
| CCR1 | HLA-DPB1 | BATF | FCER1G | ARHGEF18 | C9orf95 | RPGRIP1 | 0.9364 | 0.7568 | 0.7019 |
| CCR1 | HLA-DPB1 | BATF | FCER1G | MTCH1 | C9orf95 | RPGRIP1 | 0.9545 | 0.7534 | 0.7179 |
| CCR1 | HLA-DPB1 | BATF | C3AR1 | ARHGEF18 | C9orf95 | RPGRIP1 | 0.9091 | 0.7758 | 0.7171 |
| CCR1 | HLA-DPB1 | BATF | C3AR1 | MTCH1 | C9orf103 | RPGRIP1 | 0.9273 | 0.7508 | 0.6819 |
| CCR1 | HLA-DPB1 | BATF | C3AR1 | MTCH1 | C9orf95 | RPGRIP1 | 0.9273 | 0.7742 | 0.7308 |
| CCR1 | HLA-DPB1 | BATF | GNA15 | MTCH1 | C9orf95 | RPGRIP1 | 0.9455 | 0.7652 | 0.7340 |
| CD177 | HLA-DPB1 | BATF | GPR84 | MTCH1 | C9orf95 | RPGRIP1 | 0.9000 | 0.7470 | 0.6546 |
| CD177 | HLA-DPB1 | BATF | FCER1G | ARHGEF18 | C9orf95 | RPGRIP1 | 0.9182 | 0.7424 | 0.6474 |
| CD177 | HLA-DPB1 | BATF | FCER1G | MTCH1 | C9orf95 | RPGRIP1 | 0.9455 | 0.7436 | 0.6563 |
| CD177 | HLA-DPB1 | BATF | C3AR1 | MTCH1 | C9orf95 | RPGRIP1 | 0.9091 | 0.7625 | 0.6675 |
| CD177 | HLA-DPB1 | BATF | GNA15 | ARHGEF18 | C9orf95 | RPGRIP1 | 0.9455 | 0.7564 | 0.6563 |
| CD177 | HLA-DPB1 | BATF | GNA15 | MTCH1 | C9orf95 | RPGRIP1 | 0.9727 | 0.7538 | 0.6707 |
| CD63 | HLA-DPB1 | PLAC8 | FCER1G | MTCH1 | C9orf95 | RPGRIP1 | 0.9182 | 0.7364 | 0.7220 |
| CD63 | HLA-DPB1 | PLAC8 | GNA15 | ARHGEF18 | C9orf95 | RPGRIP1 | 0.9545 | 0.7201 | 0.7356 |
| CD63 | HLA-DPB1 | PLAC8 | GNA15 | MTCH1 | C9orf95 | RPGRIP1 | 0.9636 | 0.7277 | 0.7388 |
| CD63 | HLA-DPB1 | BATF | GPR84 | ARHGEF18 | C9orf95 | RPGRIP1 | 0.8909 | 0.7595 | 0.6803 |
| CD63 | HLA-DPB1 | BATF | GPR84 | MTCH1 | C9orf103 | RPGRIP1 | 0.9000 | 0.7242 | 0.6426 |
| CD63 | HLA-DPB1 | BATF | GPR84 | MTCH1 | C9orf95 | RPGRIP1 | 0.8909 | 0.7617 | 0.6907 |
| CD63 | HLA-DPB1 | BATF | FCER1G | ARHGEF18 | C9orf103 | RPGRIP1 | 0.9091 | 0.7258 | 0.6378 |
| CD63 | HLA-DPB1 | BATF | FCER1G | ARHGEF18 | C9orf95 | RPGRIP1 | 0.9273 | 0.7545 | 0.6907 |
| CD63 | HLA-DPB1 | BATF | FCER1G | MTCH1 | C9orf103 | RPGRIP1 | 0.9364 | 0.7250 | 0.6482 |
| CD63 | HLA-DPB1 | BATF | FCER1G | MTCH1 | C9orf95 | RPGRIP1 | 0.9364 | 0.7542 | 0.6995 |
| CD63 | HLA-DPB1 | BATF | C3AR1 | MTCH1 | C9orf95 | RPGRIP1 | 0.9091 | 0.7678 | 0.7204 |
| CD63 | HLA-DPB1 | BATF | GNA15 | ARHGEF18 | C9orf95 | RPGRIP1 | 0.9273 | 0.7511 | 0.7083 |
| CD63 | HLA-DPB1 | BATF | GNA15 | MTCH1 | C9orf95 | RPGRIP1 | 0.9364 | 0.7519 | 0.7196 |
| EMR1 | HLA-DPB1 | BATF | FCER1G | ARHGEF18 | C9orf95 | RPGRIP1 | 0.9182 | 0.7470 | 0.6691 |
| EMR1 | HLA-DPB1 | BATF | FCER1G | MTCH1 | C9orf103 | RPGRIP1 | 0.9545 | 0.7201 | 0.3814 |
| EMR1 | HLA-DPB1 | BATF | FCER1G | MTCH1 | C9orf95 | RPGRIP1 | 0.9455 | 0.7420 | 0.6779 |
| EMR1 | HLA-DPB1 | BATF | C3AR1 | ARHGEF18 | C9orf95 | RPGRIP1 | 0.9091 | 0.7648 | 0.6803 |
| EMR1 | HLA-DPB1 | BATF | C3AR1 | MTCH1 | C9orf95 | RPGRIP1 | 0.9182 | 0.7663 | 0.6979 |
| EMR1 | HLA-DPB1 | BATF | GNA15 | MTCH1 | C9orf95 | RPGRIP1 | 0.9273 | 0.7489 | 0.6875 |
| FCER1G | HLA-DPB1 | PLAC8 | C3AR1 | MTCH1 | C9orf95 | RPGRIP1 | 0.9182 | 0.7462 | 0.7412 |
| FCER1G | HLA-DPB1 | PLAC8 | GNA15 | ARHGEF18 | C9orf95 | RPGRIP1 | 0.9455 | 0.7500 | 0.7300 |
| FCER1G | HLA-DPB1 | PLAC8 | GNA15 | MTCH1 | C9orf95 | RPGRIP1 | 0.9636 | 0.7481 | 0.7396 |
| FCER1G | HLA-DPB1 | BATF | GPR84 | ARHGEF18 | C9orf95 | RPGRIP1 | 0.8909 | 0.7708 | 0.6731 |
| FCER1G | HLA-DPB1 | BATF | GPR84 | MTCH1 | SEPHS2 | RPGRIP1 | 0.8909 | 0.7462 | 0.6410 |
| FCER1G | HLA-DPB1 | BATF | GPR84 | MTCH1 | C9orf95 | RPGRIP1 | 0.9091 | 0.7693 | 0.6811 |
| FCER1G | HLA-DPB1 | BATF | C3AR1 | MTCH1 | SEPHS2 | RPGRIP1 | 0.9182 | 0.7428 | 0.6827 |
| FCER1G | HLA-DPB1 | BATF | C3AR1 | MTCH1 | C9orf95 | RPGRIP1 | 0.9000 | 0.7674 | 0.7196 |
| FCER1G | HLA-DPB1 | BATF | GNA15 | ARHGEF18 | C9orf95 | RPGRIP1 | 0.9455 | 0.7659 | 0.7035 |
| FCER1G | HLA-DPB1 | BATF | GNA15 | MTCH1 | SEPHS2 | RPGRIP1 | 0.9727 | 0.7383 | 0.6659 |
| FCER1G | HLA-DPB1 | BATF | GNA15 | MTCH1 | C9orf95 | RPGRIP1 | 0.9636 | 0.7598 | 0.7155 |
| OSTalpha | HLA-DPB1 | BATF | GNA15 | ARHGEF18 | C9orf95 | RPGRIP1 | 0.9273 | 0.7780 | 0.6522 |
| OSTalpha | HLA-DPB1 | BATF | GNA15 | MTCH1 | SEPHS2 | RPGRIP1 | 0.9364 | 0.7538 | 0.6314 |
| OSTalpha | HLA-DPB1 | BATF | GNA15 | MTCH1 | C9orf95 | RPGRIP1 | 0.9364 | 0.7788 | 0.6595 |
| SIGLEC9 | HLA-DPB1 | BATF | GNA15 | MTCH1 | C9orf95 | RPGRIP1 | 0.9455 | 0.8000 | 0.6931 |
| ANXA3 | HLA-DPB1 | BATF | GNA15 | MTCH1 | C9orf95 | RPGRIP1 | 0.9182 | 0.7470 | 0.6859 |
| FES | HLA-DPB1 | PLAC8 | GPR84 | MTCH1 | C9orf95 | RPGRIP1 | 0.9000 | 0.7803 | 0.7003 |
| FES | HLA-DPB1 | PLAC8 | FCER1G | MTCH1 | C9orf95 | RPGRIP1 | 0.9273 | 0.7519 | 0.7091 |
| FES | HLA-DPB1 | PLAC8 | GNA15 | ARHGEF18 | C9orf103 | RPGRIP1 | 0.9273 | 0.7394 | 0.6635 |
| FES | HLA-DPB1 | PLAC8 | GNA15 | ARHGEF18 | C9orf95 | RPGRIP1 | 0.9364 | 0.7727 | 0.7212 |
| FES | HLA-DPB1 | PLAC8 | GNA15 | MTCH1 | C9orf95 | RPGRIP1 | 0.9455 | 0.7686 | 0.7356 |
| FES | HLA-DPB1 | BATF | GPR84 | MTCH1 | C9orf95 | RPGRIP1 | 0.8909 | 0.7917 | 0.6819 |
| FES | HLA-DPB1 | BATF | FCER1G | ARHGEF18 | C9orf103 | RPGRIP1 | 0.9000 | 0.7500 | 0.6298 |
| FES | HLA-DPB1 | BATF | FCER1G | MTCH1 | C9orf103 | RPGRIP1 | 0.9364 | 0.7436 | 0.6394 |
| FES | HLA-DPB1 | BATF | C3AR1 | MTCH1 | C9orf95 | RPGRIP1 | 0.9091 | 0.7989 | 0.7188 |
| FES | HLA-DPB1 | BATF | GNA15 | ARHGEF18 | C9orf95 | RPGRIP1 | 0.9273 | 0.7936 | 0.7035 |
| FES | HLA-DPB1 | BATF | GNA15 | MTCH1 | C9orf95 | RPGRIP1 | 0.9455 | 0.7924 | 0.7139 |
| S100A12 | HLA-DPB1 | PLAC8 | FCER1G | MTCH1 | C9orf95 | RPGRIP1 | 0.9000 | 0.7299 | 0.7011 |
| S100A12 | HLA-DPB1 | BATF | GPR84 | ARHGEF18 | C9orf95 | RPGRIP1 | 0.8909 | 0.7621 | 0.6659 |
| S100A12 | HLA-DPB1 | BATF | GPR84 | MTCH1 | C9orf95 | RPGRIP1 | 0.8909 | 0.7583 | 0.6731 |
| S100A12 | HLA-DPB1 | BATF | FCER1G | ARHGEF18 | C9orf95 | RPGRIP1 | 0.9091 | 0.7451 | 0.6787 |
| S100A12 | HLA-DPB1 | BATF | FCER1G | MTCH1 | C9orf95 | RPGRIP1 | 0.9182 | 0.7390 | 0.6899 |
| S100A12 | HLA-DPB1 | BATF | C3AR1 | MTCH1 | C9orf95 | RPGRIP1 | 0.9000 | 0.7602 | 0.6987 |
| S100A12 | HLA-DPB1 | BATF | GNA15 | ARHGEF18 | C9orf95 | RPGRIP1 | 0.9273 | 0.7527 | 0.6915 |
| S100A12 | HLA-DPB1 | BATF | GNA15 | MTCH1 | C9orf103 | RPGRIP1 | 0.9545 | 0.7254 | 0.6514 |
| S100A12 | HLA-DPB1 | BATF | GNA15 | MTCH1 | C9orf95 | RPGRIP1 | 0.9364 | 0.7481 | 0.7043 |
| C3AR1 | HLA-DPB1 | BATF | GNA15 | ARHGEF18 | C9orf95 | RPGRIP1 | 0.9273 | 0.7856 | 0.7220 |
| C3AR1 | HLA-DPB1 | BATF | GNA15 | MTCH1 | C9orf95 | RPGRIP1 | 0.9455 | 0.7830 | 0.7340 |

| Discovery set | | | | | | | | |
|---|---|---|---|---|---|---|---|---|
| | Glue | Glue | Glue | Glue | Glue | | | |
| | Grant | Grant | Grant | Grant | Grant | | | |
| | Buffy | Buffy | Buffy | Buffy | Buffy | Validation set | | |
| | coat, | coat, | coat, | coat, | coat, | | | E- |
| GSE | day | day | Day | day | day | GSE | GSE | MEXP- |
| 66099 | [1-3) | [3-6) | [6-10) | [10-18) | [18-24) | 65682 | 74224 | 3589 |
| 0.7554 | 0.8410 | 0.8992 | 0.9440 | 0.8182 | 1.0000 | 0.7525 | 0.8714* | 0.6327 |
| 0.7526 | 0.8462 | 0.9085 | 0.9480 | 0.8295 | 1.0000 | 0.7534 | 0.8945* | 0.6582 |
| 0.7302 | 0.8444 | 0.9169 | 0.9507 | 0.8636 | 1.0000 | 0.5794 | 0.8836* | 0.6531 |
| 0.7471 | 0.8735 | 0.9085* | 0.9507 | 0.8864 | 0.9583 | 0.7678 | 0.8980* | 0.6531 |
| 0.7744 | 0.8615 | 0.8926 | 0.9627 | 0.8864 | 1.0000 | 0.7432 | 0.8840* | 0.6378 |
| 0.7764 | 0.8632 | 0.9029 | 0.9680 | 0.8864 | 1.0000 | 0.7390 | 0.8993* | 0.6429 |
| 0.7814 | 0.8376 | 0.9001 | 0.9453 | 0.8864 | 1.0000 | 0.7405 | 0.9189* | 0.6735 |
| 0.7640 | 0.8667 | 0.9094 | 0.9413 | 0.8864 | 1.0000 | 0.7627 | 0.9381* | 0.6735 |
| 0.7843 | 0.8376 | 0.9085 | 0.9480 | 0.8864 | 1.0000 | 0.7300 | 0.9329* | 0.6888 |
| 0.7941 | 0.8308 | 0.9244 | 0.9667 | 0.9091 | 1.0000 | 0.7348 | 0.9289* | 0.6071 |
| 0.7310 | 0.8684 | 0.8786 | 0.9400 | 0.8295 | 1.0000 | 0.7378 | 0.8540* | 0.6378 |
| 0.7605 | 0.8957 | 0.8403 | 0.9280 | 0.8182 | 1.0000 | 0.7474 | 0.8614* | 0.6786 |
| 0.7576 | 0.9009 | 0.8459* | 0.9360 | 0.8182 | 1.0000 | 0.7489 | 0.8727* | 0.6684 |
| 0.7709 | 0.8923 | 0.8590 | 0.9293 | 0.8295 | 1.0000 | 0.7444 | 0.9176* | 0.6786 |
| 0.7787 | 0.8752 | 0.8646 | 0.9600 | 0.8409 | 1.0000 | 0.7387 | 0.8862* | 0.6684 |
| 0.7784 | 0.8821 | 0.8702 | 0.9640 | 0.8409 | 1.0000 | 0.7399 | 0.8997* | 0.6735 |
| 0.7266 | 0.8923 | 0.9197 | 0.9200 | 0.8409 | 1.0000 | 0.7579 | 0.9394* | 0.6173 |
| 0.7596 | 0.8427 | 0.9458 | 0.9387 | 0.8523 | 1.0000 | 0.7477 | 0.9507* | 0.6173 |
| 0.7611 | 0.8496 | 0.9524 | 0.9440 | 0.8295 | 1.0000 | 0.7477 | 0.9568* | 0.6173 |
| 0.7427 | 0.8547 | 0.8982 | 0.9400 | 0.8068 | 1.0000 | 0.7270 | 0.8836* | 0.6531 |
| 0.7233 | 0.8735 | 0.9020 | 0.9467 | 0.8068 | 0.9583 | 0.7489 | 0.8849* | 0.6224 |
| 0.7430 | 0.8564 | 0.8982 | 0.9440 | 0.7955 | 1.0000 | 0.7279 | 0.8958* | 0.6378 |
| 0.7395 | 0.8957 | 0.8898 | 0.9413 | 0.8182 | 0.9583 | 0.7621 | 0.9037* | 0.6378 |
| 0.7704 | 0.8838 | 0.8525 | 0.9493 | 0.8182* | 1.0000 | 0.7405 | 0.9067* | 0.6786 |
| 0.7327 | 0.8991 | 0.8908 | 0.9453 | 0.8295 | 0.9583 | 0.7609 | 0.9102* | 0.6480 |
| 0.7717 | 0.8923 | 0.8665 | 0.9560 | 0.8068* | 1.0000 | 0.7372 | 0.9167* | 0.6633 |
| 0.7841 | 0.8718 | 0.8730 | 0.9413 | 0.8295 | 1.0000 | 0.7285 | 0.9446* | 0.6990 |
| 0.7901 | 0.8632 | 0.8758 | 0.9613 | 0.8295 | 1.0000 | 0.7303 | 0.9285* | 0.6633 |
| 0.7871 | 0.8650 | 0.8861 | 0.9653 | 0.8182 | 1.0000 | 0.7300 | 0.9416* | 0.6429 |
| 0.7580 | 0.8650 | 0.8908 | 0.9507 | 0.8864 | 1.0000 | 0.7378 | 0.8806* | 0.6276 |
| 0.7256 | 0.8615 | 0.8898 | 0.9467 | 0.8977 | 0.9583 | 0.7642 | 0.8840* | 0.6531 |
| 0.7601 | 0.8667 | 0.8954 | 0.9547 | 0.8977 | 1.0000 | 0.7366 | 0.8923* | 0.6378 |
| 0.7735 | 0.8530 | 0.8908 | 0.9307 | 0.9091 | 1.0000 | 0.7363 | 0.9185* | 0.6837 |
| 0.7760 | 0.8530 | 0.8992 | 0.9320 | 0.8977 | 1.0000 | 0.7330 | 0.9289* | 0.6837 |
| 0.7765 | 0.8393 | 0.9225 | 0.9520 | 0.9318 | 1.0000 | 0.7267 | 0.9180* | 0.6071 |
| 0.7571 | 0.8581 | 0.8936* | 0.9013 | 0.8295 | 1.0000 | 0.7465 | 0.9429* | 0.6888 |
| 0.7675 | 0.8530 | 0.9328 | 0.9293 | 0.8636 | 1.0000 | 0.7495 | 0.9307* | 0.5969 |
| 0.7685 | 0.8462 | 0.9430 | 0.9373 | 0.8523 | 1.0000 | 0.7486 | 0.9425* | 0.6122 |
| 0.7462 | 0.8547 | 0.8852 | 0.9347 | 0.8068 | 1.0000 | 0.7270 | 0.8605* | 0.6224 |
| 0.7410 | 0.8427 | 0.8618 | 0.9400 | 0.8409 | 1.0000 | 0.7249 | 0.8492* | 0.6378 |
| 0.7467 | 0.8598 | 0.8936 | 0.9360 | 0.7841 | 1.0000 | 0.7246 | 0.8701* | 0.6378 |
| 0.7906 | 0.8615 | 0.8301 | 0.9307 | 0.8750 | 1.0000 | 0.7291 | 0.9355* | 0.6990 |
| 0.7898 | 0.8752 | 0.8721 | 0.9293 | 0.8523 | 1.0000 | 0.7276 | 0.9307* | 0.6990 |
| 0.7960 | 0.8718 | 0.8665 | 0.9547 | 0.8523 | 1.0000 | 0.7336 | 0.9106* | 0.6633 |
| 0.7879 | 0.8427 | 0.8497 | 0.9613 | 0.8750 | 1.0000 | 0.7267 | 0.9124* | 0.6429 |
| 0.7925 | 0.8803 | 0.8814 | 0.9627 | 0.8409 | 1.0000 | 0.7324 | 0.9220* | 0.6378 |
| 0.7374 | 0.8991 | 0.9076 | 0.9400 | 0.8636 | 0.9583 | 0.7459 | 0.8779* | 0.6429 |
| 0.7231 | 0.8906 | 0.9010* | 0.9400 | 0.9091 | 0.9583 | 0.7459 | 0.8710* | 0.6429 |
| 0.7355 | 0.9026 | 0.9020 | 0.9387 | 0.8750 | 0.9583 | 0.7462 | 0.8897* | 0.6582 |
| 0.7352 | 0.8821 | 0.8263 | 0.9373 | 0.8636 | 1.0000 | 0.7537 | 0.9259* | 0.6837 |
| 0.7817 | 0.8564 | 0.8964 | 0.9467 | 0.8750 | 1.0000 | 0.7375 | 0.8823* | 0.6786 |
| 0.7286 | 0.8564 | 0.8786 | 0.9107 | 0.8182 | 1.0000 | 0.7510 | 0.9032* | 0.6480 |
| 0.7466 | 0.8838 | 0.8870 | 0.9173 | 0.8295 | 1.0000 | 0.7615 | 0.9316* | 0.6582 |
| 0.7332 | 0.8667 | 0.9160 | 0.9387 | 0.8750 | 0.9583 | 0.7627 | 0.9394* | 0.6020 |
| 0.7643 | 0.8530 | 0.9029* | 0.9293 | 0.8636 | 1.0000 | 0.7510 | 0.9407* | 0.6378 |
| 0.7662 | 0.8530 | 0.9076* | 0.9400 | 0.8750 | 1.0000 | 0.7492 | 0.9494* | 0.6429 |
| 0.7484 | 0.8462 | 0.8777 | 0.9387 | 0.8295 | 1.0000 | 0.7300 | 0.8710* | 0.6786 |
| 0.7462 | 0.8821 | 0.8543 | 0.9413 | 0.8409 | 0.9583 | 0.7606 | 0.8806* | 0.6837 |
| 0.7430 | 0.8889 | 0.8553 | 0.9453 | 0.8523 | 0.9583 | 0.7615 | 0.8945* | 0.6837 |
| 0.7824 | 0.8701 | 0.8385 | 0.9373 | 0.8523 | 1.0000 | 0.7249 | 0.9303* | 0.7449 |
| 0.7901 | 0.8650 | 0.8497* | 0.9480 | 0.8409 | 1.0000 | 0.7342 | 0.9089* | 0.6939 |
| 0.7899 | 0.8718 | 0.8525* | 0.9573 | 0.8409 | 1.0000 | 0.7333 | 0.9250* | 0.6837 |
| 0.7320 | 0.8752 | 0.9346* | 0.9147 | 0.8182 | 1.0000 | 0.7600 | 0.8980* | 0.6122 |
| 0.7420 | 0.8479 | 0.9001 | 0.9293 | 0.7955 | 1.0000 | 0.7261 | 0.8435* | 0.6327 |
| 0.7402 | 0.8513 | 0.8982 | 0.9293 | 0.7841 | 1.0000 | 0.7225 | 0.8548* | 0.6378 |
| 0.7737 | 0.8872 | 0.8852 | 0.9520 | 0.8295 | 1.0000 | 0.7360 | 0.8514* | 0.6378 |
| 0.7735 | 0.8957 | 0.8964 | 0.9587 | 0.8068 | 1.0000 | 0.7360 | 0.8710* | 0.6327 |
| 0.7881 | 0.8667 | 0.8898 | 0.9307 | 0.8636* | 1.0000 | 0.7240 | 0.9106* | 0.6888 |
| 0.7950 | 0.8581 | 0.9094 | 0.9520 | 0.8523 | 1.0000 | 0.7276 | 0.8888* | 0.6327 |
| 0.7625 | 0.8872 | 0.9374 | 0.9547 | 0.8636 | 0.9583 | 0.7513 | 0.8976* | 0.6224 |
| 0.7953 | 0.8718 | 0.9150 | 0.9560 | 0.8409 | 1.0000 | 0.7243 | 0.9010* | 0.6327 |
| 0.8017 | 0.8513 | 0.8749 | 0.9360 | 0.8864 | 1.0000 | 0.7195 | 0.9381* | 0.7143 |
| 0.8030 | 0.8427 | 0.8898 | 0.9373 | 0.8750 | 1.0000 | 0.7177 | 0.9459* | 0.7041 |

*indicates p-value from DeLong's test in comparison with the new 6 gene panels less than 0.05.

TABLE 6
22 new six gene panels with higher performance in the validation sets. Among 73 six gene panels that
had higher performance than the lower bound of 95% confidence intervals of the original Stanford11
panel in all discovery datasets, 22 panels had even higher performance in two independent datasets.

| | Interferon- | | | | | | | |
|---|---|---|---|---|---|---|---|---|
| | gamma, | | | | | | | |
| Chemotaxis | antigen | | PLC, | | | | | |
| angiogenesis, | processing, | Transcrip | phosphorylation, | | | | | E- |
| adhesion, | immune | by RNA | platelet | | | | | MEXP- |
| migration | response | pol II | activation | Apoptosis | Metabolism | GSE65682 | GSE74224 | 3589 |
| CCR1 | HLA-DPB1 | BATF | C3AR1 | ARHGEF18 | C9orf95 | 0.7807 | 0.8854 | 0.6633 |
| CCR1 | HLA-DPB1 | BATF | C3AR1 | MTCH1 | C9orf103 | 0.8170 | 0.9058 | 0.6990 |
| CCR1 | HLA-DPB1 | BATF | C3AR1 | MTCH1 | C9orf95 | 0.8095 | 0.8967 | 0.6786 |
| CCR1 | HLA-DPB1 | BATF | GNA15 | MTCH1 | C9orf95 | 0.8080 | 0.8893 | 0.6378 |
| CD177 | HLA-DPB1 | BATF | C3AR1 | MTCH1 | C9orf95 | 0.8014 | 0.8827 | 0.6684 |
| CD63 | HLA-DPB1 | PLAC8 | FCER1G | MTCH1 | C9orf95 | 0.8158 | 0.9128 | 0.6122 |
| CD63 | HLA-DPB1 | PLAC8 | GNA15 | ARHGEF18 | C9orf95 | 0.7852 | 0.9220 | 0.5663 |
| CD63 | HLA-DPB1 | PLAC8 | GNA15 | MTCH1 | C9orf95 | 0.8107 | 0.9333 | 0.5918 |
| CD63 | HLA-DPB1 | BATF | C3AR1 | MTCH1 | C9orf95 | 0.8080 | 0.9067 | 0.6633 |
| CD63 | HLA-DPB1 | BATF | GNA15 | MTCH1 | C9orf95 | 0.8029 | 0.8915 | 0.6378 |
| EMR1 | HLA-DPB1 | BATF | C3AR1 | MTCH1 | C9orf95 | 0.8149 | 0.8963 | 0.6582 |
| EMR1 | HLA-DPB1 | BATF | GNA15 | MTCH1 | C9orf95 | 0.8044 | 0.8836 | 0.6071 |
| FCER1G | HLA-DPB1 | PLAC8 | C3AR1 | MTCH1 | C9orf95 | 0.8080 | 0.9241 | 0.6531 |
| FCER1G | HLA-DPB1 | PLAC8 | GNA15 | ARHGEF18 | C9orf95 | 0.7861 | 0.9098 | 0.5408 |
| FCER1G | HLA-DPB1 | PLAC8 | GNA15 | MTCH1 | C9orf95 | 0.8092 | 0.9185 | 0.5816 |
| FCER1G | HLA-DPB1 | BATF | C3AR1 | MTCH1 | SEPHS2 | 0.8086 | 0.8867 | 0.6582 |
| FCER1G | HLA-DPB1 | BATF | C3AR1 | MTCH1 | C9orf95 | 0.8071 | 0.8963 | 0.6480 |
| FES | HLA-DPB1 | PLAC8 | FCER1G | MTCH1 | C9orf95 | 0.8089 | 0.8945 | 0.6173 |
| FES | HLA-DPB1 | PLAC8 | GNA15 | ARHGEF18 | C9orf103 | 0.7870 | 0.9098 | 0.6122 |
| FES | HLA-DPB1 | PLAC8 | GNA15 | MTCH1 | C9orf95 | 0.8005 | 0.9150 | 0.6071 |
| FES | HLA-DPB1 | BATF | C3AR1 | MTCH1 | C9orf95 | 0.8026 | 0.8806 | 0.6786 |
| C3AR1 | HLA-DPB1 | BATF | GNA15 | MTCH1 | C9orf95 | 0.7999 | 0.9133 | 0.6480 |

Though removing RPGRIP1 involved in cell development and proliferation process from the original Stanford11 panel did not decrease the overall diagnostic performance (Table 4), we tested the effect of adding RPGRIP1 to the new 6 gene panels. The results showed that adding RPGRIP1 to the 6-gene panel only improved the performance in one of the validation datasets, GSE74224, but not in the other two datasets (Table 7).

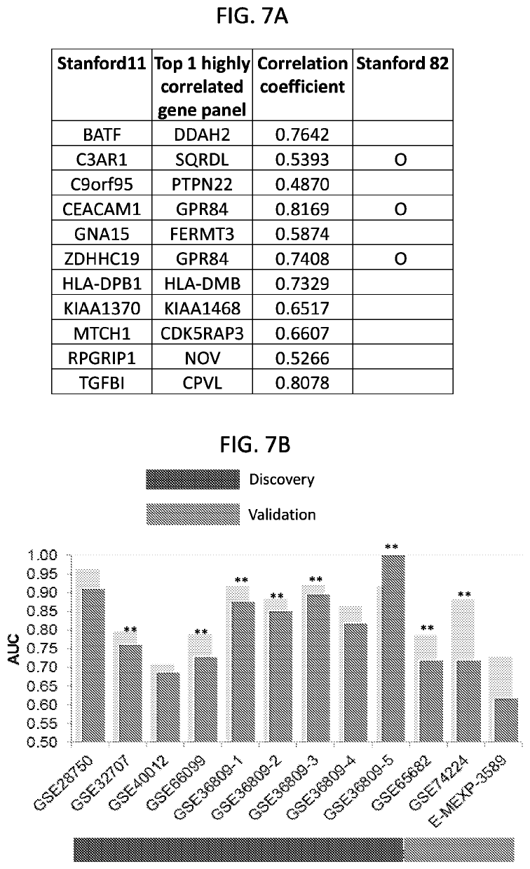

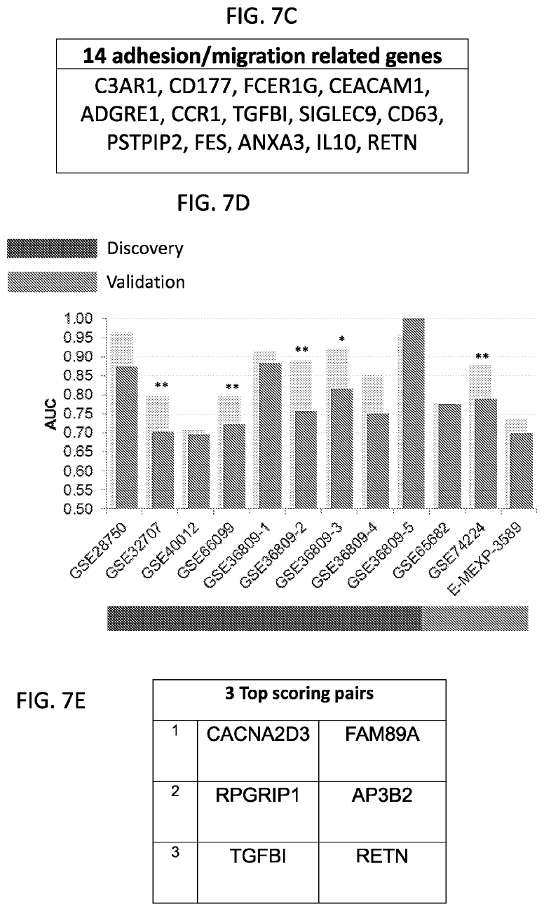

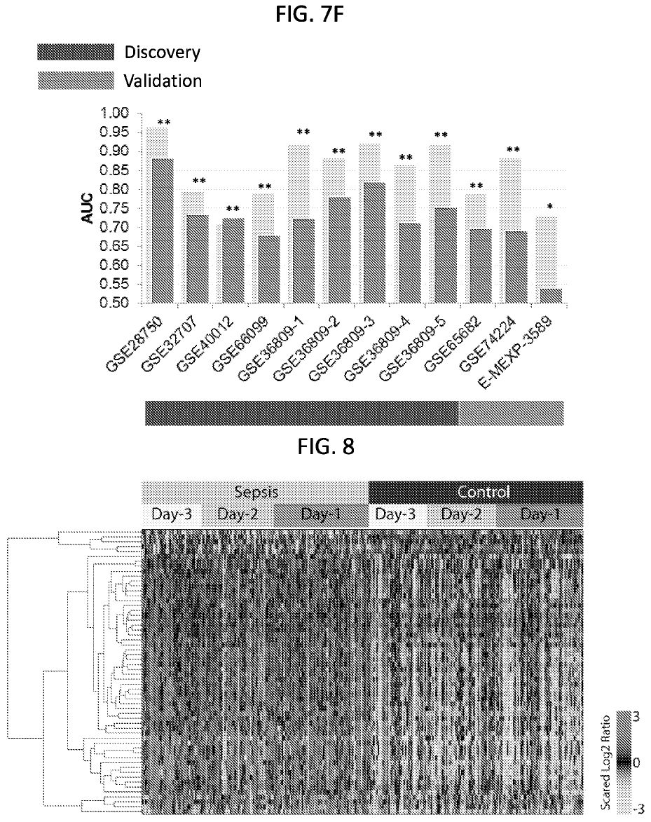

The performance differences between biological function-based gene substitution and expression correlation-based substitution were tested. Based on expression profiles of all the discovery datasets, the highest correlated genes with features in the Stanford11 panel were selected (referred to as panel-HC), regardless of the biological functions (FIG. 7A). Most of the highest correlated genes with features in the Stanford11 panel were not from Stanford82 nor involved in the same GOBPs represented by Stanfordl 1. For instance, BATF of Stanford11 is involved in transcription by pol II, but the highest correlated gene, DDAH2 is involved in nitric oxide biosynthetic process. The average AUCs of the panel-HC in the discovery and validation sets were 0.8238 and 0.6846, respectively, which are 3.58% and 14.40% lower than the Stanford11 (average AUC of 0.8544 and 0.7997, respectively, in the discovery and validation sets, p-value from DeLong's test of less than 0.01 in two validation datasets, GSE65682 and GSE74224, and less than 0.1 in one validation dataset, E-MEXP-3589, FIG. 7B), which suggests maintaining biological processes associated with disease condition is more critical to generating high performance diagnostic panels than maintaining features with highly correlated expression profiles.

364 panels (referred to as panel-AM) were also generated by randomly selecting 11 genes from 14 genes involved in adhesion/migration process (FIG. 7C). The performance of panel-AM was also lower than Stanford11 (average AUC of 0.7999 and 0.7546 in the discovery and validation sets, respectively, and p-value from DeLong's test of less than 0.01 in GSE74224 in FIG. 7D), which suggests genes from one biological process do not deliver sufficient diagnostic performance.

Lastly, TSP and k-TSP algorithms were applied to identify a small set of paired genes. TSP identified two genes that have only 67% classification accuracy in the three independent validation sets. Therefore, we applied k-TSP to increase performance by including additional pairs of genes. It resulted in three pairs which include six genes in total (referred to as panel-kTSP, FIG. 7E). The panel-kTSP also showed lower performance in independent datasets (AUC of 0.6408 and p-value from DeLong's test of less than 0.05 in all validation data sets in FIG. 7F).

### Example 2

### Identification of Gene Panels to Predict Development of Sepsis

Peripheral blood samples were collected from elective surgery patients (n=155) prior to surgery (Pre-Op) and daily up until one or two days post-sepsis diagnosis (Post-Op). The patients had undergone a wide range of surgeries, with the most common being large bowel resection, vascular surgery, and pancreatic surgery. The control group included an equal number of patients matched by age, sex, and surgical procedure who did not develop sepsis. The sepsis patients, as well as matched controls, were divided into three separate groups: Discovery, Test, and Validation sample sets (Table 8). Samples from the Discovery set were used to identify various differentially expressed genes (DEGs) and corresponding biomarker panel candidates. The Test set and the Validation set were used to compare performances of the candidate panels and to assess the ability of the selected panel based on the test set to predict sepsis, respectively.

TABLE 8
Groups of surgery patients used for sepsis
prognostic biomarker discovery.

| | Discovery Set | Test Set | Validation Set | Total |
|---|---|---|---|---|
| Sepsis | 64 | 31 | 60 | 155 |
| Control | 63 | 30 | 60 | 153 |

*Numbers indicate patient numbers in each group.

Based on the study design, the longitudinal samples from each patient were organized according to the date when sepsis was diagnosed (Day 0). Using this nomenclature, the day before diagnosis date is Day-1 and one day post-diagnosis is Day+1. There were significantly fewer samples available on Day−4 (almost 50% decrease from Day−3). Since the performance and reliability of biomarkers depends on the number of samples analyzed and the purpose of this study was to find a predictive marker for risk sepsis, the analyses were concentrated on data from samples taken on Day-3 to Day-1. Total RNA from whole blood was isolated with miRNeasy® kit (Qiagen, Germantown, Md.) according to the manufacturer's instructions. Fluorescent-labeled (Cy3) probes were prepared with the single color labeling kit from Agilent (Santa Clara, Calif.). Probes were then loaded on the array hybridization chambers then hybridized at 65° C. for 17 hours. The slides were washed and scanned (Agilent, Santa Clara, Calif.). The microarray data was then normalized by quantile normalization.

To identify genes associated with the development of sepsis for the samples, three factors were considered during statistical analysis: 1) Pre-Op normalization, 2) paired vs unpaired testing, and 3) time points. By considering only probes corresponding to mRNA, eight sets of DEGs were identified (Table 9). The Pre-Op normalization factor was applied to remove transcriptome changes associated with surgery from Post-Op samples to reduce the individual differences and emphasize the gene expression changes associated with infection processes. The second factor was to explore the effect of matched sepsis-control patients individually or as a group. The third factor was to compare the gene expression profile changes between the sepsis and control groups at individual timepoints before sepsis is diagnosed (Day 0). It is reasonable to assume that a sepsis-specific signature would be more prominent as getting closer to time of diagnosis. Grouping all pre-diagnosis sepsis time points together could potentially dilute out the stronger signal present at Day-1. Statistical analysis was therefore performed comparing control and sepsis at each time-point. DEGs identified in at least two individual timepoints were considered.

Support Vector Machine was then combined with Recursive Feature Elimination (SVM-RFE) and applied in conjunction with a repeat cross-validation procedure to select genes that were most relevant for the classification between sepsis patients and controls from the selected DEGs identified from the eight different procedures. The performances of the resultant eight candidate panels were then evaluated in the test sample set (Table 9).

TABLE 9
Classification performance of gene panels identified using different statistical approaches.

| Pre-Op | Statistical | | | # | # Genes | Avg. | Day −3 | Day −2 | Day −1 |
|---|---|---|---|---|---|---|---|---|---|
| norm. | Test | Comparison | Approach | DEGs | in panel | AUC | AUC | AUC | AUC |
| With Pre- | Paired | All time | 1 | 76 | 37 | 0.7036 | 0.6198 | 0.6266 | 0.7805 |
| Op norm. | Test | points | | | | | | | |
| | | together | | | | | | | |
| | | Each time | 2 | 58 | 19 | 0.7669 | 0.7355 | 0.6842 | 0.8506 |
| | | point | | | | | | | |
| | Unpaired | All time | 3 | 77 | 39 | 0.7396 | 0.6694 | 0.6692 | 0.8299 |
| | Test | points | | | | | | | |
| | | together | | | | | | | |
| | | Each time | 4 | 62 | 21 | 0.7617 | 0.7273 | 0.6892 | 0.8322 |
| | | point | | | | | | | |
| Without | Paired | All time | 5 | 345 | 50 | 0.8475 | 0.8347 | 0.8421 | 0.8747 |
| Pre-Op | Test | points | | | | | | | |
| norm. | | together | | | | | | | |
| | | Each time | 6 | 355 | 63 | 0.8751 | 0.8347 | 0.8596 | 0.9092 |
| | | point | | | | | | | |
| | Unpaired | All time | 7 | 342 | 85 | 0.8433 | 0.8099 | 0.8321 | 0.8678 |
| | Test | points | | | | | | | |
| | | together | | | | | | | |
| | | Each time | 8 | 338 | 54 | 0.8724 | 0.7934 | 0.8521 | 0.9126 |
| | | point | | | | | | | |

Four different approaches using Pre-Op normalization were first performed (Table 9). Differentially expressed genes and their optimal feature sets from SVM-RFE were identified for each approach. Among the four approaches, 58 genes (ISB58) were identified from approach 2. A 19 gene panel (ISB19) was selected from ISB58 using SVM-RFE. ISB19 showed the highest performance from panel genes identified from the four approaches utilizing Pre-Op normalization (Approach 2 (bold in Table 9)). ISB58 is summarized in Table 10, and the overall expression profile is shown in FIG. 8.

TABLE 10
Gene list for ISB58 (81 probes)

| | | Day −3 | | Day −2 | | Day −1 | |
|---|---|---|---|---|---|---|---|
| Probe id | Gene Symbol | logFC | P. Value | logFC | P. Value | logFC | P. Value |
| A_33_P3238993 | AGFG1 | 0.86 | 5.47E−04 | 0.62 | 2.01E−03 | 0.67 | 3.91E−04 |
| A_23_P146058 | ATP6V1C1 | 0.77 | 2.27E−03 | 0.55 | 9.33E−03 | 0.69 | 1.15E−04 |
| A_23_P380614 | ATP9A | 0.99 | 5.62E−05 | 0.85 | 1.97E−04 | 0.97 | 1.49E−06 |
| A_23_P253602 | BMX | 0.69 | 4.61E−03 | 0.65 | 3.39E−03 | 0.80 | 7.55E−05 |
| A_23_P330561 | C19orf59 | 1.26 | 5.58E−03 | 0.92 | 2.34E−02 | 1.41 | 2.78E−04 |
| A_21_P0011751 | CD177 | 1.61 | 1.31E−03 | 1.33 | 7.07E−03 | 1.83 | 4.38E−05 |
| A_23_P259863 | CD177 | 1.76 | 1.45E−03 | 1.30 | 9.30E−03 | 1.88 | 4.39E−05 |
| A_33_P3232080 | CD177 | 0.60 | 5.29E−03 | 0.73 | 1.91E−03 | 0.90 | 3.41E−06 |
| A_33_P3369844 | CD24 | 0.61 | 2.63E−02 | 0.65 | 5.29E−03 | 0.71 | 5.22E−03 |
| A_33_P3389060 | CDK5RAP2 | 0.73 | 6.37E−03 | 0.67 | 4.01E−03 | 0.77 | 1.73E−04 |
| A_23_P83110 | CDK5RAP2 | 0.84 | 7.75E−03 | 0.72 | 9.41E−03 | 0.84 | 9.12E−04 |
| A_24_P382319 | CEACAM1 | 0.83 | 9.78E−04 | 0.75 | 6.56E−04 | 0.97 | 1.39E−05 |
| A_33_P3352578 | CLEC4D | 0.82 | 4.30E−03 | 0.63 | 2.14E−02 | 0.89 | 2.50E−04 |
| A_33_P3258977 | CLEC4D | 0.85 | 4.35E−03 | 0.68 | 1.28E−02 | 0.89 | 2.41E−04 |
| A_33_P3316786 | DACH1 | 0.65 | 1.56E−03 | 0.64 | 2.08E−03 | 0.77 | 6.35E−06 |
| A_23_P32577 | DACH1 | 0.68 | 2.06E−05 | 0.58 | 2.54E−04 | 0.64 | 2.29E−06 |
| A_23_P19482 | DDAH2 | 0.62 | 1.21E−02 | 0.65 | 2.11E−03 | 0.90 | 8.12E−06 |
| A_23_P56559 | DHRS9 | 0.96 | 6.34E−04 | 0.69 | 5.53E−03 | 0.90 | 5.85E−05 |
| A_33_P3301410 | EXOSC4 | 0.81 | 1.81E−04 | 0.83 | 7.14E−05 | 1.02 | 3.90E−08 |
| A_23_P208768 | FCAR | 0.73 | 3.91E−03 | 0.55 | 1.17E−02 | 0.61 | 3.04E−03 |
| A_23_P23221 | GADD45A | 0.88 | 8.47E−04 | 0.74 | 1.45E−03 | 0.97 | 1.57E−05 |
| A_23_P67847 | GALNT14 | 0.77 | 6.03E−03 | 0.67 | 1.65E−02 | 0.81 | 5.96E−04 |
| A_23_P74290 | GBP5 | −0.81 | 1.94E−04 | −0.45 | 6.16E−02 | −0.74 | 1.52E−03 |
| A_23_P25155 | GPR84 | 1.10 | 9.83E−04 | 1.02 | 2.87E−04 | 1.24 | 6.20E−07 |
| A_23_P122863 | GRB10 | 0.74 | 7.28E−03 | 0.53 | 2.03E−02 | 0.89 | 1.04E−04 |
| A_24_P235266 | GRB10 | 0.67 | 1.35E−02 | 0.60 | 9.13E−03 | 0.96 | 2.70E−05 |
| A_23_P29422 | GYG1 | 1.05 | 3.05E−04 | 0.84 | 1.65E−03 | 0.96 | 5.70E−05 |
| A_21_P0013518 | GYG1 | 0.96 | 1.99E−04 | 0.79 | 1.50E−03 | 0.92 | 2.42E−05 |
| A_23_P384517 | GYG1 | 0.91 | 5.52E−03 | 0.78 | 9.45E−03 | 1.04 | 1.39E−04 |
| A_33_P3376821 | GZMA | −0.59 | 8.29E−03 | −0.61 | 3.32E−03 | −0.46 | 2.74E−02 |
| A_23_P128993 | GZMH | −0.68 | 6.67E−03 | −0.69 | 3.37E−03 | −0.52 | 1.10E−02 |
| A_23_P213584 | HK3 | 0.83 | 3.40E−03 | 0.67 | 4.98E−03 | 0.69 | 1.30E−03 |
| A_23_P206760 | HP | 1.08 | 5.79E−03 | 0.75 | 4.17E−02 | 1.20 | 4.06E−04 |
| A_33_P3289236 | HPR | 1.07 | 4.00E−03 | 0.73 | 2.35E−02 | 1.18 | 7.87E−05 |
| A_24_P103886 | IDI1 | 0.73 | 1.50E−03 | 0.52 | 3.43E−03 | 0.61 | 4.00E−04 |
| A_33_P3251876 | IL18R1 | 0.59 | 3.99E−03 | 0.52 | 2.52E−03 | 0.82 | 1.16E−06 |
| A_33_P3211666 | IL18R1 | 0.79 | 5.69E−03 | 0.68 | 7.68E−03 | 1.19 | 5.12E−07 |
| A_24_P208567 | IL18R1 | 0.97 | 1.02E−03 | 0.78 | 1.75E−03 | 1.10 | 6.53E−06 |
| A_24_P63019 | IL1R2 | 0.93 | 1.71E−02 | 0.90 | 2.93E−03 | 1.32 | 7.98E−06 |
| A_23_P169437 | LCN2 | 0.89 | 4.13E−03 | 0.74 | 3.05E−03 | 0.96 | 6.47E−04 |
| A_23_P120902 | LGALS2 | −0.70 | 1.01E−03 | −0.66 | 2.93E−03 | −1.03 | 3.14E−06 |
| A_23_P50638 | LRG1 | 0.63 | 3.33E−03 | 0.44 | 2.10E−02 | 0.62 | 4.66E−04 |
| A_23_P40174 | MMP9 | 1.08 | 2.05E−03 | 0.81 | 8.96E−03 | 1.21 | 1.21E−04 |
| A_23_P40174 | MMP9 | 1.03 | 3.31E−03 | 0.79 | 1.22E−02 | 1.20 | 1.51E−04 |
| A_23_P40174 | MMP9 | 0.99 | 5.17E−03 | 0.78 | 1.27E−02 | 1.20 | 1.62E−04 |
| A_23_P40174 | MMP9 | 1.00 | 5.35E−03 | 0.78 | 1.26E−02 | 1.23 | 1.20E−04 |
| A_23_P40174 | MMP9 | 1.04 | 3.23E−03 | 0.77 | 1.30E−02 | 1.22 | 1.43E−04 |
| A_23_P40174 | MMP9 | 1.02 | 3.60E−03 | 0.79 | 1.07E−02 | 1.20 | 1.26E−04 |
| A_23_P40174 | MMP9 | 1.03 | 2.84E−03 | 0.79 | 8.99E−03 | 1.20 | 1.27E−04 |
| A_23_P40174 | MMP9 | 1.06 | 2.06E−03 | 0.81 | 7.56E−03 | 1.22 | 1.04E−04 |
| A_23_P40174 | MMP9 | 1.02 | 3.67E−03 | 0.79 | 9.06E−03 | 1.20 | 1.39E−04 |
| A_23_P40174 | MMP9 | 0.99 | 4.43E−03 | 0.75 | 1.30E−02 | 1.16 | 1.81E−04 |
| A_23_P161458 | OLAH | 0.66 | 1.64E−02 | 0.85 | 2.18E−03 | 0.98 | 2.58E−05 |
| A_24_P181254 | OLFM4 | 1.23 | 1.31E−03 | 0.98 | 4.41E−04 | 1.42 | 8.48E−05 |
| A_23_P170186 | OPLAH | 0.75 | 4.67E−03 | 0.72 | 3.70E−03 | 0.99 | 2.04E−05 |
| A_24_P413669 | PFKFB2 | 1.11 | 1.05E−03 | 0.97 | 1.10E−03 | 1.29 | 6.05E−06 |
| A_33_P3300635 | PFKFB2 | 0.88 | 3.82E−03 | 0.74 | 1.73E−03 | 1.11 | 9.25E−06 |
| A_24_P261259 | PFKFB3 | 0.90 | 4.65E−03 | 0.74 | 6.84E−03 | 0.99 | 1.16E−04 |
| A_23_P208747 | PGLYRP1 | 0.62 | 6.66E−03 | 0.46 | 2.03E−02 | 0.63 | 3.35E−03 |
| A_24_P183128 | PLAC8 | 0.76 | 1.76E−03 | 0.77 | 2.31E−03 | 0.90 | 3.30E−05 |
| A_23_P119222 | RETN | 1.15 | 6.90E−04 | 1.04 | 1.76E−03 | 1.24 | 1.74E−04 |
| A_33_P3350863 | RETN | 1.09 | 6.27E−03 | 0.97 | 6.58E−03 | 1.43 | 1.30E−04 |
| A_23_P306941 | RGL4 | 0.86 | 5.79E−03 | 0.76 | 4.36E−03 | 0.97 | 4.49E−04 |
| A_23_P151637 | RNASE2 | 0.66 | 5.78E−03 | 0.62 | 1.99E−03 | 0.58 | 2.37E−03 |
| A_23_P163025 | RNASE3 | 0.68 | 3.84E−04 | 0.65 | 2.19E−04 | 0.64 | 4.61E−05 |
| A_33_P3385785 | S100A12 | 1.02 | 5.23E−03 | 0.82 | 1.04E−02 | 1.13 | 1.54E−04 |
| A_23_P29005 | SAMSN1 | 0.96 | 9.04E−04 | 0.62 | 1.32E−02 | 0.87 | 3.11E−04 |
| A_24_P81900 | SLC2A3 | 0.66 | 2.72E−03 | 0.57 | 5.11E−03 | 0.63 | 3.87E−04 |
| A_23_P139669 | SLC2A3 | 0.64 | 4.30E−04 | 0.64 | 2.35E−04 | 0.54 | 3.19E−04 |
| A_23_P431388 | SPOCD1 | 0.41 | 1.61E−02 | 0.59 | 3.64E−05 | 0.60 | 1.19E−04 |
| A_33_P3275055 | ST6GALNAC3 | 0.59 | 9.78E−04 | 0.46 | 4.80E−03 | 0.64 | 6.07E−06 |
| A_23_P154605 | SULF2 | −0.73 | 7.04E−05 | −0.68 | 8.76E−04 | −0.59 | 1.61E−04 |
| A_33_P3309075 | TBC1D8 | 0.73 | 1.38E−04 | 0.62 | 9.18E−04 | 0.72 | 1.66E−05 |
| A_23_P64372 | TCN1 | 0.67 | 2.59E−04 | 0.63 | 2.13E−04 | 0.72 | 9.74E−05 |
| A_32_P208350 | TDRD9 | 0.94 | 1.94E−03 | 0.88 | 7.49E−04 | 1.10 | 5.86E−06 |
| A_23_P5392 | TP53I3 | 0.69 | 3.05E−04 | 0.48 | 7.18E−03 | 0.62 | 9.33E−05 |
| A_33_P3392077 | TP53I3 | 0.67 | 4.62E−04 | 0.49 | 4.64E−03 | 0.68 | 1.96E−04 |
| A_24_P27977 | TRPM2 | 0.61 | 5.61E−04 | 0.45 | 1.70E−03 | 0.59 | 8.09E−06 |
| A_23_P313389 | UGCG | 0.92 | 1.24E−04 | 0.71 | 1.68E−03 | 0.95 | 1.01E−05 |
| A_23_P351275 | UPP1 | 0.93 | 3.61E−04 | 0.71 | 2.65E−03 | 0.71 | 1.87E−03 |
| A_33_P3399571 | VNN1 | 0.84 | 9.70E−03 | 0.63 | 3.76E−02 | 0.88 | 8.39E−04 |

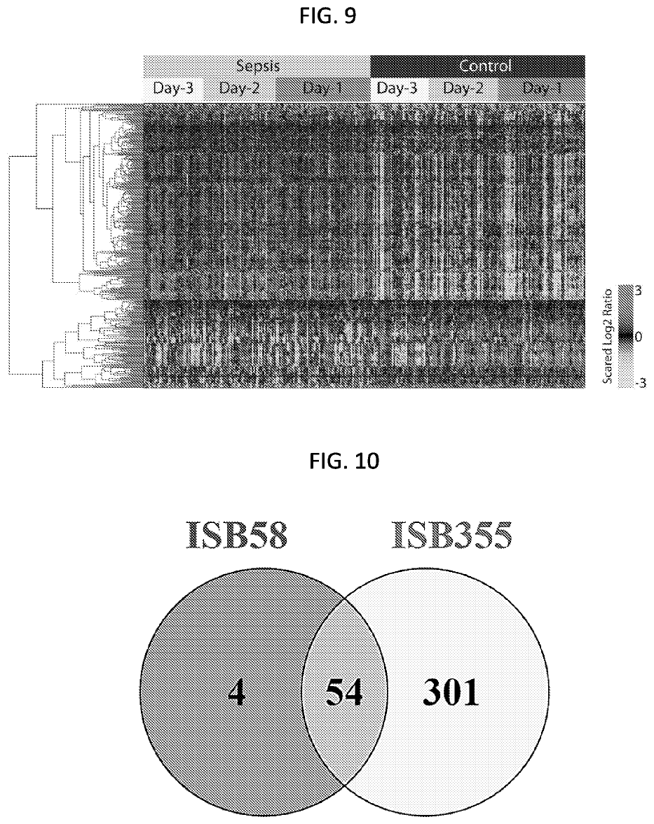

Second, from the comparisons without Pre-Op normalization, gene panels identified from approach 6 showed the best performance (Approach 6 highlighted with bold in Table 9). With this approach, 355 DEGs were identified (ISB355) (Table 11). Among the 355 genes, 63 genes (ISB63) were then identified as an optimal feature set using SVM-RFE. The overall expression profiles for the ISB355 is shown in FIG. 9.

TABLE 11
Gene list for ISB355 (503 probes)

| | | Day −3 | | Day −2 | | Day −1 | |
|---|---|---|---|---|---|---|---|
| Probe id | Gene Symbol | logFC | P. Value | logFC | P. Value | logFC | P. Value |
| A_33_P3424577 | A23747 | −0.69 | 5.95E−04 | −0.68 | 1.29E−04 | −0.81 | 1.59E−05 |
| A_23_P17242 | ABHD1 | 0.08 | 7.25E−01 | −0.67 | 8.33E−03 | −0.71 | 3.96E−04 |
| A_23_P107336 | ACAP1 | 0.57 | 1.83E−05 | 0.59 | 4.75E−07 | 0.59 | 1.85E−08 |
| A_23_P59528 | ACN9 | 0.64 | 1.24E−05 | 0.62 | 3.82E−05 | 0.72 | 4.98E−07 |
| A_23_P110212 | ACSL1 | 0.80 | 8.58E−06 | 0.75 | 2.73E−08 | 0.85 | 1.52E−08 |
| A_23_P110212 | ACSL1 | 0.79 | 2.45E−05 | 0.77 | 8.12E−09 | 0.82 | 3.35E−08 |
| A_23_P110212 | ACSL1 | 0.80 | 2.34E−05 | 0.75 | 2.26E−08 | 0.85 | 2.33E−09 |
| A_23_P110212 | ACSL1 | 0.78 | 2.05E−05 | 0.74 | 6.12E−08 | 0.84 | 1.01E−08 |
| A_23_P110212 | ACSL1 | 0.81 | 1.84E−05 | 0.76 | 1.15E−08 | 0.83 | 3.77E−08 |
| A_23_P110212 | ACSL1 | 0.83 | 1.06E−05 | 0.75 | 1.80E−08 | 0.82 | 9.08E−09 |
| A_23_P110212 | ACSL1 | 0.81 | 2.60E−05 | 0.75 | 2.08E−08 | 0.81 | 1.52E−08 |
| A_23_P110212 | ACSL1 | 0.81 | 2.11E−05 | 0.75 | 1.04E−08 | 0.82 | 4.49E−08 |
| A_23_P110212 | ACSL1 | 0.80 | 4.98E−05 | 0.76 | 1.36E−08 | 0.83 | 1.36E−08 |
| A_23_P110212 | ACSL1 | 0.81 | 1.26E−05 | 0.76 | 1.01E−08 | 0.84 | 1.64E−08 |
| A_23_P217564 | ACSL4 | 0.73 | 6.00E−07 | 0.71 | 1.70E−09 | 0.74 | 5.99E−11 |
| A_33_P3256848 | ADAM12 | −0.10 | 7.01E−01 | −0.75 | 3.68E−04 | −0.61 | 1.79E−03 |
| A_33_P3245489 | ADAMTSL5 | −0.63 | 1.12E−02 | −0.59 | 3.18E−03 | −0.68 | 5.82E−04 |
| A_33_P3238997 | AGFG1 | 0.62 | 2.96E−06 | 0.55 | 6.53E−08 | 0.69 | 3.18E−11 |
| A_33_P3238993 | AGFG1 | 1.00 | 3.67E−07 | 0.91 | 5.08E−08 | 0.96 | 5.73E−10 |
| A_33_P3279470 | AGRP | 0.01 | 9.77E−01 | −0.64 | 4.28E−03 | −0.65 | 1.93E−03 |
| A_23_P169278 | AGTPBP1 | 0.66 | 1.82E−05 | 0.65 | 1.71E−08 | 0.63 | 3.39E−07 |
| A_33_P3215797 | AHDC1 | 0.03 | 8.87E−01 | −0.78 | 4.00E−04 | −0.73 | 4.55E−04 |
| A_32_P44394 | AIM2 | 0.40 | 1.54E−02 | 0.61 | 1.20E−04 | 0.67 | 5.39E−06 |
| A_23_P104464 | ALOX5 | 0.78 | 1.83E−04 | 0.72 | 1.46E−07 | 0.70 | 1.27E−06 |
| A_23_P104464 | ALOX5 | 0.75 | 9.19E−06 | 0.73 | 1.36E−07 | 0.70 | 3.28E−07 |
| A_23_P104464 | ALOX5 | 0.73 | 1.81E−05 | 0.71 | 1.02E−07 | 0.66 | 1.10E−06 |
| A_23_P104464 | ALOX5 | 0.73 | 2.15E−05 | 0.69 | 2.26E−07 | 0.67 | 2.38E−06 |
| A_23_P104464 | ALOX5 | 0.72 | 1.87E−05 | 0.72 | 2.48E−08 | 0.67 | 3.10E−07 |
| A_23_P104464 | ALOX5 | 0.68 | 4.07E−05 | 0.72 | 2.33E−08 | 0.66 | 9.83E−07 |
| A_23_P104464 | ALOX5 | 0.77 | 5.64E−06 | 0.69 | 1.02E−07 | 0.68 | 3.93E−07 |
| A_23_P104464 | ALOX5 | 0.69 | 1.18E−04 | 0.71 | 3.68E−07 | 0.69 | 2.36E−07 |
| A_23_P104464 | ALOX5 | 0.75 | 2.22E−05 | 0.72 | 7.63E−08 | 0.69 | 2.40E−07 |
| A_23_P104464 | ALOX5 | 0.74 | 2.63E−05 | 0.74 | 2.37E−08 | 0.69 | 1.88E−07 |
| A_24_P347378 | ALOX5AP | 0.61 | 9.85E−04 | 0.47 | 1.76E−03 | 0.69 | 2.80E−06 |
| A_24_P353619 | ALPL | 0.81 | 3.00E−05 | 0.85 | 1.80E−06 | 0.77 | 3.34E−05 |
| A_23_P156748 | ANICS1A | 0.66 | 1.84E−05 | 0.56 | 1.37E−08 | 0.67 | 5.49E−11 |
| A_23_P94501 | ANXA1 | 0.63 | 5.27E−05 | 0.71 | 3.99E−07 | 0.55 | 1.81E−05 |
| A_23_P121716 | ANXA3 | 1.06 | 2.36E−05 | 1.03 | 8.35E−07 | 1.14 | 1.58E−07 |
| A_23_P121716 | ANXA3 | 1.08 | 1.52E−05 | 1.04 | 1.21E−06 | 1.14 | 1.60E−07 |
| A_23_P121716 | ANXA3 | 1.08 | 1.68E−05 | 1.03 | 8.55E−07 | 1.11 | 3.31E−07 |
| A_23_P121716 | ANXA3 | 1.07 | 2.39E−05 | 1.05 | 8.57E−07 | 1.12 | 2.42E−07 |
| A_23_P121716 | ANXA3 | 1.10 | 1.87E−05 | 1.03 | 3.96E−07 | 1.11 | 1.70E−07 |
| A_23_P121716 | ANXA3 | 1.12 | 6.39E−06 | 1.04 | 6.47E−07 | 1.12 | 1.62E−07 |
| A_23_P121716 | ANXA3 | 1.10 | 2.04E−05 | 1.02 | 7.71E−07 | 1.13 | 2.06E−07 |
| A_23_P121716 | ANXA3 | 1.11 | 2.07E−05 | 1.03 | 5.49E−07 | 1.12 | 2.29E−07 |
| A_23_P121716 | ANXA3 | 1.09 | 1.57E−05 | 1.04 | 6.23E−07 | 1.12 | 2.16E−07 |
| A_23_P121716 | ANXA3 | 1.11 | 1.71E−05 | 1.04 | 6.80E−07 | 1.11 | 3.40E−07 |
| A_33_P3352382 | ARG1 | 1.11 | 3.03E−04 | 1.21 | 1.56E−06 | 1.46 | 1.47E−07 |
| A_33_P3319967 | ARG1 | 1.03 | 1.39E−03 | 1.19 | 8.05E−06 | 1.51 | 1.45E−07 |
| A_23_P143016 | ARID5A | 0.60 | 5.05E−05 | 0.58 | 2.47E−06 | 0.73 | 7.00E−10 |
| A_23_P217712 | ARSD | −0.01 | 9.65E−01 | −0.68 | 4.76E−03 | −0.62 | 5.37E−03 |
| A_23_P216094 | ASPH | 0.58 | 3.37E−04 | 0.69 | 2.77E−08 | 0.81 | 6.30E−11 |
| A_24_P295245 | ASPH | 0.71 | 1.65E−04 | 0.79 | 5.17E−08 | 0.98 | 4.23E−11 |
| A_24_P161973 | ATP11A | 0.59 | 6.42E−06 | 0.50 | 1.23E−07 | 0.66 | 2.56E−09 |
| A_23_P212522 | ATP11B | 0.64 | 3.33E−06 | 0.70 | 5.00E−08 | 0.74 | 7.05E−11 |
| A_24_P405205 | ATP2B4 | 0.58 | 1.10E−04 | 0.60 | 1.24E−07 | 0.66 | 7.41E−11 |
| A_23_P146058 | ATP6V1C1 | 0.98 | 2.85E−06 | 0.86 | 1.26E−06 | 1.01 | 1.41E−09 |
| A_33_P3380897 | ATP6V1C1 | 0.76 | 3.49E−05 | 0.75 | 1.21E−06 | 0.92 | 1.75E−10 |
| A_23_P380614 | ATP9A | 0.92 | 4.51E−05 | 0.93 | 8.45E−06 | 1.10 | 4.78E−09 |
| A_23_P18372 | B3GNT5 | 0.64 | 8.59E−06 | 0.58 | 1.17E−05 | 0.73 | 2.72E−09 |
| A_24_P239731 | B4GALT5 | 0.86 | 1.61E−07 | 0.92 | 3.63E−10 | 1.05 | 4.34E−13 |
| A_23_P213385 | BASP1 | 0.68 | 3.73E−05 | 0.65 | 2.37E−07 | 0.66 | 2.07E−06 |
| A_23_P128974 | BATF | 0.48 | 1.14E−03 | 0.60 | 2.79E−06 | 0.73 | 3.42E−09 |
| A_23_P152002 | BCL2A1 | 0.82 | 2.13E−05 | 0.76 | 2.65E−06 | 0.84 | 3.74E−07 |
| A_23_P57856 | BCL6 | 0.81 | 9.14E−05 | 0.92 | 2.38E−11 | 0.89 | 8.26E−08 |
| A_23_P310911 | BLMH | 0.83 | 6.56E−05 | 0.78 | 4.50E−06 | 0.89 | 3.57E−08 |
| A_23_P253602 | BMX | 0.73 | 1.51E−04 | 0.79 | 1.66E−06 | 0.91 | 3.84E−08 |
| A_23_P131785 | BPI | 0.71 | 2.32E−04 | 0.65 | 5.72E−04 | 0.85 | 2.74E−05 |
| A_33_P3245389 | C14orf101 | 0.59 | 2.90E−04 | 0.59 | 5.06E−06 | 0.68 | 1.77E−09 |
| A_23_P26557 | C16orf59 | −0.12 | 6.21E−01 | −0.62 | 5.07E−03 | −0.75 | 2.90E−04 |
| A_23_P330561 | C19orf59 | 1.38 | 4.51E−05 | 1.35 | 2.20E−06 | 1.78 | 1.56E−09 |
| A_24_P297078 | C20orf3 | 0.74 | 8.60E−05 | 0.62 | 7.13E−06 | 0.61 | 3.11E−05 |
| A_21_P0000149 | C2orf3 | −0.07 | 7.89E−01 | −0.71 | 9.00E−03 | −0.71 | 1.88E−03 |
| A_33_P3347869 | C3 | 0.02 | 9.50E−01 | −0.73 | 7.90E−03 | −0.88 | 7.80E−04 |
| A_23_P259506 | C5orf32 | 0.96 | 2.79E−05 | 1.07 | 7.74E−08 | 1.13 | 5.48E−08 |
| A_33_P3431595 | C8orf31 | 0.02 | 9.38E−01 | −0.73 | 9.11E−03 | −0.74 | 6.72E−03 |
| A_23_P123732 | C9orf103 | −0.15 | 3.38E−01 | 0.59 | 2.38E−03 | 0.63 | 1.65E−04 |
| A_23_P123732 | C9orf103 | 0.10 | 5.78E−01 | 0.64 | 4.66E−04 | 0.70 | 2.68E−05 |
| A_23_P123732 | C9orf103 | 0.32 | 5.26E−02 | 0.67 | 1.01E−04 | 0.69 | 5.77E−06 |
| A_23_P123732 | C9orf103 | 0.12 | 4.53E−01 | 0.65 | 1.91E−04 | 0.63 | 3.00E−05 |
| A_33_P3359223 | C9orf173 | −0.54 | 3.28E−02 | −0.75 | 3.71E−04 | −0.94 | 1.07E−06 |
| A_33_P3282614 | C9orf173 | −0.34 | 1.12E−01 | −0.66 | 4.79E−04 | −0.80 | 4.71E−06 |
| A_23_P20804 | C9orf25 | −0.63 | 3.06E−03 | −0.37 | 4.90E−02 | −0.66 | 9.36E−04 |
| A_23_P4096 | CA4 | 0.70 | 4.63E−04 | 0.66 | 1.12E−04 | 0.79 | 1.97E−06 |
| A_23_P79426 | CAB39 | 0.68 | 1.08E−06 | 0.65 | 2.26E−08 | 0.71 | 5.46E−10 |
| A_33_P3228612 | CACNA1E | 0.59 | 3.17E−04 | 0.75 | 3.45E−07 | 0.71 | 3.78E−09 |
| A_23_P408830 | CAMKK2 | 0.60 | 8.95E−05 | 0.60 | 2.46E−05 | 0.57 | 2.19E−05 |
| A_33_P3281816 | CAP1 | 0.59 | 8.98E−05 | 0.66 | 9.74E−08 | 0.69 | 4.95E−08 |
| A_33_P3340847 | CARD6 | 0.51 | 1.60E−03 | 0.62 | 6.84E−06 | 0.89 | 1.02E−10 |
| A_23_P41854 | CARD6 | 0.67 | 5.31E−05 | 0.64 | 1.90E−05 | 0.87 | 1.65E−10 |
| A_23_P344884 | CARNS1 | −0.34 | 6.24E−02 | −0.69 | 2.29E−04 | −0.70 | 4.89E−06 |
| A_23_P155306 | CBS | 0.44 | 1.51E−02 | 0.70 | 9.15E−06 | 0.76 | 1.60E−05 |
| A_33_P3246613 | CCDC78 | −0.66 | 1.99E−02 | −0.67 | 5.17E−03 | −0.98 | 5.21E−05 |
| A_24_P148717 | CCR1 | 0.63 | 1.13E−03 | 0.60 | 3.26E−04 | 0.58 | 2.35E−04 |
| A_23_P250302 | CCR3 | −0.38 | 9.50E−02 | −0.59 | 1.82E−03 | −0.98 | 2.85E−06 |
| A_23_P33723 | CD163 | 0.77 | 1.26E−03 | 0.45 | 2.68E−02 | 0.77 | 5.22E−06 |
| A_24_P380536 | CD164 | 0.49 | 1.99E−05 | 0.48 | 8.75E−06 | 0.63 | 3.14E−12 |
| A_23_P254756 | CD164 | 0.59 | 1.10E−05 | 0.56 | 1.18E−07 | 0.51 | 8.99E−08 |
| A_21_P0011751 | CD177 | 1.91 | 9.92E−06 | 1.98 | 5.61E−07 | 2.38 | 1.43E−08 |
| A_23_P259863 | CD177 | 1.87 | 5.48E−05 | 1.77 | 5.98E−06 | 2.21 | 1.43E−07 |
| A_33_P3232080 | CD177 | 0.65 | 1.48E−04 | 0.80 | 3.24E−05 | 0.92 | 2.24E−07 |
| A_33_P3375541 | CD3D | −0.63 | 1.90E−03 | −0.84 | 1.89E−06 | −0.95 | 8.63E−08 |
| A_33_P3294509 | CD44 | 0.65 | 5.42E−04 | 0.63 | 5.40E−06 | 0.58 | 6.31E−07 |
| A_24_P188377 | CD55 | 0.64 | 1.78E−04 | 0.81 | 3.54E−08 | 0.91 | 7.33E−10 |
| A_24_P270144 | CD63 | 0.59 | 7.45E−05 | 0.64 | 4.70E−06 | 0.76 | 1.31E−08 |
| A_23_P107735 | CD79A | −0.79 | 3.79E−03 | −0.54 | 4.90E−03 | −0.79 | 7.88E−05 |
| A_23_P1782 | CD82 | 0.52 | 1.53E−05 | 0.59 | 9.87E−08 | 0.66 | 4.35E−10 |
| A_33_P3389060 | CDK5RAP2 | 0.88 | 7.32E−05 | 0.97 | 3.11E−07 | 1.09 | 9.85E−10 |
| A_23_P83110 | CDK5RAP2 | 0.98 | 1.22E−04 | 1.03 | 1.92E−06 | 1.21 | 8.99E−09 |
| A_33_P3268507 | CEACAM1 | 0.79 | 1.41E−06 | 0.64 | 2.18E−05 | 0.78 | 9.58E−09 |
| A_24_P382319 | CEACAM1 | 1.12 | 5.80E−06 | 1.05 | 3.03E−06 | 1.25 | 2.12E−09 |
| A_24_P120115 | CFLAR | 0.66 | 8.29E−07 | 0.60 | 3.88E−09 | 0.63 | 2.44E−08 |
| A_23_P137665 | CHI3L1 | −0.83 | 3.49E−03 | −0.96 | 2.47E−04 | −0.75 | 1.66E−03 |
| A_24_P945293 | CHMP3 | −0.35 | 1.60E−01 | −0.79 | 2.31E−04 | −0.77 | 3.17E−04 |
| A_23_P48056 | CKAP4 | 0.71 | 7.08E−05 | 0.81 | 2.26E−07 | 0.92 | 5.54E−08 |
| A_23_P128470 | CLEC12A | 0.83 | 7.92E−04 | 0.70 | 4.78E−03 | 0.55 | 4.81E−03 |
| A_33_P3352578 | CLEC4D | 1.07 | 1.36E−05 | 1.08 | 1.92E−06 | 1.29 | 5.58E−10 |
| A_33_P3258977 | CLEC4D | 1.00 | 2.43E−05 | 0.99 | 1.12E−05 | 1.15 | 4.45E−08 |
| A_23_P411113 | CNTNAP1 | −0.80 | 3.31E−03 | −0.63 | 5.82E−03 | −0.55 | 2.77E−03 |
| A_33_P3216448 | COL11A2 | −0.35 | 1.64E−01 | −0.81 | 4.30E−04 | −0.80 | 3.93E−04 |
| A_23_P258164 | CORT | −0.25 | 2.97E−01 | −0.65 | 8.71E−03 | −0.94 | 1.74E−05 |
| A_23_P251937 | CPEB4 | 0.61 | 2.69E−05 | 0.54 | 1.28E−06 | 0.72 | 5.06E−09 |
| A_23_P256821 | CR1 | 0.77 | 5.81E−06 | 0.93 | 2.06E−09 | 0.99 | 3.02E−11 |
| A_33_P3319126 | CR1L | 0.75 | 6.38E−07 | 0.87 | 1.07E−09 | 0.86 | 5.73E−12 |
| A_23_P149892 | CSGALNACT2 | 0.89 | 3.62E−07 | 0.91 | 9.16E−10 | 0.94 | 2.28E−10 |
| A_33_P3402526 | CSGALNACT2 | 0.79 | 2.18E−06 | 0.80 | 8.75E−09 | 0.99 | 4.05E−13 |
| A_23_P68601 | CST7 | 0.69 | 8.01E−03 | 0.76 | 9.89E−05 | 0.88 | 3.11E−06 |
| A_23_P94533 | CTSL1 | 0.03 | 8.89E−01 | −0.60 | 6.58E−03 | −0.59 | 2.85E−03 |
| A_23_P209625 | CYP1B1 | 0.77 | 2.49E−04 | 0.77 | 1.45E−04 | 0.92 | 9.86E−08 |
| A_33_P3290343 | CYP1B1 | 0.90 | 6.13E−06 | 0.83 | 6.07E−05 | 0.91 | 1.01E−07 |
| A_33_P3361422 | CYP27A1 | −0.74 | 2.07E−04 | −0.80 | 5.48E−06 | −0.58 | 1.29E−04 |
| A_33_P3316786 | DACH1 | 0.81 | 3.38E−06 | 0.88 | 1.54E−07 | 1.00 | 5.35E−11 |
| A_23_P32577 | DACH1 | 0.76 | 7.46E−08 | 0.77 | 3.93E−07 | 0.80 | 2.00E−09 |
| A_23_P19482 | DDAH2 | 1.04 | 7.45E−06 | 1.20 | 9.46E−11 | 1.32 | 9.34E−12 |
| A_23_P66719 | DHRS13 | 0.56 | 1.27E−03 | 0.59 | 1.31E−05 | 0.65 | 3.50E−07 |
| A_23_P56559 | DHRS9 | 1.34 | 3.99E−06 | 1.04 | 1.40E−05 | 1.31 | 6.05E−09 |
| A_21_P0011611 | DNAH17 | −0.65 | 2.31E−02 | −1.04 | 1.11E−04 | −0.95 | 3.79E−05 |
| A_33_P3253144 | DOK3 | 0.56 | 1.10E−03 | 0.62 | 4.21E−06 | 0.73 | 1.53E−08 |
| A_23_P99163 | DRAM1 | 0.63 | 1.58E−05 | 0.64 | 1.29E−06 | 0.76 | 1.17E−10 |
| A_23_P39931 | DYSF | 0.86 | 2.28E−06 | 0.80 | 2.30E−08 | 0.91 | 9.25E−10 |
| A_23_P401606 | EDIL3 | −0.37 | 1.71E−01 | −0.90 | 3.24E−03 | −0.99 | 1.03E−04 |
| A_23_P126241 | EIF4G3 | 0.63 | 7.25E−07 | 0.60 | 7.25E−09 | 0.74 | 4.61E−12 |
| A_24_P322635 | ELMO2 | 0.55 | 2.96E−05 | 0.60 | 5.84E−09 | 0.59 | 1.05E−09 |
| A_33_P3359900 | EMB | 0.74 | 9.32E−06 | 0.86 | 4.39E−09 | 1.00 | 1.49E−13 |
| A_24_P684186 | EMB | 0.61 | 2.26E−05 | 0.61 | 8.87E−08 | 0.69 | 1.91E−09 |
| A_23_P27315 | EMILIN2 | 0.61 | 3.81E−04 | 0.54 | 2.42E−04 | 0.67 | 3.70E−08 |
| A_23_P106145 | ERO1L | 0.60 | 3.10E−05 | 0.44 | 1.71E−04 | 0.59 | 2.27E−07 |
| A_24_P314179 | ETS2 | 0.84 | 4.91E−08 | 0.70 | 1.78E−07 | 0.75 | 5.06E−09 |
| A_33_P3301410 | EXOSC4 | 0.78 | 5.97E−05 | 0.84 | 1.78E−06 | 1.04 | 5.36E−11 |
| A_23_P133438 | FAM105A | 0.64 | 5.20E−06 | 0.55 | 4.34E−06 | 0.62 | 5.56E−10 |
| A_23_P334864 | FAM126B | 0.68 | 1.21E−05 | 0.71 | 4.04E−08 | 0.77 | 3.51E−08 |
| A_32_P108254 | FAM20A | 0.65 | 5.55E−04 | 0.66 | 5.94E−04 | 0.86 | 7.22E−07 |
| A_23_P214026 | FBN2 | 0.18 | 5.49E−01 | −0.79 | 3.85E−03 | −0.77 | 2.29E−03 |
| A_33_P3329549 | FBRS | −0.46 | 2.59E−02 | −0.67 | 1.06E−03 | −0.72 | 3.17E−04 |
| A_23_P208768 | FCAR | 0.90 | 6.42E−06 | 0.84 | 7.12E−07 | 0.92 | 4.16E−08 |
| A_24_P348265 | FCAR | 0.75 | 4.88E−06 | 0.74 | 6.18E−08 | 0.94 | 2.40E−10 |
| A_23_P103765 | FCER1A | −0.69 | 1.29E−04 | −0.65 | 7.66E−06 | −0.92 | 2.24E−07 |
| A_23_P103765 | FCER1A | −0.59 | 5.06E−03 | −0.59 | 1.69E−04 | −0.95 | 1.51E−06 |
| A_23_P103765 | FCER1A | −0.49 | 1.11E−02 | −0.62 | 1.56E−05 | −0.94 | 1.14E−07 |
| A_23_P103765 | FCER1A | −0.49 | 7.16E−03 | −0.66 | 9.88E−06 | −0.89 | 2.22E−07 |
| A_21_P0010561 | FCGR1B | 0.95 | 4.43E−06 | 0.85 | 2.38E−05 | 0.72 | 3.23E−05 |
| A_21_P0010728 | FCGR1B | 0.91 | 9.34E−06 | 0.83 | 1.13E−05 | 0.72 | 1.88E−05 |
| A_23_P63390 | FCGR1B | 0.83 | 1.56E−04 | 0.87 | 7.95E−06 | 0.77 | 1.81E−05 |
| A_33_P3298810 | FFAR3 | 0.63 | 2.70E−03 | 0.68 | 3.39E−04 | 0.84 | 7.53E−07 |
| A_23_P217319 | FGF13 | 0.55 | 2.84E−02 | 0.60 | 4.46E−03 | 0.82 | 6.09E−05 |
| A_24_P38081 | FKBP5 | 1.08 | 3.82E−06 | 1.08 | 1.23E−08 | 1.19 | 3.31E−09 |
| A_23_P214603 | FLOT1 | 0.59 | 1.34E−04 | 0.54 | 8.09E−07 | 0.60 | 7.96E−08 |
| A_24_P253818 | FLOT2 | 0.80 | 7.79E−08 | 0.71 | 1.61E−07 | 0.64 | 4.19E−08 |
| A_24_P223124 | FNDC3B | 0.59 | 6.27E−06 | 0.51 | 7.47E−07 | 0.62 | 2.31E−09 |
| A_23_P23221 | GADD45A | 1.06 | 1.08E−06 | 1.16 | 8.65E−10 | 1.32 | 1.86E−11 |
| A_23_P67847 | GALNT14 | 1.07 | 1.22E−04 | 1.20 | 8.53E−08 | 1.26 | 2.31E−09 |
| A_24_P353794 | GALNT2 | 0.60 | 7.90E−06 | 0.65 | 6.78E−07 | 0.63 | 2.26E−08 |
| A_24_P82466 | GAS7 | 0.61 | 2.82E−06 | 0.63 | 1.13E−07 | 0.62 | 3.50E−11 |
| A_23_P28485 | GCA | 0.74 | 5.27E−05 | 0.64 | 4.19E−07 | 0.73 | 3.33E−07 |
| A_33_P3343155 | GNAQ | 0.60 | 2.77E−05 | 0.61 | 6.24E−09 | 0.71 | 1.73E−12 |
| A_23_P112260 | GNG10 | 0.62 | 9.66E−05 | 0.60 | 3.04E−07 | 0.64 | 1.37E−06 |
| A_23_P8640 | GPER | 0.59 | 3.45E−03 | 0.46 | 3.80E−03 | 0.71 | 4.73E−06 |
| A_23_P6943 | GPR15 | 0.09 | 7.55E−01 | −0.89 | 3.21E−03 | −0.85 | 2.48E−03 |
| A_23_P167005 | GPR160 | 0.64 | 2.72E−04 | 0.63 | 3.97E−06 | 0.70 | 2.52E−06 |
| A_23_P25155 | GPR84 | 1.25 | 3.72E−05 | 1.28 | 1.64E−07 | 1.50 | 3.40E−11 |
| A_23_P140760 | GPR97 | 0.59 | 8.37E−06 | 0.69 | 2.97E−06 | 0.76 | 4.30E−08 |
| A_33_P3331687 | GPSM1 | −0.38 | 1.00E−01 | −0.68 | 1.40E−03 | −0.76 | 1.30E−04 |
| A_23_P122863 | GRB10 | 0.79 | 3.54E−04 | 0.86 | 2.04E−06 | 1.23 | 5.13E−11 |
| A_23_P122863 | GRB10 | 0.77 | 4.92E−04 | 0.90 | 8.44E−07 | 1.21 | 1.65E−10 |
| A_23_P122863 | GRB10 | 0.89 | 1.71E−04 | 0.88 | 1.58E−06 | 1.28 | 4.32E−10 |
| A_23_P122863 | GRB10 | 0.91 | 1.62E−04 | 0.90 | 1.15E−06 | 1.23 | 1.23E−10 |
| A_23_P122863 | GRB10 | 0.79 | 3.57E−04 | 0.93 | 4.22E−07 | 1.24 | 3.57E−11 |
| A_23_P122863 | GRB10 | 0.79 | 6.06E−04 | 0.92 | 7.58E−07 | 1.26 | 5.91E−11 |
| A_23_P122863 | GRB10 | 0.80 | 3.16E−04 | 0.90 | 1.19E−06 | 1.23 | 8.09E−11 |
| A_24_P235266 | GRB10 | 1.00 | 3.49E−05 | 1.04 | 1.03E−07 | 1.32 | 4.39E−11 |
| A_23_P122863 | GRB10 | 0.81 | 2.53E−04 | 0.90 | 3.05E−07 | 1.21 | 9.48E−11 |
| A_23_P122863 | GRB10 | 0.82 | 2.17E−04 | 0.91 | 7.43E−08 | 1.21 | 5.06E−11 |
| A_23_P122863 | GRB10 | 0.82 | 2.38E−04 | 0.93 | 2.31E−07 | 1.23 | 7.15E−11 |
| A_23_P153945 | GTDC1 | 0.61 | 1.67E−05 | 0.61 | 5.57E−06 | 0.71 | 7.76E−09 |
| A_23_P29422 | GYG1 | 1.28 | 4.10E−07 | 1.22 | 9.68E−08 | 1.35 | 2.78E−11 |
| A_21_P0013518 | GYG1 | 1.18 | 1.42E−07 | 1.14 | 3.43E−08 | 1.28 | 2.09E−11 |
| A_23_P384517 | GYG1 | 1.12 | 1.46E−05 | 1.15 | 7.06E−07 | 1.36 | 5.73E−10 |
| A_33_P3376821 | GZMA | −0.62 | 1.31E−03 | −0.76 | 2.39E−05 | −0.60 | 1.87E−03 |
| A_23_P156218 | GZMK | −0.64 | 6.94E−04 | −0.65 | 2.23E−04 | −0.63 | 3.96E−04 |
| A_33_P3306624 | HCRT | −0.09 | 7.88E−01 | −0.88 | 3.36E−03 | −0.95 | 3.50E−04 |
| A_23_P47034 | HHEX | 0.57 | 7.86E−05 | 0.60 | 1.84E−05 | 0.61 | 8.46E−06 |
| A_24_P363548 | HIP1 | 0.64 | 1.22E−06 | 0.53 | 1.76E−07 | 0.71 | 9.41E−11 |
| A_23_P213584 | HK3 | 1.22 | 6.82E−08 | 1.11 | 2.91E−08 | 1.22 | 7.15E−13 |
| A_24_P50245 | HLA-DMA | −0.28 | 2.01E−02 | −0.60 | 8.98E−08 | −0.68 | 2.11E−09 |
| A_32_P351968 | HLA-DMB | −0.66 | 4.17E−04 | −0.84 | 4.52E−07 | −0.94 | 9.74E−08 |
| A_23_P30913 | HLA-DPA1 | −0.42 | 9.92E−03 | −0.70 | 5.24E−07 | −0.74 | 4.10E−06 |
| A_33_P3234277 | HLA-DPA1 | −0.32 | 2.49E−02 | −0.64 | 4.28E−06 | −0.84 | 1.34E−07 |
| A_24_P166443 | HLA-DPB1 | −0.43 | 6.24E−04 | −0.67 | 5.96E−08 | −0.66 | 4.63E−06 |
| A_33_P3271651 | HLA-DPB1 | −0.36 | 1.51E−02 | −0.61 | 2.05E−05 | −0.68 | 2.10E−05 |
| A_23_P8108 | HLA-DQB1 | −0.26 | 5.05E−02 | −0.62 | 5.67E−06 | −0.81 | 2.45E−08 |
| A_32_P87697 | HLA-DRA | −0.29 | 8.75E−02 | −0.72 | 6.77E−06 | −0.86 | 2.97E−07 |
| A_24_P343233 | HLA-DRB1 | −0.41 | 3.93E−04 | −0.60 | 7.09E−08 | −0.71 | 5.22E−08 |
| A_24_P343233 | HLA-DRB1 | −0.37 | 1.21E−03 | −0.59 | 1.29E−07 | −0.73 | 4.69E−08 |
| A_24_P343233 | HLA-DRB1 | −0.31 | 9.20E−03 | −0.68 | 1.18E−07 | −0.80 | 2.81E−09 |
| A_24_P343233 | HLA-DRB1 | −0.32 | 1.02E−02 | −0.71 | 1.76E−08 | −0.86 | 7.40E−10 |
| A_24_P343233 | HLA-DRB1 | −0.31 | 8.66E−03 | −0.71 | 4.37E−08 | −0.82 | 1.14E−09 |
| A_24_P343233 | HLA-DRB1 | −0.26 | 1.20E−02 | −0.59 | 6.36E−07 | −0.74 | 6.06E−08 |
| A_24_P343233 | HLA-DRB1 | −0.27 | 2.61E−02 | −0.74 | 2.70E−09 | −0.85 | 6.07E−10 |
| A_33_P3383912 | HLA-DRB3 | −0.18 | 2.21E−01 | −0.61 | 2.43E−06 | −0.68 | 1.57E−06 |
| A_33_P3394605 | HMG20B | −0.37 | 1.35E−01 | −0.62 | 2.72E−04 | −0.59 | 7.82E−04 |
| A_23_P155765 | HMGB2 | 0.52 | 3.74E−03 | 0.65 | 3.78E−06 | 0.77 | 9.47E−08 |
| A_23_P206760 | HP | 1.39 | 1.21E−04 | 1.33 | 6.27E−05 | 1.77 | 4.23E−09 |
| A_33_P3289236 | HPR | 1.31 | 9.79E−05 | 1.15 | 1.01E−04 | 1.54 | 1.32E−09 |
| A_23_P142125 | HRC | −0.18 | 4.65E−01 | −0.72 | 8.73E−04 | −0.75 | 2.35E−04 |
| A_24_P103886 | IDI1 | 0.82 | 1.01E−05 | 0.73 | 6.78E−07 | 0.70 | 2.01E−06 |
| A_23_P52266 | IFIT1 | −0.60 | 4.99E−02 | −0.80 | 1.18E−03 | −1.07 | 9.52E−05 |
| A_33_P3224809 | IL17RA | 0.60 | 3.03E−06 | 0.47 | 6.85E−07 | 0.59 | 1.31E−08 |
| A_23_P17706 | IL17RA | 0.65 | 1.88E−06 | 0.58 | 2.38E−09 | 0.67 | 1.01E−10 |
| A_33_P3540143 | IL17RA | 0.61 | 4.77E−05 | 0.57 | 3.65E−07 | 0.73 | 3.86E−10 |
| A_33_P3251876 | IL18R1 | 0.75 | 1.72E−04 | 0.85 | 2.07E−07 | 1.05 | 1.93E−12 |
| A_33_P3211666 | IL18R1 | 0.97 | 4.81E−04 | 1.09 | 4.00E−06 | 1.44 | 8.00E−12 |
| A_24_P208567 | IL18R1 | 1.20 | 2.07E−05 | 1.25 | 1.63E−07 | 1.47 | 3.69E−12 |
| A_23_P28334 | IL18RAP | 1.02 | 6.51E−05 | 1.11 | 5.60E−08 | 1.28 | 2.88E−09 |
| A_33_P3221960 | IL18RAP | 1.06 | 3.41E−05 | 1.09 | 3.61E−08 | 1.28 | 2.04E−09 |
| A_24_P63019 | IL1R2 | 1.56 | 1.24E−06 | 1.54 | 1.51E−08 | 1.81 | 2.94E−10 |
| A_23_P170857 | IL1RAP | 0.85 | 2.91E−07 | 0.64 | 1.59E−05 | 0.61 | 4.27E−05 |
| A_23_P129556 | IL4R | 0.67 | 8.61E−04 | 0.78 | 1.20E−07 | 0.83 | 2.40E−07 |
| A_33_P3349045 | IL4R | 0.64 | 2.40E−06 | 0.60 | 1.15E−07 | 0.63 | 3.03E−09 |
| A_23_P109907 | ILDR1 | 0.32 | 2.75E−01 | 0.62 | 1.92E−03 | 0.61 | 9.38E−04 |
| A_23_P109907 | ILDR1 | 0.27 | 3.46E−01 | 0.60 | 2.28E−03 | 0.61 | 8.51E−04 |
| A_23_P18119 | IMPG2 | −0.23 | 3.61E−01 | −0.69 | 1.75E−03 | −0.79 | 1.80E−04 |
| A_23_P73780 | IRAK1 | −0.39 | 7.29E−02 | −0.61 | 1.11E−03 | −0.71 | 1.17E−04 |
| A_23_P162300 | IRAK3 | 0.86 | 9.29E−06 | 0.88 | 5.97E−09 | 1.15 | 4.94E−12 |
| A_23_P128084 | ITGA7 | 0.64 | 2.46E−03 | 0.68 | 6.30E−04 | 0.86 | 2.54E−05 |
| A_23_P124108 | ITGAM | 0.71 | 1.00E−05 | 0.58 | 2.22E−06 | 0.69 | 8.34E−08 |
| A_23_P124108 | ITGAM | 0.69 | 4.16E−05 | 0.57 | 2.94E−06 | 0.70 | 3.94E−08 |
| A_23_P124108 | ITGAM | 0.70 | 2.00E−05 | 0.57 | 3.09E−06 | 0.67 | 1.12E−07 |
| A_23_P124108 | ITGAM | 0.87 | 6.18E−04 | 0.58 | 3.11E−06 | 0.72 | 6.37E−08 |
| A_23_P124108 | ITGAM | 0.69 | 2.76E−05 | 0.62 | 4.64E−07 | 0.71 | 2.89E−08 |
| A_23_P124108 | ITGAM | 0.69 | 1.70E−05 | 0.59 | 1.34E−06 | 0.74 | 1.48E−08 |
| A_23_P124108 | ITGAM | 0.71 | 1.14E−05 | 0.58 | 2.60E−06 | 0.72 | 3.98E−08 |
| A_23_P124108 | ITGAM | 0.69 | 1.28E−05 | 0.59 | 1.71E−06 | 0.72 | 2.40E−08 |
| A_23_P124108 | ITGAM | 0.70 | 1.13E−05 | 0.60 | 1.71E−06 | 0.71 | 4.26E−08 |
| A_23_P124108 | ITGAM | 0.73 | 1.05E−05 | 0.59 | 1.33E−06 | 0.72 | 2.11E−08 |
| A_24_P59667 | JAK3 | 0.62 | 4.52E−04 | 0.69 | 1.47E−07 | 0.85 | 1.64E−11 |
| A_33_P3338793 | KCNC3 | −0.16 | 4.60E−01 | −0.68 | 8.58E−04 | −0.73 | 9.52E−05 |
| A_23_P109026 | KCNK15 | −0.45 | 2.55E−02 | −0.71 | 6.23E−05 | −0.71 | 8.15E−05 |
| A_23_P109026 | KCNK15 | −0.46 | 1.62E−02 | −0.64 | 3.56E−04 | −0.72 | 1.14E−04 |
| A_23_P109026 | KCNK15 | −0.26 | 2.90E−01 | −0.69 | 2.38E−04 | −0.69 | 2.11E−04 |
| A_23_P123393 | KCNQ3 | −0.60 | 2.30E−02 | −0.75 | 5.04E−03 | −0.74 | 7.23E−04 |
| A_23_P123393 | KCNQ3 | −0.95 | 1.29E−03 | −0.27 | 2.95E−01 | −0.68 | 6.81E−03 |
| A_23_P123393 | KCNQ3 | −0.79 | 4.91E−03 | −0.25 | 3.30E−01 | −0.63 | 6.95E−03 |
| A_23_P123393 | KCNQ3 | −0.79 | 5.05E−03 | −0.31 | 2.12E−01 | −0.64 | 7.98E−03 |
| A_23_P123393 | KCNQ3 | −0.40 | 8.32E−02 | −0.70 | 3.34E−03 | −0.76 | 6.79E−04 |
| A_23_P123393 | KCNQ3 | −0.27 | 3.35E−01 | −0.90 | 1.10E−04 | −0.92 | 1.72E−04 |
| A_23_P123393 | KCNQ3 | −0.57 | 5.90E−02 | −0.95 | 9.64E−05 | −1.12 | 1.29E−05 |
| A_23_P201287 | KIF1B | 0.85 | 1.29E−06 | 0.81 | 6.98E−09 | 0.89 | 2.71E−11 |
| A_24_P145066 | KIF1B | 0.71 | 6.77E−10 | 0.54 | 9.37E−08 | 0.68 | 2.63E−13 |
| A_24_P649624 | KIF1B | 0.76 | 6.79E−07 | 0.66 | 3.54E−06 | 0.80 | 9.10E−11 |
| A_23_P104741 | KIRREL3 | −0.07 | 7.91E−01 | −0.80 | 9.75E−04 | −0.84 | 1.13E−04 |
| A_23_P104741 | KIRREL3 | −0.19 | 4.81E−01 | −0.69 | 5.05E−03 | −0.76 | 6.15E−04 |
| A_23_P104741 | KIRREL3 | −0.54 | 4.95E−02 | −0.96 | 7.95E−05 | −0.95 | 5.36E−05 |
| A_23_P104741 | KIRREL3 | −0.37 | 1.85E−01 | −0.70 | 4.96E−03 | −0.95 | 1.85E−04 |
| A_23_P72503 | KLHL2 | 0.90 | 1.22E−06 | 0.88 | 1.85E−08 | 0.97 | 4.57E−09 |
| A_23_P68851 | KREMEN1 | 0.70 | 2.51E−04 | 0.66 | 2.31E−06 | 0.81 | 1.33E−06 |
| A_23_P211401 | KREMEN1 | 0.63 | 1.88E−06 | 0.60 | 2.47E−07 | 0.57 | 3.86E−07 |
| A_23_P107465 | KRT31 | −0.12 | 5.89E−01 | −0.63 | 6.33E−03 | −0.64 | 7.12E−04 |
| A_33_P3315303 | KRT73 | −0.31 | 3.45E−01 | −0.91 | 5.82E−04 | −0.97 | 3.82E−05 |
| A_33_P3357651 | KRTAP10-12 | −0.58 | 2.30E−02 | −0.93 | 4.50E−05 | −0.86 | 2.74E−05 |
| A_33_P3247473 | KRTAP23-1 | −0.24 | 3.15E−01 | −0.71 | 7.98E−04 | −0.59 | 3.02E−03 |
| A_23_P116765 | LALBA | −0.06 | 8.43E−01 | −0.88 | 8.47E−04 | −0.94 | 3.87E−05 |
| A_23_P116765 | LALBA | −0.24 | 4.47E−01 | −0.95 | 1.89E−04 | −0.90 | 3.77E−04 |
| A_23_P116765 | LALBA | −0.57 | 4.61E−02 | −1.03 | 5.99E−05 | −1.03 | 3.54E−05 |
| A_23_P103361 | LCK | −0.57 | 1.41E−03 | −0.59 | 2.10E−04 | −0.64 | 1.20E−04 |
| A_23_P169437 | LCN2 | 0.90 | 2.10E−03 | 0.66 | 3.30E−03 | 0.94 | 9.15E−04 |
| A_23_P47565 | LDHA | 0.85 | 1.34E−05 | 0.77 | 1.10E−06 | 0.84 | 1.06E−08 |
| A_24_P117029 | LDLR | 0.67 | 2.23E−05 | 0.58 | 2.77E−06 | 0.70 | 6.63E−08 |
| A_23_P120902 | LGALS2 | −0.81 | 2.94E−02 | −1.18 | 9.96E−04 | −1.30 | 2.79E−05 |
| A_23_P142205 | LILRA2 | 0.76 | 1.32E−05 | 0.62 | 4.74E−06 | 0.57 | 3.03E−06 |
| A_23_P79094 | LILRA3 | 0.80 | 1.95E−03 | 0.61 | 5.84E−03 | 0.84 | 5.65E−05 |
| A_23_P90497 | LILRA4 | 0.70 | 5.83E−06 | 0.60 | 1.57E−06 | 0.53 | 5.69E−07 |
| A_24_P370172 | LILRA5 | 0.96 | 3.14E−06 | 0.95 | 2.70E−08 | 1.01 | 1.16E−09 |
| A_32_P70158 | LILRB3 | 0.70 | 2.15E−04 | 0.64 | 2.00E−07 | 0.73 | 7.20E−08 |
| A_23_P376088 | LIME1 | −0.31 | 1.78E−01 | −0.71 | 8.11E−05 | −0.73 | 5.36E−05 |
| A_24_P353300 | LIMK2 | 0.74 | 1.54E−06 | 0.76 | 1.40E−11 | 0.73 | 1.51E−09 |
| A_33_P3311285 | LMNA | −0.36 | 1.25E−01 | −0.62 | 2.70E−03 | −0.72 | 7.25E−05 |
| A_23_P50638 | LRG1 | 0.64 | 2.17E−04 | 0.50 | 3.11E−04 | 0.68 | 4.11E−07 |
| A_33_P3306948 | LRP6 | −0.22 | 3.44E−01 | −0.63 | 2.51E−03 | −0.66 | 1.04E−03 |
| A_23_P29851 | LRPAP1 | 0.64 | 3.45E−05 | 0.58 | 9.76E−06 | 0.62 | 4.30E−08 |
| A_23_P41664 | LRRC70 | 0.61 | 2.69E−04 | 0.76 | 2.61E−07 | 0.69 | 1.67E−07 |
| A_23_P166848 | LTF | 1.18 | 1.62E−03 | 0.86 | 4.21E−03 | 1.07 | 2.53E−03 |
| A_23_P136870 | MAGEA6 | −0.16 | 4.91E−01 | −0.64 | 1.31E−03 | −0.64 | 8.54E−04 |
| A_23_P162211 | MANSC1 | 0.66 | 1.84E−05 | 0.58 | 1.14E−04 | 0.65 | 1.33E−06 |
| A_33_P3300308 | MAP1LC3A | −0.23 | 2.37E−01 | −0.63 | 3.62E−03 | −0.72 | 2.21E−04 |
| A_23_P207445 | MAP2K6 | 0.62 | 1.04E−04 | 0.53 | 5.64E−05 | 0.63 | 8.42E−07 |
| A_24_P283288 | MAPK14 | 0.96 | 5.48E−07 | 0.95 | 3.20E−10 | 0.95 | 4.09E−10 |
| A_33_P3272527 | MAVS | −0.19 | 4.39E−01 | −0.81 | 4.21E−03 | −0.71 | 3.18E−03 |
| A_24_P244944 | MCTP2 | 0.73 | 5.32E−06 | 0.76 | 2.25E−09 | 0.88 | 1.65E−12 |
| A_23_P65789 | MCTP2 | 0.76 | 8.23E−06 | 0.84 | 6.55E−10 | 0.87 | 6.21E−12 |
| A_33_P3341676 | MEF2A | 0.67 | 1.30E−04 | 0.63 | 4.40E−06 | 0.82 | 8.08E−10 |
| A_23_P103104 | MFNG | −0.33 | 2.44E−01 | −0.81 | 6.46E−03 | −0.90 | 1.21E−04 |
| A_23_P42897 | MGAM | 0.80 | 2.38E−05 | 0.83 | 2.38E−08 | 0.90 | 1.73E−08 |
| A_33_P3324884 | MICAL1 | 0.69 | 4.18E−06 | 0.72 | 5.77E−09 | 0.69 | 2.65E−10 |
| A_33_P3289541 | MLLT1 | 0.56 | 6.80E−06 | 0.60 | 1.39E−06 | 0.64 | 1.12E−09 |
| A_33_P3282394 | MLLT1 | 0.79 | 1.20E−05 | 0.75 | 1.98E−07 | 0.85 | 3.50E−11 |
| A_23_P9823 | MLXIP | 0.68 | 6.16E−08 | 0.62 | 1.98E−08 | 0.72 | 1.74E−11 |
| A_23_P40174 | MMP9 | 1.20 | 5.43E−05 | 1.11 | 6.58E−06 | 1.46 | 1.85E−09 |
| A_23_P40174 | MMP9 | 1.15 | 8.33E−05 | 1.09 | 7.06E−06 | 1.45 | 1.56E−09 |
| A_23_P40174 | MMP9 | 1.17 | 1.15E−04 | 1.13 | 4.55E−06 | 1.48 | 1.46E−09 |
| A_23_P40174 | MMP9 | 1.16 | 1.91E−04 | 1.12 | 8.16E−06 | 1.48 | 1.34E−09 |
| A_23_P40174 | MMP9 | 1.21 | 6.16E−05 | 1.11 | 4.48E−06 | 1.49 | 1.17E−09 |
| A_23_P40174 | MMP9 | 1.18 | 7.30E−05 | 1.13 | 3.03E−06 | 1.45 | 1.43E−09 |
| A_23_P40174 | MMP9 | 1.19 | 7.41E−05 | 1.12 | 2.17E−06 | 1.44 | 2.15E−09 |
| A_23_P40174 | MMP9 | 1.20 | 5.89E−05 | 1.13 | 2.02E−06 | 1.46 | 1.44E−09 |
| A_23_P40174 | MMP9 | 1.21 | 6.39E−05 | 1.15 | 1.77E−06 | 1.46 | 1.32E−09 |
| A_23_P40174 | MMP9 | 1.18 | 7.54E−05 | 1.12 | 2.34E−06 | 1.45 | 1.50E−09 |
| A_23_P141173 | MPO | 0.55 | 2.93E−03 | 0.61 | 3.10E−03 | 0.72 | 2.38E−03 |
| A_23_P75769 | MS4A4A | 0.95 | 4.78E−05 | 0.92 | 3.31E−05 | 1.00 | 3.08E−07 |
| A_23_P217778 | MSL3 | 0.61 | 2.74E−04 | 0.53 | 4.98E−05 | 0.69 | 3.49E−08 |
| A_23_P61426 | MSRA | 0.63 | 3.96E−05 | 0.63 | 4.85E−05 | 0.71 | 1.46E−07 |
| A_33_P3417281 | MUC4 | −0.41 | 3.81E−02 | −0.76 | 1.08E−04 | −0.68 | 3.69E−04 |
| A_24_P417706 | MXD3 | 0.66 | 1.69E−06 | 0.60 | 1.20E−09 | 0.83 | 7.70E−05 |
| A_23_P360240 | MYEOV | −0.23 | 3.19E−01 | −0.64 | 6.62E−03 | −0.60 | 8.30E−04 |
| A_23_P110473 | NAIP | 0.93 | 2.30E−05 | 0.86 | 1.80E−06 | 1.00 | 4.08E−08 |
| A_23_P110473 | NAIP | 0.93 | 1.87E−05 | 0.83 | 3.82E−06 | 1.02 | 3.62E−08 |
| A_23_P110473 | NAIP | 0.87 | 5.38E−05 | 0.82 | 1.35E−05 | 1.08 | 3.33E−08 |
| A_23_P110473 | NAIP | 0.91 | 2.12E−05 | 0.83 | 3.19E−06 | 1.01 | 1.69E−08 |
| A_21_P0012992 | NAIP | 0.90 | 6.87E−07 | 0.91 | 9.83E−08 | 0.94 | 2.93E−10 |
| A_23_P110473 | NAIP | 0.92 | 5.40E−05 | 0.83 | 1.03E−05 | 1.01 | 2.29E−08 |
| A_23_P110473 | NAIP | 0.95 | 1.51E−05 | 0.84 | 2.98E−06 | 1.02 | 3.11E−08 |
| A_23_P110473 | NAIP | 0.94 | 2.45E−05 | 0.87 | 1.62E−06 | 1.00 | 3.30E−08 |
| A_23_P110473 | NAIP | 0.95 | 1.51E−05 | 0.84 | 4.99E−06 | 1.01 | 2.48E−08 |
| A_23_P110473 | NAIP | 1.06 | 1.54E−05 | 0.85 | 2.84E−06 | 0.99 | 3.65E−08 |
| A_23_P110473 | NAIP | 0.94 | 2.26E−05 | 0.88 | 1.62E−06 | 1.00 | 2.85E−08 |
| A_21_P0013998 | NAIP | 1.14 | 8.49E−07 | 1.02 | 1.85E−08 | 1.06 | 3.59E−09 |
| A_33_P3364864 | NAMPT | 0.57 | 1.36E−03 | 0.67 | 6.35E−06 | 0.64 | 2.43E−05 |
| A_23_P87329 | NAT10 | −0.63 | 2.97E−03 | −0.33 | 9.38E−02 | −0.63 | 1.89E−03 |
| A_33_P3341970 | NEGR1 | −0.28 | 2.74E−01 | −0.73 | 4.87E−03 | −0.83 | 1.12E−04 |
| A_23_P119835 | NLRC4 | 0.86 | 2.82E−05 | 0.84 | 8.46E−08 | 1.05 | 2.49E−10 |
| A_23_P47579 | NLRP14 | −0.05 | 8.41E−01 | −0.63 | 5.94E−03 | −0.61 | 3.93E−03 |
| A_23_P82929 | NOV | −0.60 | 2.73E−04 | −0.68 | 2.74E−07 | −0.75 | 1.82E−07 |
| A_23_P58953 | NQO2 | 0.73 | 2.92E−04 | 0.87 | 4.27E−07 | 0.76 | 3.43E−06 |
| A_24_P7121 | NSUN7 | 0.64 | 1.04E−03 | 0.74 | 8.66E−06 | 0.90 | 7.03E−09 |
| A_23_P423331 | NTNG2 | 0.53 | 1.03E−03 | 0.68 | 3.74E−08 | 0.81 | 7.30E−10 |
| A_23_P161458 | OLAH | 0.78 | 1.08E−03 | 1.05 | 1.51E−05 | 1.15 | 9.99E−09 |
| A_24_P181254 | OLFM4 | 1.47 | 2.04E−04 | 1.20 | 2.07E−04 | 1.72 | 9.61E−06 |
| A_23_P170186 | OPLAH | 0.98 | 7.45E−06 | 1.10 | 2.64E−08 | 1.34 | 2.50E−12 |
| A_33_P3376090 | OR1J4 | 0.03 | 9.02E−01 | −0.61 | 4.53E−03 | −0.65 | 2.28E−03 |
| A_23_P94647 | OR1L3 | 0.03 | 9.26E−01 | −0.97 | 1.66E−03 | −0.91 | 1.19E−03 |
| A_23_P166408 | OSM | 0.98 | 4.63E−06 | 0.77 | 7.87E−06 | 0.89 | 1.66E−07 |
| A_23_P124003 | P2RX2 | −0.29 | 1.45E−01 | −0.73 | 6.63E−04 | −0.60 | 3.11E−03 |
| A_23_P124003 | P2RX2 | 0.08 | 7.74E−01 | −0.73 | 2.34E−03 | −0.72 | 1.14E−03 |
| A_24_P187970 | PADI2 | 0.61 | 1.01E−04 | 0.39 | 2.22E−02 | 0.64 | 5.21E−08 |
| A_33_P3216890 | PAG1 | 0.78 | 9.41E−08 | 0.70 | 6.46E−10 | 0.84 | 9.56E−13 |
| A_32_P61684 | PAG1 | 0.82 | 1.85E−07 | 0.75 | 2.45E−09 | 0.84 | 2.88E−09 |
| A_23_P252681 | PCYT1A | 0.59 | 4.03E−06 | 0.66 | 5.26E−09 | 0.69 | 4.42E−11 |
| A_23_P58396 | PDGFC | 0.41 | 1.54E−02 | 0.62 | 1.71E−04 | 0.78 | 2.02E−07 |
| A_23_P161152 | PDSS1 | 0.66 | 8.22E−06 | 0.55 | 8.82E−05 | 0.65 | 1.26E−08 |
| A_23_P91140 | PECR | 0.59 | 1.24E−05 | 0.64 | 6.82E−08 | 0.74 | 5.11E−12 |
| A_24_P413669 | PFKFB2 | 1.40 | 5.56E−06 | 1.44 | 2.86E−08 | 1.59 | 6.10E−11 |
| A_33_P3300635 | PFKFB2 | 1.19 | 2.32E−05 | 1.09 | 2.80E−07 | 1.36 | 2.02E−09 |
| A_24_P261259 | PFKFB3 | 1.24 | 9.92E−06 | 1.30 | 2.77E−09 | 1.47 | 4.16E−12 |
| A_24_P206604 | PFKFB3 | 0.54 | 1.02E−05 | 0.60 | 2.89E−07 | 0.78 | 1.10E−09 |
| A_23_P126623 | PGD | 0.80 | 7.62E−06 | 0.67 | 7.38E−06 | 0.72 | 7.32E−08 |
| A_23_P34510 | PHC2 | 0.61 | 4.51E−07 | 0.61 | 1.51E−07 | 0.69 | 3.45E−08 |
| A_24_P393864 | PHTF1 | 0.61 | 1.17E−06 | 0.52 | 1.05E−06 | 0.64 | 9.48E−10 |
| A_33_P3376234 | PHTF1 | 0.63 | 7.36E−06 | 0.67 | 1.27E−06 | 0.77 | 1.86E−10 |
| A_24_P183128 | PLAC8 | 0.93 | 3.77E−05 | 1.00 | 1.20E−05 | 1.12 | 9.92E−10 |
| A_23_P56356 | PLB1 | 0.88 | 1.41E−04 | 0.87 | 3.13E−07 | 0.93 | 2.65E−08 |
| A_33_P3400763 | PLIN4 | 0.83 | 2.09E−05 | 0.84 | 4.91E−08 | 1.04 | 2.43E−11 |
| A_23_P39251 | PLIN5 | 0.72 | 8.03E−05 | 0.68 | 2.59E−07 | 0.86 | 1.81E−08 |
| A_24_P74932 | PLP2 | 0.67 | 1.74E−04 | 0.62 | 8.40E−07 | 0.51 | 7.50E−06 |
| A_23_P69109 | PLSCR1 | 0.94 | 7.63E−06 | 0.81 | 4.76E−06 | 0.86 | 2.16E−06 |
| A_33_P3220422 | POM121L12 | 0.41 | 1.93E−01 | −0.74 | 7.00E−03 | −0.79 | 8.87E−04 |
| A_24_P29723 | POR | 0.84 | 4.89E−06 | 0.72 | 4.03E−06 | 0.76 | 1.07E−08 |
| A_33_P3835524 | POU2F2 | −0.28 | 1.85E−01 | −0.63 | 6.26E−04 | −0.78 | 1.59E−05 |
| A_23_P362759 | PRDM5 | 0.55 | 1.93E−04 | 0.60 | 1.44E−05 | 0.82 | 7.46E−12 |
| A_24_P97342 | PROK2 | 0.70 | 4.24E−04 | 0.71 | 1.85E−05 | 0.66 | 1.13E−04 |
| A_24_P322353 | PSTPIP2 | 0.62 | 2.71E−07 | 0.56 | 1.22E−06 | 0.61 | 6.45E−11 |
| A_23_P48676 | PYGL | 0.63 | 1.69E−03 | 0.59 | 1.57E−06 | 0.68 | 1.60E−06 |
| A_23_P12463 | QSOX1 | 0.55 | 2.54E−04 | 0.63 | 1.51E−07 | 0.83 | 5.34E−11 |
| A_24_P373174 | RAB27A | 0.67 | 5.70E−07 | 0.61 | 3.42E−08 | 0.62 | 2.76E−08 |
| A_24_P303480 | RAB32 | 0.61 | 3.56E−05 | 0.52 | 1.11E−04 | 0.61 | 5.59E−08 |
| A_24_P337746 | RABGEF1 | 0.72 | 2.96E−07 | 0.68 | 1.10E−07 | 0.80 | 3.27E−12 |
| A_33_P3421571 | RAPH1 | −0.69 | 1.64E−02 | −0.70 | 3.75E−03 | −0.87 | 5.23E−05 |
| A_23_P1962 | RARRES3 | −0.55 | 1.73E−03 | −0.65 | 2.21E−05 | −0.62 | 1.21E−04 |
| A_24_P384397 | RAVER1 | −0.67 | 2.26E−02 | −0.76 | 1.66E−03 | −0.98 | 8.52E−05 |
| A_23_P132910 | RBM47 | 0.70 | 2.93E−06 | 0.61 | 4.95E−08 | 0.69 | 8.01E−10 |
| A_33_P3271490 | RBMS1 | 0.62 | 1.42E−05 | 0.67 | 1.58E−07 | 0.76 | 3.76E−10 |
| A_23_P119222 | RETN | 1.37 | 2.08E−05 | 1.34 | 1.82E−05 | 1.52 | 6.33E−08 |
| A_33_P3350863 | RETN | 1.50 | 5.17E−06 | 1.52 | 8.78E−07 | 1.83 | 3.97E−09 |
| A_23_P306941 | RGL4 | 1.05 | 3.39E−05 | 1.10 | 3.06E−09 | 1.20 | 1.35E−08 |
| A_23_P151637 | RNASE2 | 0.75 | 1.16E−05 | 0.59 | 6.44E−04 | 0.61 | 2.95E−04 |
| A_23_P163025 | RNASE3 | 0.69 | 2.28E−06 | 0.59 | 1.55E−04 | 0.62 | 1.73E−05 |
| A_23_P257201 | RNF146 | 0.61 | 2.80E−04 | 0.59 | 1.79E−06 | 0.61 | 1.06E−06 |
| A_33_P3271316 | RPP25 | −0.02 | 9.65E−01 | −0.91 | 3.63E−03 | −1.01 | 8.51E−04 |
| A_24_P808522 | RPS14 | −0.55 | 1.92E−07 | −0.60 | 6.43E−08 | −0.65 | 5.46E−10 |
| A_23_P417331 | RPS6KA3 | 0.48 | 1.90E−04 | 0.63 | 2.17E−09 | 0.69 | 1.06E−12 |
| A_23_P120566 | RRBP1 | 0.65 | 7.80E−07 | 0.55 | 3.54E−07 | 0.73 | 1.15E−12 |
| A_33_P3211804 | RUNX1 | 0.49 | 3.42E−04 | 0.60 | 3.36E−07 | 0.78 | 5.49E−10 |
| A_23_P74001 | S100A12 | 0.84 | 5.71E−04 | 0.93 | 2.01E−06 | 1.09 | 2.11E−08 |
| A_33_P3385785 | S100A12 | 1.04 | 1.04E−04 | 1.08 | 7.82E−07 | 1.30 | 2.57E−09 |
| A_23_P23048 | S100A9 | 0.87 | 3.07E−05 | 0.81 | 2.10E−06 | 0.80 | 1.36E−06 |
| A_23_P29005 | SAMSN1 | 1.25 | 3.17E−07 | 1.13 | 1.20E−08 | 1.28 | 3.26E−10 |
| A_23_P145006 | SCGB3A2 | −0.19 | 3.73E−01 | −0.63 | 1.71E−03 | −0.69 | 3.72E−04 |
| A_23_P152548 | SCPEP1 | 0.60 | 6.32E−04 | 0.52 | 4.18E−04 | 0.59 | 7.46E−06 |
| A_21_P0012051 | SEPT14 | 0.64 | 4.85E−06 | 0.73 | 4.13E−09 | 0.80 | 8.70E−09 |
| A_21_P0010748 | SEPT14 | 0.68 | 7.47E−06 | 0.82 | 1.20E−09 | 0.89 | 4.88E−09 |
| A_21_P0011898 | SEPT14 | 0.57 | 1.03E−04 | 0.67 | 2.20E−08 | 0.72 | 2.32E−07 |
| A_21_P0011897 | SEPT14 | 0.75 | 5.28E−06 | 0.80 | 5.63E−11 | 0.86 | 2.75E−11 |
| A_21_P0013195 | SEPT14 | 0.70 | 1.32E−06 | 0.83 | 4.15E−11 | 0.84 | 1.07E−12 |
| A_23_P214330 | SERPINB1 | 0.81 | 2.05E−05 | 0.82 | 1.12E−07 | 1.00 | 3.98E−11 |
| A_24_P148750 | SH3BP5 | 0.77 | 1.40E−06 | 0.67 | 1.05E−07 | 0.68 | 2.34E−09 |
| A_33_P3298356 | SH3GLB1 | 0.54 | 4.74E−04 | 0.63 | 1.90E−06 | 0.77 | 1.34E−07 |
| A_23_P137470 | SIPA1L2 | 0.89 | 5.35E−07 | 0.89 | 7.93E−10 | 0.97 | 3.57E−10 |
| A_33_P3764802 | SIRT5 | 0.59 | 1.04E−04 | 0.61 | 6.77E−06 | 0.71 | 1.21E−08 |
| A_23_P363313 | SLC16A11 | 0.13 | 5.60E−01 | −0.67 | 8.35E−03 | −0.62 | 6.39E−03 |
| A_24_P286114 | SLC1A3 | 0.69 | 3.10E−05 | 0.46 | 6.47E−04 | 0.69 | 1.52E−07 |
| A_23_P156180 | SLC22A4 | 0.58 | 6.61E−05 | 0.62 | 1.99E−07 | 0.61 | 5.01E−08 |
| A_23_P106258 | SLC25A47 | −0.15 | 5.23E−01 | −0.63 | 6.21E−04 | −0.65 | 1.39E−04 |
| A_23_P30950 | SLC26A8 | 0.60 | 1.45E−05 | 0.57 | 5.41E−07 | 0.76 | 2.21E−12 |
| A_24_P81900 | SLC2A3 | 0.98 | 2.08E−07 | 1.00 | 3.57E−10 | 1.07 | 1.09E−11 |
| A_23_P139669 | SLC2A3 | 0.87 | 9.03E−08 | 0.92 | 1.79E−09 | 0.87 | 5.37E−10 |
| A_33_P3251093 | SLC36A1 | 0.51 | 3.58E−03 | 0.63 | 9.44E−07 | 0.89 | 1.97E−10 |
| A_23_P95130 | SLC37A3 | 0.71 | 4.51E−04 | 0.80 | 1.94E−07 | 0.94 | 9.40E−09 |
| A_24_P295963 | SLC38A2 | 0.62 | 6.77E−07 | 0.51 | 1.27E−06 | 0.64 | 1.70E−07 |
| A_33_P3242458 | SLC41A3 | −0.37 | 1.21E−01 | −0.74 | 2.69E−04 | −0.78 | 1.41E−04 |
| A_24_P58054 | SLC9A8 | 0.53 | 3.41E−05 | 0.59 | 5.41E−08 | 0.67 | 5.92E−10 |
| A_32_P154342 | SLCO4C1 | 0.59 | 5.90E−05 | 0.57 | 2.30E−05 | 0.71 | 2.47E−08 |
| A_24_P277807 | SNX3 | 0.67 | 3.39E−07 | 0.64 | 2.93E−07 | 0.51 | 8.39E−05 |
| A_23_P207058 | SOCS3 | 0.97 | 1.79E−04 | 0.94 | 9.87E−07 | 1.06 | 2.45E−07 |
| A_33_P3409625 | SORBS3 | −0.59 | 4.11E−02 | −0.70 | 6.50E−03 | −0.96 | 1.46E−04 |
| A_24_P325520 | SORT1 | 0.88 | 1.96E−06 | 0.86 | 8.42E−07 | 0.94 | 8.01E−11 |
| A_23_P117546 | SOS2 | 0.53 | 5.88E−05 | 0.57 | 1.12E−08 | 0.65 | 7.28E−10 |
| A_23_P117546 | SOS2 | 0.53 | 5.46E−05 | 0.58 | 5.68E−09 | 0.68 | 8.69E−10 |
| A_23_P117546 | SOS2 | 0.58 | 5.34E−05 | 0.58 | 6.11E−09 | 0.64 | 1.08E−09 |
| A_23_P117546 | SOS2 | 0.51 | 1.02E−04 | 0.55 | 3.05E−08 | 0.69 | 2.16E−09 |
| A_23_P117546 | SOS2 | 0.54 | 2.70E−05 | 0.57 | 5.48E−09 | 0.69 | 6.91E−10 |
| A_23_P117546 | SOS2 | 0.47 | 4.44E−04 | 0.57 | 1.41E−07 | 0.68 | 7.44E−10 |
| A_23_P117546 | SOS2 | 0.52 | 6.85E−05 | 0.58 | 6.22E−09 | 0.66 | 9.95E−10 |
| A_23_P117546 | SOS2 | 0.51 | 1.29E−04 | 0.57 | 9.94E−09 | 0.67 | 3.73E−10 |
| A_23_P117546 | SOS2 | 0.53 | 5.01E−05 | 0.59 | 3.48E−09 | 0.54 | 3.97E−04 |
| A_23_P117546 | SOS2 | 0.53 | 6.92E−05 | 0.58 | 8.96E−09 | 0.69 | 2.13E−10 |
| A_24_P385611 | SP100 | 0.61 | 3.59E−07 | 0.63 | 1.25E−10 | 0.72 | 2.31E−14 |
| A_23_P431388 | SPOCD1 | 0.53 | 1.07E−02 | 0.64 | 4.14E−05 | 0.64 | 2.86E−04 |
| A_33_P3214943 | SPOCK2 | −0.50 | 3.14E−02 | −0.90 | 9.28E−06 | −0.94 | 6.17E−06 |
| A_33_P3222139 | SREBF1 | −0.76 | 3.63E−03 | −0.61 | 3.18E−03 | −0.79 | 2.46E−04 |
| A_23_P19543 | SRPK1 | 0.57 | 5.04E−04 | 0.64 | 2.01E−07 | 0.67 | 1.50E−07 |
| A_23_P429560 | SSH1 | 0.61 | 4.65E−06 | 0.72 | 1.68E−09 | 0.85 | 1.64E−13 |
| A_24_P181055 | ST3GAL4 | 0.62 | 3.58E−04 | 0.59 | 1.97E−05 | 0.71 | 3.23E−05 |
| A_33_P3275055 | ST6GALNAC3 | 0.59 | 1.52E−04 | 0.60 | 1.24E−05 | 0.75 | 5.91E−10 |
| A_33_P3356220 | STARD3 | −0.47 | 3.72E−02 | −0.59 | 2.62E−03 | −0.74 | 4.31E−05 |
| A_24_P141214 | STOM | 0.95 | 5.85E−08 | 0.87 | 7.22E−07 | 0.88 | 5.79E−08 |
| A_23_P154605 | SULF2 | −0.70 | 5.25E−05 | −0.81 | 3.72E−06 | −0.72 | 1.14E−05 |
| A_33_P3335920 | SYNE1 | −0.40 | 5.80E−02 | −0.73 | 1.45E−04 | −0.74 | 2.03E−04 |
| A_23_P70733 | TAAR2 | −0.39 | 1.64E−01 | −0.79 | 1.50E−03 | −0.75 | 2.82E−03 |
| A_24_P148590 | TACR1 | 0.13 | 5.57E−01 | −0.61 | 9.51E−03 | −0.83 | 4.65E−05 |
| A_33_P3378659 | TARP | −0.40 | 3.61E−02 | −0.64 | 1.46E−03 | −0.68 | 2.63E−04 |
| A_33_P3309075 | TBC1D8 | 0.98 | 1.45E−07 | 0.98 | 2.53E−08 | 1.08 | 2.42E−13 |
| A_23_P64372 | TCN1 | 0.75 | 3.10E−04 | 0.68 | 2.17E−04 | 0.79 | 3.43E−05 |
| A_32_P208350 | TDRD9 | 1.19 | 1.30E−05 | 1.28 | 1.59E−08 | 1.43 | 7.32E−11 |
| A_23_P143845 | TIPARP | 0.78 | 3.84E−06 | 0.74 | 4.07E−07 | 0.72 | 5.53E−10 |
| A_23_P85903 | TLR5 | 0.83 | 1.26E−05 | 0.84 | 3.24E−09 | 1.12 | 1.63E−10 |
| A_23_P73837 | TLR8 | 0.60 | 1.49E−04 | 0.55 | 1.20E−06 | 0.61 | 8.30E−08 |
| A_33_P3257279 | TMEM145 | −0.29 | 2.02E−01 | −0.67 | 3.85E−03 | −0.75 | 6.91E−04 |
| A_33_P3411612 | TMEM221 | 0.06 | 8.12E−01 | −0.61 | 5.83E−03 | −0.60 | 2.48E−03 |
| A_23_P126844 | TNFRSF25 | −0.59 | 5.43E−04 | −0.58 | 1.37E−04 | −0.64 | 8.61E−05 |
| A_33_P3286157 | TNFRSF4 | −0.31 | 1.10E−01 | −0.64 | 5.32E−05 | −0.67 | 7.97E−06 |
| A_33_P3364582 | TNXB | −0.04 | 8.73E−01 | −0.71 | 3.45E−03 | −0.72 | 8.17E−04 |
| A_23_P5392 | TP53I3 | 0.83 | 2.67E−06 | 0.66 | 6.22E−05 | 0.78 | 2.14E−08 |
| A_33_P3392077 | TP53I3 | 0.83 | 4.55E−06 | 0.69 | 2.54E−05 | 0.83 | 9.84E−08 |
| A_33_P3223980 | TPM3 | 0.58 | 2.17E−07 | 0.59 | 1.57E−08 | 0.61 | 1.97E−10 |
| A_24_P27977 | TRPM2 | 0.77 | 1.44E−06 | 0.63 | 3.13E−06 | 0.77 | 2.06E−10 |
| A_33_P3413216 | TSPAN4 | −0.38 | 8.06E−02 | −0.84 | 6.95E−05 | −0.79 | 1.97E−05 |
| A_33_P3251148 | TSPO | 0.76 | 1.03E−04 | 0.70 | 5.88E−06 | 0.78 | 1.72E−08 |
| A_24_P11436 | TTC22 | −0.22 | 2.93E−01 | −0.62 | 6.57E−04 | −0.63 | 2.95E−04 |
| A_32_P148796 | UBXN2B | 0.66 | 1.00E−05 | 0.55 | 2.73E−07 | 0.63 | 1.33E−09 |
| A_23_P17330 | UCKL1 | −0.22 | 4.23E−01 | −0.71 | 3.86E−03 | −1.06 | 6.10E−06 |
| A_23_P313389 | UGCG | 1.17 | 1.07E−07 | 1.12 | 3.26E−09 | 1.29 | 7.12E−13 |
| A_24_P112160 | UPK3B | −0.13 | 6.18E−01 | −0.66 | 2.87E−03 | −0.62 | 4.86E−03 |
| A_23_P351275 | UPP1 | 1.03 | 1.38E−06 | 0.92 | 6.89E−07 | 1.02 | 1.02E−08 |
| A_33_P3399571 | VNN1 | 1.27 | 3.39E−05 | 1.20 | 5.27E−06 | 1.39 | 3.69E−09 |
| A_32_P10396 | WDFY3 | 0.73 | 2.67E−06 | 0.63 | 5.64E−09 | 0.67 | 1.88E−08 |
| A_33_P3411925 | WDR18 | −0.45 | 4.73E−02 | −0.82 | 2.57E−05 | −0.84 | 1.98E−05 |
| A_23_P4353 | WSB1 | 0.68 | 2.15E−05 | 0.67 | 5.55E−08 | 0.85 | 2.48E−11 |
| A_32_P178945 | YOD1 | 0.69 | 1.63E−03 | 0.63 | 2.62E−03 | 0.59 | 3.10E−03 |
| A_23_P99397 | ZDHHC20 | 0.80 | 2.74E−07 | 0.77 | 2.22E−07 | 0.96 | 3.41E−14 |
| A_33_P3376449 | ZDHHC23 | −0.47 | 3.24E−02 | −0.81 | 2.06E−05 | −0.80 | 4.65E−06 |
| A_24_P351420 | ZDHHC3 | 0.61 | 9.15E−06 | 0.49 | 1.18E−05 | 0.60 | 1.49E−08 |
| A_23_P27424 | ZNF418 | −0.26 | 3.19E−01 | −0.63 | 6.88E−03 | −0.77 | 4.62E−04 |
| A_23_P161156 | ZNF438 | 0.70 | 4.37E−05 | 0.72 | 5.15E−08 | 0.81 | 8.60E−10 |
| A_33_P3263756 | ZNF446 | −0.10 | 6.04E−01 | −0.60 | 1.49E−03 | −0.66 | 1.27E−04 |
| A_33_P3345132 | ZNF578 | −0.05 | 8.43E−01 | −0.72 | 7.57E−03 | −0.74 | 3.74E−04 |

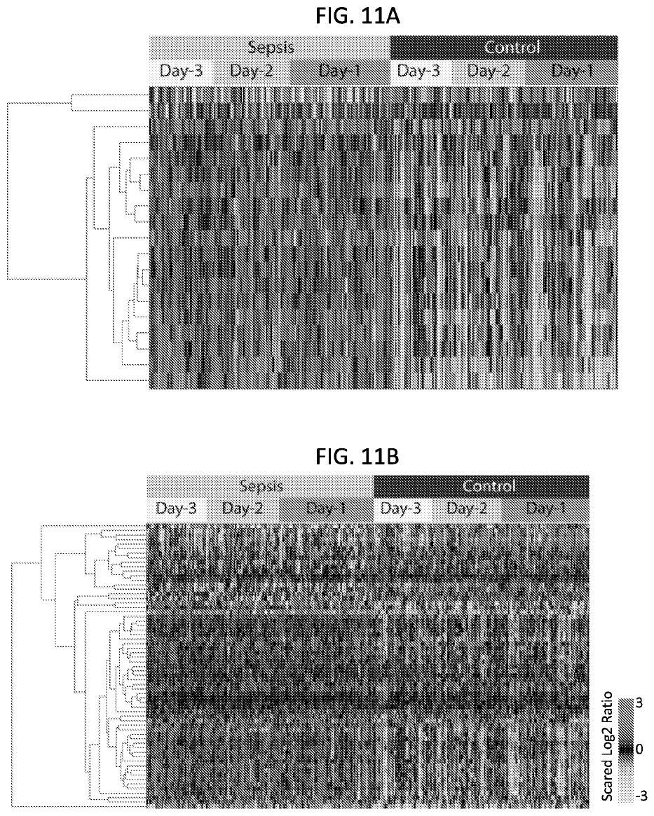

There is a significant overlap (54 genes) between the genes in ISB 58 and ISB 355 (FIG. 10). The genes included in the ISB19 and ISB63 panels are listed in Table 12 and the overall expression profiles for those genes are shown in FIGS. 11A and 11B. Exemplary, non-limiting GenBank Accession numbers are provided for each gene in Table 1. Additional sequences for each gene can be identified using publicly available databases.

TABLE 12
List of genes included in ISB19 and ISB63

| Gene | GenBank | | |
|---|---|---|---|
| Symbol | Acc. No. | ISB19 | ISB63 |
| ADAMTSL5 | NM_2136C4 | | X |
| ARID5A | NM_212481 | | X |
| ATP11B | NM_014616 | | X |
| ATP6V1C1 | NM_001695 | X | X |
| ATP9A | NM_006045 | X | |
| B4GALT5 | NM_004776 | | X |
| BMX | NM_001721 | X | |
| CA4 | NM_000717 | | X |
| CARNS1 | NM_020811 | | X |
| CD164 | NM_006016 | | X |
| CD55 | NM_000574 | | X |
| CD63 | NM_001780 | | X |
| CD82 | NM_002231 | | X |
| CFLAR | NM_003879 | | X |
| CLEC12A | NM_138337 | | X |
| CST7 | NM_003650 | | X |
| CYP27A1 | NM_000784 | | X |
| DOK3 | NM_024872 | | X |
| ETS2 | NM_005239 | | X |
| FCAR | NM_002000 | X | |
| FFAR3 | NM_005304 | | X |
| GALNT14 | NM_024572 | X | |
| GBP5 | NM_052942 | X | |
| GPER | NM_001505 | | X |
| GPR15 | NM_005290 | | X |
| HK3 | NM_002115 | X | |
| HLA-DRA | NM_019111 | | X |
| IL18R1 | NM_003855 | X | |
| IL1R2 | NM_004633 | X | |
| IL1RAP | NM_002182 | | X |
| KREMEN1 | NM_032045 | | X |
| LDLR | NM_000527 | | X |
| LGALS2 | NM_006498 | X | X |
| LRG1 | NM_052972 | X | X |
| LTF | NM_002343 | | X |
| MFNG | NM_002405 | | X |
| MICAL1 | NM_022765 | | X |
| MSL3 | NM_078628 | | X |
| NQO2 | NM_000904 | | X |
| NSUN7 | NM_024677 | | X |
| OLAH | NM_018324 | | X |
| P2RX2 | NM_016318 | | X |
| PDGFC | NM_016205 | | X |
| PDSS1 | NM_014317 | | X |
| PFKFB3 | NM_004566 | X | X |
| PGLYRP1 | NM_005091 | X | |
| PLIN4 | NM_001080400 | | X |
| POM121L12 | NM_182595 | | X |
| POU2F2 | NM_002698 | | X |
| PYGL | NM_002863 | | X |
| RBM47 | NM_019027 | | X |
| RGL4 | NM_153615 | X | |
| RNASE3 | NM_002935 | | X |
| RNF146 | NM_030963 | | X |
| RRBP1 | NM_004587 | | X |
| RUNX1 | NM_001754 | | X |
| SCGB3A2 | NM_054023 | | X |
| SEPT14 | NM_207366 | | X |
| SH3BP5 | NM_004844 | | X |
| SLC2A3 | NM_006931 | X | X |
| SPOCK2 | NM_014767 | | X |
| ST6GALNAC3 | NM_152996 | | X |
| STOM | NM_004099 | | X |
| SYNE1 | NM_033071 | | X |
| TAAR2 | NM_014626 | | X |
| TARP | NM_001003799 | | X |
| TCN1 | NM_001062 | X | X |
| TP53I3 | NM_004881 | X | |
| TPM3 | NM_153649 | | X |
| UCKL1 | NM_017859 | | X |
| UGCG | NM_003358 | X | |
| UPP1 | NM_003364 | X | |
| WSB1 | NM_015626 | | X |
| YOD1 | NM_018566 | | X |
| ZDHHC23 | NM_173570 | | X |
| ZNF446 | NM_017908 | | X |

### Example 3

### Validation of Gene Panels for Predicting Development of Sepsis

An SVM model for each panel that was trained using the Discovery sample set (Example 2) was generated and subsequently tested in the Validation sample set. Classification performance was summarized and listed with four metrics: AUC, Accuracy, Sensitivity, and Specificity in Table 13. Both panels provided high predictive performance. For example, the AUC values of ISB19 are >0.7 and the AUC values of ISB63 were even higher, at >0.8 at all time points. ISB63 showed greater than 77% of predictive accuracy for the development of sepsis three days prior to sepsis diagnosis (Day-3). The two panels ISB19 and ISB63, showed very little overlap of genes in the panel (Table 11).

TABLE 13
Classification performances of ISB19
and ISB63 in Validation sample set.

| | ISB19 | | | ISB63 | | |
|---|---|---|---|---|---|---|
| | Day −3 | Day −2 | Day −1 | Day −3 | Day −2 | Day −1 |
| AUC | 0.7538 | 0.7690 | 0.8005 | 0.8897 | 0.8263 | 0.8558 |
| Accuracy | 0.6622 | 0.6866 | 0.7457 | 0.7703 | 0.7313 | 0.7341 |
| Sensitivity | 0.6486 | 0.7391 | 0.7701 | 0.8649 | 0.7681 | 0.8161 |
| Specificity | 0.6757 | 0.6308 | 0.7209 | 0.6757 | 0.6923 | 0.6512 |

### Example 4

### Assessing Ability of ISB Panels to Diagnose Sepsis Using Diverse Datasets

Even though the ISB panels were developed using pre-sepsis diagnosis gene expression data to predict the development of sepsis, the ability of the ISB19 and ISB63 gene panels (Example 2) were assessed to diagnose patients with sepsis using 19 publicly available sepsis related datasets derived from 1,636 patients (Table 14). The public datasets can be grouped based on patient information, such as bacterial or viral infection, or adult or pediatric sepsis. With this information, the datasets were grouped into 6 groups based on clinical parameters: 1) Sepsis/severe sepsis, 2) Pediatric sepsis, 3) Neonatal sepsis, 4) Sepsis associated with bacterial infection, 5) Sepsis associated with bacterial/viral infection, and 6) Sepsis associated with viral infection.

TABLE 14
Public datasets used to assess the diagnostic performance of ISB19 and ISB63 panels

| Subcategory | Accession | Platform | Clinical Comparison | # Control | # Cases |
|---|---|---|---|---|---|
| Sepsis or | GSE69528 | Illumina HumanHT-12 | Adults with sepsis, | | 83 |
| severe sepsis | | V4.0 expression | many from | | |
| vs Control | | beadchip | burkholderia | | |
| | GSE57065 | Affymetrix Human | Septic shock at | 25 | 82 |
| | | Genome U133 Plus 2.0 | admission, 24 | | |
| | | Array | hr, 48 hr | | |
| | GSE28750 | Affymetrix Human | Community-acquired | 20 | 10 |
| | | Genome U133 Plus 2.0 | sepsis at | | |
| | | Array | admission to ICU | | |
| Pediatric | GSE66099 | Affymetrix Human | Pediatric sepsis | 47 | 199 |
| sepsis vs | | Genome U133 Plus 2.0 | | | |
| Control | | Array | | | |
| | E-MEXP-3567 | Affymetrix Human | Children with meningococcal | 3 | 12 |
| | | Genome U133A Array | sepsis ± HIV coinfection | | |
| | GSE11755 | Affymetrix Human | Children with meningococcal | 3 | 6 |
| | | Genome U133 Plus 2.0 | sepsis | | |
| | | Array | | | |
| Neonatal | GSE25504 | Illumina HumanHT-12 | Neonatal sepsis | 35 | 28 |
| sepsis vs | | V3.0 expression | | | |
| Control | | beadchip | | | |
| | GSE25504 | Affymetrix Human | Neonatal sepsis | 6 | 14 |
| | | Genome U219 Array | | | |
| | GSE25504 | Affymetrix Human | Neonatal sepsis | 3 | 2 |
| | | Genome U133 Plus 2.0 | | | |
| | | Array | | | |
| Bacterial | GSE65682 | Affymetrix Human | Adults in ICU with CAP | 42 | 101 |
| infection | | Genome U219 Array | | | |
| with sepsis | GSE33341 | Affymetrix Human | Bloodstream infection | 43 | 51 |
| vs Control | | Genome U133A 2.0 | Staphylococcus aureus or | | |
| | | Array | Escherichia coli | | |
| Bacterial or | GSE40396 | Illumina HumanHT-12 | Children with | 22 | 30 |
| viral | | V4.0 expression | infection + | | |
| infection | | beadchip | fever | | |
| with sepsis | E-MEXP-3589 | Agilent-026652 Whole | Hospitalized chronic | 4 | 14 |
| vs Control | | Human Genome | obstructive pulmonary disease | | |
| | | Microarray 4x44K v2 | patients with infection | | |
| Viral | GSE68310 | Illumina HumanHT-12 | Outpatients with acute viral | 243 | 258 |
| infection | | V4.0 expression | illness at days 0 and 2 | | |
| with sepsis | | beadchip | | | |
| vs Control | GSE17156 | Affymetrix Human | Viral challenge peak | 56 | 27 |
| | | Genome U133A 2.0 | symptoms | | |
| | | Array | | | |
| | GSE21802 | Illumina human-6 v2.0 | Severe H1N1 influenza with | 4 | 12 |
| | | expression beadchip | mechanical ventilation | | |
| | GSE27131 | Illumina human-6 v2.0 | Severe H1N1 influenza with | 4 | 12 |
| | | expression beadchip | mechanical ventilation | | |
| | E-MTAB-3162 | Affymetrix Human | Dengue fever (±severe) | 15 | 30 |
| | | Genome U133 Plus 2.0 | within 48 hr of fever | | |
| | | Array | | | |
| | GSE51808 | Affymetrix HT HG- | Dengue fever (±severe) at | 9 | 28 |
| | | U133 + PM Array Plate | admission | | |

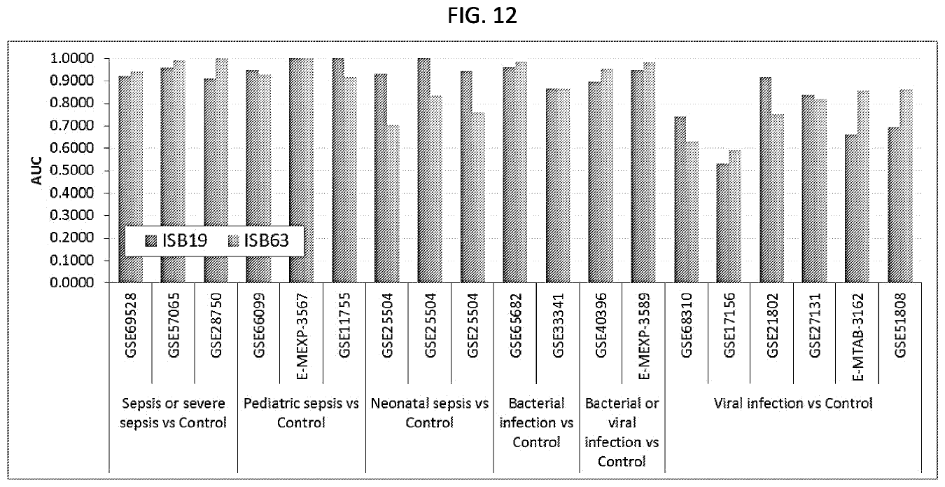

Even though the ISB19 and ISB63 panels were developed for predicting the development of sepsis, these panels still showed good performance in diagnosing patients with sepsis in unrelated and independent datasets obtained from the public domain (FIG. 12). Importantly, these datasets were generated with different sample preparation methods, measurement platforms, and diverse patient cohorts. The results shown in FIG. 12 suggest that the measurement platform, disease condition and sample preparation method have very little effect on the performance of the ISB19 and ISB63 panels. Also, both panels showed high classification performance in most sepsis datasets and bacterial or bacterial/viral infection datasets (AUC>0.8), except for ISB63 in neonatal sepsis and ISB19 in one of the bacterial/viral infection datasets). However, both panels showed lower performance in two of the viral infection datasets, GSE68310 and GSE17156. Importantly, viral infection is not a common cause of sepsis post-surgery.

### Example 5

### ISB19 and ISB63 Panels are Both Prognostic and Diagnostic for Sepsis

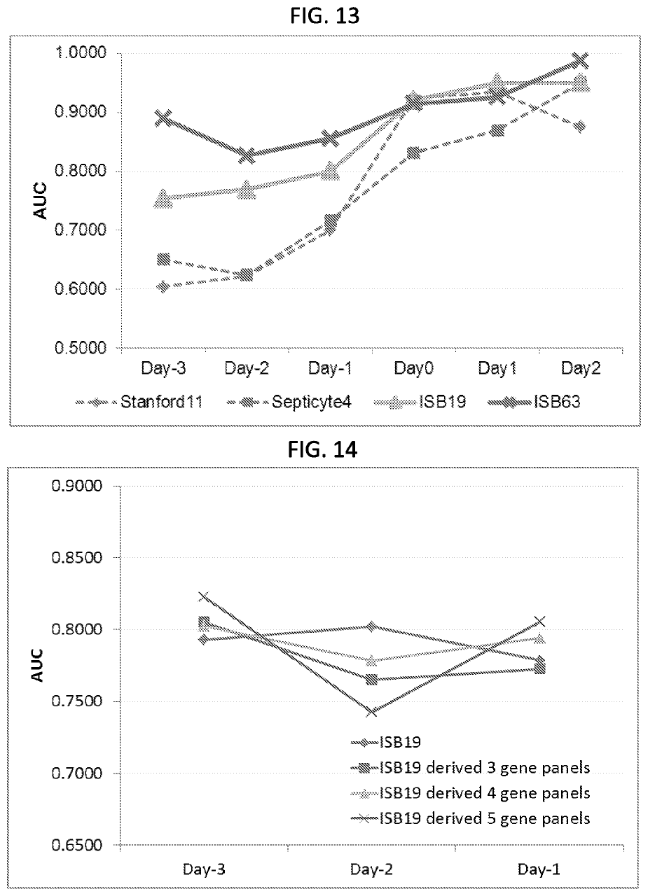

To determine the utility of ISB19 and ISB63 panels, the ability of ISB panels to predict the development and diagnose sepsis was assessed and compared with panels reported in literature (Stanford11) and approved by FDA (Septicyte4). The results show that the previously identified sepsis panels, Stanford11 and Septicyte4, were able to accurately diagnose but not predict sepsis development (FIG. 13). In contrast, the ISB panels performed well to predict the development of sepsis in the pre-symptomatic period (from Day-3 to Day-1) (FIG. 13). In addition, the panels, in particular ISB63, showed better or similar ability as the Stanford11 and Septicyte4 panels to diagnose (Day 0) and monitor (Day 1 and Day 2) sepsis. These findings suggest that the ISB panels described herein can not only predict the development of sepsis at the pre-symptomatic period, but also diagnose and monitor the sepsis condition in patients.

### Example 6

### Biological Function-Based Approach to Optimize Panels

The methods described in Example 1 to optimize gene panels were applied to the ISB19 and ISB63 panels. The biological processes associated with ISB19 and ISB 63 panels were first determined. The Gene Ontology (GO) enrichment test showed a total of 89 GOBPs associated with genes in the ISB19 panel and 326 GOPBs associated with the ISB63. The EnrichmentMap tool summarized the 89 GOBPs associated with ISB19 into three representative functional terms: immune response, signal transduction, and metabolism. Similarly, the 326 GOBPs associated with ISB63 were summarized into six representative terms: immune response, signal transduction, metabolism, apoptosis, transcription, and adhesion/migration (Table 15).

Genes from the original ISB58 and ISB355 sets that shared the same functional terms and similar direction of concentration changes with genes already included in the ISB19 and ISB63 were then used. From ISB58 gene set, 13, 3, and 4 genes were identified that have the same direction of concentration changes with genes in ISB19 and are associated with immune response, signal transduction, and metabolism, respectively. From ISB355 gene set, 67, 55, 41, 53, 7 and 40 genes were identified that have the same direction of concentration changes with genes in ISB63 and are associated with immune response, signal transduction, metabolism, apoptosis, transcription and adhesion/migration, respectively (Table 15).

TABLE 15
List of functional terms associated with ISB19 and ISB63
and available alternative candidates from ISB58 and
ISB355 for genes in the ISB19 and ISB63 panels

| Functional term | ISB19 | ISB63 |
|---|---|---|
| Immune response | 13 | 67 |
| Signal transduction | 3 | 55 |
| Metabolism | 4 | 40 |
| Apoptosis | | 41 |
| Transcription | | 53 |
| Adhesion/Migration | | 7 |

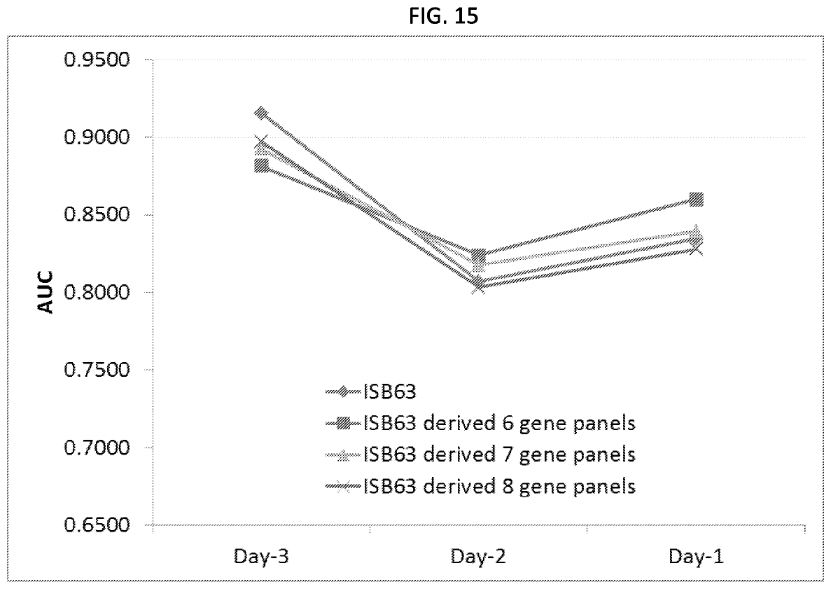

The genes identified from ISB58 and ISB355 (number of available genes for individual functional terms are listed in Table 15) were then used to compute classification performance of three-gene combinations that represent three functional terms associated with ISB19 and six gene combinations for ISB63. In total, the performance of 156 three-gene combinations and randomly selected 100,000 (out of 2.24×10⁹ possibilities) six-gene combinations, respectively, were assessed. The top 10 performing three (from ISB19) and six (from ISB63) gene panels are listed in Tables 16 and 17, respectively. The results showed slightly higher or similar average performance for the ISB19 derived three gene panels than the original ISB19 at all three time points (FIG. 14). For ISB63 derived six gene panels, slightly lower performances at all time points were observed compared to the original ISB63 (FIG. 15). Since some of the genes in the ISB58 or ISB355 that were not in the biological processes associated with ISB19 or 1063 can still contribute to the classification performance, additional genes were added (one gene at a time) to the top 10 ISB19 derived 3-gene or ISB63 derived 6-gene panels. The results showed a significant improvement of overall performance when adding one or two genes for both ISB19 and ISB63 derived panels (Tables 16 and 17, and FIGS. 14 and 15). The slight decrease in performance of the panels with smaller number of features than the original 19 or 63 gene panels is a trade-off that provides advantages with respect to future development and application in a clinical setting.

TABLE 16
The top 10 performance of 3, 4 and 5 gene panels
derived from ISB19 based on biological function.

| Panels | Composition | Day-3 | Day-2 | Day-1 |
|---|---|---|---|---|
| ISB19 | | 0.7929 | 0.8021 | 0.7786 |
| ISB19 | LCN2, SLC2A3, BMX | 0.8284 | 0.8279 | 0.8233 |
| derived 3 | LCN2, SLC2A3, GRB10 | 0.7959 | 0.7885 | 0.8002 |
| gene | LCN2, PFKFB3, GRB10 | 0.8033 | 0.7355 | 0.7759 |
| panels | LCN2, PFKFB3, BMX | 0.8003 | 0.7351 | 0.7599 |
| | IL1R2, HK3, BMX | 0.7722 | 0.7690 | 0.7284 |
| | LCN2, HK3, BMX | 0.8358 | 0.7944 | 0.7645 |
| | LCN2, HK3, GRB10 | 0.8225 | 0.7794 | 0.7722 |
| | GZMA, HK3, BMX | 0.8343 | 0.7654 | 0.7577 |
| | FCAR, PFKFB2, BMX | 0.7737 | 0.7178 | 0.7737 |
| | LCN2, PFKFB3, IL18R1 | 0.7870 | 0.7396 | 0.7716 |
| ISB19 | LCN2, PFKFB3, GRB10, ST6GALNAC3 | 0.8047 | 0.7255 | 0.7845 |
| derived 4 | LCN2, SLC2A3, BMX, LGALS2 | 0.8136 | 0.8374 | 0.8291 |
| gene | IL1R2, SLC2A3, BMX, TCN1 | 0.8018 | 0.8478 | 0.8143 |
| panels | LCN2, SLC2A3, GRB10, ST6GALNAC3 | 0.7737 | 0.7758 | 0.8245 |
| | FCAR, PFKFB2, BMX, CEACAM1 | 0.7840 | 0.7088 | 0.7746 |
| | IL1R2, HK3, BMX, CD24 | 0.7870 | 0.7763 | 0.7457 |
| | IL1R2, PFKFB3, BMX, CD24 | 0.8121 | 0.7332 | 0.7703 |
| | BMX, SLC2A3, GRB10, CD24 | 0.8136 | 0.7939 | 0.8143 |
| | IL1R2, HK3, BMX, CEACAM1 | 0.7959 | 0.7722 | 0.7445 |
| | GZMA, SLC2A3, BMX, CD24 | 0.8388 | 0.8143 | 0.8381 |
| ISB19 | LCN2, PFKFB3, GRB10, ST6GALNAC3, RNASE3 | 0.8018 | 0.7373 | 0.7993 |
| derived 5 | LCN2, PFKFB3, GRB10, RNASE2, ST6GALNAC3 | 0.8077 | 0.7355 | 0.7996 |
| gene | IL1R2, PFKFB3, GRB10, CD24, ST6GALNAC3 | 0.8240 | 0.7400 | 0.8014 |
| panels | GZMA, PFKFB3, GRB10, ST6GALNAC3, CD24 | 0.8402 | 0.7450 | 0.8159 |
| | HK3, PFKFB3, GRB10, ST6GALNAC3, CD24 | 0.8402 | 0.7554 | 0.8107 |
| | IL1R2, PFKFB3, GRB10, ST6GALNAC3, TCN1 | 0.8047 | 0.7767 | 0.8033 |
| | SLC2A3, PFKFB3, GRB10, ST6GALNAC3, TCN1 | 0.8077 | 0.7966 | 0.8384 |
| | LCN2, PFKFB3, GRB10, ST6GALNAC3, DACH1 | 0.8047 | 0.7364 | 0.7922 |
| | SLC2A3, PFKFB3, GRB10, ST6GALNAC3, CD24 | 0.8299 | 0.7831 | 0.8381 |
| | SLC2A3, HK3, BMX, SPOCD1, LGALS2 | 0.7811 | 0.7853 | 0.7925 |

Exemplary GenBank Accession Nos. are provided for the genes in Table 16 either in Table 12, or as follows: GRB10—GenBank Accession No. NM_001350814; GZMA—GenBank Accession No. NM_006144; PFKFB2—GenBank Accession No. NM_006212; CD24—GenBank Accession No. NM_037362; RNASE2—GenBank Accession No. NM_002934; DACH1—GenBank Accession No. NM_004392; SPOCD1—GenBank Accession No. NM_144569

TABLE 17
The top 10 performance of 6, 7 and 8 gene panels derived from ISB63 based on biological function

| Panels | Composition | Day-3 | Day-2 | Day-1 |
|---|---|---|---|---|
| ISB63 | | 0.9157 | 0.8071 | 0.8347 |
| ISB63 | RPS6KA3, BCL6, TPM3, GNG10, STOM, MPO | 0.8772 | 0.8216 | 0.8590 |
| derived 6 | RPS6KA3, MAVS, TPM3, BCL6, STOM, MPO | 0.8772 | 0.8243 | 0.8575 |
| gene | RPS6KA3, BCL6, TPM3, BMX, STOM, MPO | 0.8861 | 0.8170 | 0.8571 |
| panels | RPS6KA3, LTF, TPM3, BCL6, STOM, PDGFC | 0.8891 | 0.8333 | 0.8655 |
| | RPS6KA3, BCL6, TPM3, CD55, STOM, MPO | 0.8802 | 0.8184 | 0.8575 |
| | RPS6KA3, BCL6, TPM3, GNG10, STOM, PDGFC | 0.8654 | 0.8211 | 0.8565 |
| | RPS6KA3, LTF, TPM3, BCL6, STOM, CYP1B1 | 0.8905 | 0.8342 | 0.8685 |
| | LTF, BCL6, TPM3, CD55, STOM, PDGFC | 0.8905 | 0.8270 | 0.8633 |
| | RPS6KA3, BCL6, TPM3, TLR8, STOM, MPO | 0.8817 | 0.8197 | 0.8584 |
| | RPS6KA3, BCL6, TPM3, HLA-DRA, GNG10, MPO | 0.8476 | 0.7649 | 0.8350 |
| ISB63 | RPS6KA3, MLLT1, TPM3, BCL6, GNG10, CYP27A1, YOD1 | 0.8728 | 0.8143 | 0.8402 |
| derived 7 | RPS6KA3, MLLT1, TPM3, BCL6, GNG10, PDGFC, YOD1 | 0.8891 | 0.8066 | 0.8285 |
| gene | RPS6KA3, GAS7, TPM3, BCL6, RRBP1, MPO, YOD1 | 0.9009 | 0.8202 | 0.8313 |
| panels | RPS6KA3, MLLT1, TPM3, BCL6, GNG10, MPO, LGALS2 | 0.9157 | 0.8125 | 0.8399 |
| | RPS6KA3, GAS7, TPM3, BCL6, GNG10, MPO, YOD1 | 0.8891 | 0.8179 | 0.8307 |
| | LTF, MLLT1, TPM3, BCL6, GNG10, PDGFC, YOD1 | 0.9157 | 0.8179 | 0.8334 |
| | RPS6KA3, BCL6, TPM3, GNG10, LILRA2, CYP27A1, YOD1 | 0.8654 | 0.8225 | 0.8347 |
| | RPS6KA3, BCL6, TPM3, GNG10, STOM, MPO, LGALS2 | 0.8876 | 0.8279 | 0.8534 |
| | RPS6KA3, MLLT1, TPM3, BCL6, GNG10, MPO, YOD1 | 0.8935 | 0.8134 | 0.8414 |
| | RPS6KA3, BCL6, TPM3, CD55, STOM, MPO, IL17RA | 0.8964 | 0.8220 | 0.8605 |
| ISB63 | RPS6KA3, MLLT1, TPM3, BCL6, GNG10, PDGFC, YOD1, LGALS2 | 0.8876 | 0.8098 | 0.8282 |
| derived 8 | RPS6KA3, MLLT1, TPM3, BCL6, GNG10, PDGFC, YOD1, LILRA4 | 0.8920 | 0.8030 | 0.8300 |
| gene | RPS6KA3, MLLT1, TPM3, BCL6, GNG10, PDGFC, YOD1, IL17RA | 0.8979 | 0.8111 | 0.8285 |
| panels | RPS6KA3, MLLT1, TPM3, BCL6, GNG10, PDGFC, YOD1, TCN1 | 0.9172 | 0.8252 | 0.8374 |
| | RPS6KA3, MLLT1, TPM3, BCL6, GNG10, PDGFC, YOD1, RNASE3 | 0.8950 | 0.8084 | 0.8307 |
| | RPS6KA3, MLLT1, TPM3, BCL6, GNG10, PDGFC, YOD1, NOV | 0.8876 | 0.7971 | 0.8264 |
| | RPS6KA3, MLLT1, TPM3, BCL6, GNG10, PDGFC, YOD1, RNASE2 | 0.8920 | 0.8066 | 0.8304 |
| | LTF, MLLT1, TPM3, BCL6, GNG10, PDGFC, YOD1, FAM105A | 0.9112 | 0.8116 | 0.8331 |
| | RPS6KA3, BCL6, TPM3, HLA-DRA, RRBP1, MPO, ERO1L, YOD1 | 0.8950 | 0.7799 | 0.8177 |
| | LTF, MLLT1, TPM3, BCL6, GNG10, PDGFC, YOD1, C14orf101 | 0.9112 | 0.8211 | 0.8353 |

Exemplary GenBank Accession Nos. are provided for the genes in Table 17 either in Table 12, with respect to Table 16, or as follows: RPS6KA3—GenBank Accession No. NM_004586; BCL6—GenBank Accession No. NM_001706; GNG10—GenBank Accession No. NM_001017998; MPO—GenBank Accession No. NM_000250; MAVS—GenBank Accession No. NM_020746; CYPIB1—GenBank Accession No. NM_000104; TLR8—GenBank Accession No. NM_016610; MLLT1—GenBank Accession No. NM_005934; GAS7—GenBank Accession No. NM_003644; LILRA2—GenBank Accession No. NM_006866; IL17RA—GenBank Accession No. NM_014339; LILRA4—GenBank Accession No. NM_012276; NOV—GenBank Accession No. NM_002514; FAM105A—GenBank Accession No. NM_019018; ERO1L—GenBank Accession No. GenBank Accession No. NM_014584; C14orf101—GenBank Accession No. NM_017799.

### Example 7

### Diagnostic Performance of Reduced Size Panels

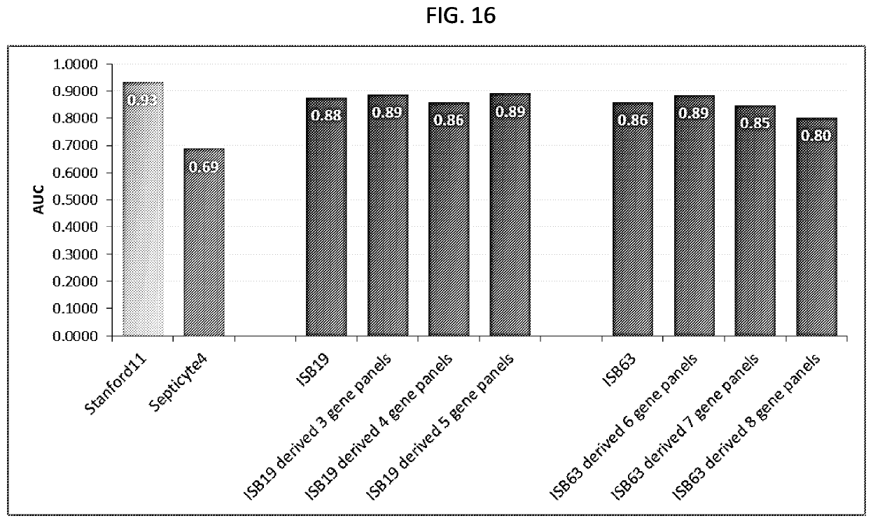

Since the ISB predictive panels ISB19 and ISB63 also showed good diagnostic performance, public domain data were used to assess the ability of smaller gene panels to accurately diagnose sepsis. The average AUCs of the 19 datasets are summarized in FIG. 16. Even though the public datasets consist of post-symptomatic samples, the ISB biomarker panels still showed good performance compared to other sepsis diagnostic panels including Stanford11 and the recently FDA approved Septicyte 4. This finding suggests that like the ISB19 and ISB63 panels, the performance of smaller panels were not significantly impacted by sample preparation methods, measurement platforms and patient cohort demographics.

### Example 8

### Integrating Clinical Information Enhances Performance of Biomarker Panels

The Sequential Organ Failure Assessment (SOFA) score is used to determine the extent of a patient's organ failure. C-reactive protein (CRP) levels are commonly used clinically as a non-specific indicator of infection. These two clinical parameters are often utilized as part of the sepsis diagnosis, and for most patients in this study this clinical information was available. SOFA score or CRP levels alone did not provide good performance in either diagnosing or predicting the development of sepsis (Table 18). However, integrating these two clinical parameters with the ISB gene panels slightly increased sensitivity, but not specificity (Table 18).

TABLE 18
Performance summaries of ISB gene panels integrated with SOFA and CRP levels

| | AUC | | | ACCURACY | | | Sensitivity | | | Specificity | | |
|---|---|---|---|---|---|---|---|---|---|---|---|---|
| Panel and combination | Day −3 | Day −2 | Day −1 | Day −3 | Day −2 | Day −1 | Day −3 | Day −2 | Day −1 | Day −3 | Day −2 | Day −1 |
| SOFA | 0.65 | 0.65 | 0.64 | 0.59 | 0.61 | 0.61 | 0.58 | 0.63 | 0.53 | 0.60 | 0.59 | 0.68 |
| CRP | 0.47 | 0.41 | 0.50 | 0.54 | 0.61 | 0.46 | 1.00 | 1.00 | 1.00 | 0.00 | 0.00 | 0.00 |
| SOFA + CRP | 0.47 | 0.67 | 0.78 | 0.41 | 0.60 | 0.66 | 0.56 | 0.64 | 0.79 | 0.25 | 0.53 | 0.55 |
| ISB19 derived 3 | 0.81 | 0.77 | 0.77 | 0.73 | 0.69 | 0.72 | 0.74 | 0.70 | 0.75 | 0.73 | 0.69 | 0.69 |
| gene panels | | | | | | | | | | | | |
| ISB19 derived 3 | 0.79 | 0.74 | 0.76 | 0.63 | 0.60 | 0.66 | 0.77 | 0.72 | 0.78 | 0.50 | 0.48 | 0.52 |
| gene panels + SOFA | | | | | | | | | | | | |
| ISB19 derived 3 | 0.83 | 0.75 | 0.85 | 0.62 | 0.58 | 0.70 | 0.87 | 0.80 | 0.91 | 0.38 | 0.35 | 0.48 |
| gene panels + CRP | | | | | | | | | | | | |
| ISB19 derived 3 | 0.82 | 0.73 | 0.81 | 0.57 | 0.56 | 0.64 | 0.90 | 0.78 | 0.91 | 0.24 | 0.33 | 0.35 |
| gene panels + | | | | | | | | | | | | |
| SOFA + CRP | | | | | | | | | | | | |
| ISB19 derived 4 | 0.80 | 0.78 | 0.79 | 0.72 | 0.72 | 0.73 | 0.70 | 0.71 | 0.74 | 0.73 | 0.73 | 0.72 |
| gene panels | | | | | | | | | | | | |
| ISB19 derived 4 | 0.79 | 0.76 | 0.78 | 0.63 | 0.62 | 0.66 | 0.73 | 0.72 | 0.78 | 0.52 | 0.52 | 0.53 |
| gene panels + SOFA | | | | | | | | | | | | |
| ISB19 derived 4 | 0.79 | 0.76 | 0.86 | 0.61 | 0.58 | 0.71 | 0.87 | 0.77 | 0.91 | 0.35 | 0.38 | 0.50 |
| gene panels + CRP | | | | | | | | | | | | |
| ISB19 derived 4 | 0.81 | 0.74 | 0.83 | 0.55 | 0.56 | 0.65 | 0.87 | 0.77 | 0.91 | 0.23 | 0.35 | 0.37 |
| gene panels + | | | | | | | | | | | | |
| SOFA + CRP | | | | | | | | | | | | |
| ISB19 derived 5 | 0.81 | 0.76 | 0.81 | 0.74 | 0.71 | 0.74 | 0.75 | 0.70 | 0.79 | 0.73 | 0.72 | 0.69 |
| gene panels | | | | | | | | | | | | |
| ISB19 derived 5 | 0.80 | 0.74 | 0.80 | 0.63 | 0.60 | 0.68 | 0.77 | 0.70 | 0.81 | 0.50 | 0.50 | 0.54 |
| gene panels + SOFA | | | | | | | | | | | | |
| ISB19 derived 5 | 0.82 | 0.77 | 0.88 | 0.62 | 0.60 | 0.71 | 0.87 | 0.80 | 0.92 | 0.38 | 0.39 | 0.49 |
| gene panels + CRP | | | | | | | | | | | | |
| ISB19 derived 5 | 0.83 | 0.74 | 0.85 | 0.58 | 0.56 | 0.66 | 0.89 | 0.77 | 0.91 | 0.26 | 0.35 | 0.39 |
| gene panels + | | | | | | | | | | | | |
| SOFA + CRP | | | | | | | | | | | | |
| ISB63 derived 6 | 0.88 | 0.82 | 0.86 | 0.77 | 0.77 | 0.78 | 0.80 | 0.78 | 0.81 | 0.74 | 0.76 | 0.76 |
| gene panels | | | | | | | | | | | | |
| ISB63 derived 6 | 0.87 | 0.78 | 0.83 | 0.68 | 0.63 | 0.68 | 0.83 | 0.75 | 0.83 | 0.52 | 0.51 | 0.53 |
| gene panels + | | | | | | | | | | | | |
| SOFA | | | | | | | | | | | | |
| ISB63 derived 6 | 0.90 | 0.79 | 0.89 | 0.65 | 0.63 | 0.71 | 0.92 | 0.83 | 0.93 | 0.37 | 0.43 | 0.48 |
| gene panels + | | | | | | | | | | | | |
| CRP | | | | | | | | | | | | |
| ISB63 derived 6 | 0.88 | 0.77 | 0.85 | 0.58 | 0.60 | 0.64 | 0.90 | 0.83 | 0.91 | 0.26 | 0.36 | 0.35 |
| gene panels + | | | | | | | | | | | | |
| SOFA + CRP | | | | | | | | | | | | |
| ISB63 derived 7 | 0.89 | 0.82 | 0.84 | 0.81 | 0.75 | 0.78 | 0.81 | 0.73 | 0.78 | 0.81 | 0.77 | 0.78 |
| gene panels | | | | | | | | | | | | |
| ISB63 derived 7 | 0.87 | 0.79 | 0.81 | 0.68 | 0.65 | 0.68 | 0.80 | 0.78 | 0.81 | 0.55 | 0.52 | 0.54 |
| gene panels + SOFA | | | | | | | | | | | | |
| ISB63 derived 7 | 0.92 | 0.80 | 0.88 | 0.68 | 0.62 | 0.71 | 0.93 | 0.82 | 0.89 | 0.43 | 0.41 | 0.53 |
| gene panels + CRP | | | | | | | | | | | | |
| ISB63 derived 7 | 0.88 | 0.78 | 0.84 | 0.58 | 0.59 | 0.65 | 0.91 | 0.82 | 0.89 | 0.26 | 0.36 | 0.39 |
| gene panels + SOFA + CRP | | | | | | | | | | | | |
| ISB63 derived 8 | 0.90 | 0.81 | 0.83 | 0.80 | 0.74 | 0.76 | 0.81 | 0.73 | 0.76 | 0.80 | 0.74 | 0.76 |
| gene panels | | | | | | | | | | | | |
| ISB63 derived 8 | 0.87 | 0.79 | 0.80 | 0.65 | 0.65 | 0.68 | 0.81 | 0.79 | 0.80 | 0.49 | 0.51 | 0.56 |
| gene panels + SOFA | | | | | | | | | | | | |
| ISB63 derived 8 | 0.92 | 0.80 | 0.87 | 0.68 | 0.61 | 0.72 | 0.92 | 0.82 | 0.88 | 0.43 | 0.40 | 0.55 |
| gene panels + CRP | | | | | | | | | | | | |
| ISB63 derived 8 | 0.87 | 0.78 | 0.83 | 0.57 | 0.59 | 0.65 | 0.89 | 0.83 | 0.89 | 0.24 | 0.35 | 0.39 |
| gene panels + SOFA + CRP | | | | | | | | | | | | |

### Example 9

### Prognostic Performance of Reduced Panels in Patients with Different Levels of Severity with and without Integrated Clinical Performance

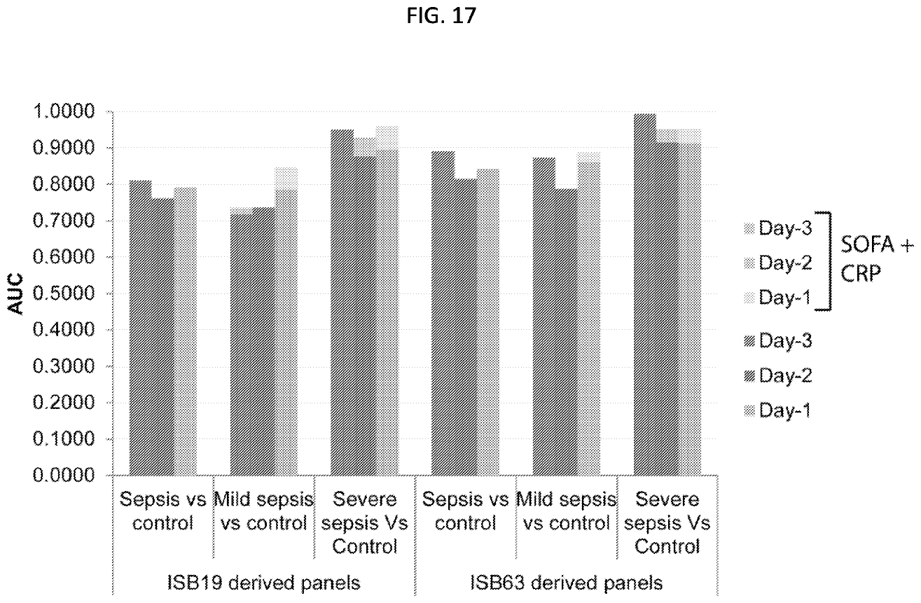

To determine if there were differences in prediction performance based on the severity of sepsis, patients who required vasopressor support and who met septic shock criteria in all iterations of the sepsis guidelines as severe sepsis were identified. All remaining patients were classified as mild sepsis. Based on this stratification, the classification performance of 60 small gene panels derived from ISB19 and ISB63 (Tables 16 and 17) were evaluated for all Sepsis samples, Mild Sepsis and Severe Sepsis compared to Controls. The ISB panels showed higher classification performance between Severe Sepsis and Control (FIG. 17). This result suggested that the prognostic performance of the panels is particularly accurate in identifying patients who are at risk of developing severe sepsis. The classification performance of each panel integrated with clinical information (SOFA score and CRP level) was then computed. Similar to what was observed earlier, integrating with clinical parameters in the panel enhanced classification performance especially in Severe Sepsis compared to Control (AUC: 0.9522 at Day-1) (FIG. 17).

### Example 10

### Pre-Symptomatic Blood Biomarker Panels for Sepsis

The current study utilized longitudinal blood samples collected from patients undergoing elective surgery to identify a blood mRNA-based panel that could diagnose sepsis prior to onset of detectable clinical symptoms, allowing for much earlier therapeutic intervention. Using a combination of differential gene expression analysis, machine learning tools, and a biological function-based biomarker panel optimization process, we have identified and validated biomarker panels consisting of only three or six genes that can identify patients developing sepsis three days prior to the onset of symptoms.

### Methods

Patient and sample description. The patients were recruited across eight hospitals in England and Germany: Guys' and St Thomas' Hospital (London, U.K.), Heartlands Hospital (Birmingham, U.K.), North Bristol NHS Trust (Bristol, U.K.), Queen Elizabeth Hospital (Birmingham, U.K.), The Leeds Teaching Hospitals Trust (Leeds, U.K.), The Royal Liverpool University Hospital (Liverpool, U.K.), University College Hospital (London, U.K.), and University Hospital Frankfurt (Frankfurt DE). All subjects gave written informed consent to participate and the study was approved by Southampton & South West Hampshire Research Ethics Committee with reference number 06/Q1702/152.

The patients underwent a wide range of major elective surgeries, with the most common being large bowel resection, vascular surgery, and pancreatic surgery (detailed patient information is shown in Table 19). Peripheral blood samples were collected from patients prior to surgery (Pre-Op) and daily up to five days post-sepsis diagnosis (Post-Op). Each patient's demographic parameters, vital signs, hematology, clinical chemistry, and pathogen detection results were recorded. The control group included age, sex, and surgical procedure matched patients who did not develop sepsis. Consensus evaluations were made by nine physicians for the study. This multi-year project was initiated prior to the release of the Sepsis-3 definition; therefore, all patients were diagnosed based on the Sepsis-2 criteria.

Whole blood RNA isolation and microarray analysis. Total RNA from RNAlater-preserved whole blood was isolated with miRNeasy kit (Qiagen, Germantown, Md.) according to the manufacturer's instructions. The quantity and quality of RNA were assessed with the Agilent 2100 Bioanalyzer (Santa Clara, Calif.) and NanoDrop 1000 spectrophotometer (Thermo Scientific, Wilmington, Del.). Whole blood gene expression profiling experiments were performed using Agilent Human Whole Genome 8×60 microarrays and fluorescent probes were prepared using Agilent QuickAmp Labeling Kit according to the manufacturer's instructions (Santa Clara, Calif.). Gene expression information was obtained with Agilent's Feature Extractor and processed with in-house SLIM pipeline (Marzolf et al., Source Code Biol. Med. 1:5, 2006).

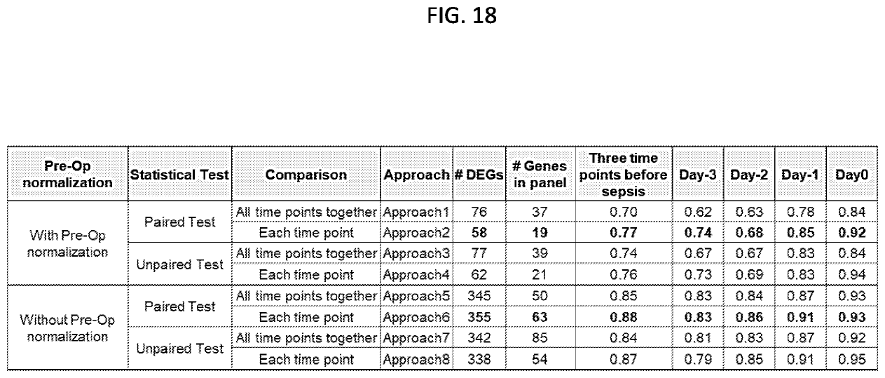

Differential expression analysis. The microarray data were normalized using a quantile method. Probes having signals lower than the global mean were removed from further analysis as it is known that low abundance transcripts have higher detection variabilities. To identify genes associated with the development of sepsis, three factors were considered during analysis: 1) the use of Pre-Op data, 2) paired or unpaired analysis between controls and sepsis samples, and 3) combining time point data prior to Day0 or not. Combinations of these 3 different factors resulted in 8 different analysis approaches (Approach 1 to 8, FIG. 18), DEGs from each analysis approach were identified based on the criteria of p-value <0.01 and absolute log 2-fold-change >0.585 (i.e., more than 1.5 fold concentration change). When comparisons were made using individual time points, only genes showing differential expression in at least two time points were selected.

Feature selection. To identify a biomarker panel consisting of a subset of genes among DEGs, support vector machine with recursive feature elimination (SVM-RFE) (Guyon et al., Machine Learning 46:389-422, 2002) was applied with 5-fold cross-validation using pathClass R package (Johannes et al., Bioinformatics 27:1442-1443, 2011). From each loop of the cross-validation procedure, an optimal set of features was selected by SVM-RFE, therefore, five different sets of features (genes) could be identified. Considering the randomness of shuffling and partitioning of samples in a cross-validation procedure, the cross-validation procedure was repeated 100 times, resulting in a total of 500 sets of features. The importance of each feature to the classification was determined as the frequency of how often each feature was selected among the 500 sets. The features were sorted in order of their importance. SVM models with different numbers of features were constructed by adding features from the most important to the least and average AUCs were computed by repeating the 5-fold cross-validation procedure 100 times. The final optimal feature set was determined at the highest average AUC.

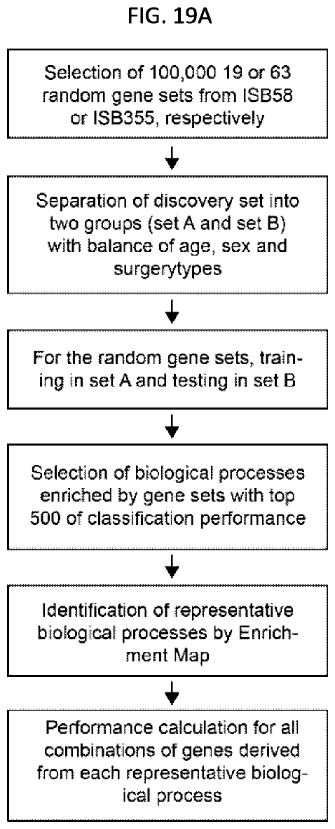

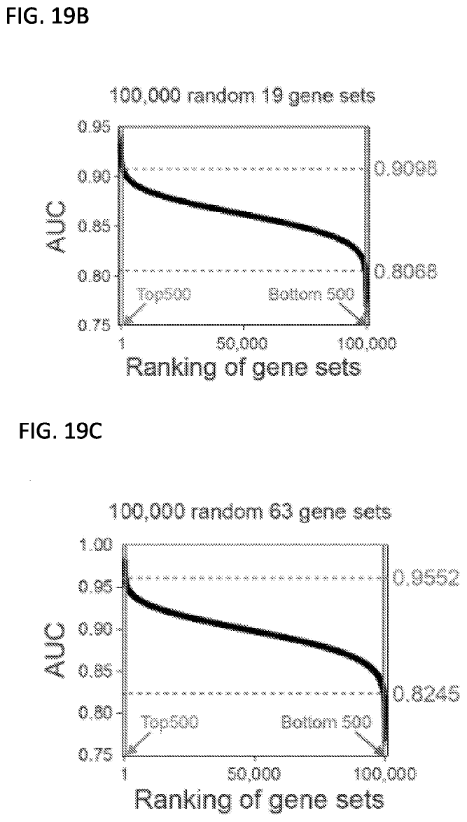

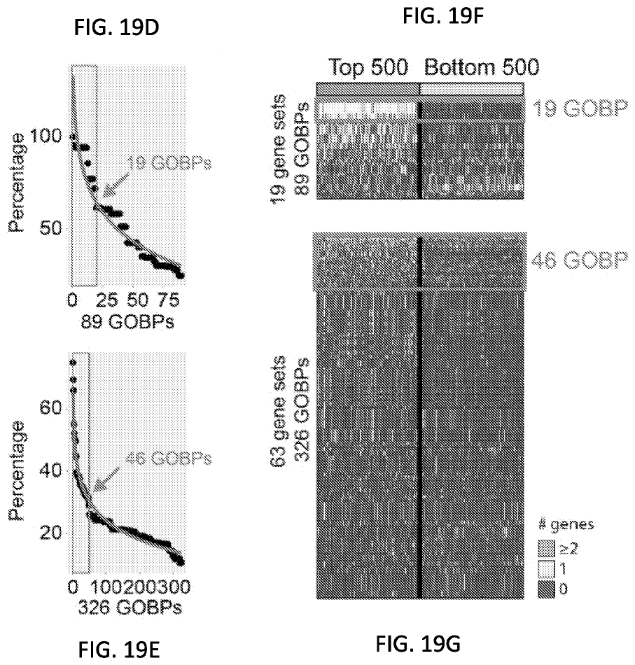

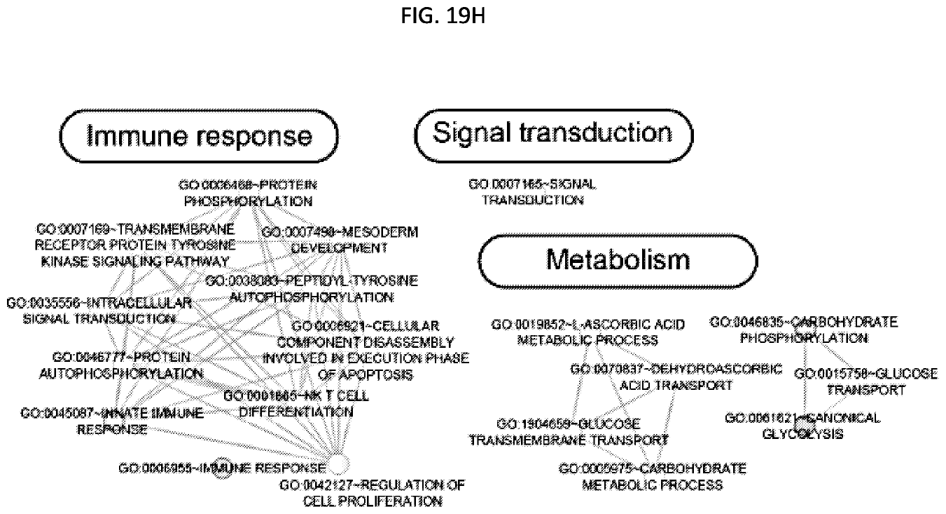

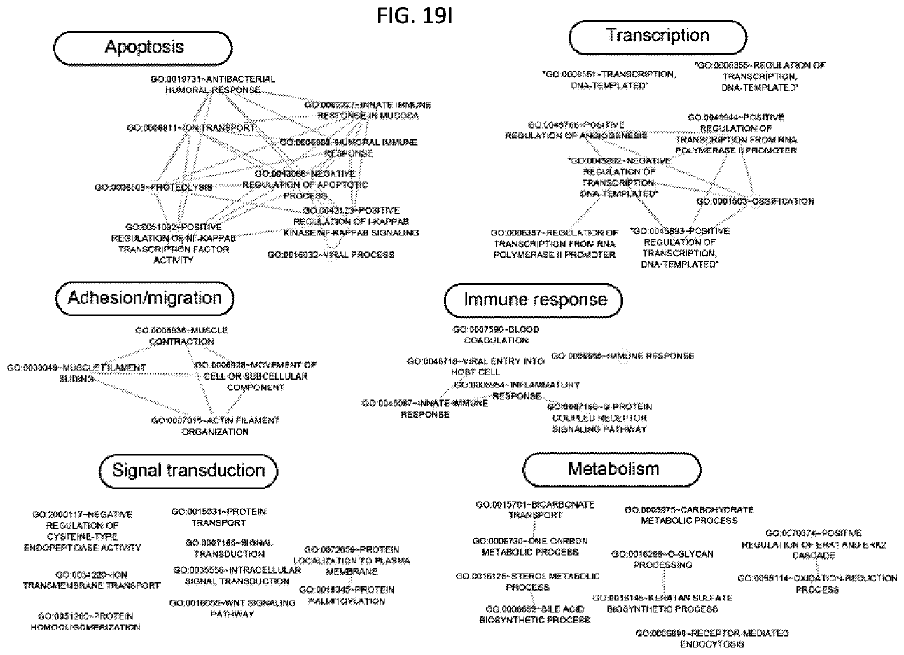

Panel optimization. To determine the biological processes associated with the classification, the Gene Ontology (GO) terms associated with the genes (features) in the top performing diagnostic panels were determined using the database for annotation visualization and integrated discovery (DAVID) (Huang et al., Nat. Protoc. 4:44-57, 2009). Then we tried to select core biological processes that have higher discriminatory power (FIG. 19A). We generated 100,000 panels that consist of the same number of genes with the panels identified by SVM-RFE. The genes were randomly selected from DEGs. The Discovery set was divided into two sets of equal size. One set was used to train random 19- or 63-gene panels and the other set was used to compute their classification performance. Among the random 100,000 panels, the top 500 high performing and bottom 500 low performing panels (1% of the random 100,000 panels) were selected. For each panel, the number of genes involved in the biological processes associated with ISB19 and ISB63 were counted. For each GOBP, the ratio of panels that have more than one gene in the corresponding GOBP were computed and sorted in decreasing order. The number of core biological processes were selected based on the Elbow method. Then the selected core biological processes were summarized to representative functional terms with EnrichmentMap tool (Merico et al., PLoS One 5:e13984, 2010). Genes in each of the DEG sets that shared the same functional terms and had the same directional changes between sepsis and control were identified as substitutable genes representing each functional term. To optimize the panel, one gene from each functional term was assembled and trained using all the samples in the Discovery sample set, and the classification performances were calculated using the Test sample set.

Throughout the analyses, classification performance of a biomarker panel was evaluated by the area under the curve (AUC) of receiver operating characteristic (ROC) curve. Statistical significance of the difference between AUCs from different panels was computed with DeLong's method (Robin et al., BMC Bioinformatics 12:77, 2011).

qPCR validation. The Fluidigm 96.96 integrated fluidic circuit (IFC) plates were used for qPCR validation of the 3- and 6-gene panels. The analysis included 665 blood RNA samples from 155 sepsis and 153 controls from the Discovery, Test, and Validation sets. The Discovery and Test sets were used to train classifiers using the panels and the Validation set was used to calculate their performances. The samples were randomized across 8 IFC chips and each chip included four wells of a pooled sample to measure inter-chip variability and two wells of no template controls. The whole blood cDNA was synthesized from 100 ng of RNA using the Reverse Transcription Master mix (Fluidigm PN 100-6297). Pre-amplification was performed using a pool of all assays using the Preamp Master Mix (Fluidigm PN 100-5744). The preamp reaction was then cleaned up using Exonuclease 1 (New England Biolabs PN M0293 S). The amplified cDNA was diluted 1:10 using DNA suspension buffer, and then the Fluidigm chips were run on the Biomark HD machine according to the manufacturer's instructions using the GE 96.96 Standard v1 protocol. Ct values from all eight chips were combined using the Biomark Data Analysis software. BRK1 and RNF181 were used as references genes. The two reference genes were selected based on the following procedure: 1) a set of invariant genes was selected in which each gene had less than 5% of coefficient of variation in each of sepsis and control datasets, below bottom 5% of fold-change distribution between sepsis and control, and within top 5% of an intensity distribution of all genes. Genes that were not defined well or were suspected to be saturated in intensity were not considered in further steps. 2) A total of 41 genes were selected from the first step and used as an input for RefFinder that provides a comprehensive ranking of genes by comparing four computational programs (delta-CT, BestKeeper, NormFinder, and geNorm) for a reference gene identification. In RefFinder, sepsis and control datasets were analyzed separately and gene rankings from each dataset were averaged. Top two ranked genes, BRK1 and RNF181, were finally selected and used as reference genes.

TABLE 19
Characteristics of Discovery, Test, and Validation cohorts.

| Sample set | | | Discovery | | Test | | Validation | |
|---|---|---|---|---|---|---|---|---|
| Characteristics | | | Control | Sepsis | Control | Sepsis | Control | Sepsis |
| Mean (Standard Deviation) | | | (n = 63) | (n = 64) | (n = 30) | (n = 31) | (n = 60) | (n = 60) |
| Age | | | 65.9 (8.0) | 64.9 (9.6) | 62.6 (14.8) | 64.2 (13.0) | 61.1 (13.5) | 61.1 (13.8) |
| Gender | Female | | 23 | 23 | 6 | 6 | 11 | 11 |
| | Male | | 40 | 41 | 24 | 25 | 49 | 49 |
| Race | Black or Black British | | | 1 | 2 | | 2 | 3 |
| | Asian or Asian British | | | | 1 | 1 | | 1 |
| | Chinese or other ethnic group | | | | | 2 | | 2 |
| | White | | 52 | 63 | 27 | 27 | 56 | 52 |
| | Not recorded | | 1 | | | 1 | 2 | 2 |
| Surgery type, N | | | | | | | | |
| Abdomen | APR | | 2 | 3 | 2 | 2 | 4 | 4 |
| | Biliary surgery | | 3 | 2 | 1 | 1 | 1 | |
| | Bowel resection/anastomosis | | 15 | 14 | 4 | 4 | 11 | 10 |
| | Cystectomy | | 2 | 2 | | | 1 | 1 |
| | Cystoprostatectomy | | | | | | | 1 |
| | Esophagectomy/Gastrectomy | | 12 | 12 | 8 | 9 | 22 | 22 |
| | Hernia/abdominal wall reconstruction | | | | | | | 1 |
| | Liver surgery | | 3 | 3 | 4 | 4 | 4 | 4 |
| | Nephrectomy | | | | 1 | 1 | 1 | |
| | Pancreaticodudenectmomy | | 12 | 13 | 5 | 5 | 8 | 9 |
| | Vascular surgery | | 10 | 9 | 2 | 2 | 3 | 2 |
| Neck | Mitrofanoff/Max fax | | 2 | 2 | 1 | 1 | | |
| Thorax | Lung resection/vascular surgery | | 2 | 4 | 2 | 2 | 5 | 6 |
| Patient Outcome, N | | | | | | | | |
| | Death | caused by sepsis | | 1 | | 1 | | 5 |
| | | not caused by sepsis | | | | | | 1 |

| | Discharged | 25 | 19 | 10 | 6 | 27 | 16 |
|---|---|---|---|---|---|---|---|
| | Inpatient | 8 | 18 | 12 | 19 | 16 | 31 |
| | Not recorded | 30 | 26 | 8 | 5 | 17 | 7 |
| Microbiology, N (%) | | | | | | | |
| Blood | Positive | | 10 (16%) | | 5 (16%) | | 8 (13%) |
| | Negative | 1 (2%) | 16 (25%) | | 14 (45%) | 2 (3%) | 27 (45%) |
| Sputum | Positive | 1 (2%) | 25 (6%) | | 12 (39%) | | 11 (18%) |
| | Negative | 3 (5%) | 4 (6%) | | 1 (3%) | 3 (5%) | 9 (15%) |
| Urine | Positive | | 3 (5%) | | | | 8 (13%) |
| | Negative | 6 (10%) | 7 (11%) | 1 (3%) | 12 (39%) | 1 (2%) | 9 (15%) |
| Throat | Positive | | 2 (3%) | | | | 1 (2%) |
| | Negative | 1 (2%) | 3 (5%) | 3 (10%) | 1 (3%) | 1 (2%) | 2 (3%) |
| Wound Site | Positive | 2 (3%) | 27 (42%) | | 9 (29%) | | 21 (35%) |
| | Negative | 1 (2%) | 2 (3%) | 1 (3%) | 1 (3%) | | 4 (7%) |

APR indicates abdominoperineal excision of rectum & end colostomy.

### Results

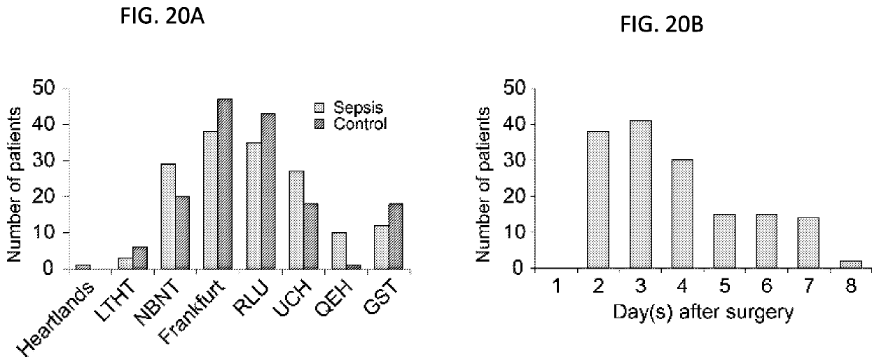

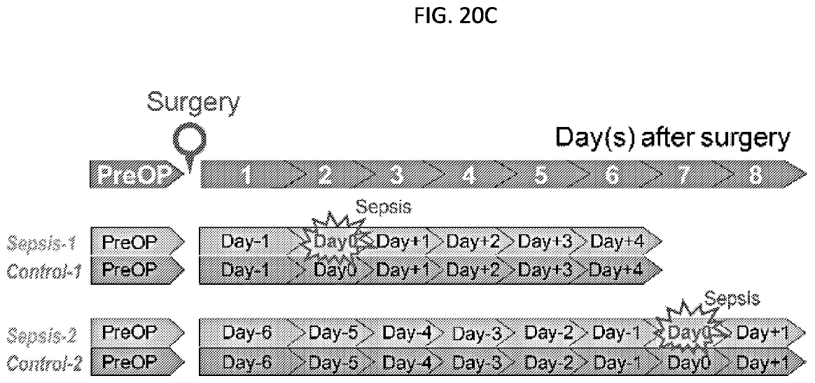

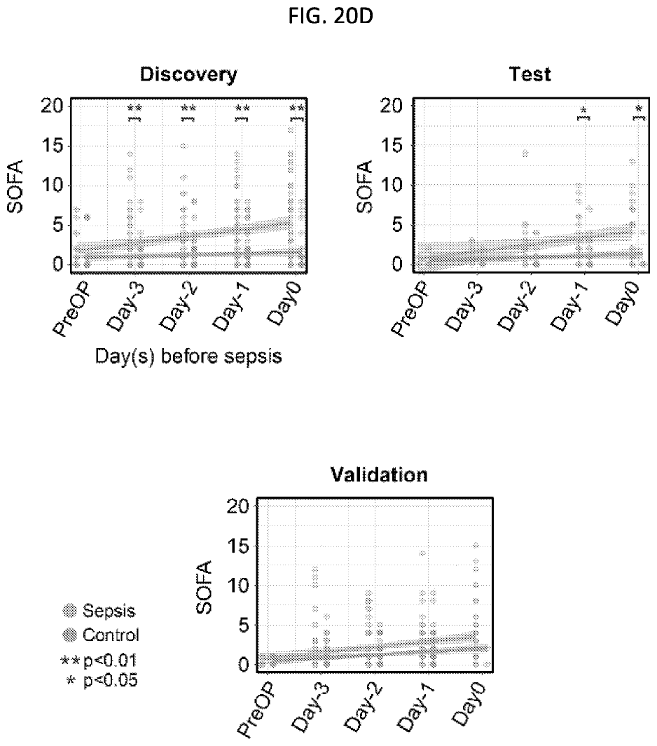

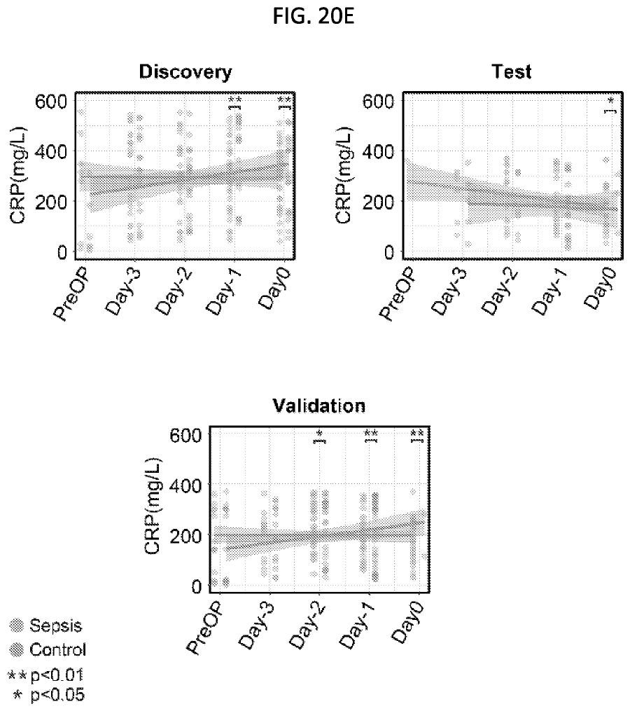

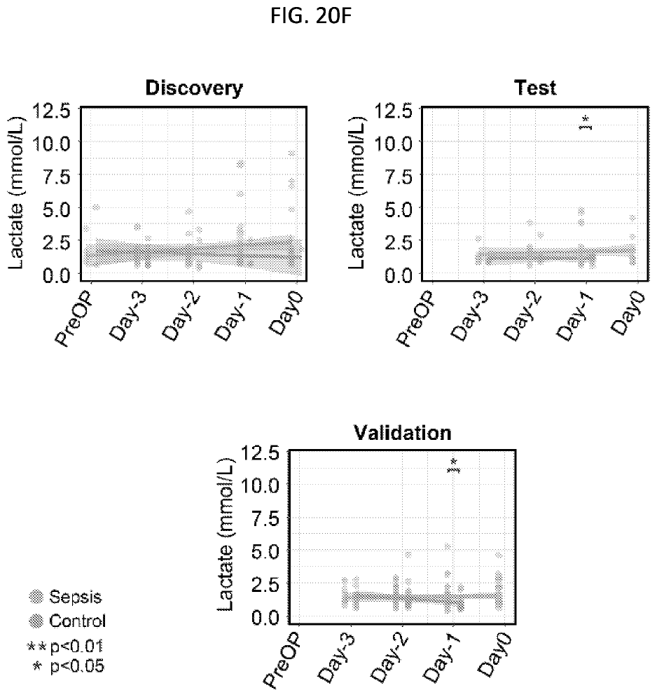

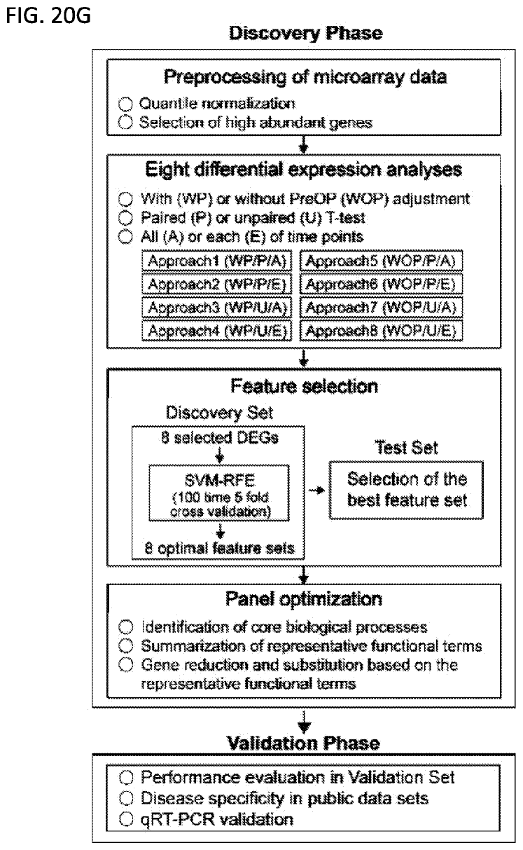

Patient cohort and study design. Surgery patients were recruited across eight hospitals located in England and Germany (FIG. 20A). Detailed patient information is shown in Table 19. Peripheral blood samples were collected from elective surgery patients prior to surgery (Pre-Op) and then daily up to four or five days post-sepsis diagnosis (Post-Op). About 4,000 patients consented and participated in the study, and approximately 4% of the enrolled patients developed sepsis (n=155) with most developing the disease two to seven days after surgery (FIG. 20B). Sepsis patients were diagnosed by a panel of nine clinicians using the Sepsis-2 criteria. The sepsis-related mortality rate in this cohort was approximately 5% (7/155 sepsis patients). The control group included age, sex, and surgical procedure matched patients who did not develop sepsis.

The sepsis patients and matched controls were each split into three groups: Discovery (64 sepsis and 63 control), Test (31 sepsis and 30 control), and Validation (60 sepsis and 60 control) sets. Samples from the Discovery set were used to identify biomarker panel(s) based on differentially expressed genes (DEGs) from whole blood between sepsis patients and controls. The Test samples were used to optimize the biomarker panel(s) and the Validation set was used to verify the panels' classification performance.

To identify pre-symptomatic biomarkers, the longitudinal samples from each patient were organized such that the day sepsis was diagnosed was labeled as Day0 (FIG. 20C). One day before the sepsis diagnosis date was set as Day-1 while one day post-diagnosis was designated as Day+1. Analysis for pre-symptomatic biomarker detection was focused on Day-3 to Day-1 time points (three to one days prior to the day of sepsis diagnosis, respectively). We could not examine earlier time points due to an insufficient number of available samples earlier than Day-3 (Table 20). There were no significant differences in clinical parameters commonly used for sepsis including Sequential Organ Failure Assessment (SOFA) Score, blood C reactive protein (CRP), and lactate levels among the three sample group populations (Discovery, Test, Validation) (FIG. 20D-20F).

TABLE 20
Number of samples in time points rearranged
by sepsis diagnosis day

| | Discovery Set | | Test Set | | Validation Set | |
|---|---|---|---|---|---|---|
| Time point | sepsis | control | sepsis | control | sepsis | control |
| Pre-Operation | 64 | 63 | 31 | 30 | 60 | 60 |
| Sepsis Day −7 | 1 | 2 | | | | |
| Sepsis Day −6 | 8 | 9 | | | 1 | 1 |
| Sepsis Day −5 | 16 | 17 | | | 1 | 1 |
| Sepsis Day −4 | 20 | 21 | 1 | 1 | 1 | 1 |
| Sepsis Day −3 | 39 | 38 | 11 | 11 | 26 | 26 |
| Sepsis Day −2 | 48 | 46 | 21 | 19 | 48 | 46 |
| Sepsis Day −1 | 62 | 57 | 29 | 30 | 58 | 56 |
| Sepsis Day 0 | 55 | 49 | 27 | 5 | 54 | 6 |
| Sepsis Day +1 | 44 | 40 | 23 | 4 | 46 | 5 |
| Sepsis Day +2 | 31 | 26 | 4 | 5 | 4 | 5 |
| Sepsis Day +3 | 21 | 17 | 4 | 4 | 4 | 4 |
| Sepsis Day +4 | 9 | 10 | 4 | 3 | 3 | 2 |
| Sepsis Day +5 | 3 | 6 | 3 | 2 | | 1 |
| Total | 421 | 401 | 158 | 114 | 306 | 214 |

Identification of pre-symptomatic sepsis diagnostic panels. Because of the study design, different approaches were used to identify gene expression changes associated with sepsis development (FIG. 20G). Specifically, variables included 1) adjusting the Post-Op whole blood transcriptome data with Pre-Op data, 2) individual patient-control pairs comparison vs. unpaired all patients vs. all controls analysis, and 3) individual time points vs. combining all pre-sepsis diagnosis time points. The use of Pre-Op data to adjust Post-Op array data reduces the variation among individuals by controlling potential confounding variables from an individual and emphasizing the gene expression changes associated with surgery and infection. The paired and unpaired t-test was used to determine whether to analyze individual patient-control pairs or group the patient and control samples together. The third factor was to compare the gene expression profile changes between the sepsis and control groups either at individual time points (e.g., Day-3 sepsis vs. Day-3 control) or by grouping all three time points (Day-3+Day-2+Day-1) from each condition together. In total, eight different approaches were used (FIG. 18). As expected, there were fewer DEGs when using Pre-Op adjusted data (Approaches 1 to 4, FIG. 18) and most of the DEGs identified with Pre-Op adjusted data were also included in data without Pre-Op adjustment (Approaches 5 to 8, FIG. 18). However, a few DEGs were observed only in Pre-Op adjusted data. For example GBP5 (Guanylate Binding Protein 5) and GZMH (Granzyme H) were identified as DEGs with Approaches 1 to 4 but not with Approaches 5 to 8.

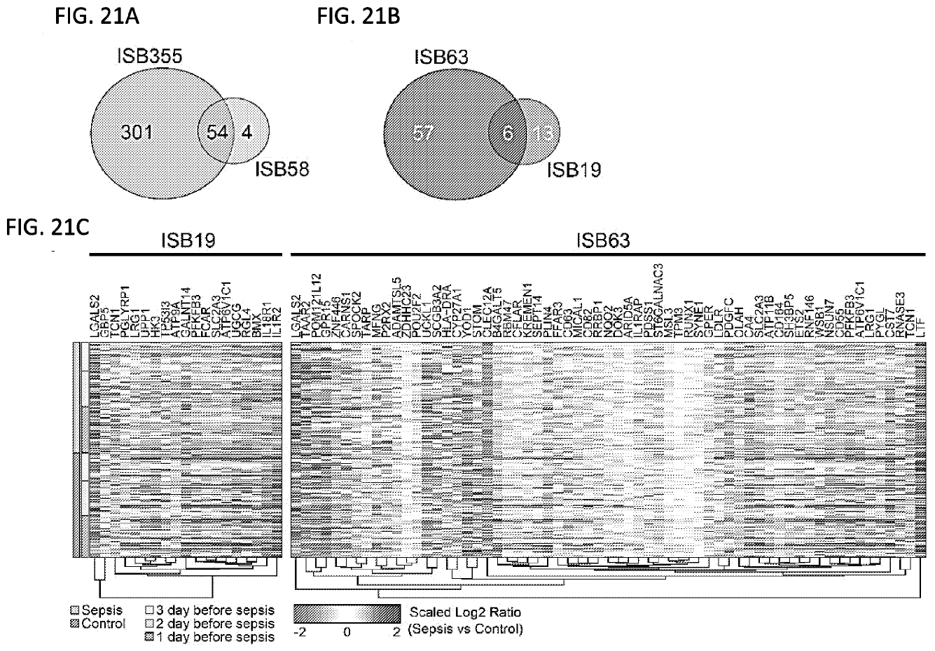

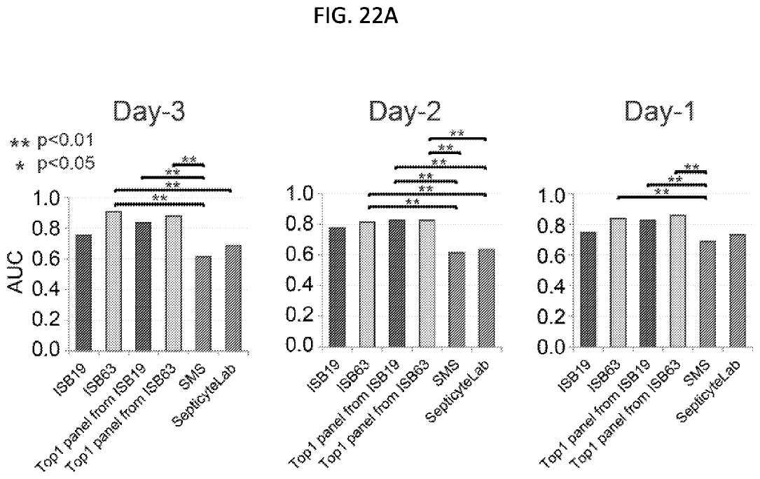

Using the DEGs identified from the Discovery sample set, a classifier capable of separating pre-symptomatic sepsis patients from control patients was identified using support vector machine combined with recursive feature elimination (SVM-RFE) in conjunction with a repeat cross-validation procedure for each of the DEG sets from different analysis approaches. The performances (indicated as area under the receiver operating characteristic curve (AUC) for each day prior to the day of sepsis diagnosis) of the panels were then computed (summarized in FIG. 18). In general, panels identified from the datasets without Pre-Op adjustment (from Approaches 5-8) showed higher performance than the panels from the Pre-Op adjusted datasets. Among the four different Pre-Op adjusted datasets, the 19-gene panel (ISB19) derived from 58 DEGs (ISB58) that were identified by Approach 2 showed the highest performance based on the average AUC of three time points (Day-3 to Day-1) (average AUC=0.77). The 63-gene panel (ISB63) derived from 355 DEGs (ISB355) that were identified by Approach 6 gave the highest performance based on the average AUC of three time points among datasets without Pre-Op adjustment (average AUC=0.88). Apart from the difference with or without Pre-Op adjustment, Approaches 2 and 6 both tested individual patient-control pairs and individual time points. We observed significant overlap (54 genes) in DEGs between the ISB58 and ISB355 sets (FIG. 21A and Table 21); however, the panels (ISP19 and ISB63) derived from these DEGs contained only 6 genes in common (FIG. 21B and Table 21). The genes included in the two panels are listed in Table 21 and the overall expression profiles of those genes among samples in the Discovery set are shown in FIG. 21C

TABLE 21
Differentially expressed genes (DEGs) identified from approach 2 (ISB58) and approach 6 (ISB355)

| | | PreOP | | PreOP | | PreOP | | | | | | | | | | | |
|---|---|---|---|---|---|---|---|---|---|---|---|---|---|---|---|---|---|
| | | norm. | | norm. | | norm. | | | | | | | | | | | |
| | Gene | Day −3 | P | Day −2 | P | Day −1 | P | Day −3 | P | Day −2 | P | Day −1 | P | Approach2 | Approach6 | | |
| Probe ID | Symbol | logFC | Value | logFC | Value | logFC | Value | logFC | Value | logFC | Value | logFC | Value | (ISB58) | (ISB355) | ISB19 | ISB63 |
| A_23_P13765 | FCER1A | −0.54 | 0.19 | −0.33 | 0.14 | −0.69 | 0.36 | −0.70 | 0.13 | −0.65 | 0.00 | −0.92 | 0.00 | | X | | |
| A_24_P11729 | LDLR | 0.49 | 0.22 | 0.38 | 0.62 | 0.47 | 0.72 | 0.67 | 0.22 | 0.58 | 0.28 | 0.70 | 0.00 | | X | | X |
| A_23_P117546 | SOS2 | 0.31 | 0.62 | 0.24 | 0.75 | 0.38 | 0.17 | 0.53 | 0.59 | 0.57 | 0.00 | 0.65 | 0.73 | | X | | |
| A_23_P14464 | ALOX5 | 0.47 | 0.58 | 0.39 | 0.19 | 0.43 | 0.15 | 0.78 | 0.18 | 0.73 | 0.00 | 0.72 | 0.00 | | X | | |
| A_24_P82466 | GAS7 | 0.75 | 0.29 | 0.71 | 0.15 | 0.46 | 0.26 | 0.61 | 0.00 | 0.63 | 0.00 | 0.62 | 0.00 | | X | | |
| A_23_P122863 | GRB1 | 0.60 | 0.28 | 0.55 | 0.26 | 0.97 | 0.47 | 0.79 | 0.35 | 0.86 | 0.24 | 1.23 | 0.00 | | X | | |
| A_23_P411113 | CNTNAP1 | −0.33 | 0.27 | −0.12 | 0.59 | −0.66 | 0.65 | −0.83 | 0.34 | −0.64 | 0.58 | −0.55 | 0.28 | | X | | |
| A_33_P3352382 | ARG1 | 0.97 | 0.16 | 0.73 | 0.23 | 1.87 | 0.85 | 1.11 | 0.33 | 1.21 | 0.00 | 1.46 | 0.00 | | X | | |
| A_23_P61426 | MSRA | 0.55 | 0.19 | 0.54 | 0.37 | 0.62 | 0.26 | 0.63 | 0.40 | 0.63 | 0.48 | 0.77 | 0.00 | | X | | |
| A_23_P2676 | HP | 1.85 | 0.58 | 0.75 | 0.42 | 1.21 | 0.46 | 1.39 | 0.12 | 1.33 | 0.63 | 1.77 | 0.00 | X | X | | |
| A_23_P121716 | ANXA3 | 0.75 | 0.32 | 0.55 | 0.77 | 0.69 | 0.12 | 1.61 | 0.24 | 1.33 | 0.00 | 1.14 | 0.00 | | X | | |
| A_23_P17735 | CD79A | −0.18 | 0.54 | 0.83 | 0.67 | −0.40 | 0.28 | −0.79 | 0.38 | −0.54 | 0.49 | −0.80 | 0.79 | | X | | |
| A_23_P21287 | KIF1B | 0.58 | 0.62 | 0.47 | 0.68 | 0.57 | 0.46 | 0.85 | 0.00 | 0.89 | 0.00 | 0.89 | 0.00 | | X | | |
| A_23_P12418 | ITGAM | 0.52 | 0.15 | 0.37 | 0.36 | 0.45 | 0.80 | 0.71 | 0.13 | 0.58 | 0.00 | 0.69 | 0.00 | | X | | |
| A_23_P47565 | LDHA | 0.61 | 0.62 | 0.47 | 0.97 | 0.55 | 0.18 | 0.85 | 0.13 | 0.77 | 0.00 | 0.84 | 0.00 | | X | | |
| A_23_P12847 | CLEC12A | 0.38 | 0.62 | 0.35 | 0.16 | 0.15 | 0.17 | 0.83 | 0.79 | 0.70 | 0.48 | 0.55 | 0.49 | | X | | X |
| A_32_P18254 | FAM2A | 0.45 | 0.42 | 0.32 | 0.17 | 0.44 | 0.66 | 0.66 | 0.55 | 0.66 | 0.59 | 0.86 | 0.00 | | X | | |
| A_23_P82929 | NOV | −0.56 | 0.62 | −0.49 | 0.12 | −0.53 | 0.79 | −0.62 | 0.27 | −0.68 | 0.00 | −0.75 | 0.00 | | X | | |
| A_23_P129556 | IL4R | 0.42 | 0.76 | 0.37 | 0.55 | 0.45 | 0.22 | 0.67 | 0.87 | 0.78 | 0.00 | 0.83 | 0.00 | | X | | |
| A_23_P14464 | ALOX5 | 0.49 | 0.21 | 0.42 | 0.12 | 0.44 | 0.57 | 0.75 | 0.00 | 0.73 | 0.00 | 0.70 | 0.00 | | X | | |
| A_24_P385611 | SR1 | 0.24 | 0.46 | 0.16 | 0.17 | 0.27 | 0.23 | 0.61 | 0.00 | 0.63 | 0.13 | 0.72 | 0.00 | | X | | |
| A_33_P321689 | PAG1 | 0.37 | 0.18 | 0.23 | 0.54 | 0.42 | 0.39 | 0.79 | 0.00 | 0.74 | 0.65 | 0.84 | 0.00 | | X | | |
| A_23_P121716 | ANXA3 | 0.77 | 0.27 | 0.56 | 0.70 | 0.68 | 0.13 | 1.85 | 0.15 | 1.42 | 0.00 | 1.14 | 0.00 | | X | | |
| A_23_P161458 | OLAH | 0.66 | 0.16 | 0.85 | 0.22 | 0.99 | 0.26 | 0.78 | 0.18 | 1.54 | 0.16 | 1.15 | 0.00 | X | X | | X |
| A_23_P152548 | SCPEP1 | 0.42 | 0.51 | 0.32 | 0.54 | 0.40 | 0.44 | 0.65 | 0.63 | 0.52 | 0.42 | 0.59 | 0.00 | | X | | |
| A_23_P123732 | C9orf13 | −0.14 | 0.43 | 0.44 | 0.47 | 0.44 | 0.83 | −0.15 | 0.34 | 0.59 | 0.24 | 0.63 | 0.16 | | X | | |
| A_23_P122863 | GRB1 | 0.55 | 0.39 | 0.50 | 0.32 | 0.86 | 0.21 | 0.77 | 0.49 | 0.90 | 0.00 | 1.29 | 0.16 | | X | | |
| A_33_P3361422 | CYP27A1 | −0.51 | 0.50 | −0.45 | 0.78 | −0.32 | 0.35 | −0.74 | 0.27 | −0.83 | 0.00 | −0.58 | 0.13 | | X | | X |
| A_23_P9451 | ANXA1 | 0.37 | 0.49 | 0.36 | 0.23 | 0.28 | 0.39 | 0.63 | 0.53 | 0.79 | 0.00 | 0.55 | 0.19 | | X | | |
| A_33_P3352578 | CLEC4D | 0.82 | 0.43 | 0.63 | 0.21 | 0.89 | 0.25 | 1.72 | 0.14 | 1.82 | 0.00 | 1.29 | 0.56 | X | X | | |
| A_23_P79426 | CAB39 | 0.43 | 0.34 | 0.27 | 0.28 | 0.38 | 0.64 | 0.68 | 0.00 | 0.65 | 0.00 | 0.71 | 0.55 | | X | | |
| A_33_P3421571 | RAPH1 | −0.14 | 0.64 | −0.13 | 0.59 | −0.52 | 0.12 | −0.69 | 0.16 | −0.70 | 0.37 | −0.87 | 0.52 | | X | | |
| A_23_P119222 | RETN | 1.15 | 0.69 | 1.43 | 0.18 | 1.24 | 0.17 | 1.37 | 0.28 | 1.34 | 0.18 | 1.52 | 0.00 | X | X | | |
| A_24_P343233 | HLA-DRB1 | −0.27 | 0.56 | −0.24 | 0.26 | −0.53 | 0.17 | −0.41 | 0.39 | −0.62 | 0.00 | −0.71 | 0.00 | | X | | |
| A_23_P121716 | ANXA3 | 0.79 | 0.26 | 0.56 | 0.78 | 0.67 | 0.16 | 1.88 | 0.17 | 1.32 | 0.00 | 1.11 | 0.00 | | X | | |
| A_23_P122863 | GRB1 | 0.72 | 0.18 | 0.51 | 0.26 | 0.97 | 0.50 | 0.89 | 0.17 | 0.88 | 0.00 | 1.28 | 0.43 | | X | | |
| A_23_P123393 | KCNQ3 | −0.20 | 0.47 | −0.26 | 0.27 | −0.22 | 0.22 | −0.61 | 0.23 | −0.75 | 0.54 | −0.74 | 0.72 | | X | | |
| A_24_P363548 | HIP1 | 0.41 | 0.48 | 0.28 | 0.14 | 0.42 | 0.13 | 0.64 | 0.00 | 0.53 | 0.00 | 0.71 | 0.00 | | X | | |
| A_23_P12418 | ITGAM | 0.55 | 0.15 | 0.37 | 0.38 | 0.48 | 0.41 | 0.69 | 0.42 | 0.57 | 0.00 | 0.73 | 0.00 | | X | | |
| A_24_P819 | SLC2A3 | 0.66 | 0.27 | 0.57 | 0.52 | 0.63 | 0.39 | 0.98 | 0.00 | 1.00 | 0.36 | 1.67 | 0.00 | X | X | X | X |
| A_24_P59667 | JAK3 | 0.19 | 0.34 | 0.19 | 0.18 | 0.45 | 0.83 | 0.62 | 0.45 | 0.69 | 0.00 | 0.85 | 0.00 | | X | | |
| A_24_P314179 | ETS2 | 0.56 | 0.16 | 0.50 | 0.84 | 0.45 | 0.16 | 0.84 | 0.00 | 0.73 | 0.00 | 0.75 | 0.00 | | X | | X |
| A_33_P3316786 | DACH1 | 0.65 | 0.16 | 0.64 | 0.28 | 0.77 | 0.00 | 0.81 | 0.00 | 0.89 | 0.00 | 1.42 | 0.00 | X | X | | |
| A_33_P337682L | GZMA | −0.59 | 0.83 | −0.61 | 0.33 | −0.46 | 0.27 | −0.62 | 0.13 | −0.76 | 0.24 | −0.65 | 0.19 | X | X | | |
| A_23_P12566 | RRBP1 | 0.28 | 0.36 | 0.18 | 0.11 | 0.37 | 0.13 | 0.65 | 0.00 | 0.55 | 0.00 | 0.73 | 0.00 | | X | | X |
| A_23_P123393 | KCNQ3 | −0.37 | 0.21 | 0.23 | 0.32 | −0.42 | 0.57 | −0.95 | 0.13 | −0.27 | 0.30 | −0.68 | 0.69 | | X | | |
| A_23_P7253 | KLHL2 | 0.62 | 0.15 | 0.43 | 0.39 | 0.55 | 0.55 | 0.92 | 0.00 | 0.88 | 0.00 | 0.97 | 0.00 | | X | | |
| A_23_P68851 | KREMEN1 | 0.24 | 0.29 | 0.25 | 0.29 | 0.34 | 0.95 | 0.75 | 0.26 | 0.66 | 0.00 | 0.87 | 0.13 | | X | | X |
| A_33_P329459 | CD44 | 0.49 | 0.67 | 0.45 | 0.14 | 0.35 | 0.66 | 0.65 | 0.54 | 0.63 | 0.00 | 0.58 | 0.00 | | X | | |
| A_23_P3451 | PHC2 | 0.26 | 0.82 | 0.29 | 0.85 | 0.25 | 0.56 | 0.67 | 0.00 | 0.61 | 0.00 | 0.69 | 0.00 | | X | | |
| A_23_P32577 | DACH1 | 0.68 | 0.26 | 0.58 | 0.25 | 0.64 | 0.00 | 0.76 | 0.00 | 0.77 | 0.00 | 0.83 | 0.00 | X | X | | |
| A_23_P64372 | TCN1 | 0.67 | 0.26 | 0.63 | 0.21 | 0.72 | 0.97 | 0.75 | 0.31 | 0.68 | 0.22 | 0.79 | 0.34 | X | X | X | X |
| A_33_P322489 | IL17RA | 0.28 | 0.54 | 0.18 | 0.18 | 0.24 | 0.38 | 0.60 | 0.00 | 0.47 | 0.00 | 0.59 | 0.00 | | X | | |
| A_23_P1476 | GPR97 | 0.57 | 0.13 | 0.58 | 0.20 | 0.54 | 0.46 | 0.59 | 0.00 | 0.69 | 0.00 | 0.76 | 0.00 | | X | | |
| A_23_P14464 | ALOX5 | 0.47 | 0.22 | 0.49 | 0.12 | 0.48 | 0.87 | 0.73 | 0.19 | 0.79 | 0.00 | 0.66 | 0.00 | | X | | |
| A_33_P3424577 | A23747 | −0.49 | 0.23 | −0.38 | 0.57 | −0.58 | 0.23 | −0.69 | 0.59 | −0.68 | 0.13 | −0.81 | 0.16 | | X | | |
| A_23_P123732 | C9orf13 | −0.83 | 0.73 | 0.35 | 0.75 | 0.48 | 0.15 | 0.14 | 0.58 | 0.64 | 0.47 | 0.70 | 0.27 | | X | | |
| A_23_P251937 | CPEB4 | 0.35 | 0.59 | 0.19 | 0.14 | 0.42 | 0.99 | 0.66 | 0.27 | 0.54 | 0.00 | 0.72 | 0.00 | | X | | |
| A_33_P3258977 | CLEC4D | 0.86 | 0.43 | 0.68 | 0.13 | 0.90 | 0.24 | 1.43 | 0.24 | 0.99 | 0.11 | 1.15 | 0.00 | X | X | | |
| A_23_P14658 | ATP6V1C1 | 0.77 | 0.23 | 0.55 | 0.93 | 0.69 | 0.11 | 0.98 | 0.00 | 0.86 | 0.13 | 1.62 | 0.00 | X | X | X | X |
| A_23_P117546 | SOSA | 0.42 | 0.34 | 0.33 | 0.37 | 0.47 | 0.26 | 0.53 | 0.55 | 0.58 | 0.00 | 0.69 | 0.87 | | X | | |
| A_21_P1251 | SEPT14 | 0.15 | 0.29 | 0.13 | 0.22 | 0.36 | 0.93 | 0.64 | 0.00 | 0.73 | 0.00 | 0.80 | 0.00 | | X | | X |
| A_23_P9513 | SLC37A3 | 0.52 | 0.34 | 0.46 | 0.60 | 0.68 | 0.26 | 0.79 | 0.45 | 0.83 | 0.00 | 0.94 | 0.00 | | X | | |
| A_32_P7158 | LILRB3 | 0.27 | 0.13 | 0.17 | 0.23 | 0.35 | 0.18 | 0.72 | 0.22 | 0.64 | 0.00 | 0.74 | 0.00 | | X | | |
| A_23_P28334 | IL18RAP | 0.62 | 0.58 | 0.59 | 0.26 | 0.76 | 0.29 | 1.23 | 0.65 | 1.11 | 0.00 | 1.28 | 0.00 | | X | | |
| A_23_P14464 | ALOX5 | 0.47 | 0.18 | 0.44 | 0.13 | 0.43 | 0.76 | 0.73 | 0.22 | 0.69 | 0.00 | 0.67 | 0.00 | | X | | |
| A_24_P74932 | PLP2 | 0.52 | 0.13 | 0.48 | 0.15 | 0.38 | 0.89 | 0.67 | 0.17 | 0.62 | 0.00 | 0.51 | 0.00 | | X | | |
| A_23_P11473 | NAIP | 0.46 | 0.98 | 0.23 | 0.29 | 0.45 | 0.28 | 0.93 | 0.23 | 0.86 | 0.00 | 1.19 | 0.00 | | X | | |
| A_23_P4174 | MMP9 | 1.79 | 0.25 | 0.81 | 0.90 | 1.21 | 0.13 | 1.22 | 0.54 | 1.11 | 0.00 | 1.46 | 0.00 | X | X | | |
| A_23_P29625 | CYP1B1 | 0.30 | 0.19 | 0.24 | 0.23 | 0.51 | 0.56 | 0.77 | 0.25 | 0.77 | 0.14 | 0.93 | 0.00 | | X | | |
| A_24_P223124 | FNDC3B | 0.36 | 0.19 | 0.15 | 0.21 | 0.24 | 0.30 | 0.59 | 0.00 | 0.51 | 0.00 | 0.62 | 0.00 | | X | | |
| A_21_P1748 | SEPT14 | 0.13 | 0.41 | 0.15 | 0.25 | 0.30 | 0.25 | 0.68 | 0.00 | 0.82 | 0.00 | 0.89 | 0.00 | | X | | X |
| A_33_P33896 | CDK5RAP2 | 0.73 | 0.64 | 0.67 | 0.49 | 0.77 | 0.17 | 0.88 | 0.73 | 0.97 | 0.00 | 1.86 | 0.98 | X | X | | |
| A_21_P1561 | FCGR1B | 0.32 | 0.17 | 0.35 | 0.12 | 0.46 | 0.86 | 0.95 | 0.00 | 0.85 | 0.24 | 0.72 | 0.32 | | X | | |
| A_23_P25362 | BMX | 0.69 | 0.47 | 0.66 | 0.34 | 0.86 | 0.76 | 0.73 | 0.16 | 0.79 | 0.00 | 0.91 | 0.00 | X | X | X | |
| A_21_P1728 | FCGR1B | 0.23 | 0.29 | 0.25 | 0.23 | 0.25 | 0.92 | 0.91 | 0.00 | 0.83 | 0.11 | 0.72 | 0.19 | | X | | |
| A_33_P322196 | IL18RAP | 0.67 | 0.39 | 0.58 | 0.23 | 0.77 | 0.23 | 1.62 | 0.35 | 1.91 | 0.00 | 1.28 | 0.00 | | X | | |
| A_23_P11212 | ACSL1 | 0.49 | 0.38 | 0.29 | 0.13 | 0.39 | 0.29 | 0.82 | 0.00 | 0.75 | 0.00 | 0.85 | 0.00 | | X | | |
| A_24_P27787 | SNX3 | 0.54 | 0.32 | 0.31 | 0.37 | 0.64 | 0.64 | 0.67 | 0.00 | 0.64 | 0.00 | 0.55 | 0.84 | | X | | |
| A_23_P117546 | SOS2 | 0.37 | 0.34 | 0.27 | 0.46 | 0.37 | 0.17 | 0.58 | 0.53 | 0.58 | 0.00 | 0.64 | 0.00 | | X | | |
| A_23_P119835 | NLRC4 | 0.53 | 0.39 | 0.36 | 0.93 | 0.65 | 0.16 | 0.86 | 0.28 | 0.84 | 0.00 | 1.55 | 0.25 | | X | | |
| A_23_P14464 | ALOX5 | 0.41 | 0.43 | 0.37 | 0.23 | 0.38 | 0.17 | 0.72 | 0.19 | 0.72 | 0.00 | 0.67 | 0.00 | | X | | |
| A_24_P14566 | KIF1B | 0.56 | 0.43 | 0.26 | 0.28 | 0.39 | 0.36 | 0.78 | 0.68 | 0.54 | 0.00 | 0.68 | 0.00 | | X | | |
| A_23_P3913 | HLA-DPA1 | −0.29 | 0.13 | −0.37 | 0.15 | −0.58 | 0.26 | −0.42 | 0.99 | −0.70 | 0.00 | −0.74 | 0.00 | | X | | |
| A_33_P3253144 | DOK3 | 0.41 | 0.44 | 0.39 | 0.20 | 0.53 | 0.66 | 0.56 | 0.11 | 0.62 | 0.00 | 0.73 | 0.00 | | X | | X |
| A_33_P3251148 | TSPO | 0.57 | 0.83 | 0.50 | 0.74 | 0.62 | 0.79 | 0.76 | 0.13 | 0.72 | 0.00 | 0.78 | 0.00 | | X | | |
| A_24_P37172 | LILRA5 | 0.63 | 0.12 | 0.52 | 0.22 | 0.65 | 0.15 | 0.96 | 0.00 | 0.95 | 0.00 | 1.95 | 0.00 | | X | | |
| A_23_P133438 | FAM15A | 0.54 | 0.89 | 0.28 | 0.46 | 0.34 | 0.26 | 0.64 | 0.00 | 0.55 | 0.00 | 0.62 | 0.56 | | X | | |
| A_23_P11212 | ACSL1 | 0.42 | 0.90 | 0.24 | 0.25 | 0.34 | 0.54 | 0.79 | 0.25 | 0.77 | 0.00 | 0.82 | 0.00 | | X | | |
| A_23_P4856 | CKAP4 | 0.43 | 0.46 | 0.43 | 0.29 | 0.65 | 0.57 | 0.77 | 0.78 | 0.81 | 0.00 | 0.92 | 0.00 | | X | | |
| A_24_P353619 | ALPL | 0.60 | 0.16 | 0.56 | 0.23 | 0.43 | 0.82 | 0.86 | 0.33 | 0.85 | 0.00 | 0.77 | 0.33 | | X | | |
| A_24_P41776 | MXD3 | 0.33 | 0.34 | 0.23 | 0.50 | 0.45 | 0.42 | 0.66 | 0.00 | 0.60 | 0.00 | 0.83 | 0.77 | | X | | |
| A_33_P34763 | PLIN4 | 0.43 | 0.39 | 0.39 | 0.99 | 0.53 | 0.26 | 0.83 | 0.29 | 0.85 | 0.00 | 1.37 | 0.00 | | X | | X |
| A_33_P326857 | CEACAM1 | 0.56 | 0.72 | 0.45 | 0.29 | 0.60 | 0.85 | 0.79 | 0.00 | 0.64 | 0.22 | 0.78 | 0.00 | | X | | |
| A_23_P121716 | ANXA3 | 0.77 | 0.30 | 0.57 | 0.67 | 0.67 | 0.16 | 1.73 | 0.24 | 1.53 | 0.00 | 1.12 | 0.00 | | X | | |
| A_23_P169278 | AGTPBP1 | 0.42 | 0.37 | 0.31 | 0.40 | 0.33 | 0.29 | 0.66 | 0.18 | 0.65 | 0.00 | 0.63 | 0.00 | | X | | |
| A_23_P5392 | TP53I3 | 0.69 | 0.35 | 0.48 | 0.72 | 0.62 | 0.93 | 0.83 | 0.00 | 0.66 | 0.62 | 0.78 | 0.00 | X | X | X | |
| A_33_P3298356 | SH3GLB1 | 0.27 | 0.17 | 0.29 | 0.78 | 0.44 | 0.33 | 0.54 | 0.47 | 0.63 | 0.00 | 0.77 | 0.00 | | X | | |
| A_24_P393864 | PHTF1 | 0.46 | 0.75 | 0.27 | 0.24 | 0.39 | 0.46 | 0.68 | 0.00 | 0.52 | 0.00 | 0.64 | 0.95 | | X | | |
| A_24_P384397 | RAVER1 | 0.16 | 0.62 | 0.11 | 0.66 | −0.31 | 0.19 | −0.67 | 0.23 | −0.76 | 0.17 | −0.99 | 0.85 | | X | | |
| A_23_P14464 | ALOX5 | 0.41 | 0.48 | 0.40 | 0.17 | 0.44 | 0.14 | 0.68 | 0.47 | 0.72 | 0.00 | 0.66 | 0.00 | | X | | |
| A_32_P1396 | WDFY3 | 0.43 | 0.35 | 0.23 | 0.17 | 0.30 | 0.36 | 0.73 | 0.00 | 0.63 | 0.00 | 0.67 | 0.00 | | X | | |
| A_23_P23221 | GADD45A | 0.88 | 0.85 | 0.74 | 0.15 | 0.97 | 0.16 | 1.63 | 0.00 | 1.16 | 0.87 | 1.32 | 0.00 | X | X | | |
| A_23_P169437 | LCN2 | 0.89 | 0.41 | 0.74 | 0.35 | 0.96 | 0.65 | 0.99 | 0.21 | 0.66 | 0.33 | 0.94 | 0.92 | X | X | | |
| A_24_P166443 | HLA-DPB1 | −0.24 | 0.12 | −0.30 | 0.27 | −0.46 | 0.19 | −0.43 | 0.62 | −0.67 | 0.00 | −0.66 | 0.00 | | X | | |
| A_23_P56559 | DHRS9 | 0.96 | 0.63 | 0.69 | 0.55 | 0.90 | 0.59 | 1.34 | 0.00 | 1.45 | 0.14 | 1.31 | 0.00 | X | X | | |
| A_23_P11212 | ACSL1 | 0.48 | 0.55 | 0.28 | 0.15 | 0.40 | 0.24 | 0.80 | 0.23 | 0.75 | 0.00 | 0.85 | 0.00 | | X | | |
| A_23_P16145 | ERO1L | 0.36 | 0.24 | 0.21 | 0.35 | 0.33 | 0.97 | 0.60 | 0.40 | 0.44 | 0.17 | 0.59 | 0.00 | | X | | |
| A_23_P25956 | C5orf32 | 0.70 | 0.15 | 0.57 | 0.22 | 0.76 | 0.22 | 0.97 | 0.28 | 1.70 | 0.00 | 1.13 | 0.00 | | X | | |
| A_23_P11226 | GNG1 | 0.28 | 0.14 | 0.15 | 0.32 | 0.26 | 0.75 | 0.62 | 0.97 | 0.64 | 0.00 | 0.64 | 0.00 | | X | | |
| A_23_P1675 | GPR16 | 0.55 | 0.13 | 0.38 | 0.35 | 0.53 | 0.33 | 0.64 | 0.27 | 0.63 | 0.00 | 0.70 | 0.00 | | X | | |
| A_33_P325193 | SLC36A1 | 0.27 | 0.14 | 0.33 | 0.29 | 0.59 | 0.32 | 0.58 | 0.36 | 0.63 | 0.00 | 0.89 | 0.20 | | X | | |
| A_23_P73837 | TLR8 | 0.35 | 0.51 | 0.25 | 0.78 | 0.32 | 0.19 | 0.63 | 0.15 | 0.55 | 0.00 | 0.66 | 0.00 | | X | | |
| A_23_P11212 | ACSL1 | 0.46 | 0.52 | 0.27 | 0.16 | 0.38 | 0.31 | 0.79 | 0.25 | 0.74 | 0.00 | 0.84 | 0.00 | | X | | |
| A_33_P338897 | ATP6V1C1 | 0.56 | 0.69 | 0.43 | 0.18 | 0.61 | 0.36 | 0.76 | 0.35 | 0.75 | 0.00 | 0.92 | 0.17 | | X | | X |
| A_23_P11212 | ACSL1 | 0.45 | 0.64 | 0.23 | 0.22 | 0.34 | 0.59 | 0.87 | 0.18 | 0.76 | 0.00 | 0.83 | 0.00 | | X | | |
| A_23_P213584 | HK3 | 0.83 | 0.34 | 0.67 | 0.50 | 0.69 | 0.13 | 1.22 | 0.00 | 1.16 | 0.00 | 1.22 | 0.00 | X | X | X | |
| A_23_P28485 | GCA | 0.45 | 0.44 | 0.25 | 0.16 | 0.46 | 0.62 | 0.74 | 0.53 | 0.64 | 0.00 | 0.73 | 0.00 | | X | | |
| A_24_P141214 | STOM | 0.69 | 0.91 | 0.48 | 0.14 | 0.34 | 0.79 | 0.95 | 0.00 | 0.87 | 0.00 | 0.88 | 0.00 | | X | | X |
| A_23_P11473 | NAIP | 0.47 | 0.86 | 0.24 | 0.28 | 0.47 | 0.26 | 0.93 | 0.19 | 0.83 | 0.38 | 1.15 | 0.00 | | X | | |
| A_23_P15618 | SLC22A4 | 0.38 | 0.41 | 0.38 | 0.38 | 0.26 | 0.52 | 0.58 | 0.67 | 0.62 | 0.00 | 0.68 | 0.00 | | X | | |
| A_33_P3238997 | AGFG1 | 0.40 | 0.19 | 0.23 | 0.38 | 0.45 | 0.54 | 0.62 | 0.00 | 0.55 | 0.00 | 0.69 | 0.00 | | X | | |
| A_32_P178945 | YOD1 | 0.53 | 0.11 | 0.32 | 0.85 | 0.19 | 0.57 | 0.69 | 0.16 | 0.63 | 0.26 | 0.59 | 0.40 | | X | | X |
| A_24_P181254 | OLFM4 | 1.23 | 0.14 | 0.98 | 0.45 | 1.42 | 0.85 | 1.47 | 0.24 | 1.22 | 0.27 | 1.72 | 0.00 | X | X | | |
| A_33_P3251876 | IL18R1 | 0.59 | 0.40 | 0.52 | 0.25 | 0.82 | 0.00 | 0.75 | 0.17 | 0.85 | 0.00 | 1.48 | 0.00 | X | X | X | |
| A_23_P59528 | ACN9 | 0.52 | 0.44 | 0.46 | 0.70 | 0.56 | 0.26 | 0.64 | 0.12 | 0.62 | 0.38 | 0.72 | 0.00 | | X | | |
| A_24_P253818 | FLOT2 | 0.58 | 0.17 | 0.49 | 0.17 | 0.36 | 0.94 | 0.80 | 0.00 | 0.71 | 0.00 | 0.65 | 0.00 | | X | | |
| A_24_P413669 | PFKFB2 | 1.16 | 0.15 | 0.97 | 0.11 | 1.29 | 0.00 | 1.40 | 0.00 | 1.44 | 0.00 | 1.59 | 0.00 | X | X | | |
| A_23_P864 | GPER | 0.43 | 0.85 | 0.29 | 0.13 | 0.52 | 0.16 | 0.59 | 0.34 | 0.46 | 0.38 | 0.79 | 0.00 | | X | | X |
| A_24_P5854 | SLC9A8 | 0.23 | 0.96 | 0.27 | 0.23 | 0.32 | 0.49 | 0.53 | 0.34 | 0.59 | 0.00 | 0.67 | 0.59 | | X | | |
| A_23_P217319 | FGF13 | 0.42 | 0.87 | 0.48 | 0.23 | 0.70 | 0.19 | 0.55 | 0.28 | 0.63 | 0.45 | 0.82 | 0.69 | | X | | |
| A_24_P188377 | CD55 | 0.47 | 0.16 | 0.49 | 0.16 | 0.54 | 0.82 | 0.65 | 0.18 | 0.90 | 0.00 | 0.91 | 0.73 | | X | | X |
| A_23_P58396 | PDGFC | 0.39 | 0.17 | 0.56 | 0.55 | 0.68 | 0.00 | 0.47 | 0.15 | 0.62 | 0.18 | 0.78 | 0.00 | | X | | X |
| A_23_P149892 | CSGALNACT2 | 0.51 | 0.87 | 0.38 | 0.90 | 0.46 | 0.58 | 0.89 | 0.00 | 0.98 | 0.92 | 0.94 | 0.23 | | X | | |
| A_23_P123732 | C9orf13 | 0.13 | 0.58 | 0.47 | 0.37 | 0.52 | 0.58 | 0.32 | 0.53 | 0.67 | 0.17 | 0.69 | 0.00 | | X | | |
| A_24_P6319 | IL1R2 | 0.93 | 0.17 | 0.90 | 0.29 | 1.32 | 0.00 | 1.56 | 0.00 | 1.54 | 0.00 | 1.81 | 0.29 | X | X | X | |
| A_33_P33599 | EMB | 0.45 | 0.86 | 0.33 | 0.28 | 0.58 | 0.31 | 0.74 | 0.00 | 0.86 | 0.00 | 1.41 | 0.00 | | X | | |
| A_23_P42897 | MGAM | 0.52 | 0.42 | 0.37 | 0.66 | 0.48 | 0.12 | 0.84 | 0.24 | 0.83 | 0.00 | 0.94 | 0.00 | | X | | |
| A_23_P11212 | ACSL1 | 0.59 | 0.39 | 0.28 | 0.15 | 0.37 | 0.35 | 0.83 | 0.16 | 0.76 | 0.00 | 0.82 | 0.00 | | X | | |
| A_23_P1997 | ILDR1 | −0.44 | 0.13 | −0.33 | 0.17 | −0.17 | 0.45 | 0.32 | 0.28 | 0.62 | 0.19 | 0.69 | 0.94 | | X | | |
| A_23_P11212 | ACSL1 | 0.46 | 0.58 | 0.26 | 0.18 | 0.36 | 0.43 | 0.85 | 0.27 | 0.75 | 0.00 | 0.81 | 0.00 | | X | | |
| A_23_P423331 | NTNG2 | 0.16 | 0.29 | 0.22 | 0.95 | 0.42 | 0.65 | 0.53 | 0.13 | 0.68 | 0.00 | 0.88 | 0.73 | | X | | |
| A_33_P3376234 | PHTF1 | 0.43 | 0.85 | 0.38 | 0.17 | 0.50 | 0.69 | 0.63 | 0.00 | 0.67 | 0.00 | 0.77 | 0.19 | | X | | |
| A_24_P684186 | EMB | 0.48 | 0.16 | 0.35 | 0.21 | 0.45 | 0.39 | 0.65 | 0.23 | 0.61 | 0.00 | 0.69 | 0.00 | | X | | |
| A_23_P121716 | ANXA3 | 0.78 | 0.27 | 0.55 | 0.77 | 0.66 | 0.16 | 1.96 | 0.19 | 1.34 | 0.00 | 1.18 | 0.00 | | X | | |
| A_23_P29422 | GYG1 | 1.46 | 0.35 | 0.84 | 0.16 | 0.96 | 0.57 | 1.28 | 0.00 | 1.22 | 0.00 | 1.36 | 0.00 | X | X | | |
| A_23_P117546 | SOS2 | 0.31 | 0.56 | 0.24 | 0.89 | 0.43 | 0.82 | 0.59 | 0.12 | 0.55 | 0.00 | 0.69 | 0.00 | | X | | |
| A_23_P139669 | SLC2A3 | 0.65 | 0.44 | 0.64 | 0.24 | 0.54 | 0.32 | 0.87 | 0.00 | 0.92 | 0.00 | 0.87 | 0.54 | X | X | X | X |
| A_21_P11751 | CD177 | 1.68 | 0.14 | 1.33 | 0.78 | 1.83 | 0.44 | 1.98 | 0.00 | 1.98 | 0.00 | 2.38 | 0.00 | X | X | | |
| A_33_P3399571 | VNN1 | 0.84 | 0.97 | 0.63 | 0.38 | 0.88 | 0.84 | 1.27 | 0.34 | 1.23 | 0.00 | 1.39 | 0.00 | X | X | | |
| A_33_P3222139 | SREBF1 | −0.11 | 0.70 | 0.73 | 0.97 | −0.39 | 0.48 | −0.76 | 0.36 | −0.61 | 0.32 | −0.79 | 0.25 | | X | | |
| A_21_P13518 | GYG1 | 0.96 | 0.20 | 0.79 | 0.15 | 0.92 | 0.24 | 1.18 | 0.00 | 1.14 | 0.00 | 1.28 | 0.00 | X | X | | |
| A_23_P31911 | BLMH | 0.56 | 0.15 | 0.44 | 0.83 | 0.66 | 0.14 | 0.83 | 0.66 | 0.78 | 0.00 | 0.89 | 0.00 | | X | | |
| A_33_P3341676 | MEF2A | 0.41 | 0.50 | 0.31 | 0.69 | 0.48 | 0.19 | 0.68 | 0.13 | 0.63 | 0.44 | 0.82 | 0.88 | | X | | |
| A_24_P322353 | PSTPIP2 | 0.40 | 0.21 | 0.37 | 0.19 | 0.45 | 0.83 | 0.62 | 0.00 | 0.56 | 0.00 | 0.67 | 0.00 | | X | | |
| A_23_P11473 | NAIP | 0.47 | 0.13 | 0.23 | 0.32 | 0.58 | 0.13 | 0.87 | 0.54 | 0.82 | 0.14 | 1.88 | 0.00 | | X | | |
| A_24_P343233 | HLA-DBB1 | −0.21 | 0.13 | −0.22 | 0.47 | −0.52 | 0.96 | −0.37 | 0.13 | −0.59 | 0.00 | −0.73 | 0.00 | | X | | |
| A_21_P11898 | SEPT14 | 0.99 | 0.53 | 0.62 | 0.60 | 0.19 | 0.14 | 0.57 | 0.13 | 0.68 | 0.00 | 0.72 | 0.00 | | X | | X |
| A_33_P33141 | EXOSC4 | 0.81 | 0.18 | 0.83 | 0.71 | 1.22 | 0.00 | 0.78 | 0.60 | 0.84 | 0.00 | 1.41 | 0.00 | X | X | | |
| A_23_P13765 | FCER1A | −0.38 | 0.14 | −0.26 | 0.34 | −0.70 | 0.73 | −0.59 | 0.56 | −0.59 | 0.17 | −0.95 | 0.00 | | X | | |
| A_23_P217564 | ACSL4 | 0.40 | 0.93 | 0.33 | 0.12 | 0.35 | 0.59 | 0.74 | 0.00 | 0.71 | 0.00 | 0.74 | 0.00 | | X | | |
| A_24_P261259 | PFKFB3 | 0.96 | 0.46 | 0.74 | 0.68 | 0.99 | 0.12 | 1.24 | 0.99 | 1.30 | 0.00 | 1.48 | 0.00 | X | X | X | X |
| A_23_P1997 | ILDR1 | −0.53 | 0.78 | −0.39 | 0.54 | −0.17 | 0.44 | 0.27 | 0.35 | 0.60 | 0.23 | 0.66 | 0.85 | | X | | |
| A_23_P166848 | LTF | 0.95 | 0.18 | 0.75 | 0.19 | 0.87 | 0.13 | 1.18 | 0.16 | 0.86 | 0.42 | 1.74 | 0.25 | | X | | X |
| A_24_P343233 | HLA-DRB1 | −0.26 | 0.96 | −0.43 | 0.26 | −0.57 | 0.11 | −0.38 | 0.92 | −0.68 | 0.00 | −0.83 | 0.00 | | X | | |
| A_23_P1962 | RARRES3 | −0.47 | 0.63 | −0.25 | 0.19 | −0.34 | 0.48 | −0.55 | 0.17 | −0.65 | 0.22 | −0.62 | 0.13 | | X | | |
| A_33_P3286157 | TNFRSF4 | 0.97 | 0.64 | −0.16 | 0.28 | −0.20 | 0.16 | −0.39 | 0.11 | −0.64 | 0.53 | −0.67 | 0.00 | | X | | |
| A_24_P27977 | TRPM2 | 0.69 | 0.57 | 0.45 | 0.17 | 0.60 | 0.00 | 0.77 | 0.00 | 0.63 | 0.00 | 0.78 | 0.26 | X | X | | |
| A_33_P3289236 | HPR | 1.73 | 0.43 | 0.73 | 0.23 | 1.18 | 0.79 | 1.36 | 0.98 | 1.15 | 0.11 | 1.54 | 0.00 | X | X | | |
| A_23_P14464 | ALOX5 | 0.50 | 0.17 | 0.38 | 0.22 | 0.46 | 0.12 | 0.77 | 0.00 | 0.69 | 0.00 | 0.68 | 0.00 | | X | | |
| A_23_P131785 | BPI | 0.49 | 0.14 | 0.53 | 0.42 | 0.74 | 0.19 | 0.77 | 0.23 | 0.65 | 0.57 | 0.85 | 0.27 | | X | | |
| A_21_P11897 | SEPT14 | 0.36 | 0.67 | 0.37 | 0.38 | 0.41 | 0.28 | 0.75 | 0.00 | 0.80 | 0.00 | 0.87 | 0.00 | | X | | X |
| A_23_P42956 | SSH1 | 0.43 | 0.24 | 0.44 | 0.72 | 0.57 | 0.00 | 0.61 | 0.00 | 0.72 | 0.00 | 0.85 | 0.00 | | X | | |
| A_23_P4174 | MMP9 | 1.28 | 0.33 | 0.79 | 0.12 | 1.20 | 0.15 | 1.15 | 0.83 | 1.88 | 0.00 | 1.45 | 0.00 | X | X | | |
| A_23_P122863 | GRB1 | 0.74 | 0.73 | 0.53 | 0.23 | 0.89 | 0.14 | 0.96 | 0.16 | 0.90 | 0.00 | 1.23 | 0.12 | X | X | | |
| A_23_P126241 | EIF4G3 | 0.28 | 0.69 | 0.16 | 0.64 | 0.32 | 0.13 | 0.63 | 0.00 | 0.61 | 0.00 | 0.74 | 0.00 | | X | | |
| A_23_P122863 | GRB1 | 0.58 | 0.29 | 0.52 | 0.27 | 0.91 | 0.67 | 0.79 | 0.36 | 0.93 | 0.00 | 1.24 | 0.00 | | X | | |
| A_33_P3369844 | CD24 | 0.66 | 0.26 | 0.65 | 0.53 | 0.71 | 0.52 | 0.58 | 0.19 | 0.53 | 0.20 | 0.55 | 0.17 | X | | | |
| A_33_P334945 | IL4R | 0.42 | 0.12 | 0.27 | 0.35 | 0.33 | 0.22 | 0.64 | 0.00 | 0.67 | 0.00 | 0.63 | 0.00 | | X | | |
| A_32_P61684 | PAG1 | 0.48 | 0.12 | 0.39 | 0.39 | 0.46 | 0.12 | 0.82 | 0.00 | 0.75 | 0.00 | 0.84 | 0.00 | | X | | |
| A_23_P161152 | PDSS1 | 0.62 | 0.14 | 0.50 | 0.13 | 0.57 | 0.00 | 0.66 | 0.00 | 0.55 | 0.88 | 0.65 | 0.00 | | X | | X |
| A_23_P4174 | MMP9 | 0.99 | 0.52 | 0.78 | 0.13 | 1.21 | 0.16 | 1.17 | 0.12 | 1.14 | 0.00 | 1.48 | 0.00 | X | X | | |
| A_23_P212522 | ATP11B | 0.53 | 0.56 | 0.44 | 0.19 | 0.48 | 0.48 | 0.64 | 0.00 | 0.70 | 0.00 | 0.74 | 0.00 | | X | | X |
| A_23_P11473 | NAIP | 0.49 | 0.85 | 0.26 | 0.25 | 0.49 | 0.22 | 0.91 | 0.21 | 0.83 | 0.00 | 1.13 | 0.00 | | X | | |
| A_23_P14464 | ALOX5 | 0.44 | 0.37 | 0.42 | 0.15 | 0.47 | 0.40 | 0.69 | 0.12 | 0.76 | 0.00 | 0.69 | 0.00 | | X | | |
| A_23_P17336 | ACAP1 | 0.45 | 0.24 | 0.47 | 0.23 | 0.35 | 0.22 | 0.57 | 0.18 | 0.59 | 0.00 | 0.59 | 0.00 | | X | | |
| A_23_P334864 | FAM126B | 0.22 | 0.19 | 0.96 | 0.51 | 0.19 | 0.17 | 0.68 | 0.12 | 0.71 | 0.00 | 0.77 | 0.00 | | X | | |
| A_21_P12992 | NAIP | 0.60 | 0.41 | 0.45 | 0.23 | 0.52 | 0.13 | 0.91 | 0.00 | 0.91 | 0.00 | 0.94 | 0.29 | | X | | |
| A_23_P17465 | KRT31 | −0.35 | 0.90 | −0.39 | 0.64 | −0.28 | 0.27 | −0.12 | 0.59 | −0.63 | 0.63 | −0.64 | 0.71 | | X | | |
| A_23_P33561 | C19orf59 | 1.26 | 0.56 | 0.92 | 0.23 | 1.47 | 0.28 | 1.38 | 0.45 | 1.35 | 0.00 | 1.78 | 0.00 | X | X | | |
| A_23_P213385 | BASP1 | 0.45 | 0.32 | 0.33 | 0.74 | 0.40 | 0.16 | 0.68 | 0.37 | 0.65 | 0.00 | 0.66 | 0.00 | | X | | |
| A_23_P123393 | KCNQ3 | −0.13 | 0.74 | 0.38 | 0.90 | −0.11 | 0.52 | −0.79 | 0.49 | −0.25 | 0.33 | −0.63 | 0.70 | | X | | |
| A_23_P17186 | OPLAH | 0.75 | 0.47 | 0.72 | 0.37 | 1.00 | 0.24 | 0.98 | 0.00 | 1.15 | 0.00 | 1.34 | 0.00 | X | X | | |
| A_23_P12418 | ITGAM | 0.52 | 0.14 | 0.36 | 0.39 | 0.45 | 0.66 | 0.74 | 0.20 | 0.57 | 0.00 | 0.67 | 0.00 | | X | | |
| A_23_P18372 | B3GNT5 | 0.49 | 0.13 | 0.29 | 0.16 | 0.39 | 0.36 | 0.64 | 0.00 | 0.58 | 0.12 | 0.73 | 0.00 | | X | | |
| A_23_P123732 | C9orf13 | −0.15 | 0.94 | 0.46 | 0.15 | 0.47 | 0.67 | 0.12 | 0.45 | 0.65 | 0.19 | 0.63 | 0.32 | | X | | |
| A_23_P4883 | CAMKK2 | 0.40 | 0.46 | 0.33 | 0.78 | 0.33 | 0.54 | 0.60 | 0.90 | 0.60 | 0.25 | 0.57 | 0.22 | | X | | |
| A_33_P329343 | CYP1B1 | 0.52 | 0.15 | 0.40 | 0.56 | 0.52 | 0.80 | 0.90 | 0.00 | 0.83 | 0.67 | 0.97 | 0.00 | | X | | |
| A_33_P3311285 | LMNA | 0.15 | 0.59 | −0.93 | 0.65 | −0.29 | 0.94 | −0.36 | 0.13 | −0.62 | 0.27 | −0.72 | 0.73 | | X | | |
| A_23_P13361 | LCK | −0.49 | 0.48 | −0.42 | 0.43 | −0.44 | 0.23 | −0.57 | 0.14 | −0.59 | 0.21 | −0.64 | 0.12 | | X | | |
| A_23_P29851 | LRPAP1 | 0.49 | 0.25 | 0.46 | 0.17 | 0.41 | 0.84 | 0.64 | 0.34 | 0.58 | 0.00 | 0.62 | 0.00 | | X | | |
| A_23_P58953 | NQO2 | 0.44 | 0.26 | 0.48 | 0.14 | 0.40 | 0.75 | 0.73 | 0.29 | 0.87 | 0.43 | 0.76 | 0.00 | | X | | X |
| A_23_P4174 | MMP9 | 1.00 | 0.53 | 0.78 | 0.13 | 1.23 | 0.12 | 1.16 | 0.19 | 1.12 | 0.00 | 1.48 | 0.00 | X | X | | |
| A_32_P87697 | HLA-DBA | −0.23 | 0.20 | −0.37 | 0.20 | −0.62 | 0.22 | −0.29 | 0.88 | −0.72 | 0.00 | −0.86 | 0.00 | | X | | X |
| A_33_P335622 | STARD3 | 0.12 | 0.66 | 0.28 | 0.89 | −0.27 | 0.12 | −0.48 | 0.37 | −0.59 | 0.26 | −0.74 | 0.44 | | X | | |
| A_23_P117546 | SOS2 | 0.34 | 0.33 | 0.25 | 0.64 | 0.45 | 0.83 | 0.54 | 0.27 | 0.57 | 0.00 | 0.69 | 0.69 | | X | | |
| A_23_P123393 | KCNQ3 | −0.24 | 0.40 | 0.18 | 0.40 | −0.26 | 0.16 | −0.79 | 0.55 | −0.31 | 0.21 | −0.64 | 0.80 | | X | | |
| A_23_P123393 | KCNQ3 | −0.76 | 0.78 | −0.24 | 0.23 | −0.23 | 0.23 | −0.40 | 0.83 | −0.74 | 0.33 | −0.76 | 0.68 | | X | | |
| A_23_P4174 | MMP9 | 1.38 | 0.32 | 0.78 | 0.13 | 1.22 | 0.14 | 1.28 | 0.62 | 1.11 | 0.00 | 1.50 | 0.00 | X | X | | |
| A_24_P29723 | POR | 0.68 | 0.12 | 0.52 | 0.79 | 0.55 | 0.66 | 0.84 | 0.49 | 0.72 | 0.00 | 0.76 | 0.00 | | X | | |
| A_23_P117546 | SOS2 | 0.29 | 0.20 | 0.25 | 0.13 | 0.43 | 0.72 | 0.47 | 0.44 | 0.57 | 0.00 | 0.68 | 0.74 | | X | | |
| A_23_P11473 | NAIP | 0.54 | 0.83 | 0.27 | 0.23 | 0.55 | 0.13 | 0.92 | 0.54 | 0.83 | 0.13 | 1.12 | 0.00 | | X | | |
| A_33_P335863 | RETN | 1.89 | 0.63 | 0.97 | 0.66 | 1.44 | 0.13 | 1.53 | 0.00 | 1.52 | 0.00 | 1.83 | 0.00 | X | X | | |
| A_33_P334197 | NEGR1 | −0.19 | 0.45 | −0.48 | 0.19 | −0.48 | 0.79 | −0.28 | 0.27 | −0.73 | 0.49 | −0.83 | 0.11 | | X | | |
| A_33_P3281816 | CAP1 | 0.21 | 0.90 | 0.23 | 0.48 | 0.30 | 0.66 | 0.60 | 0.90 | 0.66 | 0.00 | 0.69 | 0.00 | | X | | |
| A_33_P3289541 | MLLT1 | 0.46 | 0.66 | 0.44 | 0.54 | 0.39 | 0.15 | 0.56 | 0.68 | 0.60 | 0.00 | 0.64 | 0.00 | | X | | |
| A_23_P295 | SAMSN1 | 0.96 | 0.94 | 0.62 | 0.13 | 0.87 | 0.31 | 1.25 | 0.00 | 1.13 | 0.00 | 1.28 | 0.33 | X | X | | |
| A_24_P343233 | HLA-DRB1 | −0.21 | 0.19 | −0.40 | 0.47 | −0.62 | 0.00 | −0.32 | 0.12 | −0.76 | 0.00 | −0.86 | 0.74 | | X | | |
| A_23_P155765 | HMGB2 | 0.35 | 0.12 | 0.30 | 0.18 | 0.62 | 0.16 | 0.53 | 0.37 | 0.65 | 0.00 | 0.77 | 0.00 | | X | | |
| A_33_P334847 | CARD6 | 0.32 | 0.80 | 0.30 | 0.18 | 0.67 | 0.00 | 0.51 | 0.16 | 0.62 | 0.00 | 0.90 | 0.12 | | X | | |
| A_23_P417331 | RPS6KA3 | 0.33 | 0.14 | 0.38 | 0.77 | 0.46 | 0.00 | 0.48 | 0.19 | 0.63 | 0.00 | 0.69 | 0.00 | | X | | |
| A_23_P67847 | GALNT14 | 0.77 | 0.63 | 0.67 | 0.16 | 0.87 | 0.60 | 1.69 | 0.12 | 1.23 | 0.00 | 1.26 | 0.00 | X | X | X | |
| A_33_P3338 | MAP1LC3A | 0.12 | 0.65 | −0.16 | 0.43 | −0.17 | 0.37 | −0.23 | 0.24 | −0.64 | 0.36 | −0.72 | 0.22 | | X | | |
| A_24_P244944 | MCTP2 | 0.60 | 0.24 | 0.42 | 0.73 | 0.57 | 0.19 | 0.73 | 0.00 | 0.76 | 0.00 | 0.88 | 0.00 | | X | | |
| A_33_P3211666 | ILI8R1 | 0.79 | 0.57 | 0.68 | 0.77 | 1.19 | 0.00 | 0.97 | 0.48 | 1.88 | 0.00 | 1.44 | 0.00 | X | X | X | |
| A_33_P3343155 | GNAQ | 0.27 | 0.33 | 0.27 | 0.48 | 0.36 | 0.25 | 0.60 | 0.28 | 0.61 | 0.00 | 0.71 | 0.00 | | X | | |
| A_32_P351968 | HLA-DMB | −0.56 | 0.62 | −0.53 | 0.95 | −0.67 | 0.53 | −0.66 | 0.42 | −0.84 | 0.00 | −0.94 | 0.00 | | X | | |
| A_23_P9823 | MLXIP | 0.25 | 0.17 | 0.23 | 0.14 | 0.23 | 0.91 | 0.68 | 0.00 | 0.62 | 0.00 | 0.72 | 0.00 | | X | | |
| A_23_P3624 | MYEOV | 0.92 | 0.69 | −0.28 | 0.28 | −0.15 | 0.33 | −0.23 | 0.32 | −0.64 | 0.66 | −0.60 | 0.83 | | X | | |
| A_24_P38536 | CD164 | 0.35 | 0.50 | 0.29 | 0.18 | 0.42 | 0.14 | 0.49 | 0.20 | 0.48 | 0.00 | 0.63 | 0.00 | | X | | X |
| A_23_P13747 | SIPA1L2 | 0.44 | 0.13 | 0.25 | 0.15 | 0.47 | 0.63 | 0.89 | 0.00 | 0.89 | 0.79 | 0.97 | 0.36 | | X | | |
| A_23_P25155 | GPR84 | 1.97 | 0.98 | 1.19 | 0.29 | 1.24 | 0.00 | 1.25 | 0.37 | 1.28 | 0.00 | 1.54 | 0.00 | X | X | | |
| A_24_P18155 | ST3GAL4 | 0.50 | 0.14 | 0.39 | 0.28 | 0.47 | 0.17 | 0.62 | 0.36 | 0.59 | 0.20 | 0.71 | 0.32 | | X | | |
| A_23_P2758 | SOCS3 | 0.66 | 0.20 | 0.45 | 0.74 | 0.65 | 0.11 | 0.97 | 0.18 | 0.94 | 0.00 | 1.57 | 0.00 | | X | | |
| A_23_P12463 | QSOX1 | 0.34 | 0.59 | 0.32 | 0.27 | 0.59 | 0.47 | 0.55 | 0.25 | 0.63 | 0.00 | 0.83 | 0.00 | | X | | |
| A_23_P121716 | ANXA3 | 0.84 | 0.16 | 0.58 | 0.67 | 0.67 | 0.13 | 1.12 | 0.00 | 1.40 | 0.00 | 1.12 | 0.00 | | X | | |
| A_23_P25721 | RNF146 | 0.37 | 0.54 | 0.28 | 0.57 | 0.26 | 0.69 | 0.67 | 0.28 | 0.59 | 0.00 | 0.66 | 0.00 | | X | | X |
| A_33_P3345132 | ZNF578 | 0.20 | 0.45 | −0.34 | 0.22 | −0.13 | 0.55 | −0.54 | 0.84 | −0.72 | 0.76 | −0.74 | 0.37 | | X | | |
| A_33_P3245389 | C14orf11 | 0.47 | 0.11 | 0.36 | 0.19 | 0.50 | 0.14 | 0.59 | 0.29 | 0.59 | 0.00 | 0.68 | 0.00 | | X | | |
| A_23_P13765 | FCER1A | −0.30 | 0.16 | −0.26 | 0.18 | −0.67 | 0.34 | −0.49 | 0.11 | −0.62 | 0.16 | −0.94 | 0.00 | | X | | |
| A_23_P21433 | SERPINB1 | 0.52 | 0.17 | 0.42 | 0.33 | 0.62 | 0.42 | 0.89 | 0.25 | 0.82 | 0.00 | 1.00 | 0.00 | | X | | |
| A_24_P4525 | ATP2B4 | 0.24 | 0.17 | 0.17 | 0.17 | 0.23 | 0.36 | 0.59 | 0.20 | 0.64 | 0.00 | 0.66 | 0.00 | | X | | |
| A_33_P337554L | CD3D | −0.38 | 0.65 | −0.48 | 0.98 | −0.56 | 0.58 | −0.63 | 0.19 | −0.84 | 0.00 | −0.95 | 0.00 | | X | | |
| A_23_P2532 | CCR3 | −0.28 | 0.25 | −0.24 | 0.24 | −0.72 | 0.17 | −0.38 | 0.95 | −0.59 | 0.18 | −0.98 | 0.00 | | X | | |
| A_33_P3378659 | TARP | −0.13 | 0.51 | −0.28 | 0.12 | −0.34 | 0.66 | −0.42 | 0.37 | −0.64 | 0.15 | −0.68 | 0.26 | | X | | X |
| A_33_P3234277 | HLA-DPA1 | −0.27 | 0.26 | −0.32 | 0.52 | −0.63 | 0.22 | −0.32 | 0.25 | −0.64 | 0.00 | −0.84 | 0.00 | | X | | |
| A_33_P342526 | CSGALNACT2 | 0.54 | 0.42 | 0.34 | 0.33 | 0.59 | 0.60 | 0.79 | 0.22 | 0.85 | 0.00 | 0.99 | 0.00 | | X | | |
| A_23_P818 | HLA-DQB1 | −0.17 | 0.37 | −0.47 | 0.24 | −0.62 | 0.12 | −0.26 | 0.55 | −0.62 | 0.00 | −0.88 | 0.00 | | X | | |
| A_23_P15465 | SULF2 | −0.73 | 0.74 | −0.68 | 0.88 | −0.59 | 0.16 | −0.70 | 0.53 | −0.81 | 0.00 | −0.72 | 0.11 | X | X | | |
| A_23_P28768 | FCAR | 0.73 | 0.40 | 0.55 | 0.12 | 0.61 | 0.34 | 0.94 | 0.00 | 0.84 | 0.00 | 0.92 | 0.00 | X | X | X | |
| A_23_P48676 | PYGL | 0.44 | 0.92 | 0.29 | 0.13 | 0.47 | 0.74 | 0.63 | 0.17 | 0.59 | 0.00 | 0.68 | 0.00 | | X | | X |
| A_33_P322398 | TPM3 | 0.23 | 0.88 | 0.16 | 0.54 | 0.18 | 0.18 | 0.58 | 0.00 | 0.59 | 0.00 | 0.67 | 0.20 | | X | | X |
| A_23_P12418 | ITGAM | 0.70 | 0.19 | 0.37 | 0.34 | 0.50 | 0.47 | 0.87 | 0.62 | 0.58 | 0.00 | 0.72 | 0.00 | | X | | |
| A_23_P496 | CA4 | 0.68 | 0.14 | 0.55 | 0.19 | 0.74 | 0.42 | 0.74 | 0.46 | 0.66 | 0.11 | 0.80 | 0.00 | | X | | X |
| A_24_P27144 | CD63 | 0.39 | 0.22 | 0.43 | 0.80 | 0.58 | 0.18 | 0.59 | 0.74 | 0.64 | 0.00 | 0.76 | 0.00 | | X | | X |
| A_23_P8593 | TLR5 | 0.57 | 0.24 | 0.45 | 0.68 | 0.68 | 0.28 | 0.83 | 0.13 | 0.84 | 0.00 | 1.12 | 0.16 | | X | | |
| A_23_P4353 | WSB1 | 0.33 | 0.12 | 0.19 | 0.27 | 0.49 | 0.14 | 0.68 | 0.22 | 0.67 | 0.00 | 0.85 | 0.00 | | X | | X |
| A_33_P321184 | RUNX1 | 0.33 | 0.45 | 0.40 | 0.15 | 0.60 | 0.00 | 0.49 | 0.34 | 0.60 | 0.00 | 0.78 | 0.55 | | X | | X |
| A_33_P3359223 | C9orf173 | 0.67 | 0.80 | −0.89 | 0.66 | −0.43 | 0.15 | −0.54 | 0.33 | −0.75 | 0.38 | −0.94 | 0.00 | | X | | |
| A_23_P6339 | FCGR1B | 0.89 | 0.67 | 0.19 | 0.37 | 0.85 | 0.96 | 0.84 | 0.16 | 0.87 | 0.00 | 0.77 | 0.18 | | X | | |
| A_23_P21694 | ASPH | 0.28 | 0.14 | 0.24 | 0.14 | 0.52 | 0.35 | 0.58 | 0.34 | 0.70 | 0.00 | 0.87 | 0.00 | | X | | |
| A_23_P122863 | GRB1 | 0.59 | 0.36 | 0.50 | 0.38 | 0.91 | 0.15 | 0.79 | 0.66 | 0.92 | 0.00 | 1.26 | 0.00 | | X | | |
| A_23_P258164 | CORT | −0.23 | 0.92 | −0.22 | 0.34 | −0.48 | 0.11 | −0.26 | 0.30 | −0.65 | 0.87 | −0.94 | 0.17 | | X | | |
| A_23_P128974 | BATF | 0.30 | 0.68 | 0.34 | 0.23 | 0.47 | 0.44 | 0.48 | 0.11 | 0.60 | 0.00 | 0.73 | 0.00 | | X | | |
| A_33_P3282614 | C9orf173 | 0.21 | 0.36 | −0.88 | 0.62 | −0.36 | 0.27 | −0.34 | 0.11 | −0.66 | 0.48 | −0.82 | 0.00 | | X | | |
| A_23_P6861 | CST7 | 0.60 | 0.37 | 0.56 | 0.24 | 0.63 | 0.62 | 0.69 | 0.89 | 0.76 | 0.99 | 0.89 | 0.32 | | X | | X |
| A_23_P117546 | SOS2 | 0.29 | 0.75 | 0.23 | 0.86 | 0.40 | 0.82 | 0.52 | 0.68 | 0.58 | 0.00 | 0.66 | 0.99 | | X | | |
| A_23_P121716 | ANXA3 | 0.78 | 0.27 | 0.54 | 0.80 | 0.67 | 0.15 | 1.95 | 0.24 | 1.19 | 0.00 | 1.13 | 0.00 | | X | | |
| A_24_P3348 | RAB32 | 0.55 | 0.69 | 0.45 | 0.43 | 0.44 | 0.56 | 0.69 | 0.36 | 0.52 | 0.11 | 0.70 | 0.00 | | X | | |
| A_33_P3364864 | NAMPT | 0.45 | 0.80 | 0.58 | 0.97 | 0.85 | 0.52 | 0.58 | 0.14 | 0.67 | 0.00 | 0.64 | 0.24 | | X | | |
| A_24_P14859 | TACR1 | 0.43 | 0.97 | −0.18 | 0.44 | −0.29 | 0.16 | 0.13 | 0.56 | −0.61 | 0.95 | −0.84 | 0.46 | | X | | |
| A_33_P3835524 | POU2F2 | −0.30 | 0.92 | −0.28 | 0.77 | −0.46 | 0.28 | −0.28 | 0.19 | −0.63 | 0.63 | −0.79 | 0.16 | | X | | X |
| A_24_P2664 | PFKFB3 | 0.29 | 0.36 | 0.21 | 0.99 | 0.43 | 0.29 | 0.54 | 0.12 | 0.60 | 0.00 | 0.78 | 0.00 | | X | | X |
| A_24_P343233 | HLA-DRB1 | −0.20 | 0.15 | −0.40 | 0.37 | −0.57 | 0.00 | −0.31 | 0.87 | −0.71 | 0.00 | −0.82 | 0.00 | | X | | |
| A_23_P11212 | ACSL1 | 0.47 | 0.57 | 0.24 | 0.21 | 0.35 | 0.52 | 0.81 | 0.22 | 0.75 | 0.00 | 0.82 | 0.00 | | X | | |
| A_23_P33723 | CD163 | 0.45 | 0.12 | 0.12 | 0.66 | 0.35 | 0.93 | 0.77 | 0.13 | 0.46 | 0.27 | 0.77 | 0.00 | | X | | |
| A_23_P384517 | GYG1 | 0.95 | 0.55 | 0.79 | 0.95 | 1.37 | 0.14 | 1.12 | 0.15 | 1.15 | 0.00 | 1.36 | 0.57 | X | X | | |
| A_24_P12115 | CFLAR | 0.25 | 0.20 | 0.13 | 0.28 | 0.11 | 0.32 | 0.66 | 0.00 | 0.64 | 0.00 | 0.63 | 0.00 | | X | | X |
| A_33_P3324884 | MICAL1 | 0.37 | 0.19 | 0.41 | 0.22 | 0.33 | 0.20 | 0.69 | 0.00 | 0.72 | 0.00 | 0.69 | 0.27 | | X | | X |
| A_33_P376482 | SIRT5 | 0.53 | 0.73 | 0.47 | 0.12 | 0.60 | 0.13 | 0.59 | 0.14 | 0.62 | 0.00 | 0.77 | 0.00 | | X | | |
| A_23_P256821 | CR1 | 0.49 | 0.15 | 0.50 | 0.72 | 0.66 | 0.60 | 0.77 | 0.00 | 0.93 | 0.00 | 0.99 | 0.00 | | X | | |
| A_23_P116765 | LALBA | 0.29 | 0.26 | −0.35 | 0.12 | −0.39 | 0.38 | −0.55 | 0.84 | −0.88 | 0.85 | −0.94 | 0.39 | | X | | |
| A_33_P3329549 | FBRS | −0.83 | 0.64 | −0.25 | 0.12 | −0.36 | 0.44 | −0.46 | 0.26 | −0.67 | 0.16 | −0.72 | 0.32 | | X | | |
| A_23_P66719 | DHRS13 | 0.45 | 0.18 | 0.37 | 0.34 | 0.46 | 0.39 | 0.56 | 0.13 | 0.59 | 0.13 | 0.65 | 0.00 | | X | | |
| A_32_P154342 | SLCO4C1 | 0.59 | 0.48 | 0.39 | 0.13 | 0.55 | 0.88 | 0.59 | 0.59 | 0.57 | 0.23 | 0.72 | 0.00 | | X | | |
| A_23_P7429 | GBPS | −0.81 | 0.19 | −0.45 | 0.62 | −0.74 | 0.15 | −0.37 | 0.49 | −0.31 | 0.14 | −0.38 | 0.58 | X | | X | |
| A_23_P56356 | PLB1 | 0.49 | 0.45 | 0.36 | 0.76 | 0.54 | 0.26 | 0.89 | 0.15 | 0.88 | 0.00 | 0.93 | 0.00 | | X | | |
| A_23_P57856 | BCL6 | 0.25 | 0.30 | 0.21 | 0.28 | 0.29 | 0.11 | 0.89 | 0.91 | 0.92 | 0.00 | 0.89 | 0.00 | | X | | |
| A_32_P148796 | UBXN2B | 0.34 | 0.69 | 0.18 | 0.18 | 0.23 | 0.32 | 0.66 | 0.14 | 0.55 | 0.00 | 0.63 | 0.00 | | X | | |
| A_23_P14741 | KIRREL3 | 0.36 | 0.30 | −0.29 | 0.22 | −0.22 | 0.24 | −0.66 | 0.79 | −0.80 | 0.98 | −0.84 | 0.11 | | X | | |
| A_33_P3257279 | TMEM145 | 0.64 | 0.79 | −0.16 | 0.39 | −0.28 | 0.16 | −0.29 | 0.22 | −0.67 | 0.39 | −0.75 | 0.69 | | X | | |
| A_23_P259863 | CD177 | 1.76 | 0.14 | 1.30 | 0.93 | 1.88 | 0.44 | 1.87 | 0.55 | 1.77 | 0.60 | 2.29 | 0.00 | X | X | | |
| A_23_P75769 | MS4A4A | 0.66 | 0.13 | 0.53 | 0.35 | 0.63 | 0.12 | 0.95 | 0.48 | 0.92 | 0.34 | 1.13 | 0.00 | | X | | |
| A_23_P8311 | CDK5RAP2 | 0.84 | 0.78 | 0.72 | 0.94 | 0.84 | 0.91 | 0.98 | 0.12 | 1.27 | 0.00 | 1.25 | 0.00 | X | X | | |
| A_23_P121716 | ANXA3 | 0.82 | 0.22 | 0.55 | 0.72 | 0.67 | 0.16 | 1.18 | 0.27 | 1.39 | 0.00 | 1.12 | 0.00 | | X | | |
| A_23_P17857 | IL1RAP | 0.49 | 0.25 | 0.18 | 0.23 | 0.22 | 0.15 | 0.85 | 0.00 | 0.64 | 0.16 | 0.70 | 0.43 | | X | | X |
| A_23_P741 | S1A12 | 0.88 | 0.28 | 0.67 | 0.23 | 0.93 | 0.89 | 0.85 | 0.57 | 0.93 | 0.00 | 1.89 | 0.00 | | X | | |
| A_24_P295245 | ASPH | 0.58 | 0.29 | 0.47 | 0.30 | 0.74 | 0.17 | 0.78 | 0.17 | 0.79 | 0.00 | 0.98 | 0.00 | | X | | |
| A_23_P252681 | PCYT1A | 0.38 | 0.26 | 0.35 | 0.15 | 0.45 | 0.00 | 0.59 | 0.00 | 0.66 | 0.00 | 0.69 | 0.00 | | X | | |
| A_23_P351275 | UPP1 | 0.93 | 0.37 | 0.71 | 0.27 | 0.77 | 0.19 | 1.28 | 0.00 | 0.93 | 0.00 | 1.24 | 0.00 | X | X | X | |
| A_23_P13291 | RBM47 | 0.31 | 0.76 | 0.13 | 0.34 | 0.23 | 0.64 | 0.70 | 0.00 | 0.69 | 0.00 | 0.69 | 0.81 | | X | | X |
| A_23_P12418 | ITGAM | 0.53 | 0.12 | 0.49 | 0.17 | 0.48 | 0.44 | 0.69 | 0.28 | 0.62 | 0.00 | 0.71 | 0.00 | | X | | |
| A_33_P33975 | TBC1D8 | 0.73 | 0.14 | 0.62 | 0.92 | 0.72 | 0.17 | 0.98 | 0.00 | 0.98 | 0.00 | 1.82 | 0.00 | X | X | | |
| A_23_P1292 | LGALS2 | −0.70 | 0.11 | −0.66 | 0.29 | −1.33 | 0.00 | −0.88 | 0.29 | −1.18 | 1.00 | −1.36 | 0.28 | X | X | X | X |
| A_24_P3881 | FKBP5 | 0.76 | 0.15 | 0.57 | 0.28 | 0.73 | 0.30 | 1.78 | 0.00 | 1.76 | 0.00 | 1.19 | 0.00 | | X | | |
| A_23_P11212 | ACSL1 | 0.47 | 0.60 | 0.26 | 0.17 | 0.38 | 0.36 | 0.82 | 0.50 | 0.76 | 0.00 | 0.83 | 0.00 | | X | | |
| A_33_P3338793 | KCNC3 | 0.37 | 0.11 | −0.85 | 0.64 | −0.28 | 0.44 | −0.16 | 0.46 | −0.68 | 0.86 | −0.73 | 0.95 | | X | | |
| A_23_P4734 | HHEX | 0.50 | 0.62 | 0.35 | 0.25 | 0.33 | 0.16 | 0.57 | 0.79 | 0.62 | 0.18 | 0.62 | 0.00 | | X | | |
| A_23_P217778 | MSL3 | 0.46 | 0.33 | 0.29 | 0.85 | 0.45 | 0.27 | 0.69 | 0.27 | 0.53 | 0.50 | 0.69 | 0.00 | | X | | X |
| A_33_P333592 | SYNE1 | −0.47 | 0.81 | −0.36 | 0.59 | −0.46 | 0.22 | −0.45 | 0.58 | −0.73 | 0.14 | −0.74 | 0.23 | | X | | X |
| A_33_P32328 | CD177 | 0.60 | 0.53 | 0.73 | 0.19 | 0.93 | 0.00 | 0.65 | 0.15 | 0.84 | 0.32 | 0.92 | 0.00 | X | X | | |
| A_23_P27424 | ZNF418 | 0.88 | 0.73 | −0.18 | 0.38 | −0.39 | 0.98 | −0.26 | 0.32 | −0.63 | 0.69 | −0.77 | 0.46 | | X | | |
| A_23_P14741 | KIRREL3 | 0.24 | 0.31 | −0.18 | 0.43 | −0.19 | 0.32 | −0.19 | 0.49 | −0.70 | 0.55 | −0.76 | 0.61 | | X | | |
| A_33_P327555 | ST6GALNAC3 | 0.59 | 0.98 | 0.46 | 0.48 | 0.64 | 0.00 | 0.59 | 0.15 | 0.60 | 0.12 | 0.75 | 0.59 | X | X | | X |
| A_23_P41854 | CARD6 | 0.45 | 0.60 | 0.37 | 0.64 | 0.58 | 0.29 | 0.67 | 0.54 | 0.64 | 0.19 | 0.87 | 0.17 | | X | | |
| A_23_P9497 | LILRA4 | 0.36 | 0.19 | 0.40 | 0.16 | 0.23 | 0.49 | 0.74 | 0.58 | 0.60 | 0.00 | 0.53 | 0.00 | | X | | |
| A_33_P3228612 | CACNA1E | 0.28 | 0.58 | 0.44 | 0.14 | 0.42 | 0.81 | 0.59 | 0.32 | 0.76 | 0.00 | 0.71 | 0.00 | | X | | |
| A_23_P156218 | GZMK | −0.33 | 0.54 | −0.35 | 0.36 | −0.47 | 0.57 | −0.65 | 0.69 | −0.65 | 0.22 | −0.63 | 0.40 | | X | | |
| A_23_P1776 | IL17RA | 0.17 | 0.25 | 0.13 | 0.36 | 0.19 | 0.97 | 0.65 | 0.00 | 0.58 | 0.00 | 0.67 | 0.17 | | X | | |
| A_33_P3331687 | GPSM1 | 0.21 | 0.37 | −0.82 | 0.64 | −0.23 | 0.27 | −0.38 | 0.15 | −0.68 | 0.14 | −0.76 | 0.14 | | X | | |
| A_23_P39931 | DYSF | 0.55 | 0.13 | 0.44 | 0.19 | 0.53 | 0.25 | 0.86 | 0.00 | 0.82 | 0.00 | 0.91 | 0.93 | | X | | |
| A_24_P286114 | SLC1A3 | 0.56 | 0.25 | 0.28 | 0.64 | 0.55 | 0.49 | 0.69 | 0.40 | 0.46 | 0.65 | 0.69 | 0.00 | | X | | |
| A_23_P6919 | PLSCR1 | 0.59 | 0.12 | 0.32 | 0.11 | 0.34 | 0.85 | 0.94 | 0.00 | 0.81 | 0.00 | 0.86 | 0.00 | | X | | |
| A_33_P3385785 | S1A12 | 1.17 | 0.52 | 0.82 | 0.15 | 1.13 | 0.15 | 1.36 | 0.14 | 1.89 | 0.00 | 1.30 | 0.00 | X | X | | |
| A_23_P4174 | MMP9 | 1.21 | 0.37 | 0.79 | 0.17 | 1.25 | 0.13 | 1.18 | 0.73 | 1.13 | 0.00 | 1.45 | 0.00 | X | X | | |
| A_23_P13765 | FCER1A | −0.35 | 0.15 | −0.35 | 0.12 | −0.64 | 0.46 | −0.49 | 0.72 | −0.66 | 0.00 | −0.89 | 0.00 | | X | | |
| A_23_P12418 | ITGAM | 0.51 | 0.12 | 0.38 | 0.28 | 0.49 | 0.45 | 0.69 | 0.17 | 0.59 | 0.00 | 0.74 | 0.00 | | X | | |
| A_23_P1782 | CD82 | 0.35 | 0.13 | 0.34 | 0.95 | 0.43 | 0.65 | 0.52 | 0.15 | 0.59 | 0.00 | 0.66 | 0.44 | | X | | X |
| A_24_P649624 | KIF1B | 0.63 | 0.12 | 0.45 | 0.96 | 0.54 | 0.19 | 0.76 | 0.00 | 0.66 | 0.35 | 0.83 | 0.00 | | X | | |
| A_23_P117546 | SOS2 | 0.34 | 0.62 | 0.25 | 0.62 | 0.42 | 0.42 | 0.58 | 0.13 | 0.57 | 0.00 | 0.67 | 0.37 | | X | | |
| A_23_P431388 | SPOCD1 | 0.41 | 0.17 | 0.59 | 0.36 | 0.65 | 0.12 | 0.53 | 0.17 | 0.64 | 0.41 | 0.64 | 0.29 | X | X | | |
| A_33_P3364582 | TNXB | 0.16 | 0.49 | −0.38 | 0.78 | −0.29 | 0.12 | −0.39 | 0.87 | −0.75 | 0.34 | −0.72 | 0.82 | | X | | |
| A_24_P18797 | PADI2 | 0.37 | 0.24 | 0.13 | 0.38 | 0.32 | 0.82 | 0.61 | 0.11 | 0.39 | 0.22 | 0.64 | 0.00 | | X | | |
| A_21_P13195 | SEPT14 | 0.40 | 0.12 | 0.41 | 0.23 | 0.41 | 0.14 | 0.70 | 0.00 | 0.83 | 0.00 | 0.84 | 0.00 | | X | | X |
| A_23_P4174 | MMP9 | 1.32 | 0.28 | 0.79 | 0.90 | 1.20 | 0.13 | 1.20 | 0.75 | 1.13 | 0.00 | 1.45 | 0.00 | X | X | | |
| A_23_P16648 | OSM | 0.67 | 0.61 | 0.33 | 0.14 | 0.53 | 0.11 | 0.99 | 0.00 | 0.77 | 0.00 | 0.89 | 0.00 | | X | | |
| A_33_P3263756 | ZNF446 | 0.73 | 0.67 | −0.30 | 0.72 | −0.30 | 0.24 | −1.00 | 0.64 | −0.60 | 0.15 | −0.66 | 0.13 | | X | | X |
| A_23_P99397 | ZDHHC2 | 0.54 | 0.42 | 0.41 | 0.78 | 0.63 | 0.00 | 0.82 | 0.00 | 0.77 | 0.00 | 0.96 | 0.00 | | X | | |
| A_23_P143845 | TIPARP | 0.64 | 0.56 | 0.48 | 0.18 | 0.49 | 0.26 | 0.78 | 0.38 | 0.74 | 0.00 | 0.72 | 0.55 | | X | | |
| A_23_P122863 | GRB1 | 0.62 | 0.19 | 0.52 | 0.25 | 0.92 | 0.72 | 0.80 | 0.32 | 0.96 | 0.00 | 1.23 | 0.00 | | X | | |
| A_33_P3282394 | MLLT1 | 0.40 | 0.19 | 0.35 | 0.12 | 0.49 | 0.60 | 0.79 | 0.12 | 0.75 | 0.00 | 0.85 | 0.00 | | X | | |
| A_23_P1243 | P2RX2 | 0.86 | 1.00 | −0.48 | 0.17 | −0.34 | 0.98 | −0.29 | 0.15 | −0.73 | 0.66 | −0.60 | 0.32 | | X | | X |
| A_23_P14316 | ARID5A | 0.45 | 0.29 | 0.36 | 0.84 | 0.51 | 0.89 | 0.64 | 0.55 | 0.58 | 0.00 | 0.73 | 0.70 | | X | | X |
| A_24_P28567 | IL18R1 | 0.97 | 0.12 | 0.78 | 0.17 | 1.12 | 0.00 | 1.22 | 0.27 | 1.26 | 0.00 | 1.47 | 0.00 | X | X | X | |
| A_24_P353794 | GALNT2 | 0.49 | 0.12 | 0.54 | 0.65 | 0.48 | 0.32 | 0.60 | 0.00 | 0.65 | 0.00 | 0.63 | 0.00 | | X | | |
| A_23_P39251 | PLIN5 | 0.34 | 0.80 | 0.26 | 0.99 | 0.49 | 0.46 | 0.72 | 0.83 | 0.68 | 0.00 | 0.86 | 0.00 | | X | | |
| A_23_P99163 | DRAM1 | 0.43 | 0.11 | 0.40 | 0.51 | 0.50 | 0.71 | 0.63 | 0.16 | 0.64 | 0.00 | 0.76 | 0.12 | | X | | |
| A_23_P11473 | NAIP | 0.52 | 0.58 | 0.28 | 0.21 | 0.54 | 0.15 | 0.95 | 0.16 | 0.84 | 0.00 | 1.16 | 0.00 | | X | | |
| A_23_P38614 | ATP9A | 0.99 | 0.56 | 0.85 | 0.20 | 0.98 | 0.00 | 0.92 | 0.46 | 0.93 | 0.00 | 1.14 | 0.00 | X | X | X | |
| A_23_P363313 | SLC16A11 | 0.23 | 0.38 | −0.44 | 0.71 | −0.23 | 0.27 | 0.13 | 0.56 | −0.67 | 0.83 | −0.62 | 0.64 | | X | | |
| A_23_P121716 | ANNA3 | 0.79 | 0.22 | 0.56 | 0.64 | 0.69 | 0.13 | 1.93 | 0.16 | 1.45 | 0.00 | 1.12 | 0.00 | | X | | |
| A_23_P52266 | IFIT1 | −0.72 | 0.27 | −0.79 | 0.27 | −1.58 | 0.13 | −0.60 | 0.50 | −0.80 | 0.12 | −1.72 | 0.95 | | X | | |
| A_33_P3319957 | ARG1 | 0.88 | 0.28 | 0.75 | 0.30 | 1.15 | 0.59 | 1.28 | 0.14 | 1.19 | 0.00 | 1.51 | 0.00 | | X | | |
| A_33_P354143 | IL17RA | 0.25 | 0.16 | 0.17 | 0.22 | 0.39 | 0.22 | 0.62 | 0.48 | 0.57 | 0.00 | 0.73 | 0.39 | | X | | |
| A_33_P33635 | PFKFB2 | 0.88 | 0.38 | 0.74 | 0.17 | 1.17 | 0.00 | 1.19 | 0.23 | 1.95 | 0.00 | 1.36 | 0.00 | X | X | | |
| A_23_P4174 | MMP9 | 1.62 | 0.26 | 0.85 | 0.76 | 1.22 | 0.14 | 1.20 | 0.59 | 1.14 | 0.00 | 1.46 | 0.00 | X | X | | |
| A_23_P161156 | ZNF438 | 0.41 | 0.38 | 0.30 | 0.70 | 0.45 | 0.17 | 0.70 | 0.44 | 0.72 | 0.00 | 0.81 | 0.86 | | X | | |
| A_23_P87329 | NAT1 | −0.14 | 0.57 | 0.63 | 0.74 | −0.32 | 0.82 | −0.63 | 0.30 | −0.33 | 0.94 | −0.63 | 0.19 | | X | | |
| A_21_P149 | C2orf3 | 0.13 | 0.59 | −0.39 | 0.13 | −0.30 | 0.16 | −0.70 | 0.79 | −0.78 | 0.90 | −0.71 | 0.19 | | X | | |
| A_33_P339277 | TP53I3 | 0.67 | 0.46 | 0.49 | 0.46 | 0.68 | 0.20 | 0.83 | 0.00 | 0.69 | 0.25 | 0.84 | 0.00 | X | X | X | |
| A_23_P2348 | S1A9 | 0.44 | 0.76 | 0.37 | 0.95 | 0.53 | 0.50 | 0.87 | 0.37 | 0.90 | 0.00 | 0.80 | 0.00 | | X | | |
| A_23_P6943 | GPR15 | 0.15 | 0.61 | −0.66 | 0.26 | −0.42 | 0.18 | 0.95 | 0.76 | −0.89 | 0.32 | −0.85 | 0.25 | | X | | X |
| A_23_P123393 | KCNQ3 | 0.18 | 0.43 | −0.39 | 0.58 | −0.47 | 0.51 | −0.27 | 0.34 | −0.92 | 0.20 | −0.92 | 0.17 | | X | | |
| A_23_P116765 | LALBA | 0.32 | 0.23 | −0.32 | 0.15 | −0.31 | 0.16 | −0.24 | 0.45 | −0.95 | 0.19 | −0.95 | 0.38 | | X | | |
| A_33_P3216448 | COL11A2 | 0.13 | 0.57 | −0.23 | 0.25 | −0.38 | 0.36 | −0.35 | 0.16 | −0.81 | 0.43 | −0.80 | 0.39 | | X | | |
| A_24_P161973 | ATP11A | 0.26 | 0.66 | 0.13 | 0.22 | 0.34 | 0.31 | 0.59 | 0.00 | 0.52 | 0.00 | 0.66 | 0.00 | | X | | |
| A_23_P12418 | ITGAM | 0.56 | 0.66 | 0.48 | 0.20 | 0.57 | 0.29 | 0.75 | 0.11 | 0.58 | 0.26 | 0.72 | 0.00 | | X | | |
| A_23_P11473 | NAIP | 0.49 | 0.89 | 0.25 | 0.27 | 0.46 | 0.27 | 0.94 | 0.24 | 0.87 | 0.00 | 1.00 | 0.00 | | X | | |
| A_24_P337746 | RABGEF1 | 0.57 | 0.36 | 0.46 | 0.21 | 0.62 | 0.00 | 0.72 | 0.00 | 0.68 | 0.00 | 0.83 | 0.00 | | X | | |
| A_23_P128993 | GZMH | −0.68 | 0.67 | −0.69 | 0.34 | −0.52 | 0.20 | −0.42 | 0.18 | −0.52 | 0.39 | −0.33 | 0.19 | X | | | |
| A_24_P5245 | HLA-DMA | −0.11 | 0.39 | −0.28 | 0.12 | −0.44 | 0.16 | −0.28 | 0.27 | −0.64 | 0.00 | −0.68 | 0.00 | | X | | |
| A_33_P3215797 | AHDC1 | 0.39 | 0.12 | −0.34 | 0.12 | −0.24 | 0.24 | 0.35 | 0.89 | −0.78 | 0.40 | −0.73 | 0.45 | | X | | |
| A_24_P295963 | SLC38A2 | 0.34 | 0.17 | 0.17 | 0.13 | 0.32 | 0.91 | 0.62 | 0.00 | 0.51 | 0.00 | 0.64 | 0.00 | | X | | |
| A_23_P153945 | GTDC1 | 0.44 | 0.33 | 0.37 | 0.78 | 0.51 | 0.11 | 0.66 | 0.17 | 0.61 | 0.00 | 0.71 | 0.00 | | X | | |
| A_24_P32552 | SORT1 | 0.58 | 0.17 | 0.54 | 0.12 | 0.53 | 0.34 | 0.88 | 0.00 | 0.86 | 0.00 | 0.94 | 0.00 | | X | | |
| A_33_P3247473 | KRTAP23-1 | 0.34 | 0.29 | −0.22 | 0.29 | −0.12 | 0.53 | −0.24 | 0.31 | −0.78 | 0.80 | −0.59 | 0.32 | | X | | |
| A_23_P41664 | LRRC7 | 0.50 | 0.77 | 0.50 | 0.68 | 0.47 | 0.18 | 0.61 | 0.27 | 0.76 | 0.00 | 0.69 | 0.00 | | X | | |
| A_24_P183128 | PLAC8 | 0.76 | 0.18 | 0.77 | 0.24 | 0.93 | 0.33 | 0.93 | 0.38 | 1.00 | 0.12 | 1.12 | 0.99 | X | X | | |
| A_23_P217712 | ARSD | 0.13 | 0.59 | −0.46 | 0.26 | −0.35 | 0.76 | −0.19 | 0.96 | −0.68 | 0.48 | −0.62 | 0.54 | | X | | |
| A_23_P65789 | MCTP2 | 0.44 | 0.19 | 0.41 | 0.12 | 0.48 | 0.97 | 0.77 | 0.00 | 0.84 | 0.65 | 0.87 | 0.00 | | X | | |
| A_24_P3533 | LIMK2 | 0.38 | 0.17 | 0.37 | 0.93 | 0.29 | 0.19 | 0.74 | 0.00 | 0.76 | 0.00 | 0.73 | 0.00 | | X | | |
| A_23_P117546 | SOS2 | 0.31 | 0.60 | 0.25 | 0.72 | 0.26 | 0.12 | 0.53 | 0.51 | 0.60 | 0.00 | 0.54 | 0.40 | | X | | |
| A_33_P3272527 | MAVS | 0.14 | 0.64 | −0.52 | 0.33 | −0.20 | 0.43 | −0.19 | 0.44 | −0.81 | 0.43 | −0.71 | 0.32 | | X | | |
| A_23_P313389 | UGCG | 0.92 | 0.12 | 0.77 | 0.17 | 0.95 | 0.11 | 1.17 | 0.00 | 1.12 | 0.00 | 1.29 | 0.00 | X | X | X | |
| A_23_P17242 | ABHD1 | 0.15 | 0.46 | −0.46 | 0.32 | −0.40 | 0.38 | 0.79 | 0.73 | −0.67 | 0.83 | −0.71 | 0.40 | | X | | |
| A_23_P156748 | ANKS1A | 0.37 | 0.27 | 0.21 | 0.59 | 0.39 | 0.92 | 0.66 | 0.18 | 0.56 | 0.00 | 0.67 | 0.00 | | X | | |
| A_23_P254756 | CD164 | 0.32 | 0.53 | 0.25 | 0.32 | 0.17 | 0.15 | 0.60 | 0.11 | 0.57 | 0.00 | 0.58 | 0.00 | | X | | X |
| A_23_P162211 | MANSC1 | 0.44 | 0.58 | 0.31 | 0.19 | 0.45 | 0.18 | 0.66 | 0.18 | 0.58 | 0.11 | 0.65 | 0.00 | | X | | |
| A_23_P21141 | BREMEN1 | 0.37 | 0.84 | 0.37 | 0.46 | 0.26 | 0.26 | 0.63 | 0.00 | 0.65 | 0.00 | 0.58 | 0.00 | | X | | X |
| A_33_P3242458 | SLC41A3 | 0.15 | 0.70 | −0.25 | 0.22 | −0.27 | 0.20 | −0.37 | 0.13 | −0.74 | 0.27 | −0.78 | 0.14 | | X | | |
| A_33_P3431595 | C8orf31 | 0.23 | 0.47 | −0.42 | 0.16 | −0.19 | 0.45 | 0.28 | 0.94 | −0.73 | 0.92 | −0.74 | 0.67 | | X | | |
| A_23_P11473 | NAIP | 0.51 | 0.62 | 0.26 | 0.26 | 0.54 | 0.16 | 0.95 | 0.16 | 0.84 | 0.00 | 1.13 | 0.00 | | X | | |
| A_33_P3411925 | WDR18 | 0.14 | 0.58 | −0.25 | 0.17 | −0.34 | 0.71 | −0.45 | 0.47 | −0.82 | 0.26 | −0.84 | 0.20 | | X | | |
| A_33_P339465 | HMG2B | 0.43 | 0.15 | 0.44 | 0.82 | −0.84 | 0.69 | −0.37 | 0.14 | −0.62 | 0.27 | −0.59 | 0.78 | | X | | |
| A_24_P945293 | CHMP3 | 0.42 | 0.87 | −0.39 | 0.49 | −0.38 | 0.64 | −0.35 | 0.16 | −0.79 | 0.23 | −0.77 | 0.32 | | X | | |
| A_23_P116765 | LALBA | 0.84 | 0.98 | −0.53 | 0.25 | −0.45 | 0.72 | −0.57 | 0.46 | −1.32 | 0.60 | −1.33 | 0.35 | | X | | |
| A_23_P94647 | OR1L3 | 0.22 | 0.42 | −0.67 | 0.14 | −0.42 | 0.11 | 0.26 | 0.93 | −0.97 | 0.17 | −0.95 | 0.12 | | X | | |
| A_23_P126623 | PGD | 0.68 | 0.48 | 0.58 | 0.32 | 0.57 | 0.14 | 0.80 | 0.00 | 0.67 | 0.00 | 0.72 | 0.00 | | X | | |
| A_23_P137665 | CHI3L1 | −0.65 | 0.27 | −0.69 | 0.18 | −0.62 | 0.78 | −0.83 | 0.35 | −0.96 | 0.25 | −0.75 | 0.17 | | X | | |
| A_24_P14875 | SH3BP5 | 0.57 | 0.11 | 0.39 | 0.34 | 0.42 | 0.68 | 0.77 | 0.14 | 0.67 | 0.00 | 0.68 | 0.00 | | X | | X |
| A_23_P12418 | ITGAM | 0.49 | 0.14 | 0.36 | 0.39 | 0.47 | 0.52 | 0.69 | 0.13 | 0.59 | 0.00 | 0.72 | 0.00 | | X | | |
| A_23_P151637 | RNASE2 | 0.66 | 0.58 | 0.62 | 0.20 | 0.58 | 0.24 | 0.75 | 0.12 | 0.59 | 0.64 | 0.62 | 0.29 | X | X | | |
| A_33_P327149 | RBMS1 | 0.27 | 0.81 | 0.23 | 0.85 | 0.40 | 0.93 | 0.62 | 0.14 | 0.67 | 0.00 | 0.76 | 0.38 | | X | | |
| A_23_P126844 | TNFRSF25 | −0.53 | 0.17 | −0.41 | 0.24 | −0.51 | 0.54 | −0.59 | 0.54 | −0.58 | 0.14 | −0.64 | 0.86 | | X | | |
| A_23_P14741 | KIRREL3 | 0.44 | 0.88 | −0.48 | 0.43 | −0.33 | 0.17 | −0.54 | 0.49 | −0.96 | 0.79 | −0.95 | 0.54 | | X | | |
| A_33_P3411612 | TMEM221 | 0.23 | 0.37 | −0.38 | 0.64 | −0.24 | 0.20 | 0.55 | 0.81 | −0.61 | 0.58 | −0.63 | 0.25 | | X | | |
| A_23_P1926 | KCNK15 | −0.14 | 0.96 | −0.36 | 0.39 | −0.26 | 0.16 | −0.45 | 0.26 | −0.76 | 0.62 | −0.76 | 0.82 | | X | | |
| A_23_P19543 | SRPK1 | 0.26 | 0.25 | 0.18 | 0.29 | 0.30 | 0.47 | 0.57 | 0.54 | 0.64 | 0.00 | 0.67 | 0.00 | | X | | |
| A_23_P27445 | MAP2K6 | 0.48 | 0.16 | 0.30 | 0.88 | 0.64 | 0.20 | 0.62 | 0.14 | 0.53 | 0.56 | 0.63 | 0.00 | | X | | |
| A_23_P1623 | IRAK3 | 0.54 | 0.23 | 0.36 | 0.85 | 0.71 | 0.44 | 0.86 | 0.93 | 0.88 | 0.00 | 1.15 | 0.00 | | X | | |
| A_23_P1733 | UCKL1 | 0.27 | 0.92 | −0.25 | 0.26 | −0.57 | 0.27 | −0.22 | 0.42 | −0.78 | 0.39 | −1.63 | 0.00 | | X | | X |
| A_23_P14464 | ALOXS | 0.46 | 0.31 | 0.38 | 0.22 | 0.43 | 0.14 | 0.75 | 0.22 | 0.73 | 0.00 | 0.69 | 0.00 | | X | | |
| A_23_P1243 | P2RX2 | 0.22 | 0.42 | −0.50 | 0.11 | −0.37 | 0.78 | 0.79 | 0.77 | −0.73 | 0.23 | −0.72 | 0.11 | | X | | X |
| A_23_P21463 | FLOT1 | 0.44 | 0.12 | 0.37 | 0.20 | 0.44 | 0.12 | 0.59 | 0.13 | 0.54 | 0.00 | 0.60 | 0.00 | | X | | |
| A_24_P322635 | ELMO2 | 0.33 | 0.26 | 0.35 | 0.99 | 0.30 | 0.63 | 0.55 | 0.30 | 0.60 | 0.00 | 0.59 | 0.00 | | X | | |
| A_33_P336948 | LRP6 | 0.12 | 0.64 | −0.25 | 0.21 | −0.19 | 0.27 | −0.22 | 0.34 | −0.63 | 0.26 | −0.66 | 0.14 | | X | | |
| A_23_P5638 | LRG1 | 0.63 | 0.33 | 0.44 | 0.30 | 0.62 | 0.47 | 0.64 | 0.22 | 0.54 | 0.32 | 0.68 | 0.00 | X | X | X | X |
| A_33_P3417281 | MUC4 | 0.35 | 0.99 | −0.38 | 0.38 | −0.19 | 0.35 | −0.48 | 0.38 | −0.76 | 0.18 | −0.68 | 0.37 | | X | | |
| A_23_P16325 | RNASE3 | 0.68 | 0.38 | 0.65 | 0.22 | 0.64 | 0.46 | 0.69 | 0.23 | 0.59 | 0.16 | 0.62 | 0.17 | X | X | | X |
| A_23_P344884 | CARNS1 | −0.94 | 0.96 | −0.38 | 0.34 | −0.28 | 0.72 | −0.34 | 0.62 | −0.69 | 0.23 | −0.75 | 0.00 | | X | | X |
| A_33_P3319126 | CR1L | 0.54 | 0.16 | 0.50 | 0.74 | 0.51 | 0.85 | 0.75 | 0.00 | 0.87 | 0.00 | 0.87 | 0.00 | | X | | |
| A_23_P94533 | CTSL1 | 0.31 | 0.26 | −0.18 | 0.32 | −0.15 | 0.47 | 0.33 | 0.89 | −0.63 | 0.66 | −0.59 | 0.28 | | X | | |
| A_23_P36941 | RGL4 | 0.86 | 0.58 | 0.76 | 0.44 | 0.97 | 0.45 | 1.48 | 0.34 | 1.12 | 0.00 | 1.20 | 0.00 | X | X | X | |
| A_23_P12884 | ITGA7 | 0.55 | 0.29 | 0.54 | 0.16 | 0.88 | 0.24 | 0.64 | 0.25 | 0.68 | 0.63 | 0.86 | 0.25 | | X | | |
| A_33_P322422 | POM121L12 | 0.67 | 0.35 | −0.48 | 0.52 | −0.42 | 0.75 | 0.48 | 0.19 | −0.75 | 0.74 | −0.79 | 0.89 | | X | | X |
| A_23_P21426 | FBN2 | 0.53 | 0.19 | −0.34 | 0.24 | −0.17 | 0.49 | 0.18 | 0.55 | −0.79 | 0.39 | −0.77 | 0.23 | | X | | |
| A_23_P14741 | KIRREL3 | 0.49 | 0.87 | −0.17 | 0.55 | −0.30 | 0.24 | −0.37 | 0.19 | −0.71 | 0.50 | −0.95 | 0.19 | | X | | |
| A_33_P3413216 | TSPAN4 | −0.37 | 0.88 | −0.50 | 0.48 | −0.39 | 0.39 | −0.38 | 0.86 | −0.84 | 0.70 | −0.79 | 0.20 | | X | | |
| A_24_P7121 | NSUN7 | 0.44 | 0.59 | 0.43 | 0.25 | 0.66 | 0.80 | 0.64 | 0.14 | 0.74 | 0.00 | 0.90 | 0.00 | | X | | X |
| A_24_P35142 | ZDHHC3 | 0.54 | 0.32 | 0.34 | 0.63 | 0.47 | 0.96 | 0.70 | 0.00 | 0.49 | 0.12 | 0.63 | 0.00 | | X | | |
| A_23_P16636 | CBS | 0.32 | 0.12 | 0.29 | 0.99 | 0.32 | 0.57 | 0.44 | 0.16 | 0.71 | 0.00 | 0.76 | 0.16 | | X | | |
| A_24_P11436 | TTC22 | 0.65 | 0.76 | −0.32 | 0.82 | −0.24 | 0.18 | −0.22 | 0.29 | −0.62 | 0.66 | −0.63 | 0.30 | | X | | |
| A_23_P28747 | PGLYRP1 | 0.62 | 0.67 | 0.46 | 0.23 | 0.63 | 0.34 | 0.45 | 0.53 | 0.39 | 0.38 | 0.49 | 0.84 | X | | X | |
| A_24_P343233 | HLA-DRB1 | −0.18 | 0.18 | −0.32 | 0.24 | −0.54 | 0.68 | −0.26 | 0.12 | −0.59 | 0.00 | −0.74 | 0.00 | | X | | |
| A_24_P239731 | B4GALT5 | 0.28 | 0.54 | 0.28 | 0.26 | 0.38 | 0.39 | 0.86 | 0.00 | 0.92 | 0.36 | 1.48 | 0.00 | | X | | X |
| A_33_P3245489 | ADAMTSL5 | 0.11 | 0.70 | 0.12 | 0.60 | −0.18 | 0.46 | −0.63 | 0.11 | −0.59 | 0.32 | −0.68 | 0.58 | | X | | X |
| A_33_P3238993 | AGFG1 | 0.86 | 0.55 | 0.62 | 0.21 | 0.67 | 0.40 | 1.00 | 0.00 | 0.99 | 0.00 | 0.96 | 0.57 | X | X | | |
| A_24_P29778 | C2orf3 | 0.58 | 0.84 | 0.43 | 0.96 | 0.34 | 0.48 | 0.74 | 0.86 | 0.62 | 0.00 | 0.61 | 0.31 | | X | | |
| A_23_P4166 | EDIL3 | 0.15 | 0.65 | −0.43 | 0.94 | −0.34 | 0.18 | −0.37 | 0.17 | −0.94 | 0.32 | −0.99 | 0.13 | | X | | |
| A_23_P7378 | IRAK1 | −0.22 | 0.91 | −0.19 | 0.28 | −0.21 | 0.19 | −0.39 | 0.73 | −0.61 | 0.12 | −0.78 | 0.12 | | X | | |
| A_23_P14464 | ALOX5 | 0.47 | 0.26 | 0.44 | 0.74 | 0.43 | 0.64 | 0.75 | 0.26 | 0.74 | 0.00 | 0.69 | 0.00 | | X | | |
| A_24_P235266 | GRB1 | 0.67 | 0.13 | 0.60 | 0.91 | 0.96 | 0.27 | 1.00 | 0.35 | 1.37 | 0.00 | 1.32 | 0.00 | X | X | | |
| A_23_P9114 | PECR | 0.59 | 0.21 | 0.48 | 0.27 | 0.59 | 0.00 | 0.59 | 0.12 | 0.64 | 0.00 | 0.74 | 0.00 | | X | | |
| A_23_P122863 | GRB1 | 0.63 | 0.15 | 0.56 | 0.12 | 0.88 | 0.82 | 0.85 | 0.25 | 0.92 | 0.00 | 1.26 | 0.00 | | X | | |
| A_23_P123393 | KCNQ3 | −0.17 | 0.95 | −0.38 | 0.96 | −0.49 | 0.35 | −0.57 | 0.59 | −0.95 | 0.96 | −1.12 | 0.13 | | X | | |
| A_24_P347378 | ALOX5AP | 0.61 | 0.15 | 0.45 | 0.19 | 0.68 | 0.13 | 0.61 | 0.99 | 0.47 | 0.18 | 0.69 | 0.00 | | X | | |
| A_33_P3347869 | C3 | 0.32 | 0.34 | −0.44 | 0.11 | −0.27 | 0.28 | 0.18 | 0.95 | −0.73 | 0.79 | −0.88 | 0.78 | | X | | |
| A_23_P14225 | LILRA2 | 0.44 | 0.42 | 0.31 | 0.41 | 0.25 | 0.14 | 0.76 | 0.13 | 0.62 | 0.00 | 0.57 | 0.00 | | X | | |
| A_24_P348265 | FCAR | 0.47 | 0.20 | 0.27 | 0.12 | 0.53 | 0.28 | 0.75 | 0.00 | 0.74 | 0.00 | 0.94 | 0.24 | | X | | |
| A_23_P1522 | BCL2A1 | 0.63 | 0.13 | 0.39 | 0.55 | 0.55 | 0.35 | 0.82 | 0.21 | 0.76 | 0.00 | 0.84 | 0.00 | | X | | |
| A_23_P26557 | C16orf59 | 0.22 | 0.43 | −0.35 | 0.15 | −0.27 | 0.19 | −0.12 | 0.63 | −0.62 | 0.57 | −0.75 | 0.29 | | X | | |
| A_23_P141173 | MPO | 0.50 | 0.26 | 0.57 | 0.15 | 0.59 | 0.15 | 0.55 | 0.29 | 0.61 | 0.40 | 0.72 | 0.24 | | X | | |
| A_23_P395 | SLC26A8 | 0.39 | 0.79 | 0.33 | 0.20 | 0.46 | 0.12 | 0.61 | 0.14 | 0.57 | 0.00 | 0.76 | 0.00 | | X | | |
| A_23_P122863 | GRB1 | 0.64 | 0.18 | 0.56 | 0.11 | 0.92 | 0.49 | 0.82 | 0.22 | 0.91 | 0.00 | 1.22 | 0.00 | | X | | |
| A_23_P1926 | KCNK15 | 0.22 | 0.92 | −0.18 | 0.33 | −0.27 | 0.15 | −0.47 | 0.16 | −0.64 | 0.36 | −0.72 | 0.11 | | X | | |
| A_33_P33769 | ORIJ4 | 0.27 | 0.31 | −0.39 | 0.59 | −0.23 | 0.27 | 0.31 | 0.92 | −0.65 | 0.45 | −0.65 | 0.23 | | X | | |
| A_23_P4174 | MMP9 | 1.20 | 0.37 | 0.79 | 0.96 | 1.20 | 0.14 | 1.25 | 0.64 | 1.15 | 0.00 | 1.46 | 0.00 | X | X | | |
| A_23_P122863 | GRB1 | 0.64 | 0.17 | 0.54 | 0.17 | 0.91 | 0.89 | 0.82 | 0.24 | 0.93 | 0.00 | 1.23 | 0.00 | | X | | |
| A_24_P373174 | RAB27A | 0.45 | 0.55 | 0.33 | 0.18 | 0.37 | 0.43 | 0.67 | 0.00 | 0.66 | 0.00 | 0.62 | 0.00 | | X | | |
| A_33_P3271316 | RPP25 | 0.52 | 0.14 | −0.39 | 0.18 | −0.25 | 0.35 | −0.16 | 0.96 | −0.97 | 0.36 | −1.12 | 0.86 | | X | | |
| A_24_P343233 | HLA-DRB1 | −0.15 | 0.33 | −0.41 | 0.40 | −0.59 | 0.82 | −0.27 | 0.27 | −0.74 | 0.00 | −0.85 | 0.67 | | X | | |
| A_23_P12418 | ITGAM | 0.52 | 0.13 | 0.38 | 0.28 | 0.47 | 0.59 | 0.74 | 0.11 | 0.60 | 0.00 | 0.72 | 0.00 | | X | | |
| A_33_P3357651 | KRTAP1-12 | 0.37 | 0.90 | −0.24 | 0.29 | −0.32 | 0.12 | −0.58 | 0.23 | −0.93 | 0.45 | −0.86 | 0.27 | | X | | |
| A_23_P142125 | HRC | 0.13 | 0.62 | −0.39 | 0.15 | −0.26 | 0.22 | −0.18 | 0.47 | −0.72 | 0.87 | −0.75 | 0.23 | | X | | |
| A_23_P11473 | NAIP | 0.62 | 0.43 | 0.25 | 0.27 | 0.46 | 0.27 | 1.63 | 0.15 | 0.85 | 0.00 | 0.99 | 0.00 | | X | | |
| A_23_P27315 | EMILIN2 | 0.26 | 0.17 | 0.25 | 0.16 | 0.33 | 0.17 | 0.67 | 0.39 | 0.54 | 0.24 | 0.67 | 0.00 | | X | | |
| A_33_P336624 | HCRT | 0.38 | 0.29 | −0.27 | 0.40 | −0.89 | 0.73 | −0.94 | 0.79 | −0.88 | 0.34 | −0.95 | 0.36 | | X | | |
| A_23_P7994 | LILRA3 | 0.37 | 0.38 | 0.22 | 0.13 | 0.36 | 0.74 | 0.82 | 0.20 | 0.61 | 0.58 | 0.84 | 0.57 | | X | | |
| A_32_P2835 | TDRD9 | 0.94 | 0.19 | 0.88 | 0.75 | 1.14 | 0.00 | 1.19 | 0.13 | 1.28 | 0.00 | 1.43 | 0.00 | X | X | | |
| A_24_P13886 | IDI1 | 0.73 | 0.15 | 0.52 | 0.34 | 0.62 | 0.40 | 0.82 | 0.15 | 0.74 | 0.00 | 0.70 | 0.00 | X | X | | |
| A_23_P37688 | LIME1 | 0.14 | 0.56 | −0.28 | 0.11 | −0.26 | 0.16 | −0.32 | 0.18 | −0.80 | 0.81 | −0.73 | 0.54 | | X | | |
| A_23_P1314 | MFNG | −0.85 | 0.74 | −0.52 | 0.57 | −0.42 | 0.67 | −0.33 | 0.24 | −0.81 | 0.65 | −0.94 | 0.12 | | X | | X |
| A_23_P7733 | TAAR2 | 0.19 | 0.53 | −0.23 | 0.29 | −0.18 | 0.67 | −0.39 | 0.16 | −0.79 | 0.15 | −0.75 | 0.28 | | X | | X |
| A_23_P12418 | ITGAM | 0.55 | 0.96 | 0.38 | 0.30 | 0.48 | 0.45 | 0.73 | 0.15 | 0.59 | 0.00 | 0.72 | 0.00 | | X | | |
| A_24_P382319 | CEACAM1 | 0.83 | 0.98 | 0.75 | 0.66 | 0.97 | 0.14 | 1.12 | 0.58 | 1.54 | 0.00 | 1.25 | 0.00 | X | X | | |
| A_23_P1926 | KCNK15 | 0.23 | 0.39 | −0.25 | 0.17 | −0.25 | 0.28 | −0.26 | 0.29 | −0.69 | 0.24 | −0.70 | 0.21 | | X | | |
| A_23_P362759 | PRDM5 | 0.40 | 0.23 | 0.36 | 0.49 | 0.57 | 0.00 | 0.55 | 0.19 | 0.60 | 0.14 | 0.82 | 0.00 | | X | | |
| A_33_P349625 | SORBS3 | 0.33 | 0.95 | −0.41 | 0.87 | −0.52 | 0.16 | −0.59 | 0.42 | −0.70 | 0.65 | −0.96 | 0.15 | | X | | |
| A_23_P47579 | NLRP14 | 0.54 | 0.85 | −0.32 | 0.24 | −0.12 | 0.66 | −0.49 | 0.85 | −0.63 | 0.59 | −0.67 | 0.39 | | X | | |
| A_33_P3271651 | HLA-DPB1 | −0.24 | 0.16 | −0.28 | 0.67 | −0.53 | 0.12 | −0.36 | 0.15 | −0.61 | 0.25 | −0.68 | 0.30 | | X | | |
| A_23_P19482 | DDAH2 | 0.62 | 0.13 | 0.65 | 0.22 | 0.94 | 0.00 | 1.47 | 0.00 | 1.20 | 0.00 | 1.32 | 0.00 | X | X | | |
| A_24_P283288 | MARK14 | 0.62 | 0.45 | 0.47 | 0.14 | 0.46 | 0.79 | 0.96 | 0.00 | 0.95 | 0.32 | 0.95 | 0.49 | | X | | |
| A_23_P16258 | SLC25A47 | 0.42 | 0.95 | −0.34 | 0.86 | −0.24 | 0.89 | −0.15 | 0.52 | −0.64 | 0.63 | −0.65 | 0.14 | | X | | |
| A_23_P1456 | SCGB3A2 | 0.16 | 0.58 | −0.11 | 0.64 | −0.69 | 0.75 | −0.19 | 0.37 | −0.63 | 0.17 | −0.69 | 0.37 | | X | | X |
| A_23_P117546 | SOS2 | 0.32 | 0.62 | 0.24 | 0.77 | 0.41 | 0.59 | 0.53 | 0.69 | 0.58 | 0.00 | 0.69 | 0.21 | | X | | |
| A_23_P11212 | ACSL1 | 0.45 | 0.58 | 0.25 | 0.19 | 0.37 | 0.43 | 0.81 | 0.13 | 0.76 | 0.00 | 0.84 | 0.00 | | X | | |
| A_23_P121716 | ANXA3 | 0.77 | 0.39 | 0.54 | 0.75 | 0.63 | 0.23 | 1.17 | 0.17 | 1.37 | 0.00 | 1.16 | 0.00 | | X | | |
| A_33_P3256848 | ADAM12 | 0.22 | 0.37 | −0.39 | 0.53 | −0.23 | 0.33 | −0.98 | 0.71 | −0.75 | 0.37 | −0.69 | 0.18 | | X | | |
| A_33_P331533 | KRT73 | 0.13 | 0.68 | −0.34 | 0.15 | −0.32 | 0.18 | −0.37 | 0.34 | −0.91 | 0.58 | −0.97 | 0.38 | | X | | |
| A_24_P11216 | UPK3B | −0.32 | 0.93 | −0.42 | 0.58 | −0.34 | 0.20 | −0.13 | 0.62 | −0.66 | 0.29 | −0.62 | 0.49 | | X | | |
| A_21_P11611 | DNAH17 | −0.18 | 0.57 | −0.49 | 0.82 | −0.50 | 0.76 | −0.65 | 0.24 | −1.38 | 0.12 | −0.95 | 0.38 | | X | | |
| A_33_P327947 | AGRP | 0.17 | 0.54 | −0.28 | 0.27 | −0.68 | 0.78 | 0.73 | 0.98 | −0.64 | 0.43 | −0.65 | 0.19 | | X | | |
| A_33_P3246613 | CCDC78 | −0.36 | 0.99 | −0.12 | 0.96 | −0.54 | 0.17 | −0.66 | 0.20 | −0.67 | 0.52 | −0.98 | 0.52 | | X | | |
| A_24_P148717 | CCR1 | 0.14 | 0.33 | 0.13 | 0.32 | 0.14 | 0.24 | 0.63 | 0.11 | 0.63 | 0.33 | 0.58 | 0.24 | | X | | |
| A_32_P44394 | AIM2 | 0.11 | 0.58 | 0.32 | 0.61 | 0.33 | 0.32 | 0.42 | 0.15 | 0.61 | 0.12 | 0.68 | 0.00 | | X | | |
| A_33_P3214943 | SPOCK2 | 0.25 | 0.94 | −0.33 | 0.17 | −0.41 | 0.49 | −0.50 | 0.31 | −0.95 | 0.00 | −0.95 | 0.00 | | X | | X |
| A_24_P88522 | RPS14 | −0.27 | 0.20 | −0.19 | 0.57 | −0.21 | 0.32 | −0.55 | 0.00 | −0.62 | 0.00 | −0.65 | 0.55 | | X | | |
| A_23_P18119 | IMPG2 | 0.23 | 0.46 | −0.28 | 0.17 | −0.27 | 0.18 | −0.23 | 0.37 | −0.69 | 0.18 | −0.79 | 0.18 | | X | | |
| A_24_P97342 | PROK2 | 0.41 | 0.14 | 0.20 | 0.46 | 0.27 | 0.18 | 0.70 | 0.42 | 0.77 | 0.19 | 0.66 | 0.11 | | X | | |
| A_33_P3376449 | ZDHHC23 | −0.69 | 0.77 | −0.38 | 0.62 | −0.39 | 0.27 | −0.47 | 0.32 | −0.82 | 0.26 | −0.81 | 0.00 | | X | | X |
| A_23_P11473 | NAIP | 0.44 | 0.13 | 0.22 | 0.31 | 0.44 | 0.32 | 0.95 | 0.23 | 0.88 | 0.00 | 1.00 | 0.00 | | X | | |
| A_21_P13998 | NAIP | 0.76 | 0.92 | 0.46 | 0.38 | 0.56 | 0.15 | 1.14 | 0.00 | 1.20 | 0.00 | 1.64 | 0.00 | | X | | |
| A_33_P3383912 | HLA-DRB3 | −0.62 | 0.71 | −0.30 | 0.32 | −0.55 | 0.11 | −0.18 | 0.22 | −0.70 | 0.00 | −0.68 | 0.00 | | X | | |
| A_23_P284 | C9orf25 | 0.87 | 0.97 | 0.15 | 0.44 | −0.23 | 0.24 | −0.63 | 0.36 | −0.37 | 0.50 | −0.66 | 0.94 | | X | | |
| A_33_P329881 | FFAR3 | 0.52 | 0.13 | 0.57 | 0.58 | 0.74 | 0.58 | 0.63 | 0.27 | 0.68 | 0.34 | 0.84 | 0.00 | | X | | X |
| A_23_P13687 | MAGFA6 | 0.22 | 0.43 | −0.32 | 0.11 | −0.24 | 0.32 | −0.16 | 0.50 | −0.64 | 0.14 | −0.64 | 0.85 | | X | | |
| A_23_P4174 | MMP9 | 0.99 | 0.44 | 0.75 | 0.13 | 1.16 | 0.19 | 1.19 | 0.75 | 1.12 | 0.23 | 1.45 | 0.00 | X | X | | |

Optimization of the classification panels. We used a biological function-based process to reduce the number of features in the two top performing classification panels (Lee et al., Sci Rep. 9:7365, 2019; incorporated herein by reference in its entirety). There were 89 and 326 biological processes associated with at least one of the genes in ISB19 and ISB63, respectively. To determine the biological processes with higher discriminatory power, 100,000 randomly selected 19-gene or 63-gene panels were generated from ISB58 or ISB355, respectively (FIG. 19A). Among the random 100,000 panels, the biological processes associated with the top 500 high performing and bottom 500 low performing panels (1% of the random 100,000 panels) based on the Discovery sample set were determined (FIG. 19B-19C). There were 19 and 46 biological functions from the top 500 performing panels that overlapped with the ISB19 and ISB63 panels, respectively (FIG. 19D-19E). The association of ISB19 and ISB63 genes from the bottom 500 panels with the 19 and 46 biological functions were lower compared to the top 500 panels (FIG. 19F-19G).

Redundant and mutually overlapping terms from the 19 and 46 core biological functions were clustered and summarized (FIG. 19H-19I). Three functional terms were associated with features (transcripts) in the ISB19 panel: immune response, metabolism, and signal transduction, whereas 6 terms were associated with the ISB63 panel: immune response, metabolism, signal transduction, apoptosis, transcription, and adhesion/migration (FIG. 19H-19I). The terms immune response, metabolism, and signal transduction were shared between the two panels. From the 58 DEGs from which ISB19 panel was derived, there were 13, 3, and 4 genes that are associated with immune response, signal transduction, and metabolism, respectively. Among the 355 DEGs from which ISB63 was derived, there were 67, 55, 41, 53, 7 and 40 genes associated with immune response, signal transduction, apoptosis, transcription, adhesion/migration, and metabolism, respectively.

Next, it was determined whether using a transcript representing each biological function could reduce the overall number of features while preserving the diagnostic performance of the panel(s). All 3-gene combinations that represent functional terms associated with ISB19 and 6-gene combinations representing ISB63 were generated by selecting one gene from each biological process, respectively. The performances of all possible 3-gene combinations (156=13×3×4, Table 22) were assessed using the Test sample set. However, it is prohibitory to do all 6-gene panels as there are more than 2 billion combinations (2,242,101,400=67×55×41×53×7×40). To reduce the number of possible 6-gene combinations for the ISB63 derived panels, the genes that had better classification capability were first determined. The performances of randomly selected 100,000 6-gene combinations were assessed with the Test sample set and then sorted based on classification performance. For each biological process, the most frequently appearing genes among the top performing 1,000 6-gene panels (top 1% of the 100,000 combinations tested) were identified. This analysis yielded 16, 12, 8, 10, 2, and 10 genes for immune response, signal transduction, apoptosis, transcription, adhesion/migration and metabolism, respectively (Table 22). From these top performing genes, 307,200 (16×12×8×10×2×10) 6-gene panels were generated and their performances were also assessed with the Test sample set. The performances of the ISB19 and ISB63 panels were also determined with the Test sample set. Seventy-eight of the 3-gene panels and 32,540 of the 6-gene panels showed performances similar or better than the original ISB19 and ISB63 panels based on DeLong's test (p-value >0.05). Therefore, these panels could be considered alternative optimized panels for the original ISB19 and ISB63.

TABLE 22
Substitutable genes in each biological process

| Functional term | ISB19 | ISB63 |
|---|---|---|
| Immune response | IL1R2, IL18R1, FCAR, GZMA, | BCL6, HLA-DRA, HLA-DMA, |
| | PGLYRP1, GZMH, LCN2, | BPI, LCN2, MAVS, HLA- |
| | BMX, VNN1, CLEC4D, | DPB1, GNG10, PDGFC, |
| | S100A12, SLC2A3, HK3 | CD55, HLA-DPA1, TLR8, |
| | | GZMA, BMX, HLA-DRB1, |
| | | OR1J4 |
| Signal transduction | IL18R1, GRB10, BMX | STOM, RRBP1, MAVS, |
| | | MICAL1, LILRA2, SH3BP5, |
| | | GNG10, WSB1,MAPK14, |
| | | RUNX1, BMX, RAPH |
| Metabolism | PFKFB3, PFKFB2, HK3, | MPO, CYP27A1, MICAL1, |
| | SLC2A3 | CAP1, PDGFC, AL0X5, |
| | | NQO2, CD163, CYP1B1, |
| | | B3GNT5 |
| Apoptosis | | MPO, LTF, LCN2, HLA-DRA, |
| | | MAVS, RPS6KA3, CFLAR, |
| | | GCA |
| Transcription | | BCL6, MLLT1, LTF, GAS7, |
| | | MXD3, SPOCD1, ZNF446, |
| | | MAVS, BASP1, RUNX1 |
| Adhesion/Migration | | TPM3, MAPK14 |

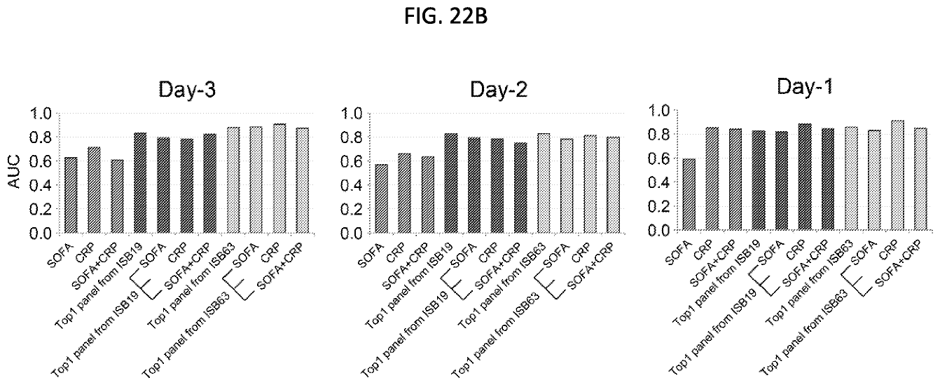

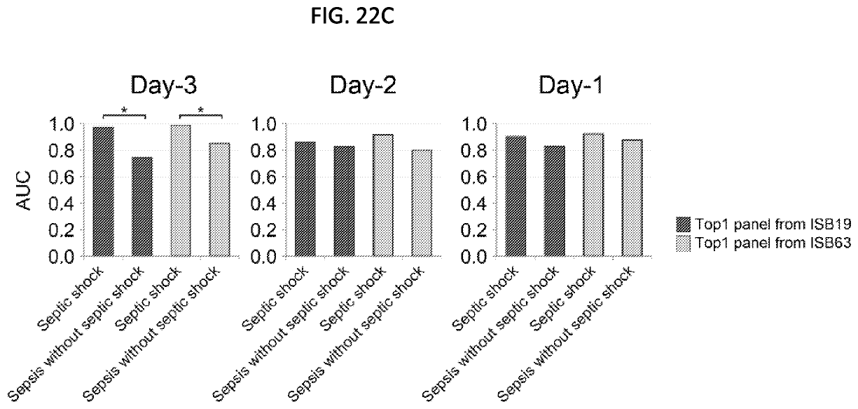

Evaluation of the biomarker panels using the Validation cohort. The diagnostic performances of the ISB19 and ISB63 panels were further assessed with the Validation cohort. Compared to their diagnostic performances in the Test cohort (FIG. 18), the panels performed similarly in the Validation cohort or produced even higher scores, except at Day-1 for both the ISB19 and ISB63 panels (FIG. 22A). Among the alternative panels, the top-performing 3-gene panel (LCN2, BMX, and SLC2A3) from ISB19 and 6-gene panel (GNG10, STOM, MPO, RPS6KA3, BCL3, and TPM3) from ISB63 were also assessed with the Validation cohort. The overall performances of the ISB3 and ISB6 panels were not significantly different from those of the original ISB19 and ISB63 panels across all time points (Day-3 to Day-1) in the Validation cohort (FIG. 22A) and ranged from AUC=0.82-0.83 and AUC=0.83-0.88 between Day-3 and Day-1, respectively. Importantly, the performances of both ISB3 and ISB6, and the original ISB19 and ISB63 were significantly better at all pre-diagnosis (Day-3, Day-2 and Day-1) time points than the SMS (AUC=0.61-0.69) or SeptiCyte™ LAB (AUC=0.63-0.73) panels (FIG. 22A).

Integration of top panel with clinical parameters does not significantly increase performance of the biomarker panels. A patient's clinical parameters such as the SOFA score and blood CRP level have been used to identify individuals having or suspected to have sepsis. Therefore, the diagnostic performances of SOFA and CRP were evaluated in the pre-symptomatic phase. In addition, the impact of integrating clinical information with the gene panels was evaluated to determine if this would increase the panels' performance. Neither the SOFA score nor the CRP level alone or combined performed well in diagnosing sepsis in the pre-symptomatic phase in the Validation cohort (FIG. 22B). Integration of either SOFA score, CRP level or both SOFA and CRP level with the gene panels did not significantly increase the performance of either the ISB3 (average AUC of 0.80 and 0.83 with and without SOFA and CRP, respectively) or ISB6 (average AUC of 0.84 and 0.85 with and without SOFA and CRP, respectively) (FIG. 22B).

The impact of disease severity on the performance of panels. To determine whether the panels' performances were affected by disease severity, patients who required vasopressors and met septic shock criteria were identified and labeled as “Septic Shock.” All remaining sepsis patients were grouped as “Sepsis” (this includes sepsis and severe sepsis categories according to Sepsis-2 criteria). The classification performances of the smaller 3- and 6-gene panels were then assessed in these groups. The panels had higher overall performances in identifying “Septic Shock” patients (average AUC of 0.91 and 0.94 from ISB3 and ISB6, respectively) prior to the onset of clinical symptoms compared to “Sepsis” patients (average AUC of 0.80 and 0.84 from ISB3 and ISB6, respectively) (FIG. 22C) and were significantly higher on Day-3 (p-value <0.05), suggesting the classifiers might be even more accurate in identifying patients at risk of developing a more severe condition.

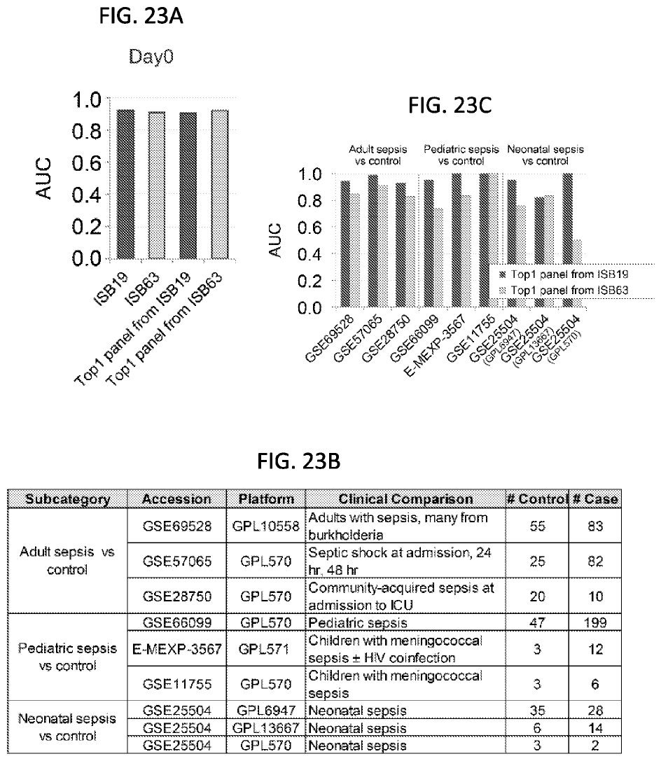

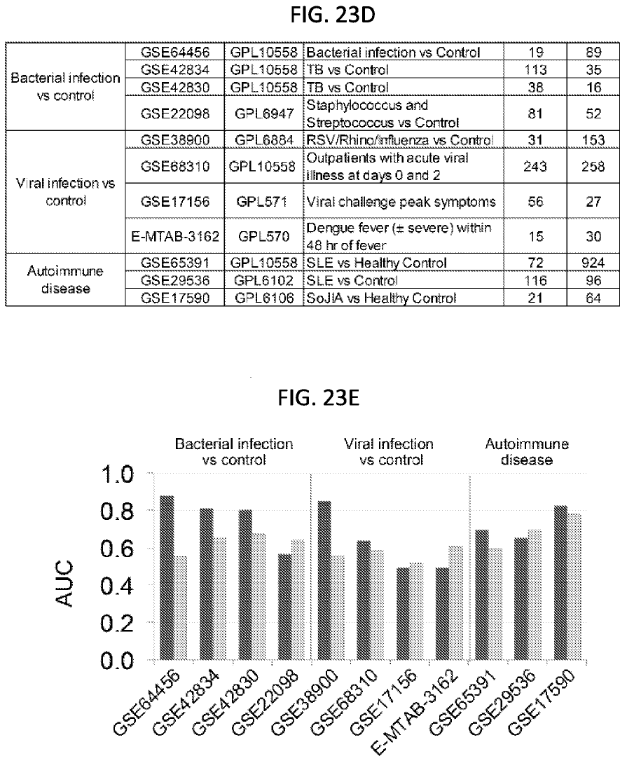

Assessing the accuracy and specificity of detecting sepsis in the symptomatic phase. Although the biomarker panels identified were aimed at detecting sepsis at the pre-symptomatic stage, the ISB19 and ISB63 panels also showed good diagnostic performance for patients with clinical symptoms of sepsis in the Test sample set (FIG. 18). Therefore, the diagnosis performance was further assessed in Validation samples where the AUCs were higher than 0.9 at Day0 (FIG. 23A). Data from nine publicly available sepsis related studies were used to further assess the panels' ability to specifically diagnose sepsis during the symptomatic phase. These datasets were obtained with different measurement platforms on different cohorts with a total of 633 individuals, including 197 controls and 436 sepsis patients (FIG. 23B). Based on the patient characteristics, we split the data into three groups: 1) Adult sepsis/severe sepsis, 2) Pediatric sepsis, 3) Neonatal sepsis. For the 3-gene panel, the average AUC=0.95 for all the samples, 0.95 for Adult sepsis, 0.98 for Pediatric sepsis and 0.92 for Neonatal sepsis. For the 6-gene panel, the average AUC=0.81 for all the samples, 0.86 for Adult sepsis, 0.86 for Pediatric sepsis, and 0.70 for Neonatal sepsis (FIG. 23C). These results are comparable to SMS (AUC=0.98) but significantly better than SeptiCyte™ LAB (average AUC=0.67).

We also assessed the specificity of the panels for sepsis diagnosis using eleven different public domain datasets including four sets representing bacterial infections without sepsis, four sets representing viral infection, and three sets representing autoimmune diseases (FIG. 23D). The panels performed significantly less well in all non-sepsis datasets. Specifically, the 3-gene panel had an average AUC=0.70 for all the samples and average AUC=0.76 for bacterial infection without sepsis, 0.62 for viral infection and 0.72 for autoimmune diseases. The 6-gene panel had an overall average AUC of 0.62, and 0.63 for bacterial infection without sepsis, 0.56 for viral infection, and 0.59 for autoimmune diseases (FIG. 23E). Collectively, these results suggest that both ISB3 and ISB6 have a much higher ability to detect bacterial-associated sepsis compared to non-sepsis bacterial infection, viral or non-infection related immune disorders.

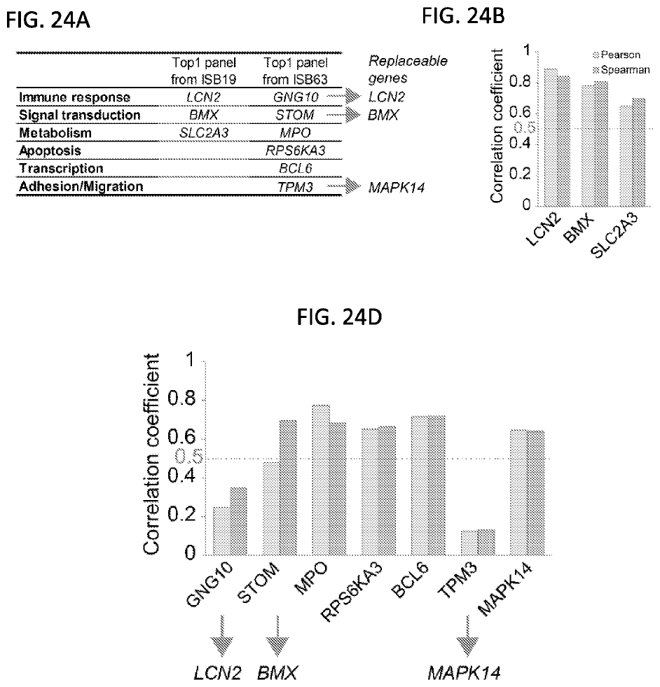

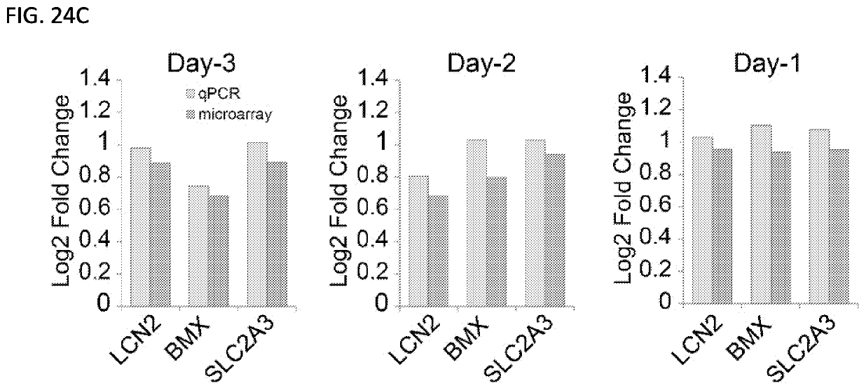

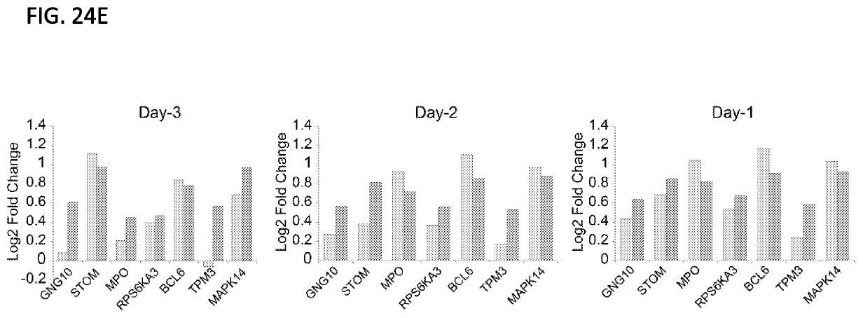

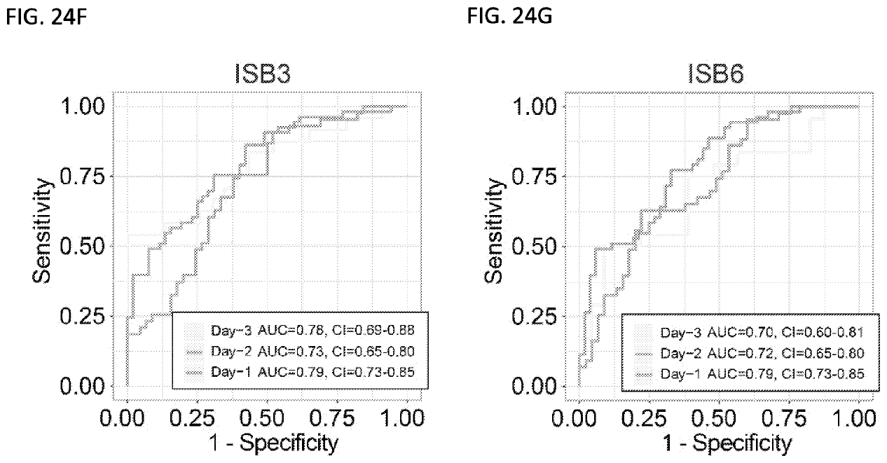

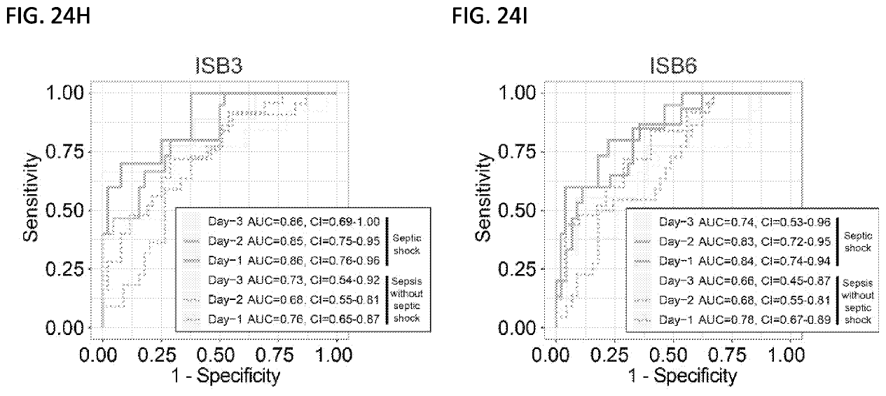

Validation of ISB3 and ISB6 using an alternative measurement platform. The performances of the 3-gene and 6-gene panels were further verified using qPCR (FIG. 24A). In the case of the 3-gene panel (ISB3), the qPCR measurements correlated well with the microarray measurements (FIG. 24B-24C) However, the qPCR measurements for GNG10 (immune response), STOM (signal transduction), and TPM3 (adhesion/migration) from the 6-gene panel (ISB6) correlated poorly with microarray measurements (FIG. 24D-24E). Since the panel optimization process also provides a list of substitutable genes (Table 22), we used this list to replace GNG10 with LCN2 (immune response gene), STOM with BMX (signal transduction), and TPM3 with MAPK14 (adhesion/migration), a process that resulted in improved correlation between the qPCR measurements and the microarray data.

Although the performances were slightly decreased compared to the microarray results (average ISB3 AUC=0.83 and average ISB6 AUC=0.85), the AUCs from qPCR results were still greater than 0.7 at all time points for both ISB3 (average AUC=0.77) and ISB6 (average AUC=0.74) (FIG. 24F). The panels also showed higher performance in patients with septic shock (average AUC of 0.85 and 0.81 for ISB3 and ISB6, respectively) compared to sepsis (average AUC of 0.72 and 0.71 for ISB3 and ISB6, respectively) (FIG. 24G).

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method, comprising measuring expression of a set of at least six genes in a sample from a subject having or suspected to have sepsis, wherein the set of at least six genes comprises or consists of:
LCN2, BMX, MPO, RPS6KA3, BCL6, MAPK14; RPS6KA3, BCL6, TPM3, GNG10, STOM, MPO; RPS6KA3, MAVS, TPM3, BCL6, STOM, MPO; RPS6KA3, BCL6, TPM3, BMX, STOM, MPO; RPS6KA3, LTF, TPM3, BCL6, STOM, PDGFC; RPS6KA3, BCL6, TPM3, CD55, STOM, MPO; RPS6KA3, BCL6, TPM3, GNG10, STOM, PDGFC; RPS6KA3, LTF, TPM3, BCL6, STOM, CYP1B1; LTF, BCL6, TPM3, CD55, STOM, PDGFC; RPS6KA3, BCL6, TPM3, TLR8, STOM, MPO; or RPS6KA3, BCL6, TPM3, HLA-DRA, GNG10, MPO;
RPS6KA3, MLLT1, TPM3, BCL6, GNG10, CYP27A1, YOD1; RPS6KA3, MLLT1, TPM3, BCL6, GNG10, PDGFC, YOD1; RPS6KA3, GAS7, TPM3, BCL6, RRBP1, MPO, YOD1; RPS6KA3, MLLT1, TPM3, BCL6, GNG10, MPO, LGALS2; RPS6KA3, GAS7, TPM3, BCL6, GNG10, MPO, YOD1; LTF, MLLT1, TPM3, BCL6, GNG10, PDGFC, YOD1; RPS6KA3, BCL6, TPM3, GNG10, LILRA2, CYP27A1, YOD1; RPS6KA3, BCL6, TPM3, GNG10, STOM, MPO, LGALS2; RPS6KA3, MLLT1, TPM3, BCL6, GNG10, MPO, YOD1; or RPS6KA3, BCL6, TPM3, CD55, STOM, MPO, IL17RA; or
RPS6KA3, MLLT1, TPM3, BCL6, GNG10, PDGFC, YOD1, LGALS2; RPS6KA3, MLLT1, TPM3, BCL6, GNG10, PDGFC, YOD1, LILRA4; RPS6KA3, MLLT1, TPM3, BCL6, GNG10, PDGFC, YOD1, IL17RA; RPS6KA3, MLLT1, TPM3, BCL6, GNG10, PDGFC, YOD1, TCN1; RPS6KA3, MLLT1, TPM3, BCL6, GNG10, PDGFC, YOD1, RNASE3; RPS6KA3, MLLT1, TPM3, BCL6, GNG10, PDGFC, YOD1, NOV; RPS6KA3, MLLT1, TPM3, BCL6, GNG10, PDGFC, YOD1, RNASE2; LTF, MLLT1, TPM3, BCL6, GNG10, PDGFC, YOD1, FAM105A; RPS6KA3, BCL6, TPM3, HLA-DRA, RRBP1, MPO, ERO1L, YOD1; or LTF, MLLT1, TPM3, BCL6, GNG10, PDGFC, YOD1, C14orf101;
wherein the expression of the set of genes is altered compared to a control.

2-4. (canceled)

5. The method of claim 1, wherein expression of one or more of LCN2, RPS6KA3, BCL6, MAPK14, TPM3, GNG10, STOM, MPO, BMX, LTF, PDGFC, CD55, CYP1B1, TLR8, MLLT1, YOD1, GAS7, RRBP1, LILRA2, IL17RA, LILRA4, TCN1, RNASE3, RNASE2, FAM105A, ERO1L, and/or C14orf101 is increased compared to the control and/or expression of one or more of MAVS, HLA-DRA, CYP27A1, LGALS2, and/or NOV is decreased compared to the control.

6. A method, comprising measuring expression of a set of at least three genes in a sample from a subject having or suspected to have sepsis, wherein the set of at least three genes comprises or consists of:
LCN2, SLC2A3, BMX; LCN2, SLC2A3, GRB10; LCN2, PFKFB3, GRB10; LCN2, PFKFB3, BMX; IL1R2, HK3, BMX; LCN2, HK3, BMX; LCN2, HK3, GRB10; GZMA, HK3, BMX; FCAR, PFKFB2, BMX; or LCN2, PFKFB3, IL18R1;
LCN2, PFKFB3, GRB10, ST6GALNAC3; LCN2, SLC2A3, BMX, LGALS2; IL1R2, SLC2A3, BMX, TCN1; LCN2, SLC2A3, GRB10, ST6GALNAC3; FCAR, PFKFB2, BMX, CEACAM1; IL1R2, HK3, BMX, CD24; IL1R2, PFKFB3, BMX, CD24; BMX, SLC2A3, GRB10, CD24; IL1R2, HK3, BMX, CEACAM1; or GZMA, SLC2A3, BMX, CD24; or LCN2, PFKFB3, GRB10, ST6GALNAC3, RNASE3; LCN2, PFKFB3, GRB10, RNASE2, ST6GALNAC3; IL1R2, PFKFB3, GRB10, CD24, ST6GALNAC3; GZMA, PFKFB3, GRB10, ST6GALNAC3, CD24; HK3, PFKFB3, GRB10, ST6GALNAC3, CD24; IL1R2, PFKFB3, GRB10, ST6GALNAC3, TCN1; SLC2A3, PFKFB3, GRB10, ST6GALNAC3, TCN1; LCN2, PFKFB3, GRB10, ST6GALNAC3, DACH1; SLC2A3, PFKFB3, GRB10, ST6GALNAC3, CD24; or SLC2A3, HK3, BMX, SPOCD1, LGALS2;
wherein the expression of the set of genes is altered compared to a control.

7-9. (canceled)

10. The method of claim 6, wherein expression of one or more of LCN2, SLC2A3, BMX, GRB10, PFKFB3, IL1R2, HK3, FCAR, PFKFB2, IL18R1, ST6GALNAC3, TCN1, CEACAM1, CD24, RNASE3, RNASE2, DACH1 and/or SPOCD1 is increased compared to the control and/or expression of GZMA and/or LGALS2 is decreased compared to the control.

11. The method of claim 1, further comprising determining a Sequential Organ Failure Assessment (SOFA) score for the subject and/or measuring C-reactive protein (CRP) level in a sample from the subject.

12. The method of claim 6,
further comprising administering one or more treatments for sepsis to the subject when expression of one or more of LCN2, SLC2A3, BMX, GRB10, PFKFB3, IL1R2, HK3, FCAR, PFKFB2, IL18R1, ST6GALNAC3, TCN1, CEACAM1, CD24, RNASE3, RNASE2, DACH1 and/or SPOCD1 is increased compared to the control and/or expression of GZMA and/or LGALS2 is decreased compared to the control.

13. The method of claim 1,
further comprising administering one or more treatments for sepsis to the subject when expression of one or more of RPS6KA3, BCL6, TPM3, GNG10, STOM, MPO, BMX, LTF, PDGFC, CD55, CYP1B1, TLR8, MLLT1, YOD1, GAS7, RRBP1, LILRA2, IL17RA, LILRA4, TCN1, RNASE3, RNASE2, FAM105A, ERO1L, and/or C14orf101 is increased compared to the control and/or expression of one or more of MAVS, HLA-DRA, CYP27A1, LGALS2, and/or NOV is decreased compared to the control.

14. The method of claim 12, wherein the treatment for sepsis comprises one or more of antibiotic treatment, vasopressors, intravenous fluids, oxygen, dialysis, and monitoring for sepsis.

15. The method of claim 1, wherein the subject does not exhibit symptoms of sepsis.

16. A method, comprising measuring expression of a set of at least six genes in a sample from a subject having or suspected to have sepsis, wherein the set of at least six genes comprises or consists of:
CCR1, HLA-DPB1, BATF, C3AR1, ARHGEF18, and C9orf95; CCR1, HLA-DPB1, BATF, C3AR1, MTCH1, and C9orf103; CCR1, HLA-DPB1, BATF, C3AR1, MTCH1, and C9orf95; CCR1, HLA-DPB1, BATF, GNA15, MTCH1, and C9orf95; CD177, HLA-DPB1, BATF, C3AR1, MTCH1, and C9orf95; CD63, HLA-DPB1, PLAC8, FCER1G, MTCH1, and C9orf95; CD63, HLA-DPB1, PLAC8, GNA15, ARHGEF18, and C9orf95; CD63, HLA-DPB1, PLAC8, GNA15, MTCH1, and C9orf95; CD63, HLA-DPB1, BATF, C3AR1, MTCH1, and C9orf95; CD63, HLA-DPB1, BATF, GNA15, MTCH1, and C9orf95; EMR1, HLA-DPB1, BATF, C3AR1, MTCH1, and C9orf95; EMR1, HLA-DPB1, BATF, GNA15, MTCH1, and C9orf95; FCER1G, HLA-DPB1, PLAC8, C3AR1, MTCH1, and C9orf95; FCER1G, HLA-DPB1, PLAC8, GNA15, ARHGEF18, and C9orf95; FCER1G, HLA-DPB1, PLAC8, GNA15, MTCH1, and C9orf95; FCER1G, HLA-DPB1, BATF, C3AR1, MTCH1, and SEPHS2; FCER1G, HLA-DPB1, BATF, C3AR1, MTCH1, and C9orf95; FES, HLA-DPB1, PLAC8, FCER1G, MTCH1, and C9orf95; FES, HLA-DPB1, PLAC8, GNA15, ARHGEF18, and C9orf103; FES, HLA-DPB1, PLAC8, GNA15, MTCH1, and C9orf95; FES, HLA-DPB1, BATF, C3AR1, MTCH1, and C9orf95; or C3AR1, HLA-DPB1, BATF, GNA15, MTCH1, and C9orf95;
wherein the expression of CCR1, C3AR1, C9orf95, BATF, C9orf103, GNA15, CD177, CD63, EMR1, FCER1G, FES, PLAC8, and/or SEPHS2 is increased compared to a control and the expression of HLA-DPB1, ARHGEF18, and/or MTCH1 is decreased compared to a control.

17-19. (canceled)

20. The method of claim 16, further comprising measuring expression of RPGRIP1, wherein the expression of RPGRIP1 is decreased compared to a control.

21. The method of claim 16,
further comprising administering one or more treatments for sepsis to the subject when the expression of CCR1, C3AR1, C9orf95, BATF, C9orf103, GNA15, CD177, CD63, EMR1, FCER1G, FES, PLAC8, and/or SEPHS2 is increased compared to a control and the expression of HLA-DPB1, ARHGEF18, and/or MTCH1 is decreased compared to a control.

22. The method of claim 21, wherein the treatment for sepsis comprises one or more of antibiotic treatment, vasopressors, intravenous fluids, oxygen, and dialysis.

23. The method of claim 1, wherein the sample is whole blood, peripheral blood mononuclear cells, serum, or plasma.

24. The method of claim 1, wherein measuring expression comprises measuring mRNA expression, protein expression, or both.

25. The method of claim 1, further comprising measuring expression of at least one housekeeping or internal control molecule.

26. The method of claim 1, wherein the control is a reference value or a sample from a healthy subject.

27. The method of claim 1, wherein measuring expression comprises real-time PCR, quantitative real-time reverse transcriptase PCR, reverse transcriptase PCR, and/or microarray analysis.

28-37. (canceled)

38. A method of identifying a mRNA biomarker panel for diagnosing pre-symptomatic sepsis, comprising:
collecting blood samples from patients prior to a surgery and daily until five days post-sepsis diagnosis;
collecting blood samples from age, gender and procedure matched control surgical subjects who did not develop sepsis;
preparing RNA from the blood samples;
performing whole blood differential gene expression analysis using clinical information on the samples with approaches comprising:
with or without using pre-surgery data;
paired or unpaired analysis between controls and sepsis samples; and
with or without combining time point data prior to Day 0 or not;
selecting mRNAs with differential expression in at least two times points; and
identifying a biomarker panel comprising utilizing support vector machine with recursive feature elimination.

39. The method of claim 16, wherein the treatment for sepsis comprises one or more of antibiotic treatment, vasopressors, intravenous fluids, oxygen, and dialysis.

40. The method of claim 16, wherein the sample is whole blood, peripheral blood mononuclear cells, serum, or plasma.

41. The method of claim 16, wherein measuring expression comprises measuring mRNA expression, protein expression, or both.

42. The method of claim 16, further comprising measuring expression of at least one housekeeping or internal control molecule.

43. The method of claim 16, wherein the control is a reference value or a sample from a healthy subject.

44. The method of claim 16, wherein measuring expression comprises real-time PCR, quantitative real-time reverse transcriptase PCR, reverse transcriptase PCR, and/or microarray analysis.
